(19) United States
(12) Patent Application Publication
(10) Pub. No.: US 2005/0261177 A1
(43) Pub. Date: Nov. 24, 2005

(54) Compound

(76) Inventors: Ayako Toda, Osaka (JP); Hiroaki Mizuno, Osaka (JP); Takahiro Matsuya, Osaka (JP); Hiroshi Matsuda, Osaka (JP); Kenji Murano, Osaka (JP); David Barrett, Osaka (JP); Takashi Ogino, Osaka (JP); Hitomi Ogino, Osaka (JP); Keiji Matsuda, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/469,233

(22) PCT Filed: Mar. 7, 2002
(86) PCT No.: PCT/JP02/02109

§ 371 Date: May 6, 2004

(30) Foreign Application Priority Data
Mar. 8, 2001 (AU) PR 3620

(51) Int. Cl. A61K 38/00

(52) U.S. Cl. 514/12; 530/300

(57) ABSTRACT

This invention relates to new polypeptide compound represented by the following general formula (I): wherein R¹, R², R³, R⁴, R⁵ and R⁶ are as defined in the description or a salt thereof which has antimicrobial activities (especially, antifungal activities), inhibitory activity on β-1,3-glucan synthase, to process for preparation thereof, to a pharmaceutical composition comprising the same, and to a method for prophylactic and/or therapeutic treatment of infectious diseases including Pneumocystis carinii infection (e.g. Pneumocystis carinii pneumonia) in a human being or an animal.

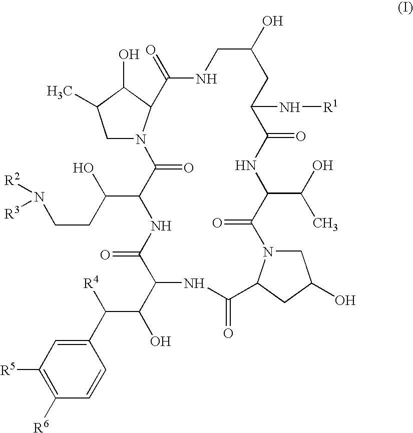

### TECHNICAL FIELD

The present invention relates to new polypeptide compounds and salts thereof which are useful as a medicament.

### BACKGROUND ART

In U.S. Pat. Nos. 5,376,634, 5,569,646, WO 96/11210 and WO 99/40108, there are disclosed the polypeptide compound and a pharmaceutically acceptable salt thereof, which have antimicrobial activities (especially antifungal activity).

### DISCLOSURE OF INVENTION

The present invention relates to new polypeptide compound and a salt thereof.

More particularly, it relates to new polypeptide compound and a salt thereof, which have antimicrobial activities [especially, antifungal activities, in which the fungi may include Aspergillus, Cryptococcus, Candida, Mucor, Actinomyces, Histoplasma, Dermatophyte, Malassezia, Fusarium and the like.], inhibitory activity on β-1,3-glucan synthase, and further which are expected to be useful for the prophylactic and/or therapeutic treatment of Pneumocystis carinii infection (e.g. Pneumocystis carinii pneumonia) in a human being or an animal, to a process for preparation thereof, to a pharmaceutical composition comprising the same, and to a method for the prophylactic and/or therapeutic treatment of infectious disease including Pneumocystis carinii infection (e.g. Pneumocystis carinii pneumonia) in a human being or an animal.

The object polypeptide compounds of the present invention are new and can be represented by the following general formula (I): 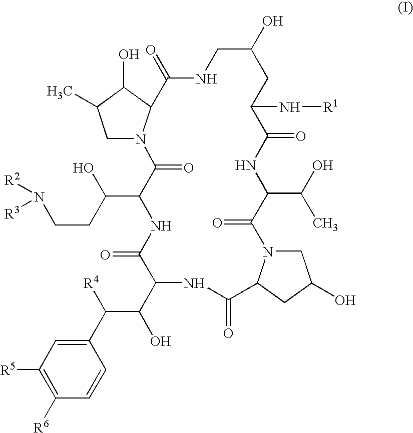
wherein
R¹ is acyl group,
R² is hydrogen or acyl group,
R³ is lower alkyl which has one or more hydroxy or protected hydroxy,
R⁴ is hydrogen or hydroxy,
R⁵ is hydrogen, hydroxy, lower alkoxy or hydroxysulfonyloxy, and
R⁶ is hydroxy or acyloxy,
   or a salt thereof.

The new polypeptide compound (I) or a salt thereof can be prepared by the process as illustrated in the following reaction schemes. 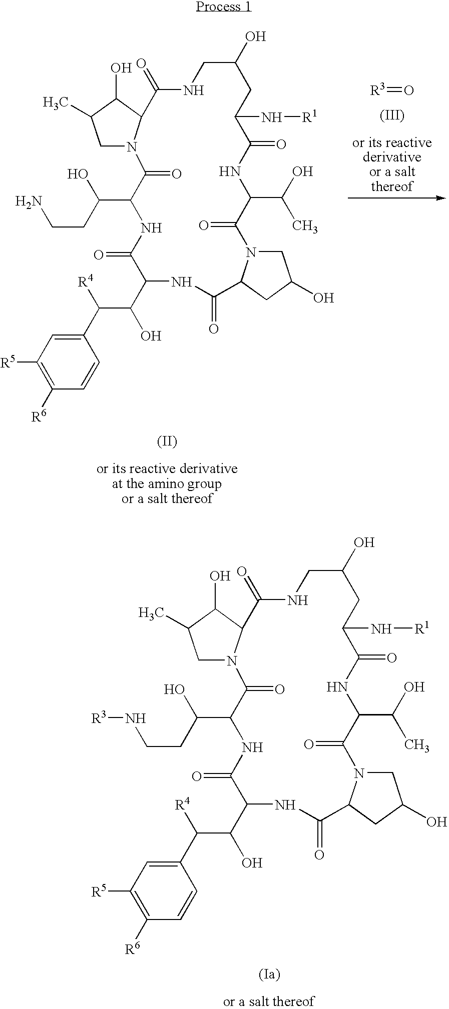 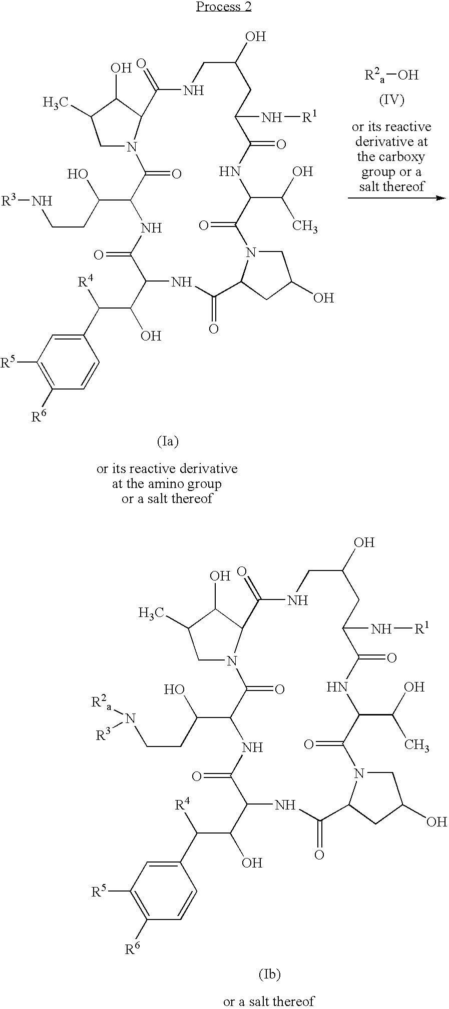 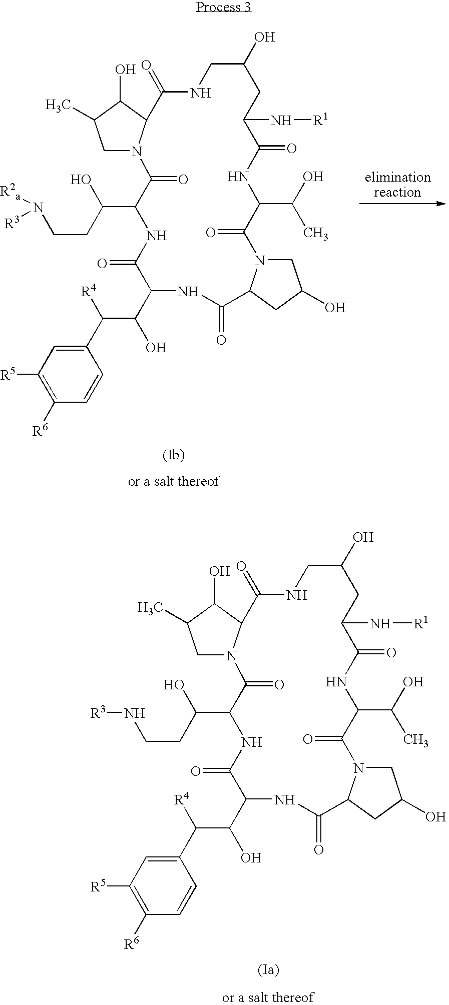 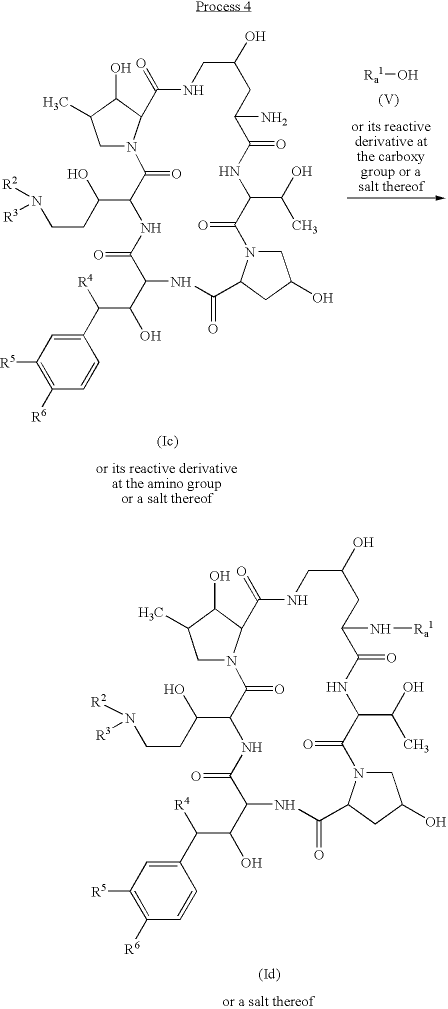
wherein
R¹, R², R³, R⁴, R⁵ and R⁶ are defined above,
Rₐ¹ is acyl group, and
Rₐ² is acyl group.

Suitable salt of the new polypeptide compound (I) is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt;
a salt with an organic base, for example, an organic amine salt (e.g., triethylamine salt, diisopropylethylamine salt, pyridine salt, picoline salt, ethanolamine salt, triethanolamine salt, dicyclohexylamine salt,
N,N′-dibenzylethylenediamine salt, 4-dimethylaminopyridine salt, etc.);
an inorganic acid addition salt (e.g., hydrochloride hydrobromide, sulfate, phosphate, etc.);
an organic carboxylic sulfonic acid addition salt (e.g., formate, acetate, trifluoroacetate, maleate, tartrate, fumarate, methanesulfonate, benzenesulfonate, toluenesulfonate, etc.);
a salt with a basic or acidic amino acid (e.g., arginine, aspartic acid, glutamic acid, etc.).

Suitable examples and illustration of the various definitions in the above and subsequent descriptions of the present specification, which the present invention intends to include within the scope thereof, are explained in detail as follows:

The term “lower” is used to intend a group having 1 to 6 carbon atom(s), unless otherwise provided.

Suitable example of “one or more” may be the number of 1 to 6, in which the preferred one may be the number of 1 to 3, and the most preferred one may be the number of 1 or 2.

Suitable example of “halogen” may be fluorine, chlorine, bromine, iodine and the like.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy and the like.

Suitable example of “higher alkoxy” may include straight or branched one such as heptyloxy, octyloxy, 3,5-dimethyloctyloxy, 3,7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like.

Suitable example of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neo-pentyl, hexyl, isohexyl and the like.

Suitable example of “higher alkyl” may include straight or branched one such as heptyl, octyl, 3,5-dimethyloctyl, 3,7-dimethyloctyl, nonyl, decyl, undecyl, dodecyl, tridecyl, tetradecyl, hexadecyl, heptadecyl, octadecyl, nonadecyl, icosyl, and the like.

Suitable example of “aryl” and “ar” moiety may include phenyl which may have lower alkyl (e.g., phenyl, mesityl, xylyl, tolyl, etc.), naphthyl, anthryl, indanyl, fluorenyl, and the like, and this “aryl” and “ar” moiety may have one or more halogen.

Suitable example of “aroyl” may include benzoyl, toluoyl, naphthoyl, anthrylcarbonyl, and the like.

Suitable example of “heterocyclic group” may include
unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g.; 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.;
saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;
unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;
unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;
saturated 3 to 8-membered (more preferably 5 or 6-membered) heteromonocyclic group containing 1 or 2 oxygen atom(s) and 1 to 3 nitrogen atom(s), for example, morpholinyl, sydnonyl, morpholino, etc.;
unsaturated condensed heterocyclic group containing 1 or 2 oxygen atom(s) and 1 to 3 nitrogen atom(s), for example, benzoxazolyl, benzoxadiazolyl, etc.;
unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;
saturated 3 to 8-membered (more preferably 5 or 6-membered) heteromonocyclic group containing 1 or 2 sulfur atom(s) and 1 to 3 nitrogen atom(s), for example thiazolidinyl, thiomorpholinyl, thiomorpholino, etc.;
unsaturated 3 to 8-membered (more preferably 5 or 6-membered) heteromonocyclic group containing 1 or 2 sulfur atom(s), for example, thienyl, dihydrodithiinyl, dihydrodithionyl, etc.;
unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;
unsaturated 3 to 8-membered (more preferably 5 or 6-membered) heteromonocyclic group containing an oxygen atom, for example, furyl etc.;
saturated 3 to 8-membered (more preferably 5 or 6-membered) heteromonocyclic group containing 1 or 2 oxygen atom(s), for example, tetrahydrofuran, tetrahydropyran, dioxacyclopentane, dioxacyclohexane, etc.;
unsaturated 3 to 8-membered (more preferably 5 or 6-membered) heteromonocyclic group containing an oxygen atom and 1 or 2 sulfur atom(s), for example, dihydrooxathiinyl, etc.;
unsaturated condensed heterocyclic group containing 1 or 2 sulfur atom(s), for example benzothienyl, benzodithiinyl, etc.;
unsaturated condensed heterocyclic group containing an oxygen atom and 1 or 2 sulfur atom(s), for example, benzoxathiinyl, etc.; and the like, and this “heterocyclic group” may have one or more suitable substituent(s) selected from the group consisting of lower alkyl, oxo, cyclo(lower)alkyl, hydroxy(lower)alkyl, carboxy(lower)alkanoyl which may have amino and heterocycliccarbonyl.

Suitable example of “cyclo(lower)alkyl” may include cyclopropyl, cyclobutyl, cyclopentyl, cyclohexyl, and the like, and this “cyclo(lower)alkyl” may have one or more lower alkyl.

Suitable example of “cyclo(lower)alkyloxy” may include cyclopropyloxy, cyclobutyloxy, cyclopentyloxy, cyclohexyloxy, and the like.

Suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.

Carboxy; carbamoyl; mono or di(lower)alkylcarbamoyl (e.g., methylcarbamoyl, dimethylcarbamoyl, ethylcarbamoyl, diethylcarbamoyl, etc.)

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);
lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.);
lower or higher alkylsulfonyl (e.g., methylsulfonyl, ethylsulfonyl, etc.);
lower or higher alkoxysulfonyl (e.g., methoxysulfonyl, ethoxysulfonyl, etc.); or the like;
Aromatic acyl such as aroyl (e.g., benzoyl, toluoyl, naphthoyl, etc.); ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];
ar(lower)alkenoyl [e.g., phenyl(C₃-C₆)alkenoyl (e.g., phenylpropenoyl, phenylbutenoyl, phenylacryloyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.] substituted with one or more suitable substituent(s);
ar(lower)alkoxycarbonyl [e.g., phenyl(C₁-C₆)alkoxycarbonyl (e.g., benzyloxycarbonyl, etc.), fluorenyl(C₁-C₆)alkoxy-carbonyl (e.g., fluorenylmethyloxycarbonyl, etc.), etc.];
aryloxycarbonyl (e.g., phenoxycarbonyl, naphthyloxycarbonyl, etc.);
aryloxy(lower)alkanoyl (e.g., phenoxyacetyl, phenoxypropionyl, etc.);
arylcarbamoyl (e.g., phenylcarbamoyl, etc.);
arylthiocarbamoyl (e.g., phenylthiocarbamoyl, etc.);
arylglyoxyloyl (e.g., phenylglyoxyloyl, naphthylglyoxyloyl, etc.);
arylsulfonyl which may have 1 to 4 lower alkyl (e.g., phenylsulfonyl, p-tolylsulfonyl, etc.);
aroyl (e.g., benzoyl, naphthoyl, etc.) substituted with one or more suitable substituent(s); or the like;
Heterocyclic acyl such as heterocycliccarbonyl;
heterocyclic(lower)alkanoyl (e.g., heterocyclicacetyl, heterocyclicpropanoyl, heterocyclicbutanoyl, heterocyclicpentanoyl, heterocyclichexanoyl, etc.);
heterocyclic(lower)alkenoyl (e.g., heterocyclicpropenoyl, heterocyclicbutenoyl, heterocyclicpentenoyl, heterocyclichexenoyl, etc.);
heterocyclicglyoxyloyl; or the like;
in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “acyl group” of R¹ can be referred to aforementioned “acyl group”, in which the preferred one may be lower alkoxy carbonyl, higher alkanoyl, phenyl(lower)alkenoyl substituted with one or more suitable substituent(s), benzoyl substituted with one or more suitable substituent(s) and naphthoyl substituted with one or more suitable substituent(s).

Suitable example of “suitable substituent(s)” in the term of “phenyl(lower)alkenoyl substituted with one or more suitable substituent(s)”, “benzoyl substituted with one or more suitable substituent(s)” or “naphthoyl substituted with one or more suitable substituent(s)” may be higher alkoxy,
lower alkoxy(higher)alkoxy,
higher alkyl,
phenyl substituted with a suitable substituent selected from the group consisting of lower alkoxy, higher alkoxy and higher alkyl,
thiadiazolyl substituted with phenyl which has a suitable substituent selected from the group consisting of piperazinyl substituted with cyclo(lower)alkyl which may have lower alkoxy(lower)alkoxy, piperazinyl substituted with lower alkoxy(higher)alkyl, piperazinyl substituted with tetrahydropyran, piperazinyl substituted with dioxaspiro(higher)alkyl which may have lower alkyl, piperazinyl substituted with lower alkyl having pyridyl, piperidyl substituted with lower alkoxy and chlorophenyl, piperidyl substituted with lower alkoxy, piperidyl substituted with lower alkoxy having cyclo(lower)alkyl, piperidyl substituted with lower alkoxy(higher)alkoxy, dioxaazaspiro(higher)alkyl, tetrahydropyrazolopyridyl substituted with phenyl, cyclo(lower)alkyloxy, piperidyloxy substituted with cyclo(lower)alkyl which may have lower alkoxy(lower)alkoxy, piperidyloxy substituted with lower alkoxy(higher)alkyl, piperidyloxy substituted with phenyl which may have lower alkoxy, piperidyl substituted with lower alkoxy higher alkyl, and piperidyl substituted with lower alkoxy(lower)alkoxy,
thiadiazolyl substituted with pyridyl having piperidyl substituted with phenyl,
imidazothiadiazolyl substituted with phenyl having lower alkoxy(lower)alkoxy(lower)alkyl,
imidazothiadiazolyl substituted with phenyl having lower alkoxy and cyclo(lower)alkyl,
imidazothiadiazolyl substituted with phenyl having piperidyloxy substituted with phenyl which may have lower alkoxy,
imidazothiadiazolyl substituted with phenyl having piperidyloxy substituted with cyclo(lower)alkyl which may have lower alkoxy(lower)alkoxy,
imidazothiadiazolyl substituted with phenyl having tetrahydropyridyl substituted with cyclo(lower)alkyl,
imidazothiadiazolyl substituted with phenyl having piperidyl substituted with lower alkoxy(lower)alkyl,
imidazothiadiazolyl substituted with phenyl having piperazinyl substituted with lower alkoxy(lower)alkyl,
imidazothiadiazolyl substituted with phenyl having lower alkoxy(higher)alkyl,
imidazothiazolyl substituted with phenyl having lower alkoxy(lower)alkoxy,
phenyl substituted with piperazinyl having phenyl substituted with lower alkoxy,
phenyl substituted with piperazinyl having phenyl substituted with piperidyloxy having lower alkoxy(lower)alkyl,
phenyl substituted with diazabicyclo(higher)alkyl having cyclo(lower)alkyl,
phenyl substituted with hexahydrodiazepinyl having cyclo(lower)alkyl,
phenyl substituted with piperidyl having phenyl,
phenyl substituted with piperazinyl having phenyl substituted with piperazinyl having lower alkoxy(lower)alkyl,
piperazinyl substituted with thiadiazolyl having phenyl substituted with lower alkoxy(higher)alkoxy,
thiazolyl substituted with phenyl having lower alkoxy,
oxadiazolyl substituted with phenyl having higher alkoxy,
oxadiazolyl substituted with phenyl having phenyl substituted with lower alkoxy,
oxadiazolyl substituted with phenyl having piperazinyl substituted with cyclo(lower)alkyl having lower alkyl,
pyrazolyl substituted with phenyl having phenyl, or
pyrazolyl substituted with phenyl having lower alkoxy,
in which the preferred one may be heptyloxy, methoxyoctyloxy,
heptyl,
phenyl substituted with a substituent selected from the group consisting of butoxy, pentyloxy, nonyloxy and heptyl,
thiadiazolyl substituted with phenyl which has a substituent selected from the group consisting of piperazinyl substituted with cyclohexyl having methyl, piperazinyl substituted with cyclopentyl, piperazinyl substituted with cycloheptyl, piperazinyl substituted with cyclohexyl having methoxyhexyloxy, piperazinyl substituted with methoxyheptyl, piperazinyl substituted with tetrahydropyran, piperazinyl substituted with dioxaspirodecan which may have dimethyl, piperazinyl substituted with methyl having pyridyl, piperidyl substituted with methoxy and chlorophenyl, piperidyl substituted with 4-methylpentyloxy, piperidyl substituted with butoxy, piperidyl substituted with pentyloxy, piperidyl substituted with methoxy having cyclohexyl, piperidyl substituted with methoxyheptyloxy, dioxaazospirodecan, tetrahydropyrazolopyridyl substituted with phenyl, cyclohexyloxy, piperidyloxy substituted with cyclohexyl which may have methoxyhexyloxy, piperidyloxy substituted with methoxyoctyl, piperidyloxy substituted with phenyl which may have methoxy, piperidyl substituted with methoxyheptyl, and piperidyl substituted with methoxyhexyloxy,
thiadiazolyl substituted with pyridyl having piperidyl substituted with phenyl,
imidazothiadiazolyl substituted with phenyl having methoxypentyloxymethyl,
imidazothiadiazolyl substituted with phenyl having methoxy and cyclohexyl,
imidazothiadiazolyl substituted with phenyl having piperidyloxy substituted with phenyl which may have methoxy,
imidazothiadiazolyl substituted with phenyl having piperidyloxy substituted with cyclohexyl which may have methoxyhexyloxy,
imidazothiadiazolyl substituted with phenyl having tetrahydropyridyl substituted with cyclohexyl,
imidazothiadiazolyl substituted with phenyl having piperidyl substituted with methoxyhexyl,
imidazothiadiazolyl substituted with phenyl having piperazinyl substituted with methoxypentyl,
imidazothiadiazolyl substituted with phenyl having piperazinyl substituted with methoxyhexyl,
imidazothiadiazolyl substituted with phenyl having methoxyheptyl,
imidazothiazolyl substituted with phenyl having methoxypentyloxy,
phenyl substituted with piperazinyl having phenyl substituted with methoxy,
phenyl substituted with piperazinyl having phenyl substituted with piperidyloxy having methoxyhexyl,
phenyl substituted with diazabicycloheptyl having cyclohexyl,
phenyl substituted with hexahydrodiazepinyl having cyclohexyl,
phenyl substituted with piperidyl having phenyl,
phenyl substituted with piperazinyl having phenyl substituted with piperazinyl having methoxyhexyl,
piperazinyl substituted with thiadiazolyl having phenyl substituted with methoxyheptyloxy,
thiazolyl substituted with phenyl having pentyloxy,
oxadiazolyl substituted with phenyl having octyloxy,
oxadiazolyl substituted with phenyl having phenyl substituted with propoxy,
oxadiazolyl substituted with phenyl having piperazinyl substituted with cyclohexyl having methyl,
pyrazolyl substituted with phenyl having phenyl, or
pyrazolyl substituted with phenyl having hexyloxy.

The more suitable example of “acyl group” may be
naphthoyl substituted with heptyloxy,
naphthoyl substituted with methoxyoctyloxy,
naphthoyl substituted with heptyl,
phenylacryloyl substituted with phenyl substituted with a substituent selected from the group consisting of butoxy and pentyloxy,
benzoyl substituted with phenyl substituted with a substituent selected from the group consisting of nonyloxy and heptyl,
benzoyl substituted with thiadiazolyl substituted with phenyl which has a substituent selected from the group consisting of piperazinyl substituted with cyclohexyl having methyl, piperazinyl substituted with cyclopentyl, piperazinyl substituted with cycloheptyl, piperazinyl substituted with cyclohexyl having methoxyhexyloxy, piperazinyl substituted with methoxyheptyl, piperazinyl substituted with tetrahydropyran, piperazinyl substituted with dioxaspirodecan which may have dimethyl, piperazinyl substituted with methyl having pyridyl, piperidyl substituted with methoxy and chlorophenyl, piperidyl substituted with 4-methylpentyloxy, piperidyl substituted with butoxy, piperidyl substituted with pentyloxy, piperidyl substituted with methoxy having cyclohexyl, piperidyl substituted with methoxyheptyloxy, dioxaazospirodecan, tetrahydropyrazolopyridyl substituted with phenyl, cyclohexyloxy, piperidyloxy substituted with cyclohexyl which may have methoxyhexyloxy, piperidyloxy substituted with methoxyoctyl, piperidyloxy substituted with phenyl which may have methoxy, piperidyl substituted with methoxyheptyl, and piperidyl substituted with methoxyhexyloxy,
benzoyl substituted with thiadiazolyl substituted with pyridyl having piperidyl substituted with phenyl,
benzoyl substituted with imidazothiadiazolyl substituted with phenyl having methoxypentyloxymethyl,
benzoyl substituted with imidazothiadiazolyl substituted with phenyl having methoxy and cyclohexyl,
benzoyl substituted with imidazothiadiazolyl substituted with phenyl having piperidyloxy substituted with phenyl which may have methoxy,
benzoyl substituted with imidazothiadiazolyl substituted with phenyl having piperidyloxy substituted with cyclohexyl which may have methoxyhexyloxy,
benzoyl substituted with has imidazothiadiazolyl substituted with phenyl having tetrahydropyridyl substituted with cyclohexyl,
benzoyl substituted with imidazothiadiazolyl substituted with phenyl having piperidyl substituted with methoxyhexyl,
benzoyl substituted with imidazothiadiazolyl substituted with phenyl having piperazinyl substituted with methoxypentyl,
benzoyl substituted with imidazothiadiazolyl substituted with phenyl having piperazinyl substituted with methoxyhexyl,
benzoyl substituted with imidazothiadiazolyl substituted with phenyl having methoxyheptyl,
benzoyl substituted with imidazothiazolyl substituted with phenyl having methoxypentyloxy,
benzoyl substituted with phenyl substituted with piperazinyl having phenyl substituted with methoxy,
benzoyl substituted with phenyl substituted with piperazinyl having phenyl substituted with piperidyloxy having methoxyhexyl,
benzoyl substituted with phenyl substituted with diazabicycloheptyl having cyclohexyl,
benzoyl substituted with phenyl substituted with hexahydrodiazepinyl having cyclohexyl,
benzoyl substituted with phenyl substituted with piperidyl having phenyl,
benzoyl substituted with phenyl substituted with piperazinyl having phenyl substituted with piperazinyl having methoxyhexyl,
benzoyl substituted with piperazinyl substituted with thiadiazolyl having phenyl substituted with methoxyheptyloxy,
benzoyl substituted with thiazolyl substituted with phenyl having pentyloxy,
benzoyl substituted with oxadiazolyl substituted with phenyl having octyloxy,
benzoyl substituted with oxadiazolyl substituted with phenyl having phenyl substituted with propoxy,
benzoyl substituted with oxadiazolyl substituted with phenyl having piperazinyl substituted with cyclohexyl having methyl,
benzoyl substituted with pyrazolyl substituted with phenyl having phenyl, or
benzoyl substituted with pyrazolyl substituted with phenyl having hexyloxy.

Suitable example of “lower alkyl” in the term of “lower alkyl which has one or more hydroxy or protected hydroxy” can be referred to aforementioned “lower alkyl”, in which the preferred one may be methyl, ethyl, propyl, isopropyl, butyl, pentyl and hexyl.

Suitable example of “hydroxy protective group” in the term of “protected hydroxy” may include acyl (e.g., lower alkanoyl, etc.) as mentioned above, phenyl(lower)alkyl which may have one or more suitable substituent(s) (e.g., benzyl, 4-methoxybenzyl, trityl, etc.), tri-substituted silyl [e.g., tri(lower)alkylsilyl(e.g., trimethylsilyl, t-butyldimethylsilyl, etc.), etc.], tetrahydropyranyl and the like.

Suitable example of “lower alkyl which has one or more hydroxy or protected hydroxy” may be dihydroxypropyl, dihydroxyisopropyl, trihydroxybutyl, tetrahydroxypentyl, pentahydroxyhexyl and diacetyloxyisopropyl.

Suitable example of “acyl group” of R² can be referred to aforementioned “acyl group”, in which the preferred one may be “amino protective group” mentioned below, and the most preferred one may be acetyl, 2-acetyloxypropionyl, methylsulfonyl, 2,5-diaminopentanoyl, benzyloxycarbonyl, fluorenylmethoxycarbonyl, allyloxycarbonyl, tert-butoxycarbonyl and (5-methyl-2-oxo-1,3-dioxol-4-yl)methoxycarbonyl.

Suitable example of “amino protective group” may be included in aforementioned “acyl group”, a conventional protective group such as ar(lower)alkoxycarbonyl and lower alkoxycarbonyl, in which the preferred one may be phenyl-(C₁-C₄)alkoxycarbonyl and fluorenyl(C₁-C₄) alkoxycarbonyl and (C₁-C₄)alkoxycarbonyl, and the most preferred one may be benzyloxycarbonyl, fluorenylmethoxycarbonyl and tert-butoxycarbonyl.

Suitable example of “acyl” moiety of “acyloxy” can be referred to aforementioned “acyl group”, in which the preferred one may be lower alkenyloxycarbonyl, and the most preferred one may be allyloxycarbonyl.

Suitable example of “acyloxy” may be lower alkenyloxycarbonyloxy, and the more preferred one may be allyloxycarbonyloxy.

Particularly, the preferred examples of the cyclic polypeptide compound (I) of the present invention are as follows:
the compound (I), wherein
R¹ is phenyl(lower)alkenoyl substituted with one or more suitable substituent(s), benzoyl substituted with one or more suitable substituent(s) or naphthoyl substituted with one or more suitable substituent (s),
R² is hydrogen,
R³ is lower alkyl which has one or more hydroxy,
R⁴ is hydrogen or hydroxy;
R⁵ is hydroxy or hydroxysulfonyloxy; and
R⁶ is hydroxy.

And, more preferred one may be the compound (I)
wherein
R¹ is naphthoyl substituted with higher alkoxy, naphthoyl substituted with lower alkoxy(higher)alkoxy,
   naphthoyl substituted with higher alkyl,
   phenyl(lower)alkenoyl substituted with phenyl substituted with lower alkoxy,
   benzoyl substituted with a suitable substituent selected from the group consisting of phenyl substituted with a suitable substituent selected from the group consisting of lower alkoxy, higher alkoxy and higher alkyl,
   thiadiazolyl substituted with phenyl which has a suitable substituent selected from the group consisting of piperazinyl substituted with cyclo(lower)alkyl which may have lower alkoxy(lower)alkoxy, piperazinyl substituted with lower alkoxy(higher)alkyl, piperazinyl substituted with tetrahydropyran, piperazinyl substituted with dioxaspiro(higher)alkyl which may have lower alkyl, piperazinyl substituted with lower alkyl having pyridyl, piperidyl substituted with lower alkoxy and chlorophenyl, piperidyl substituted with lower alkoxy, piperidyl substituted with lower alkoxy having cyclo(lower)alkyl, piperidyl substituted with lower alkoxy(higher)alkoxy, dioxaazaspiro(higher)alkyl, tetrahydropyrazolopyridyl substituted with phenyl, cyclo(lower)alkyloxy, piperidyloxy substituted with cyclo(lower)alkyl which may have lower alkoxy(lower)alkoxy, piperidyloxy substituted with lower alkoxy(higher)alkyl, piperidyloxy substituted with phenyl which may have lower alkoxy, piperidyl substituted with lower alkoxy higher alkyl, and piperidyl substituted with lower alkoxy(lower)alkoxy,
   thiadiazolyl substituted with pyridyl having piperidyl substituted with phenyl,
   imidazothiadiazolyl substituted with phenyl having lower alkoxy(lower)alkoxy(lower)alkyl,
   imidazothiadiazolyl substituted with phenyl having lower alkoxy and cyclo(lower)alkyl,
   imidazothiadiazolyl substituted with phenyl having piperidyloxy substituted with phenyl which may have lower alkoxy,
   imidazothiadiazolyl substituted with phenyl having piperidyloxy substituted with cyclo(lower)alkyl which may have lower alkoxy(lower)alkoxy,
   imidazothiadiazolyl substituted with phenyl having tetrahydropyridyl substituted with cyclo(lower)alkyl,
   imidazothiadiazolyl substituted with phenyl having piperidyl substituted with lower alkoxy(lower)alkyl,
   imidazothiadiazolyl substituted with phenyl having piperazinyl substituted with lower alkoxy(lower)alkyl, imidazothiadiazolyl substituted with phenyl having lower alkoxy(higher)alkyl,
   imidazothiazolyl substituted with phenyl having lower alkoxy(lower)alkoxy,
   phenyl substituted with piperazinyl having phenyl substituted with lower alkoxy,
   phenyl substituted with piperazinyl having phenyl substituted with piperidyloxy having lower alkoxy(lower)alkyl,
   phenyl substituted with diazabicyclo(higher)alkyl having cyclo(lower)alkyl,
   phenyl substituted with hexahydrodiazepinyl having cyclo(lower)alkyl,
   phenyl substituted with piperidyl having phenyl,
   phenyl substituted with piperazinyl having phenyl substituted with piperazinyl having lower alkoxy(lower)alkyl,
   piperazinyl substituted with thiadiazolyl having phenyl substituted with lower alkoxy(higher)alkoxy,
   thiazolyl substituted with phenyl having lower alkoxy,
   oxadiazolyl substituted with phenyl having higher alkoxy,
   oxadiazolyl substituted with phenyl having phenyl substituted with lower alkoxy,
   oxadiazolyl substituted with phenyl having piperazinyl substituted with cyclo(lower)alkyl having lower alkyl,
   pyrazolyl substituted with phenyl having phenyl, and
   pyrazolyl substituted with phenyl having lower alkoxy,
R² is hydrogen,
R³ is lower alkyl which has two hydroxy,
R⁴ is hydrogen or hydroxy;
R⁵ is hydroxy or hydroxysulfonyloxy; and
R⁶ is hydroxy.

The processes for preparing the polypeptide compound (I) of the present invention are explained in detail in the following.

Process 1

The object compound (Ia) or a salt thereof can be prepared by reacting the compound (II) or its reactive derivative at the amino group or a salt thereof with the compound (III) of the formula:
R³═O (III)
or its reactive derivative, or a salt thereof.

Suitable reactive derivative of the compound (III) may include an acid halide, an acid anhydride, an activated ester, and the like. The suitable example may be an acid chloride; acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anydride; an activated amide with imidazole, 4-substitutd imidazole, dimethylpyrazole, triazole or tetrazole; an activated ester (e.g., cyanomethyl, ester methoxymethyl ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenylthioester, p-nitrophenyl thioester, p-cresyl thioester, carboxymethyl thioester, pyranyl ester, pyridyl ester, piperidyl ester, 8-quinolyl thioester, etc.); an ester with a N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.); and the like. These reactive derivatives can optionally be selected from them according to the kind of the compound (III) to be used.

The reaction is usually carried out in a conventional solvent such as water, acetone, dioxane, acetonitrile, chloroform, methylene chloride, ethylene chloride, tetrahydrofuran, ethyl acetate, N,N-dimethylformamide, pyridine or any other organic solvent which do not adversely affect the reaction, or the mixture thereof.

When the compound (III) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide); N-cyclohexyl-N′-(4-diethylaminocyclohexyl)carbodiimide; N,N′-diisopropylcarboxiimide; N-ethyl-N′-(3-dimethylaminopropyl)carbodiimide; N,N-carbonyl-bis(2-methylimidazole); pentamethyleneketene-N-cyclohexylimine; diphenylketene-N-cyclohexylimine, ethoxyacetylene; 1-alkoxy-1-chloroethylene; trialkyl phosphite; isopropyl polyphosphate; phosphorous oxychloride (phosphoryl chloride); phosphorous trichloride; thionyl chloride; oxalyl chloride; triphenylphosphite; 2-ethyl-7-hydroxybenzisoxazolium salt; 2-ethyl-S-(m-sulfophenyl) isoxazolium hydroxide intra-molecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorous oxychloride, etc.; or the like.

The reaction may also be carried out in the presence of an organic or inorganic base such as an alkali metal bicarbonate, tri(lower)alkylamine (e.g., triethylamine, diisopropylethylamine, etc.), pyridine, di(lower)alkylaminopyridine (e.g., 4-dimethylaminopyridine, etc.) N-(lower)alkylmorphorine, N,N-di(lower)alkylbenzylamine, or the like.

The reaction temperature is not critical, and the reaction is usually carried out under cooling to heating.

Process 2

The object compound (Ib) or a salt thereof can be prepared by reacting the compound (Ia) or its reactive derivative at the amino group or a salt thereof with the compound (IV) of the formula:
Rₐ²—OH (IV)
(wherein Rₐ² is acyl group) or its reactive derivative at the carboxy group or a salt thereof.

Suitable reactive derivative of the compound (IV) may include an acid halide, an acid anhydride, an activated ester, and the like. The suitable example may be an acid chloride; acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anydride; an activated amide with imidazole, 4-substitutd imidazole, dimethylpyrazole, triazole or tetrazole; an activated ester (e.g., cyanomethyl, ester methoxymethyl ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenylthioester, p-nitrophenyl thioester, p-cresyl thioester, carboxymethyl thioester, pyranyl ester, pyridyl ester, piperidyl ester, 8-quinolyl thioester, etc.); an ester with a N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.); and the like. These reactive derivatives can optionally be selected from them according to the kind of the compound (IV) to be used.

The reaction is usually carried out in a conventional solvent such as water, acetone, dioxane, acetonitrile, chloroform, methylene chloride, ethylene chloride, tetrahydrofuran, ethyl acetate, N,N-dimethylformamide, pyridine or any other organic solvent which do not adversely affect the reaction, or the mixture thereof.

When the compound (IV) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide); N-cyclohexyl-N′-(4-diethylaminocyclohexyl)carbodiimide; N,N′-diisopropylcarboxiimide; N-ethyl-N′-(3-dimethylaminopropyl)carbodiimide; N,N-carbonyl-bis(2-methylimidazole); pentamethyleneketene-N-cyclohexylimine; diphenylketene-N-cyclohexylimine, ethoxyacetylene; 1-alkoxy-1-chloroethylene; trialkyl phosphite; isopropyl polyphosphate; phosphorous oxychloride (phosphoryl chloride); phosphorous trichloride; thionyl chloride; oxalyl chloride; triphenylphosphite; 2-ethyl-7-hydroxybenzisoxazolium salt; 2-ethyl-5-(m-sulfophenyl) isoxazolium hydroxide intra-molecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorous oxychloride, etc.; or the like.

The reaction may also be carried out in the presence of an organic or inorganic base such as an alkali metal bicarbonate, tri(lower)alkylamine (e.g., triethylamine, diisopropylethylamine, etc.), pyridine, di(lower)alkylaminopyridine (e.g., 4-dimethylaminopyridine, etc.) N-(lower)alkylmorphorine, N,N-di(lower)alkylbenzylamine, or the like.

The reaction temperature is not critical, and the reaction is usually carried out under cooling to heating.

Process 3

The object compound (Ia) or a salt thereof can be prepared by subjecting a compound (Ib) or a salt thereof to elimination reaction of the acyl group.

This reaction is carried out in accordance with a conventional method such as hydrolysis, reduction or the like.

The hydrolysis is preferably carried out in the presence of a base or an acid including Lewis acid. Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.).

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 4

The object compound (Id) or a salt thereof can be prepared by reacting the compound (Ic) or its reactive derivative at the amino group or a salt thereof with the compound (V) of the formula:
Rₐ¹—OH (V)
(wherein Rₐ¹ is acyl group) or its reactive derivative at the carboxy group or a salt thereof.

Suitable reactive derivative at the carboxy group of the compound (V) may include an acid halide, an acid anhydride, an activated amide, an activated ester, and the like. Suitable examples of the reactive derivatives may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.], dialkylphosphorous acid, sulfurous acid, thiosulfuric acid, sulfuric acid, sulfonic acid [e.g., methanesulfonic acid, etc.], aliphatic carboxylic acid [e.g., acetic acid, propionic acid, butyric acid, isobutyric acid, pivaric acid, pentanoic acid, isopentanoic acid, 2-ethylbutyric acid trichloroacetic acid, etc.]; or aromatic carboxylic acid [e.g., benzoic acid, etc.]; a symmetrical acid, anhydride; an activated amide with imidazole, 4-substituted imidazole, dimethylpyrazole, triazole, tetrazole or 1-hydroxy-1H-benzotriazole; or an activated ester [e.g., cyanomethyl ester, methoxymethyl ester, vinyl ester, propargyl ester, p-nitrophenyl ester, 2,4-dinitrophenyl ester, trichlorophenyl ester, pentachloropentyl ester, mesylphenyl ester, phenylazophenyl ester, phenyl thioester, p-nitrophenyl thioester, p-cresyl thioester, carboxymethyl thioester, pyranyl ester, pyridyl ester, piperidyl ester, 8-quinolyl thioester, etc.], or an ester with a N-hydroxy compound [e.g. N,N-dimethylhydroxylamine, 1-hydroxy-2-(1H)-pyridone, N-hydroxysuccinimide, N-hydroxyphthalimide, 1-hydroxy-1H-benzotriazole, etc.], and the like. These reactive derivatives can optionally be selected from them according to the kind of the compound (V) to be used.

Suitable salts of the compound (V) and its reactive derivative can be referred to the ones as exemplified for the polypeptide compound (I).

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvent may also be used in a mixture with water.

In this reaction, when the compound (V) is used in a free acid form or its salt form, the reaction is preferably carried out in the presence of a conventional condensing agent such as N,N-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide; N,N′-diisopropylcarbodiimide; N-ethyl-N′-(3-dimethylaminopropyl)carbodiimide; N,N-carbonylbis-(2-methylimidazole); pentamethyleneketene-N-cyclohexylimine; diphenylketene-N-cyclohexylimine, ethoxyacetylene; 1-alkoxy-2-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.]; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorous oxychloride, methanesulfonyl chloride, etc.; or the like.

The reaction may also be carried out in the presence of an inorganic or organic base such as an alkali metal carbonate, alkali metal bicarbonate, tri(lower)alkylamine (e.g., triethylamine, diisopropylethylamine, etc.), pyridine, di(lower)alkylaminopyridine (e.g., 4-dimethylaminopyridine, etc.), N-(lower)alkylmorpholine, N,N-di(lower)alkylbenzylamine, or the like.

The reaction temperature is not critical, and the reaction is usually carried out under cooling to warming.

The compounds obtained by the above Processes 1 to 4 can be isolated and purified by a conventional method such as pulverization, recrystallization, column-chromatography, high-performance liquid chromatography (HPLC), reprecipitation, desalting resin column chromatography, or the like.

The compounds obtained by the above Processes 1 to 4 may be obtained as its solvate (e.g., hydrate, ethanolate, etc.), and its solvate (e.g., hydrate, ethanolate, etc.) is included within the scope of the present invention.

It is to be noted that each of the polypeptide compound (I) may include one or more stereoisomer such as optical isomer(s) and geometrical isomer(s) due to asymmetric carbon atom(s) and double bond(s) and all such isomers and the mixture thereof are included within the scope of the present invention.

The polypeptide compound (I) or a salt thereof may include solvated compound [e.g., hydrate, ethanolate, etc.].

The polypeptide compound (I) or a salt thereof may include both its crystal form and non-crystal form.

It should be understood that the polypeptide compound (I) of the present invention may include the prodrug form.

The patent applications and publications cited herein are incorporated by reference.

In order to show the usefulness of the polypeptide compound (I) of the present invention, the biological data of the representative compound is explained in the following.

### Biological Property of the Polypeptide Compound (I) of the Present Invention

Test (Antimicrobial Activity):

In vitro antimicrobial activity of the object compound of Examples 41, 46, 53 and 56 disclosed later was determined by MICₛ in mouse serum as described below.

Test Method:

The MIC_{S} in mouse serum were determined by the microdilution method using ICR mouse serum buffered with 20 mM HEPES buffer (pH 7.3) as a test medium. Inoculum suspension of 10⁶ cells/ml were prepared by a hemocytometric procedure and diluted to obtain an inoculum size of approximately 1.0×10³ cells/ml. Microplates were incubated at 37° C. for 24 hours in 5% CO₂. The MIC_{S} were defined as the lowest concentrations at which no visible growth was observed.

Test Result:

MIC (μg/ml)

| | Test organism |
|---|---|
| Test compound | Candida albicans FP-633 |
| The object compound of | <0.3 |
| Example 41 | |
| The object compound of | <0.3 |
| Example 46 | |
| The object compound of | <0.3 |
| Example 53 | |
| The object compound of | <0.3 |
| Example 56 | |

From the test result, it is realized that the polypeptide compound (I) of the present invention has an antimicrobial activity (especially, antifungal activity).

In more details, the polypeptide compound (I) of the present invention have an antifungal activity, particularly against the following fungi.
Acremonium;
Absidia (e.g., Absidia corymbifera, etc);
Aspergillus (e.g., Aspergillus clavatus, Aspergillus flavus, Aspergillus fumigatus, Aspergillus nidulans, Aspergillus niger, Aspergillus terreus, Aspergillus versicolor, etc); Blastomyces (e.g., Blastomyces dermatitidis, etc);
Candida (e.g., Candida albicans, Candida glabrata, Candida guilliermondii, Candida kefyr, Candida krusei, Candida parapsilosis, Candida stellatoidea, Candida tropicalis, candida utilis, etc.);
Cladosporium (e.g., Cladosporium trichloides, etc);
Coccidioides (e.g., Coccidioides immitis, etc);
Cryptococcus (e.g., Cryptococcus neoformans, etc);
Cunninghamella (e.g., Cunninghamella elegans, etc);
Dermatophyte;
Exophiala (e.g., Exophiala dermatitidis, Exophiala spinifera, etc);
Epidermophyton (e.g., Epidermophyton floccosum, etc);
Fonsecaea (e.g., Fonsecaea pedrosoi, etc);
Fusarium (e.g., Fusarium solani, etc);
Geotrichum (e.g., Geotrichum candiddum, etc);
Histoplasma (e.g., Histoplasma capsulatum var. capsulatum, etc).
Malassezia (e.g., Malassezia furfur, etc);
Microsporum (e.g., Microsporum canis, Microsporum gypseum, etc); Mucor;
Paracoccidioides (e.g., Paracoccidioides brasiliensis, etc);
Penicillium (e.g., Penicillium marneffei, etc);
Phialophora;
Pneumocystis (e.g., Pneumocystis carinii, etc);
Pseudallescheria (e.g., Pseudallescheria boydii, etc);
Rhizopus (e.g., Rhizopus microsporus var. rhizopodiformis, Rhizopus oryzae, etc);
Saccharomyces (e.g., Saccharomyces cerevisiae, etc);
Scopulariopsis;
Sporothrix (e.g., Sporothrix schenckii, etc);
Trichophyton (e.g., Trichophyton mentagrophytes, Trichophyton rubrum, etc);
Trichosporon (e.g., Trichosporon asahii, Trichosporon cutaneuin, etc).

The above fungi are well-known to cause various infection diseases in skin, eye, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph doct, gastrointestine, articulation, muscle, tendon, interstitial plasma cell in lung, blood, and so on.

Therefore, the polypeptide compound (I) of the present invention are useful for preventing and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, fungemia, and so on.

The combination use of azoles such as fluconazole, voriconazole, itraconazole, ketoconazole, miconazole, ER 30346 and SCH 56592; polyenes such as amphotericin B, nystatin, liposamal and lipid forms thereof such as Abelcet, AmBisome, and Amphocil; purine or pyrimidine nucleotide inhibitors such as flucytosine; or polyxins such as nikkomycines, in particular nikkomycine Z or nikkomycine X; other chitin inhibitors; elongation factor inhibitors such as sordarin and analogs thereof; mannan inhibitors such as predamycin, bactericidal/permeability-inducing (BPI) protein products such as XMP.97 or XMP.127; or complex carbohydrate antifungal agents such as CAN-296; or the combination use of immunosuppressant such as tacrolimus with the polypeptide compound (I) or a salt thereof is effective against above infectious diseases.

The pharmaceutical composition of the present invention can be used in the form of a pharmaceutical preparation, for example, in solid, semisolid or liquid form, which contains the polypeptide compound (I) or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The polypeptide compound (I) or a pharmaceutically acceptable salt thereof is/are included in the pharmaceutical composition in an amount sufficient to produce the desired antimicrobial effect upon the process or condition of diseases.

For applying the composition to humans, it is preferable to apply it by intravenous, intramuscular, pulmonary, oral administration, eye drop administration or insufflation. While the dosage of therapeutically effective amount of the polypeptide compound (I) varies from and also depends upon the age and condition of each individual patient to be treated, in the case of intravenous administration, a daily dose of 0.01-400 mg of the polypeptide compound (I) per kg weight of human being in the case of intramuscular administration, a daily dose of 0.1-20 mg of the polypeptide compound (I) per kg weight of human being, in case of oral administration, a daily dose of 0.5-50 mg of the polypeptide compound (I) per kg weight of human being is generally given for treating or preventing infectious diseases.

Especially in case of the treatment of prevention of Pneumocystis carinii infection, the followings are to be noted.

For administration by inhalation, the compounds of the present invention are conveniently delivered in the form of an aerosol spray presentation form pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

For administration by intravenous administration, the preferred pharmaceutical composition is the lyophilized form containing the polypeptide compound (I) or its pharmaceutically acceptable salt.

The amount of the polypeptide compound (I) or its pharmaceutically acceptable salt contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 65, 70, 75, 80, 85, 90, 95 and 100 mg.

The present invention further provides the following ones.

An article of manufacture, comprising packaging material and the compound (I) identified in the above contained within said packaging material, wherein said the compound (I) is therapeutically effective for preventing or treating infectious diseases caused by pathogenic microorganism, and wherein said packaging material comprises a label or a written material which indicates that said compound (I) can or should be used for preventing or treating infectious diseases caused by pathogenic microorganism.

A commercial package comprising the pharmaceutical composition containing the compound (I) identified in the above and a written matter associated therewith, wherein the written matter states that the compound (I) can or should be used for preventing or treating infectious diseases caused by pathogenic microorganism.

The following Preparations and Examples are given for the purpose of illustrating the present invention in more detail.

Preparation 1

To a solution of cyclohexanone (706 mg) and tert-butyl 1,4-diazepane-1-carboxylate (1.2 g) in a mixed solvent of methanol (20 ml), tetrahydrofuran (15 ml) and acetic acid (1.03 ml) was added sodium cyanoborohydride (452 mg). The mixture was stirred at room temperature for 5 hours. The reaction mixture was quenched with saturated aqueous sodium hydrogen carbonate solution. To the reaction mixture was added ethyl acetate. The organic layer was taken and dried over magnesium sulfate. The magnesium sulfate was filtered off, and the filtrate was concentrated under reduced pressure to give tert-butyl 4-cyclohexylhexahydro-1H-1,4-diazepine-1-carboxylate (1.763 g).

NMR (CDCl₃, δ): 1.46 (9H, s), 0.9-2.2 (12H, m), 2.3-2.55 (1H, m), 2.6-2.8 (4H, m), 2.35-2.55 (4H, m) MASS (m/z): 283 (M⁺+H)

Preparation 2

To a solution of 8-(1-hydroxycyclohexyl)-1,4-dioxaspiro[4.5]decan-8-ol (2.75 g) and iodomethane (2.67 ml) in N,N-dimethylformamide (28 ml) was added sodium hydride (60% dispersion in mineral oil) (1.29 g) at 0° C. The solution was stirred for 30 minutes at 0° C. and at room temperature for 26 hours. The reaction mixture was added to a mixture of water and ether. The organic layer was washed with brine and dried over magnesium sulfate. The magnesium sulfate was filtered off, and the filtrate was concentrated under reduced pressure. The residue was purified by silica gel chromatography (4:1 hexane-ethyl acetate elution) to give 8-methoxy-8-(1-methoxycyclohexyl)-1,4-dioxaspiro[4.5]decane (2.398 g).

NMR (CDCl₃, δ): 1.0-2.0 (18H, m), 3.43 (3H, s), 3.44 (3H, s), 3.9-4.0 (4H, m) MASS (m/z): 307 (M⁺+23)

Preparation 3

A solution of 4-hexyloxybromobenzene (14.3 g) in tetrahydrofuran (200 ml at −60° C. under a nitrogen atmosphere was treated with 1.52M n-butyl lithium in hexane solution (43.9 ml) dropwise over 10 minutes, then stirred for 2 hours at the same temperature. Tri-isopropyl borate (12.55 g) in tetrahydrofuran (15 ml) was added dropwise over 30 minutes and after 1 hour at −60° C. the cooling bath removed and the temperature warmed to room temperature over 2 hours. Excess 1N-hydrochloric acid was added and the mixture was stirred for 30 minutes then extracted with ethyl acetate. The organic layer was washed with water (×5), saturated sodium chloride solution (×1), dried over magnesium sulfate, evaporated and the crude product triturated with hexane to afford 4-hexyloxybenzene boronic acid (6.3 g) as a white solid.

NMR (CDCl₃, δ): 0.89-0.95 (3H, m), 1.35-1.55 (6H, m), 1.76-1.86 (2H, m), 4.04 (2H, t, J=6.5 Hz), 7.00 (2H, d, J=8.5 Hz), 8.15 (2H, d, J=8.5 Hz)

Preparation 4

A solution of methyl 4-[2-[4-[4-(4-methylcyclohexyl)-1-piperazinyl]benzoyl]hydrazinocarbonyl]benzoate (2.2 g) in phosphorous oxychloride (25 ml) was heated at reflux for 6 hours then cooled, poured into water, adjusted to pH 7 with sodium hydroxide solution (1N), filtered and the precipitate was washed thoroughly with water and dried to afford methyl 4-[5-[4-[4-(4-methylcyclohexyl)-1-piperazinyl]phenyl]-1,3,4-oxadiazol-2-yl]benzoate (1.93 g) as an off-white solid.

NMR (CDCl₃, δ): 0.98 (3H, d, J=7 Hz), 1.4-2.0 (9H, m), 2.5-2.8 (1H, m), 2.8-3.2 (4H, m), 3.5-3.8 (4H, m), 3.97 (3H, s), 6.99 (2H, d, J=8.8 Hz), 8.03 (2H, d, J=8.8 Hz), 8.20 (4H, s) API-ES(+) MASS: 461.4 (MH⁺)

Preparation 5

To a solution of 1,4-dioxaspiro[4.5]decan-8-ol (3.0 g) in methanol (30 ml) was portionwise added sodium borohydride (1.45 g) with stirring at ambient temperature and the mixture was stirred at the same temperature for 1 hour. The reaction mixture was concentrated in vacuo and chromatographed on silica gel (150 ml) eluting with a mixture of n-hexane and ethyl acetate (2:1 v/v). The fractions containing the desired compound were collected and evaporated under reduced pressure to give 1,4-dioxaspiro[4.5]decan-8-ol (3.43 g).

NMR (CDCl₃, δ): 1.45-1.95 (8H, m), 3.70-3.85 (1H, m), 3.95 (4H, s) APCI MASS (m/z): 159 (M⁺+H)

Preparation 6

To a solution of tert-butyl 4-oxo-1-piperidinecarboxylate (7.0 g) in THF (35 ml) was dropwise added lithium diisopropylamine mono(tetrahydrofuran) (1.5M solution cyclohexane) (25.8 ml) at −70° C. and stirred at the same temperature for 20 minutes. To the solution was dropwise added a solution of N-phenyltrifluoromethanesulfonimide (13.43 g) in THF (35 ml) at −70° C. and the mixture was warmed up to 0° C. and stirred at 0° C. for 3 hours. The reaction mixture was concentrated in vacuo. The resulting residue was dissolved in dichloromethane (50 ml). The solution was subjected to column chromatography on Florisil (100-200 mesh) (400 ml) eluting with a mixture of hexane and ethyl acetate (9:1 v/v). The first fractions containing the object compound were collected and evaporated under reduced pressure to give the crude vinyl triflate. The residue was dissolved in a mixture of dimethoxyethane (250 ml) and aqueous sodium carbonate (Na₂CO₃ 10.4 g in water (50 ml)). To this solution were added 4-(methoxycarbonyl)phenylboric acid (8.85 g), lithium chloride (3.20 g) and tetrakis(triphenylphosphine)palladium (2.02 g) at room temperature and the mixture was refluxed for 2 hours. To the reaction mixture was added ethyl acetate (200 ml) and the solution was washed in turn with water (60 ml) and aqueous sodium chloride (60 ml), dried over magnesium sulfate and evaporated in vacuo. The resulting residue was chromatographed on silica gel (200 g) eluting with a mixture of hexane and ethyl acetate (5:1 v/v). The fractions containing the object compound were collected and evaporated under reduced pressure to give tert-butyl 4-[4-(methoxycarbonyl)phenyl]-3,6-dihydro-1(2H)-pyridinecarboxylate (4.19 g)

NMR (CDCl₃, δ): 1.49 (9H, s), 2.50-2.65 (2H, m), 3.65 (2H, t, J=5.69 Hz), 3.95 (3H, s), 4.05-4.30 (2H, m), 6.16 (1H, br s), 7.43 (2H, J=8.52 Hz), 8.00 (2H, J=8.56 Hz) ESI MASS (Positive)(m/z): 340.2 (M⁺+Na)

Preparation 7

A mixture of tert-butyl 4-[4-(methoxycarbonyl)phenyl]-3,6-dihydro-1(2H)-pyridinecarboxylate (3.68 g) and 10% palladium on carbon (50% wet) (1.8 g) in methanol (40 ml) and tetrahydrofuran (40 ml) was stirred for 5 hours at room temperature under hydrogen atmosphere. After removal of insoluble solids, the filtrate was concentrated in vacuo and the residue was purified by column chromatography on silica gel eluting with a mixture of toluene and ethyl acetate (20:1→10:1). The eluted fractions containing the desired product were collected and evaporated in vacuo to give tert-butyl 4-[4-(methoxycarbonyl)phenyl]-1-piperidinecarboxylate (2.98 g).

IR (Nujol): 1724, 1705, 1421, 1269, 1228, 1159, 1122, 1012 cm⁻¹ NMR (DMSO-d₆, δ): 1.3-1.6 (2H, m), 1.42 (9H, s), 1.7-1.9 (2H, m), 2.6-2.9 (3H, m), 3.84 (3H, s), 4.0-4.2 (2H, m), 7.3-7.5 (2H, m), 7.8-8.0 (2H, m) ESI MASS (Positive): 342.3 (M⁺+Na)

Preparation 8

A mixture of ethyl 4-(4-oxo-1-piperidyl)benzoate (12 g) and dimethylformamide dimethylacetal (12.7 g) was heated at 110° C. for 6 hours, cooled, diluted with hexane and the resulting precipitate was collected by filtration and washed with hexane to afford ethyl 4-[(3E)-3-(dimethylaminomethylene)-4-oxo-1-piperidyl]benzoate as a light yellow powder (10.5 g).

NMR (CDCl₃, δ): 1.37 (3H, t, J-7.1 Hz), 2.59 (2H, t, J=6.2 Hz), 3.16 (6H, s), 3.65 (2H, t, J=6.2 Hz), 4.32 (2H, q, J=7.1 Hz), 4.52 (2H, s), 6.77 (2H, d, J=9.1 Hz), 7.55 (1H, s), 7.93 (2H, d, J=9.1 Hz)

Preparation 9

A solution of ethyl 4-[(3E)-3-(dimethylaminomethylene)-4-oxo-1-piperidyl]benzoate (2 g) and phenylhydrazine (787 mg) in ethanol (20 ml) was heated at reflux. After 2 hours, the reaction mixture was evaporated and dried in vacuo to give an amorphous solid. This solid was dissolved in Ethanol (20 ml), and treated with hydrazine hydrate then refluxed for 40 hours, cooled then extracted with ethyl acetate then washed with water, dried over magnesium sulfate and the crude solid was triturated with ethyl acetate-hexane to give 4-(2-phenyl-2,4,6,7-tetrahydro-5H-pyrazolo[4,3-c]pyridin-5-yl)benzohydrazide as a yellow powder (1.3 g).

NMR (DMSO-d₆, δ): 2.80-3.05 (2H, m), 3.60-3.80 (2H, m), 4.36 (2H, s), 4.36-4.45 (2H, m), 7.04 (2H, d, J=9 Hz), 7.25-7.79 (8H, m), 9.47 (1H, s) APCI-MASS: 334.13 (M⁺+H)

Preparation 10

To a solution of 4-bromo cyanobenzene (3.0 g) in THF (30 ml) was added triisopropoxyborate (5.32 ml) at −70° C. with stirring and then dropwise added n-butyllithium (1.6M solution in hexane) (13.4 ml) at the same temperature. The mixture was stirred at −60˜−70° C. for 1 hour. The reaction mixture was poured into 2N HCl (25 ml) and extracted twice with ethyl acetate (100 ml), washed successively with water and saturated aqueous sodium chloride, dried over magnesium sulfate and evaporated in vacuo. The resulting residue was triturated with diisopropyl ether (50 ml). The resulting precipitates were collected by filtration and dried in vacuo to give 4-cyanophenylboric acid (1.90 g).

NMR (DMSO-d₆, δ): 7.79 (2H, d, J=8.09 Hz), 7.94 (2H, d, J=8.10 Hz)

Preparation 11

To a solution of tert-butyl 4-(trifluoromethylsulfonyloxy)-3,6-dihydro-1(2H)-pyridinecarboxylate (8.33 g) in a mixture of dimethoxyethane (160 ml) and aqueous sodium carbonate (Na₂CO₃ 10.4 g in water (50 ml)). To a solution were added 4-cyanophenylboric acid (5.16 g), lithium chloride (2.28 g) and tetrakis(triphenylphosphine)palladium (1.44 g) at room temperature and the mixture was refluxed for 2 hours and then cooled on ice bath. The reaction mixture was evaporated in vacuo and dissolved in a mixture of dichloromethane (200 ml), 2N aqueous sodium carbonate (100 ml) and conc. ammonium hydroxide (10 ml). The organic layer was separated and the aqueous layer was extracted with dichloromethane (100 ml). The extracts were washed with saturated aqueous sodium chloride, dried over magnesium sulfate and evaporated in vacuo. The resulting residue was chromatographed on silica gel (200 g) eluting with a mixture of hexane and ethyl acetate (5:1 v/v). The fractions containing the object compound were collected and evaporated under reduced pressure to give tert-butyl 4-(4-cyanophenyl)-3,6-dihydro-1(2H)pyridinecarboxylate (4.19 g).

NMR (CDCl₃, δ): 1.49 (9H, s), 2.45-2.60 (2H, m), 3.65 (2H, t, J=5.6 Hz), 4.11 (2H, q, J=2.81 Hz), 6.18 (1H, br s), 7.46 (2H, d, J=8.41 Hz), 7.62 (2H, J=8.39 Hz) ESI MASS (Positive)(m/z): 307.2 (M⁺+Na)

Preparation 12

To a solution of 4-(ethoxycarbonyl)piperidine (10.0 g) in THF (100 ml) were added triethylamine (11.5 ml) and di-tert-butyl dicarbonate (14.6 g) with stirring at ambient temperature and the mixture was stirred at the same temperature for 3 hours. The reaction mixture was concentrated in vacuo. The resulting residue was dissolved in ethyl acetate (200 ml) and the solution was washed successively with 1N hydrochloride, saturated aqueous sodium chloride, saturated aqueous sodium hydrogen carbonate and saturated aqueous sodium chloride dried over magnesium sulfate and evaporated in vacuo. The residue was chromatographed on silica gel (400 ml) eluting with a mixture of n-hexane and ethyl acetate (5:1 v/v). The fractions containing the desired compound were collected and evaporated under reduced pressure to give tert-butyl 4-(ethoxycarbonyl)-1-piperidinecarboxylate (16.09 g).

This compound was immediately used as the starting compound for the next step.

Preparation 13

To a solution of lithium aluminum hydride (1.33 g) in THF (60 ml) was dropwise added a solution of tert-butyl 4-(ethoxycarbonyl)-1-piperidinecarboxylate (6.00 g) in THF (30 ml) with stirring at 0˜−20° C. and the mixture was stirred at the same temperature for 1 hour. To a reaction mixture were added sodium fluoride (5.87 g) and then dropwise added water (1.90 ml) with stirring. After 10 minutes, the mixture was filtrated by celite and evaporated in vacuo. The resulting residue was chromatographed on silica gel (250 ml) eluting with a mixture of dichloromethane and methanol (9:1 v/v). The fractions containing the desired compound were collected and evaporated under reduced pressure to give tert-butyl 4(hydroxymethyl)-1-piperidinecarboxylate (4.13 g).

NMR (CDCl₃, δ): 1.00-1.30 (2H, m), 1.46 (9H, s), 1.50-1.80 (4H, m), 2.55-2.85 (2H, m), 3.40-3.60 (2H, m), 4.00-4.25 (2H, m) APCI MASS (Positive)(m/z): 238.4 (M⁺+Na)

Preparation 14

To a solution of oxalylchloride (2.4 g) in dichloromethane (30 ml) was dropwise added the solution of dimethylsulfoxide (2 g) in dichloromethane (10 ml) at −10° C. with stirring. The mixture was stirred at −5-10° C. for 0.5 hour. To a reaction mixture was dropwise added the solution of 4-(5-methoxypentyloxy)phenethylalcohol (3.0 g) in dichloromethane (40 ml) at −60° C. with stirring. The mixture was stirred at −60° C. for an hour and then dropwise added the triethylamine (8 g) at −60° C. with stirring. The mixture was stirred at −60° C. for an hour and stirred at room temperature for 1.5 hours. The reaction mixture was poured into ice-water and extracted with dichloromethane. The dichloromethane layer was washed with water and dried over magnesium sulfate. The magnesium sulfate was filtered off and the filtrate was concentrated under reduced pressure to give oil. The oil was subjected to column chromatography on silica gel (silica gel 60F₂₅₄, Merck) and eluted a mixture of ethyl acetate and n-hexane (1:4). The fraction containing the object compound were combined and concentrated under reduced pressure to give 4-(5-methoxypentyloxy)phenylacetaldehyde (1.0 g).

NMR (CDCl₃, δ): 1.40-1.90 (6H, m), 3.34 (3H, s), 3.40 (2H, t, J=6.2 Hz), 3.62 (2H, d, J=2.4 Hz), 3.96 (2H, t, J=6.4 Hz), 6.88 (2H, d, J=8.7 Hz), 7.11 (2H, d, J=8.7 Hz), 9.72 (1H, br s) API-ES MASS (Negative): 249 (M⁺+Na), 236 (M), 235 (M⁻−1)

Preparation 15

To a solution of 4-(5-methoxypentyloxy)-phenylacetaldehyde (0.47 g) in dichloromethane (5 ml) was dropwise added the solution of bromine (0.35 g) in dichloromethane (1 ml) at −10° C. with stirring. The mixture was stirred at room temperature for 0.5 hour and stirred at reflux for 40 minutes. The reaction mixture was concentrated under nitrogen gas at 40° C. and added the thiourea (0.15 g) and ethanol (10 ml) to the residue. The mixture was refluxed for 6 hours with stirring. The reaction mixture was concentrated under reduced pressure and added the water to the residue. The solution was adjusted to pH 8.5 using the sodium bicarbonate and extracted with the mixture was ethyl acetate and tetrahydrofuran (1:1). The organic layer was washed with saturated sodium chloride aqueous solution and dried over magnesium sulfate. The magnesium sulfate was filtered off and the filtrate was concentrated under reduced pressure to give oily. The oil was subjected to column chromatography on silica gel (silica gel 60F₂₅₄, Merck) and eluted the mixture of chloroform and methanol (10:1). The fractions containing the objective compound was combined and concentrated under reduced pressure to give 2-amino-5-(5-methoxypentyloxyphenyl)thiazole (0.32 g).

NMR (DMSO-d₆, δ): 1.40-1.90 (6H, m), 3.22 (3H, s), 3.20-3.40 (2H, m), 4.03 (2H, t, J=6.3 Hz), 6.80-7.40 (4H m), 7.10 (2H, s), 7.63 (1H, s) MASS (m/z): 371 (M⁺+Br), 293 (M⁺+H)

Preparation 16

N,N-diisopropylamine (26.2 ml) is added dropwise to a solution of butyllithium (107.5 ml:1.6M in hexane) in tetrahydrofuran (300 ml) under nitrogen atmosphere and cooled in an ice bath at 0-5° C. After maintaining the solution at 0-5° C. for an additional 30 minutes, cyclohexanecarboxylic acid (10 g) is added at once. The cooling bath is removed and the reaction solution is allowed to stir at room temperature for 4 hours. A solution of 1,4-dioxaspiro[4.5]decan-8-one (12.2 g) in tetrahydrofuran is added at once. After stirred at room temperature for 14 hours, the mixture is poured into ice water, washed once with diethyl ether, acidified to pH 1 with conc. HCl, and extracted with chloroform-methanol (9:1). The extracts are dried over magnesium sulfate, filtered, and concentrated in vacuo. The residue is triturated with a solvent mixture consisting of ethyl acetate (50 ml), diethyl ether (250 ml) and hexane (250 ml), collected by filtration, and dried to give 1′-(8-hydroxy-1,4-dioxaspiro[4.5]dec-8-yl)cyclohexanecarboxylic acid (13.068 g).

NMR (CDCl₃, δ): 0.8-2.4 (22H, m), 3.8-4.1 (4H, m) MASS (m/z): 283 (M⁺−H)

Preparation 17

Dimethylformamide dineopentylacetal was added at once to a stirred slurry of 1′-(8-hydroxy-1,4-dioxaspiro[4.5]dec-8-yl)cyclohexanecarboxylic acid (10 g) in acetonitrile at room temperature. The mixture was stirred at room temperature for 1 hour and then was heated for 21 hours at gentle reflux. The mixture was cooled, diluted with diethyl ether, washed with ice water, brain, and the organic layer was dried over magnesium sulfate. The solution is filtered and concentrated in vacuo. The residue was purified by silica gel chromatography (10:1 hexane-ethyl acetate elution) to give 8-cyclohexylidene-1,4-dioxaspiro[4.5]decane (7.19 g).

NMR (CDCl₃, δ): 1.4-1.7 (10H, m), 2.1-2.4 (8H, m), 3.97 (4H, s) MASS (m/z): 222.80 (M⁺+H)

Preparation 18

To a stirred solution of 8-cyclohexylidene-1,4-dioxaspiro[4.5]decane (5 g) in dichloromethane (200 ml) was added-dropwise the oxidant solution KMnO₄ (5.33 g), triethylbenzylammonium chloride (7.68 g) and dichloromethane (400 ml) at such a rate that the temperature was maintained at 0-3° C. under cooling with ice bath. After addition was completed, stirring was continued until permanganate ion was completely consumed. The homogeneous dark brown solution was treated with 3% sodium hydrogen carbonate solution (300 ml) at room temperature for 18 hours. The organic layer was washed with brine and dried over magnesium sulfate. The magnesium sulfate was filtered off, and the filtrate was concentrated under reduced pressure. The residue was purified by silica gel chromatography (3:1-1:1 hexane-ethyl acetate elution) to give 8-(1-hydroxycyclohexyl)-1,4-dioxaspiro[4.5]decan-8-ol (2.805 g).

NMR (CDCl₃, δ): 1.0-2.1 (20H, m), 3.85-4.0 (4H, m) MASS (m/z): 279 (M⁺+23)

Preparation 19

To a solution of 1-hydroxy-4-methylcyclohexane (13.5 g) and triethylamine (21.4 ml) in ethyl acetate (135 ml) was added dropwise with stirring methanesulfonyl chloride (20 ml) at 0° C. The mixture was then stirred for 25 hours at 0° C. The reaction mixture was added to a mixture of 1 mol/l hydrochloric acid and ethyl acetate. The organic layer was washed with water, sodium hydrogen carbonate solution and brain. The organic layer was taken and dried over magnesium sulfate. The magnesium sulfate was filtered off, and the filtrate was concentrated under reduced pressure to give cis-4-methylcyclohexyl methanesulfonate (22.87 g).

NMR (CDCl₃, δ): 0.93 (3H, d, J-6.5 Hz), 1.2-1.75 (7H, m), 1.95-2.15 (2H, m), 3.01 (3H, s), 4.9-5.0 (1H) MASS (m/z): 215 (M⁺+23)

The following compound was obtained according to a similar manner to that of Preparation 19.

Preparation 20

### Trans-4-Methylcyclohexyl methanesulfonate

NMR (CDCl₃, δ): 0.90 (3H, d, J=6.5 Hz), 0.95-1.2 (2H, m), 1.2-1.9 (5H, m), 2.05-2.25 (2H, m), 3.00 (3H, s), 5.0-5.7 (1H, m) MASS (m/z): 215 (M⁺+23)

Preparation 21

A solution of piperazine (8.96 g) in methanol was heated at 120° C. Since the solvent was disappeared, to the solution was added 4-methylcyclohexyl methanesulfonate (5 g). The solution was mixed for 4 hours at 120° C. The reaction mixture was purified by silica gel chromatography (5:1 dichloromethane-methanol elution) to give 1-(cis-4-methylcyclohexyl)piperazine (1.2 g)

NMR (CDCl₃, δ): 0.93 (3H, d, J=7.0 Hz), 1.4-2.0 (10H, m), 2.1-2.3 (1H, m), 2.45-2.65 (4H, m), 2.9-3.0 (4H, m) MASS (m/z): 183 (M⁺+H)

The following compound was obtained according to a similar manner to that of Preparation 21.

Preparation 22

### 1-(trans-4-Methylcyclohexyl)piperazine

NMR (CDCl₃, δ): 0.8-1.4 (8H, m), 1.65-2.0 (5H, m), 2.05-2.3 (1H, m), 2.45-2.6 (4H, m), 2.8-3.0 (4H, m) MASS (m/z): 183 (M⁺+H)

Preparation 23

Sodium hydride, 60% dispersion in mineral oil (950 mg) was added portionwise to a solution of methyl 4-hydroxybenzoate (3 g) in N,N-dimethylformamide (15 ml) at ambient temperature. The mixture was stirred at 60° C. for 2 hours. The mixture was added portionwise to 1,7-dibromoheptane (10.1 ml) in N,N-dimethylformamide (15 ml) at ambient temperature, and stirred for 16 hours. The reaction mixture was diluted with a mixture of ethyl acetate and water, and the organic layer was separated, washed with water and brine, dried, and evaporated under reduced pressure. The residue was chromatographed on a column of silica gel eluting with a mixture of n-hexane and ethyl acetate (20:1) to give methyl 4-(7-bromoheptyloxy)benzoate (4.19 g).

IR (KBr): 2942.8, 1710.6, 1606.6, 1251.6 cm⁻¹ NMR (CDCl₃, δ): 1.37-1.56 (6H, m), 1.74-1.91 (4H, m), 3.42 (2H, t, J=6.8 Hz), 3.88 (3H, s), 4.01 (2H, t, J=6.4 Hz), 6.86-6.93 (2H, m), 7.94-8.02 (2H, m) ESI MASS (Positive)(m/z): 351.1 (M⁺+Na)

The following compounds [Preparation 24 and 25] were obtained according to a similar manner to that of Preparation 23.

Preparation 24

### 8-(6-Bromohexyloxy)-1,4-dioxaspiro[4.5]decane

NMR (CDCl₃, δ): 1.30-1.95 (16H, m), 3.25-3.45 (5H, m), 3.94 (4H, s) ESI MASS (Positive)(m/z): 343.2 (M⁺+Na)

Preparation 25

### 4-[4-(7-Bromohexyloxy)piperidin-1-yl]benzoate

NMR (CDCl₃, δ): 1.36 (3H, t, J=7.1 Hz), 1.22-1.50 (6H, m), 1.50-2.12 (8H, m), 2.98-3.19 (2H, m), 3.32-3.57 (5H, m), 3.57-3.75 (2H, m), 4.32 (2H, q, J=7.1 Hz), 6.86 (2H, d, J=9.0 Hz), 7.91 (2H, d, J=8.9 Hz) MASS (m/z): 426, 428 (M⁺+H, M++3)

Preparation 26

A mixture of 4-(4-piperidyloxy)benzonitrile (1.5 g), iodobenzene (1 ml), palladium(II) acetate (83 mg), racemic-2,2′-bis(diphenylphosphino)-1,1′-binaphthyl (0.23 g) and cesium carbonate (4.8 g) was stirred for 14.5 hours at 90° C. under nitrogen atmosphere. After being cooled to room temperature, the reaction mixture was poured into a mixture of ethyl acetate and water. The organic layer was successively washed with water and brine and dried over magnesium sulfate. The solvent was evaporated in vacuo and the residue was purified by column chromatography on silica gel eluting with a mixture of hexane and ethyl acetate (5:1). The eluted fractions containing the desired product were collected and evaporated in vacuo to give 4-(1-phenyl-4-piperidyloxy)benzonitrile (1.26 g).

IR (KBr): 2218, 1599, 1500, 1298, 1254, 1228, 1171, 1120, 1034, 837, 754, 689 cm⁻¹ NMR (DMSO-d₆, δ): 1.6-1.9 (2H, m), 2.0-2.2 (2H, m), 3.0-3.2 (2H, m), 3.4-3.6 (2H, m), 4.6-4.8 (1H, m), 6.76 (1H, t, J=7.2 Hz), 6.96 (2H, d, J=7.9 Hz), 7.1-7.3 (4H, m), 7.76 (2H, d, J=8.9 Hz) ESI MASS (Positive): 301.2 (M⁺+H)

The following compounds [Preparation 27 to 29] were obtained according to a similar manner to that of Preparation 26.

Preparation 27

### Ethyl 4-(1-phenyl-4-piperidyloxy)benzoate

IR (Nujol): 1705, 1603, 1493, 1277, 1254, 1173, 1103, 1034 cm⁻¹ NMR (DMSO-d₆, δ): 1.30 (3H, t, J-7.1 Hz), 1.6-1.9 (2H, m), 2.0-2.2 (2%, m), 3.0-3.2 (2H, m), 3.4-3.6 (2H, m), 4.28 (2H, q, J=7.1 Hz), 4.6-4.8 (1H, m), 6.7-7.3 (7H, m), 7.90 (2H, d, J=8.8 Hz) ESI MASS (Positive): 326.3 (M⁺−H)

Preparation 28

### Ethyl 4-[1-(4-methoxyphenyl)-4-piperidyloxy]benzoate

IR (Nujol): 1701, 1508, 1252, 1171, 1103, 1034 cm⁻¹ NMR (DMSO-d₆, δ): 1.30 (3H, t, J=7.1 Hz), 1.7-1.9 (2H, m), 2.0-2.2 (2H, m), 2.8-3.0 (2H, m), 3.3-3.5 (2H, m), 3.68 (3H, s), 4.27 (2H, d, J=7.1 Hz), 4.6-4.7 (1H, m), 6.8-7.0 (4H, m), 7.09 (2H, d, J=8.8 Hz), 7.90 (2H, d, J=8.8 Hz) ESI MASS (Positive): 356.3 (M⁺+H)

Preparation 29

### 4-[1-(4-Methoxyphenyl)-4-piperidyloxy]benzonitrile

IR (Nujol): 2222, 1510, 1257 cm⁻¹ NMR (DMSO-d₆, δ): 1.6-1.9 (2H, m), 2.0-2.2 (2H, m), 2.8-3.0 (2H, m), 3.3-3.4 (2H, m), 3.68 (3H, s), 4.6-4.8 (1H, m), 6.7-7.0 (4H, m), 7.1-7.2 (2H, m), 7.7-7.8 (2H, m) ESI MASS (Positive): 331.2 (M⁺+Na)

Preparation 30

To a mixture of 4-hydroxybenzonitrile (10.7 g), tert-butyl 4-hydroxy-1-piperidinecarboxylate (27.1 g) and triphenylphosphine (35.4 g) in tetrahydrofuran (250 ml) was added diethyl azodicarboxylate (21.3 ml) at room temperature under nitrogen atmosphere. After stirring for 6 hours at room temperature under nitrogen atmosphere, the solvent was evaporated in vacuo. Then to the residue was added ethyl ether and insoluble solids were filtered off. The filtrate was evaporated in vacuo and the residue was purified by column chromatography on silica gel eluting with a mixture of hexane and ethyl acetate (10:1-+5:1). The eluted fractions containing the desired product were collected and evaporated in vacuo to give tert-butyl 4-(4-cyanophenoxy)-1-piperidinecarboxylate (26.7 g).

NMR (DMSO-d₆, δ): 1.3-1.6 (11H, m), 1.8-2.0 (2H, m), 3.0-3.3 (2H, m), 3.6-3.8 (2H, m), 4.6-4.8 (1H, m), 7.15 (2H, d, J=8.8 Hz), 7.76 (2H, d, J=8.8 Hz) ESI MASS (Positive): 325.2 (M⁺+Na)

The following compounds [Preparation 31 and 32] were obtained according to a similar manner to that of Preparation 30.

Preparation 31

### Tert-Butyl 4-4-(ethoxycarbonylphenoxy)-1-piperidinecarboxylate

IR (Nujol): 2981, 2935, 2875, 1699, 1687, 1601, 1417, 1367, 1313, 1277, 1254, 1236, 1163, 1105, 1038 cm⁻¹ NMR (DMSO-d₆, δ): 1.30 (3H, t, J=7.1 Hz), 1.41 (9H, s), 1.4-1.6 (2H, m), 1.8-2.0 (2H, m), 3.1-3.3 (2H, m), 3.6-3.8 (2H, m), 4.27 (2H, q, J=7.1 Hz), 4.6-4.8 (1H, m), 7.08 (2H, d, J=8.9 Hz), 7.89 (2H, d, J=8.8 Hz) ESI MASS (Positive): 372.3 (M⁺+H)

Preparation 32

### Tert-Butyl 4-(4-bromophenoxy)-1-piperidinecarboxylate

NMR (CDCl₃, δ): 1.47 (9H, s), 1.6-2.0 (4H, m), 3.2-3.4 (2H, m), 3.6-3.8 (2H, m), 4.3-4.5 (1H, m), 6.78 (2H, d, J=6.8 Hz), 7.36 (2H, d, J=6.8 Hz)

Preparation 33

To a solution of 4-pentyloxy-1-(tert-butoxycarbonyloxy)piperidine (6 g) in ethyl acetate (30 ml) was added dropwise 4N hydrogen chloride in ethyl acetate (28 ml) at 0-10° C., and stirred at ambient temperature for 2 hours. The reaction mixture was evaporated under reduced pressure to give 4-pentyloxy piperidine (3.87 g).

IR (KBr): 3488.6, 2944.8, 1591.0, 1091.5 cm⁻¹ NMR (CDCl₃, δ): 0.90 (3H, t, J=6.6 Hz), 1.27-2.16 (11H, m), 3.04-3.59 (7H, m) ESI MASS (Positive)(m/z): 172.07 (M⁺+H)

The following compounds [Preparation 34 and 35] were obtained according to a similar manner to that of Preparation 33.

Preparation 34

### 4-Butoxy piperidine hydrochloride salt

IR (KBr): 2966.0, 1589.1, 1110.8 cm⁻¹ NMR (CDCl₃, δ): 0.92 (3H, t, J=7.2 Hz), 1.23-1.61 (4H, m), 1.91-2.16 (5H, m), 2.15-3.63 (7H, m), 9.36 (1H, br s) ESI MASS (Positive)(m/z): 157.93 (M⁺+H) (free)

Preparation 35

### 1-(4-Bromophenyl)piperazine

NMR (CDCl₃, δ): 2.95-3.2 (8H, m), 6.7-6.9 (2H, m), 7.3-7.5 (2H, m) MASS (m/z): 241, 243 (M⁺+H)

Preparation 36

To a mixture of tert-butyl 4-(4-cyanophenoxy)-1-piperidinecarboxylate (2.3 g) and anisole (4.2 ml) in dichloromethane (23 ml) was added portionwise trifluoroacetic acid (12 ml) under ice-cooling. After stirring for 8 hours under ice-cooling, the solvent was evaporated in vacuo. The residue was poured into a mixture of ethyl acetate and water and the solution was adjusted to pH 10 with potassium carbonate. Then the organic layer was concentrated in vacuo and the residue was pulverized from isopropyl ether to give 4-(4-piperidyloxy)benzonitrile (1.55 g).

NMR (DMSO-d₆, δ): 1.5-1.8 (2H, m), 1.9-2.1 (2H, m), 2.8-3.0 (2H, m), 3.0-3.2 (2H, m), 4.6-4.8 (1H, m), 7.1-7.2 (2H, m), 7.7-7.8 (2H, m) ESI MASS (Positive): 203.2 (M⁺+H)

The following compounds [Preparation 37 to 43] were obtained according to a similar manner to that of Preparation 36.

Preparation 37

### Ethyl 4-(4-piperidyloxy)benzoate

NMR (DMSO-d₆, δ): 1.30 (3H, t, J=7.1 Hz), 1.4-1.6 (2H, m), 1.9-2.1 (2H, m), 2.6-2.8 (2H, m), 2.9-3.1 (2H, m), 3.89 (1H, br s), 4.27 (2H, q, J=7.1 Hz), 4.4-4.7 (1H, m), 7.0-7.1 (2H, m), 7.8-8.0 (2H, m) ESI MASS (Positive): 250.2 (M⁺+H)

Preparation 38

### Methyl 4-(4-piperidyl)benzoate

IR (Nujol): 1709, 1277, 1107 cm⁻¹ NMR (DMSO-d₆, δ): 1.4-1.8 (4H, m), 2.6-2.8 (3H, m), 3.0-3.1 (2H, m), 3.84 (3H, s), 7.38 (2H, d, J=8.3 Hz), 7.90 (2H, d, J=8.3 Hz) ESI MASS (Positive): 220.4 (M⁺+H)

Preparation 39

### 4-4-(Methoxybutyloxymethyl)piperidine trifluoroacetate

This compound was immediately used as the starting compound for the next step.

Preparation 40

### 4-(1,2,3,6-Tetrahydro-4-pyridyl)benzonitrile

IR (Neat): 2226, 1651, 1603, 1558, 1541, 1506, 1419 cm⁻¹ NMR (DMSO-d₆, δ): 2.3-2.4 (2H, m), 2.8-3.0 (2H, m), 3.3-3.4 (2H, m), 6.4-6.5 (1H, m), 7.5-7.7 (2H, m), 7.7-7.9 (2H, m), 7.98 (1H, s) ESI MASS (Positive): 185.2 (M⁺+H)

Preparation 41

### 4-(5-Methoxypentyloxymethyl)piperidine trifluoroacetate

This compound was immediately used as the starting compound for the next step.

Preparation 42

### 4-(4-Methylpentyloxy)piperidine trifluoroacetate

This compound was used in the next reaction without further purification.

Preparation 43

### 4-(Cyclohexylmethoxy)piperidine trifluoroacetate

This compound was used in the next reaction without further purification.

Preparation 44

To a solution of tert-butyl 4-(4-bromophenyl)-1-piperazinecarboxylate (1.1 g) in dichloromethane (11 ml) was added dropwise with stirring trifluoroacetic acid (5 ml) at 0° C. The mixture was then stirred for 2 hours at room temperature. Then the solvent was evaporated and the reaction mixture was added to a mixture of ethyl acetate and tetrahydrofuran. The organic layer was washed with sodium hydrogen carbonate solution and sodium chloride solution. The organic layer was taken and dried over magnesium sulfate. The magnesium sulfate was filtered off, and the filtrate was concentrated under reduced pressure to give 1-(4-bromophenyl)piperazine (710 mg).

NMR (CDCl₃, δ): 2.95-3.2 (8H, m), 6.7-6.9 (2H, m), 7.3-7.5 (2H, m) MASS (m/z): 241, 243 (M⁺+H)

The following compounds [Preparation 45 to 47] were obtained according to a similar manner to that of Preparation 44.

Preparation 45

### 1-Cyclohexylhexahydro-1H-1,4-diazepine

NMR (CDCl₃, δ): 0.8-2.0 (12H, m), 2.3-2.65 (2H, m), 2.7-3.1 (8H, m) MASS (m/z): 183 (M⁺+H)

Preparation 46

### 2-Cyclohexyl-2,5-diazabicyclo[2.2.1]heptane

NMR (CDCl₃, δ): 1.0-3.0 (16H, m), 3.1-3.9 (4H, m) MASS (m/z): 181 (M⁺+H)

Preparation 47

### 4-(4-Bromophenoxy)piperidine

NMR (CDCl₃, δ): 2.0-2.3 (5H, m), 3.1-3.5 (4H, m), 4.55-4.7 (1H, m), 6.79 (2H, d, J=8.9 Hz), 7.41 (2H, d, J=8.9 Hz) MASS (m/z): 256, 258 (M⁺+H)

Preparation 48

A solution of 4-fluorobenzonitrile (1.89 g), 4-4-(methoxybutyloxymethyl)piperidine trifluoroacetate (3.6 g) and potassium carbonate (4.73 g) in DMSO (40 ml) was stirred at 140-150° C. for 4 hours. The reaction mixture was poured into water (150 ml) and extracted twice with ethyl acetate (80 ml). The extracts were collected, washed with saturated aqueous sodium chloride, dried over magnesium sulfate and evaporated in vacuo. The resulting residue was chromatographed on silica gel (200 ml) eluting with a mixture of n-hexane and ethyl acetate (2:1 v/v). The fractions containing the desired compound were collected and evaporated under reduced pressure to give 4-(4-methoxybutyloxymethyl)-1-(4-cyanophenyl)piperidine (2.76 g).

NMR (CDCl₃, δ): 1.20-1.45 (2H, s), 1.55-1.75 (4H, m), 1.75-1.90 (3H, m), 2.86 (2H, dt, J=2.37, 12.5 Hz), 3.28 (2H, d, J=6.03 Hz), 3.34 (3H, s), 3.35-3.50 (4H m), 3.70-3.90 (2H, m), 5.70 (2H, br s), 6.90 (2H, d, J=8.96 Hz), 7.64 (2H, d, J=8.86 Hz) APCI MASS (Positive) (m/z): 377.3 (M⁺+H)

The following compounds [Preparation 49 to 52] were obtained according to a similar manner to that of Preparation 48.

Preparation 49

### Ethyl 4-(1,4-dioxa-8-azaspiro[4.5]dec-8-yl)benzoate

NMR (CDCl₃, δ): 1.37 (3H, t, J=7.1 Hz), 1.77-1.83 (4H, m), 3.45-3.51 (4H, m), 4.00 (4H, s), 4.32 (2H, q, J=7.1 Hz), 6.88 (2H, d, J=7 Hz), 7.90 (2H, d, J=7 Hz) APCI MASS: 292.13 (M⁺+H)

Preparation 50

### 4-[4-(5-Methoxypentyloxymethyl)-1-piperidyl]benzonitrile

NMR (CDCl₃, δ): 1.15-1.50 (4H, m), 1.50-1.70 (4H, m), 1.75-1.90 (3H, m), 2.70-2.95 (2H, m), 3.28 (2H, d, J=6.01 Hz), 3.33 (3H, s), 3.35 (2H, d, J=6.60 Hz), 3.43 (2H, d, J=6.41 Hz), 3.70-3.90 (2H, m), 6.85 (2H, d, J=9.06 Hz), 7.46 (2H, d, J=9.00 Hz) ESI MASS (Positive)(m/z): 339.3 (M⁺+Na)

Preparation 51

### Tert-Butyl 4-(4-bromophenyl)-1-piperazinecarboxylate

NMR (CDCl₃, δ): 1.48 (9H, s), 3.05-3.15 (4H, m), 3.5-3.6 (4H, m), 6.79 (2H, d, J=9.0 Hz), 7.35 (2H, d, J=9.0 Hz) MASS (m/z): 340, 342 (M⁺+H)

Preparation 52

### Ethyl 4-(4-butoxypiperidin-1-yl)benzoate

IR (KBr): 2954.4, 1695.1, 1240.0, 1112.7 cm⁻¹ NMR (CDCl₃, δ): 0.93 (3H, t, J=7.2 Hz), 1.29-2.03 (11H, m), 3.04-3.16 (2H, m), 3.44-3.55 (3H, m), 3.60-3.72 (2H, m), 4.32 (2H, q, J=7.1 Hz), 6.84-6.90 (2H, m), 7.87-7.94 (2H, m) ESI MASS (Positive)(m/z): 306.20 (M⁺+H)

Preparation 53

A solution of 8-(6-methoxyhexyloxy)-1,4-dioxaspiro[4.5]decane (1.55 g) in a mixture of THF (16 ml) and 3N hydrochloric acid (5.7 ml) was stirred at ambient temperature for 5 hours. The reaction mixture was then concentrated in vacuo. The resulting residue was dissolved in ethyl acetate (50 ml) and saturated aqueous sodium hydrogen carbonate (10 ml). The solution was washed with saturated aqueous sodium chloride, dried over magnesium sulfate and evaporated in vacuo to give 4-(6-methoxyhexyloxy)cyclohexanone (1.24 g).

NMR (CDCl₃, δ): 1.30-1.40 (4H, m), 1.45-1.65 (4H, m), 1.80-2.35 (6H, m), 2.40-2.65 (2H, m), 3.33 (3H, s), 3.37 (2H, t, J=6.58 Hz), 3.49 (2H, t, J=6.38 Hz), 3.60-3.75 (1H, m) ESI MASS (Positive)(m/z): 25.13 (M⁺+Na)

The following compounds [Preparation 54 and 55] were obtained according to a similar manner to that of Preparation 53.

Preparation 54

### Ethyl 4-(4-oxo-1-piperidyl)benzoate

NMR (CDCl₃, δ): 1.38 (3H, t, J=7.1 Hz), 2.57 (4H, t, J=6.1 Hz), 3.75 (4H, t, J=6 Hz), 4.34 (2H, q, J=7.1 Hz), 6.91 (2H, d, J=8.9 Hz), 7.97 (2H, d, J=8.9 Hz) APCI MASS: 248.2 (M⁺+H)

Preparation 55

### 1,1′-Dimethoxy-1,1′-bi(cyclohexyl)-4-one

NMR (CDCl₃, δ): 1.0-2.0 (12H, m), 2.1-2.4 (4H, m), 2.4 2.7 (2H, m), 3.44 (3H, s), 3.52 (3H, s) MASS (m/z): 263 (M⁺+23)

Preparation 56

Sodium hydride, 60% dispersion in mineral oil (3.1 g) was added slowly to a solution of 1-(tert-butoxycarbonyloxy)piperidin-4-ol (12 g) in N,N-dimethylformamide (60 ml) at ambient temperature. The mixture was stirred at 60° C. for 1.5 hours. To the reaction mixture was added dropwise 1-iodobutane (8.82 ml) at ambient temperature, and stirred for 19 hours. The reaction mixture was poured into water (400 ml), and extracted with ethyl acetate. The extract was washed with brine and dried, and evaporated under reduced pressure. The residue was chromatographed on a column of silica gel eluting with a mixture of n-hexane and ethyl acetate (4:1) to give 4-butoxy-1-(tert-butoxycarbonyloxy)piperidine (3.70 g).

IR (KBr): 2956.3, 1702.8, 1689.3, 1174.4 cm⁻¹ NMR (CDCl₃, δ): 0.92 (3H, t, J=7.2 Hz), 1.26-1.86 (17H, m), 3.01-3.82 (7H, m) ESI MASS (Positive)(m/z): 157.93 (M+−tBoc)

The following compound was obtained according to a similar manner to that of Preparation 56.

Preparation 57

### 4-Pentyloxy-1-(tert-butoxycarbonyloxy)piperidine

IR (KBr): 2933.2, 1693.2, 1105.0 cm⁻¹ NMR (CDCl₃, δ): 0.90 (3H, t, J=6.6 Hz), 1.28-1.86 (19H, m), 3.00-3.82 (7H, m) ESI MASS (Positive)(m/z): 172.00 (M+−tBoc+1)

Preparation 58

To a solution of 4-(4-butoxypiperidin-1-yl)benzohydrazide (2.55 g) and pyridine (2.61 ml) in tetrahydrofuran (76.5 ml) was added dropwise phenylchloroformate (1.82 g) with stirring under ice-cooling, and the mixture was stirred at the ambient temperature for 3.5 hours. The reaction mixture was added water (770 ml) and the resulting precipitate collected, and dried to give methyl 4-[2-[4-(4-butoxypiperidin-1-yl)benzoyl]hydrazinocarbonyl]-benzoate (3.74 g).

IR (KBr): 3263.0, 2954.4, 1724.0, 1278.6, 1108.9 cm⁻¹ NMR (CDCl₃, δ): 0.93 (3H, t, J=7.2 Hz), 1.30-1.93 (8H, m), 3.04-3.68 (7H, m), 3.94 (3H, s), 6.82-6.87 (2H, m), 7.22-7.77 (2H, m), 7.89-7.93 (2H, m), 8.04-8.08 (2H, m), 9.46 (1H, d, J=5.1 Hz), 9.98 (1H, d, J=5.5 Hz) ESI MASS (Positive)(m/z): 454.33 (M⁺+H)

Preparation 59

### Ethyl 4-[4-[2-[4-(7-methoxyheptyloxy)benzoyl]-hydrazinocarbonyl]-1-piperazinyl]benzoate (1.25 g)

IR (KBr): 3280.3, 2979.5, 1706.7, 1648.3, 1110.8 cm⁻¹ NMR (CDCl₃, δ): 1.26-1.72 (13H, m), 3.21 (3H, s), 3.26-3.60 (10H, m), 4.02 (2H, t, J=6.4 Hz), 4.25 (2H, q, J=7.1 Hz), 6.98-7.04 (4H, m), 7.78-7.87 (4H, m), 8.70 (1H, br s), 9.95 (1H, br s) ESI MASS (Positive) (m/z): 540.87 (M)+

Preparation 60

A solution of tert-butyl 4-(hydroxymethyl)-1-piperidinecarboxylate (4.12 g) in DMF (21 ml) was sodium hydride (60% in oil) (0.995 g) at ambient temperature with stirring and the mixture was stirred at 60° C. for 2 hours. The reaction mixture was dropwise added to a solution of 1,5-dibromoheptane (17.6 ml) in DMF (35 ml) with stirring at ambient temperature and stirred at the same temperature for 5 hours. The reaction mixture was poured into water (200 ml) and extracted twice with a mixture of ethyl acetate (80 ml) and n-hexane (40 ml). The extracts were washed with saturated aqueous sodium chloride, dried over magnesium sulfate and evaporated in vacuo. The resulting residue was chromatographed on silica gel (200 ml) eluting with a mixture of n-hexane and ethyl acetate (5:1 v/v). The fractions containing the desired compound were collected and evaporated under reduced pressure to give tert-butyl 4-(4-bromopentyloxymethyl)-1-piperidinecarboxylate (1.89 g).

NMR (CDCl₃, δ): 1.00-1.30 (6H, m), 1.45 (9H, s), 1.46-1.80 (7H, m), 1.80-1.96 (2H, m), 2.55-2.80 (2H, m), 3.24 (2H, d, J=6.05 Hz), 3.35-3.45 (4H, m), 4.00-4.20 (2H, m) APCI MASS (Positive)(m/z): 388.2, 386.2 (M⁺+Na)

The following compounds [Preparation 61 and 62] were obtained according to a similar manner to that of Preparation 60.

Preparation 61

### 1-Bromo-4-(hexyloxy)benzene

NMR (CDCl₃, δ): 0.87-0.93 (3H, m), 1.28-1.55 (6H, m), 1.69-1.83 (2H, m), 3.91 (2H, t, J=6.5 Hz), 6.73-6.80 (2H, m), 7.31-7.39 (2H, m) EI MASS (m/z): 256, 258 (M+, Br isotopes)

Preparation 62

### 1-(6-Bromohexyl)piperazine

NMR (DMSO-d₆, δ): 1.20-1.50 (14H, m), 1.65-1.90 (2H, m), 2.10-2.30 (4H, m), 3.20-3.35 (4H, m), 3.50 (2H, t, J=4.0 Hz) MASS: 351 (M⁺), 349 (M)

Preparation 63

To a mixture of cesium carbonate (2.53 g), palladium(II) acetate (62.3 mg) and 2,2′-bis(diphenylphosphino)-1,1′-binaphthyl (259 mg) in toluene (11 ml) was successively added methyl 4′-trifluoromethylsulfonyloxy-[1,1′-biphenyl]-4-carboxylate (2.00 g) and 4-phenylpiperidine (1.07 g) in stream of nitrogen. The mixture was stirred at ambient temperature for 30 minutes and at 110° C. for further 18 hours. After cooling to room temperature, water and acetonitrile were added to the reaction mixture. The resulting precipitate was collected by filtration and washed with water and acetonitrile and dried to give methyl 4′-(4-phenyl-1-piperidyl)-1,1′-biphenyl-4-carboxylate (393 mg).

NMR (CDCl₃, δ): 1.8-2.1 (4H, m), 2.6-3.0 (3H, m), 3.8-4.0 (5H, m), 7.06 (2H, d, J=8.9 Hz), 7.15-7.4 (5H, m), 7.5-7.7 (4H, m), 8.0-8.15 (2H, m) MASS (m/z): 372 (M⁺+H)

The following compounds [Preparation 64 to 71] were obtained according to a similar manner to that of Preparation 63.

Preparation 64

### Methyl 4′-(4-cyclohexylhexahydro-1H-1,4-diazepin-1-yl)-1,1′-biphenyl-4-carboxylate

NMR (CDCl₃, δ): 0.7-2.3 (12H, m), 2.6-3.2 (5H, m), 3.5-3.8 (4H, m), 3.93 (3H, s), 6.77 (2H, d, J=8.9 Hz), 7.54 (2H, d, J=8.9 Hz), 7.61 (2H, d, J=8.4 Hz), 8.05 (2H, d, J=8.4 Hz) MASS (m/z): 393 (M⁺+H)

Preparation 65

### Tert-Butyl 5-cyclohexyl-2,5-diazabicyclo[2.2.1]heptane-2-carboxylate

NMR (CDCl₃, δ): 1.0-1.4 (5H, m), 1.47 (9H, s), 1.5-2.6 (9H, m), 3.05-3.2 (2H, m), 3.4-3.65 (1H, m), 3.75 (1H, s), 4.15-4.4 (1H, m) MASS (m/z): 281 (M⁺+H)

Preparation 66

### Methyl 4′-(5-cyclohexyl-2,5-diazabicyclo[2.2.1]hept-2-yl)-1,1′-biphenyl-4-carboxylate

NMR (CDCl₃, δ): 1.0-1.35 (5H, m), 1.4-2.3 (8H, m), 2.45-2.6 (1H, m), 3.25-3.5 (3H, m), 3.8-4.0 (4H, m), 4.27 (1H, s), 6.64 (2H, d, J=8.7 Hz), 7.54 (2H, d, J=8.7 Hz), 7.62 (2H, d, J=8.4 Hz), 7.05 (2H, d, J=8.4 Hz) MASS (m/z): 391 (M⁺+H)

Preparation 67

### Methyl 4′-[4-(cis-4-methylcyclohexyl)-1-piperazinyl]-1,1′-biphenyl-4-carboxylate

NMR (CDCl₃, δ): 0.95 (3H, d, J=6.9 Hz), 1.4-1.85 (9H, m), 2.1-2.3 (1H, m), 2.65-2.8 (4H, m), 3.2-3.35 (4H, m), 3.93 (3H, s), 7.00 (2H, d, J=8.8 Hz), 7.56 (2H, d, J=8.8 Hz), 7.62 (2H, d, J=8.5 Hz), 8.06 (2H, d, J=8.5 Hz) MASS (m/z): 393 (M⁺+H)

Preparation 68

### Methyl 4′-[4-(trans-4-methylcyclohexyl)-1-piperazinyl]-1,1′-biphenyl-4-carboxylate

NMR (CDCl₃, δ): 0.8-1.45 (8H, m), 1.7-2.05 (4H, m), 2.2-2.4 (1H, m), 2.7-2.8 (4H, m), 3.2-3.35 (4H, m), 3.92 (3H, s), 6.99 (2H, d, J=8.8 Hz), 7.55 (2H, d, J-8.8 Hz), 7.62 (2H, d, J=8.4 Hz), 8.06 (2H, d, J=8.4 Hz) MASS (m/z): 393 (M⁺+H)

Preparation 69

### Methyl 4′-[4-4-[4-(6-methoxyhexyl)-1-piperazinyl]phenyl-1-piperazinyl]-1,1′-biphenyl-4-carboxylate

NMR (CDCl₃, δ): 1.2-2.7 (8H, m), 2.3-2.7 (5H, m), 3.05-3.5 (14H, m), 3.33 (3H, s), 3.93 (3H, s), 6.8-7.1 (6H, m), 7.5-7.7 (4H, m), 8.07 (2H, d, J=8.4 Hz) MASS (m/z): 571 (M⁺+H)

Preparation 70

### Methyl 4′-[4-4-[1-(6-methoxyhexyl)-4-piperidyloxy]phenyl-1-piperazinyl]-1,1′-biphenyl-4-carboxylate

NMR (CDCl₃, δ): 1.2-2.6 (16H, m), 2.7-2.9 (2H, m), 3.2-3.5 (13H, m), 3.93 (3H, s), 4.15-4.35 (1H, m), 6.8-7.0 (4H, m), 7.05 (2H, d, J=8.9 Hz), 7.5-7.7 (4H, m), 8.07 (2H, d, J=5 Hz) MASS (m/z): 586 (M⁺+H)

Preparation 71

### Methyl 4′-[4-4-[4-(6-methoxyhexyl)-1-piperazinyl]phenyl-1-piperazinyl]-1,1′-biphenyl-4-carboxylate

NMR (CDCl₃, δ): 1.2-2.7 (8H, m), 2.3-2.7 (5H, m), 3.05-3.5 (14H, m), 3.33 (3H, s), 3.93 (3H, s), 6.8-7.1 (6H, m), 7.5-7.7 (4H, m), 8.07 (2H, d, J=8.4 Hz) MASS (m/z): 571 (M⁺+H)

Preparation 72

To a stirred solution of 4-methylphenyl-p-toluenesulfonate (1.88 g) in DMF (20 ml) was added NaH (60% oil suspension; 405 mg) slowly at 0° C., and the suspension was stirred for 1 hour with warming to room temperature and for 1 hour at 65° C. 4-Hydroxy-1-tert-butoxycarbonyloxypiperidine (2 g) in DMF (10 ml) was added dropwise to the above solution and stirring was continued for 2 hours at this temperature. Water (10 ml) was added to the solution, and the whole was extracted with EtOAc, and the extract was washed with water and brine and dried over MgSO₄. Usual work up followed by flash chromatography (SiO₂; EtOAc:hexane=1:5) gave tert-butyl 4-(4-methylpentyloxy)-1-piperidinecarboxylate (2.09 g).

NMR (CDCl₃, δ): 0.88 (6H, d, J=6.6 Hz), 1.1-1.3 (3H, m), 1.4-1.7 (4H, m), 1.45 (9H, s), 1.7-1.9 (2H, m), 3.0-3.2 (2H, m), 3.3-3.5 (3H, m), 3.7-3.9 (2H, m) (+) APCI MASS (Positive): 186.20 (M⁺−Boc+H)

The following compounds [Preparation 73 and 74] were obtained according to a similar manner to that of Preparation 72.

Preparation 73

### Tert-Butyl 4-(methoxybutyloxymethyl)-1-piperidinecarboxylate

NMR (CDCl₃, δ): 1.45 (9H, s), 1.55-1.75 (4H, m), 2.60-2.80 (2H, m), 3.25 (2H, d, J=6.05 Hz), 3.28 (3H, s), 3.33-3.68 (4H, m), 4.00-4.10 (2H, m)

Preparation 74

### Tert-Butyl 4-(cyclohexylmethoxy)-1-piperidinecarboxylate

NMR (CDCl₃, δ): 0.8-1.9 (15H, m), 1.45 (9H, s), 3.0-3.2 (2H, m), 3.19 (2H, d, J=9.1 Hz), 3.3-3.5 (1H, m), 3.6-3.9 (2H, m) (+)APCI MASS (Positive): 198.33 (M⁺+Boc+H)

Preparation 75

A mixture of tert-butyl 1-piperazinecarboxylate (1.6 g), 5-methoxypentyl 4-methylbenzenesulfonate (2.8 g) and potassium carbonate (1.4 g) in dimethylformamide (16 ml) was stirred for 20.5 hours at room temperature. The reaction mixture was poured into a mixture of ethyl acetate and water. The organic layer was successively washed with water and brine and dried over magnesium sulfate. The solvent was evaporated in vacuo and the residue was purified by column chromatography on silica gel eluting with a mixture of dichloromethane and methanol (20:1). The eluted fractions containing the desired product were collected and evaporated in vacuo to give tert-butyl 4-(5-methoxypentyl)-1piperazinecarboxylate (1.73 g).

NMR (CDCl₃, δ): 1.3-1.7 (15H, m), 2.3-2.4 (6H, m), 3.3-3.5 (9H, m) (+)APCI MASS: 286.80 (M⁺+H)

The following compounds [Preparation 76 to 79] were obtained according to a similar manner to that of Preparation 75.

Preparation 76

### Ethyl 4-[4-(7-methoxyheptyl)-1-piperazinyl]benzoate

NMR (CDCl₃, δ): 1.3-1.7 (13H, m), 2.38 (2H, t, J=8.0 Hz), 2.58 (4H, t, J=5.0 Hz), 3.3-3.4 (9H, m), 4.33 (2H, q, J=7.2 Hz), 6.86 (2H, d, J=9.0 Hz), 7.92 (2H, d, J=9.0 Hz) (+) APCI MASS (Positive): 363.33 (M⁺+H)

Preparation 77

### 1-(4-Bromophenyl)-4-(6-methoxyhexyl)piperazine

NMR (CDCl₃, δ): 1.25-1.7 (8H, m), 2.38 (2H, t, J=7.6 Hz), 2.5-2.65 (4H, m), 3.1-3.2 (4H, m), 3.33 (3H, s), 3.37 (2H, t, J=6.5 Hz), 6.7-6.85 (2H, m), 7.25-7.4 (2H, m) MASS (m/z): 355, 357 (M⁺+H)

Preparation 78

### 4-(4-Bromophenoxy)-1-(6-methoxyhexyl)piperidine

NMR (CDCl₃, δ): 1.2-2.45 (16H, m), 2.65-2.8 (2H, m), 3.25-3.45 (5H, m), 4.2-4.35 (1H, m), 6.7-6.85 (2H, m), 7.3-7.4 (2H, m) MASS (m/z): 370, 372 (M⁺+H)

Preparation 79

### 1-(4-Bromophenyl)-4-(6-methoxyhexyl)piperazine

NMR (CDCl₃, δ): 1.25-1.7 (8H, m), 2.38 (2H, t, J=7.6 Hz), 2.5-2.65 (4H, m), 3.1-3.2 (4H, m), 3.33 (3H, s), 3.37 (2H, t, J=6.5 Hz), 6.7-6.85 (2H, m), 7.25-7.4 (2H, m) MASS (m/z): 355, 357 (M⁺+H)

Preparation 80

A solution of 4-hexyloxybenzeneboronic acid (1.15 g), 4-iodopyrazole (500 mg), pyridine (409.5 mg) and 4A° molecular sieves (powdered) (1.9 g) in methylene chloride (25 ml) was treated with anhydrous cuprous acetate (Cu(OAc)₂) and stirred 3 days at room temperature under an air atmosphere. The mixture was filtered then diluted with ethyl acetate, washed with saturated sodium chloride solution (×1), saturated sodium hydrogen carbonate solution (×1), saturated sodium chloride solution (×2), dried over magnesium sulfate and evaporated to afford a crude product that was purified by silica gel chromatography, eluting with 20:1 hexane-ethyl acetate to afford 1-(4-hexyloxyphenyl)-4-iodo-1H-pyrazole (1 g) as a white solid.

NMR (CDCl₃, δ): 0.8-1.0 (3H, m), 1.2-1.5 (6H, m), 1.73-1.83 (2H, m), 3.98 (2H, t, J=6.5 Hz), 6.95 (2H, d, J=9 Hz), 7.51 (2H, d, J=9 Hz), 7.68 (1H, s), 7.85 (1H, s) MASS (m/z): 371 (MH⁺)

The following compounds [Preparation 81 to 88] were obtained according to a similar manner to that of Preparation 80.

Preparation 81

### Ethyl 4-[4-(1,1′-biphenyl-4-yl)-1H-pyrazol-1-yl]benzoate

NMR (CDCl₃, δ): 1.43 (3H, t, J=7.1 Hz), 4.41 (2H, q, J=7.1 Hz), 7.32-7.50 (3H, m), 7.65 (4H, s), 7.61-7.71 (2H, m), 7.83 (2H, d, J=8.8 Hz), 8.08 (1H, s), 8.17 (2H, d, J=8.8 Hz), 8.27 (1H, s) MASS (m/z): 369 (MH⁺)

Preparation 82

### Ethyl 4-[4-(4-hexyloxyphenyl)-1H-pyrazol-1-yl]benzoate

NMR (CDCl₃, δ): 0.90-0.95 (3H, m), 1.20-1.57 (9H, m), 1.73-1.84 (2H, m), 3.99 (2H, t, J=6.5 Hz), 4.40 (2H, q, J=7.2 Hz), 6.94 (2H, d, J=8.6 Hz), 7.47 (2H, d, J=8.7 Hz), 7.80 (2H, d, J=7.2 Hz), 7.97 (1H, s), 8.15 (1H, s), 8.16 (2H, d, J-8.5 Hz) MASS (m/z): 393 (MH⁺)

Preparation 83

### Methyl 4-[1-4-(hexyloxyphenyl)-1H-pyrazol-4-yl]benzoate

NMR (CDCl₃, δ): 0.89-0.95 (3H, m), 1.2-1.5 (6H, m), 1.74-1.84 (2H, m), 3.93 (3H, s), 4.00 (2H, t, J=6.5 Hz), 6.98 (2H, d, J=9 Hz), 7.59-7.63 (4H, m), 8.01 (1H, s), 8.06 (2H, d, J-8.4 Hz), 8.14 (1H, s) MASS (m/z): 379 (MH⁺)

Preparation 84

### Methyl 4-[1-(8-methoxyoctyl)-4-piperidyloxy]benzoate

IR (Neat): 2927, 2856, 1720, 1605, 1508, 1458, 1437, 1309, 1282, 1244, 1169, 1117, 1103, 1043 cm⁻¹ NMR (DMSO-d₆, δ): 1.2-1.7 (14H, m), 1.9-2.1 (2H, m), 2.1-2.3 (4H, m), 2.6-2.8 (2H, m), 3.21 (3H, s), 3.29 (2H, t, J=6.4 Hz), 3.81 (3H, s), 4.4-4.6 (1H, m), 7.04 (2H, d, J=8.9 Hz), 7.88 (2H, d, J=8.8 Hz) ESI MASS (Positive): 378.3 (M⁺+H)

Preparation 85

### Methyl 4-[1-(7-methoxyheptyl)-4-piperidyl]benzoate

IR (Nujol): 1728, 1279, 1109 cm⁻¹ NMR (DMSO-d₆, δ): 1.2-1.8 (14H, m), 1.9-2.1 (2H, m), 2.2-2.4 (2H, m), 2.4-2.7 (1H, m), 2.9-3.1 (2H, m), 3.21 (3H, s), 3.2-3.4 (2H, m), 3.83 (3H, s), 7.40 (2H, d, J=8.3 Hz), 7.86 (2H, d, J=8.3 Hz) ESI MASS (Positive): 348.3 (M⁺+H)

Preparation 86

### 6-Methoxy-1-hexanol

NMR (CDCl₃, δ): 1.3-1.5 (4H, m), 1.5-1.7 (5H, m), 3.33 (3H, s), 3.3-3.5 (2H, m), 3.5-3.7 (2H, m)

Preparation 87

### Tert-Butyl 4-(7-methoxyheptyl)-1-piperazinecarboxylate

NMR (CDCl₃, δ): 1.2-1.6 (19H, m), 2.3-2.4 (6H, m), 3.33 (3H, s), 3.3-3.5 (6H, m) ESI MASS (Positive): 315.5 (M⁺+H)

Preparation 88

### 5-Methoxy-1-pentanol

NMR (CDCl₃, δ): 1.3-1.7 (6H, m), 3.3-3.5 (5H, m), 3.6-3.7 (2H, m)

Preparation 89

To a solution of 4-methoxybutanol (10 g) in a mixture of dichloromethane (100 ml), triethylamine (17.4 ml) and pyridine (20 ml) was added p-toluenesulfonyl chloride (20.1 g) and the mixture was stirred at ambient temperature overnight. The reaction mixture was concentrated in vacuo and dissolved in ethyl acetate (200 ml). The solution was washed in turn with 1N hydrochloric acid, water, saturated aqueous sodium hydrogen carbonate and saturated aqueous sodium chloride, dried over magnesium sulfate and evaporated in vacuo. The resulting residue was chromatographed on silica gel (200 ml) eluting with a mixture of n-hexane and ethyl acetate (2:1 v/v). The fractions containing the desired compound were collected and evaporated under reduced pressure to give 4-methoxybutyl-4-methylbenzenesulfonate (8.34 g).

NMR (CDCl₃, δ): 1.45-1.80 (5H, m), 2.45 (3H, s), 3.30 (3H, s), 3.36 (2H, t, J=6.10 Hz), 3.36 (2H, t, J=6.30 Hz), 7.34 (2H, d, J=8.21 Hz), 7.79 (2H, s, J=8.33 Hz) ESI MASS (Positive)(m/z): 281.2(M⁺+Na)

The following compounds [Preparation 90 to 93] were obtained according to a similar manner to that of Preparation 89.

Preparation 90

### 6-Methoxyhexyl 4-methylbenzenesulfonate

NMR (CDCl₃, δ): 1.2-1.8 (8H, m), 2.45 (3H, s), 3.31 (3H, s), 3.2-3.4 (2H, m), 4.02 (2H, t, J=6.4 Hz), 7.34 (2H, d, J=8.3 Hz), 7.79 (2H, d, J=8.3 Hz) ESI MASS (Positive): 309.3 (M⁺+Na)

Preparation 91

### 5-Methoxypentyl 4-methylbenzenesulfonate

NMR (CDCl₃, δ): 1.3-1.8 (6H, m), 2.45 (3H, s), 3.3-3.4 (5H, m), 4.02 (2H, t, J=6.4 Hz), 7.3-7.4 (2H, m), 7.7-7.9 (2H, m) ESI MASS (Positive): 295.2 (M⁺+H)

Preparation 92

### 4-Methylpentyl 4-methylbenzenesulfonate

NMR (CDCl₃, δ): 0.84 (6H, d, J=6.6 Hz), 1.1-1.3 (2H, m), 1.4-1.7 (3H, m), 2.45 (3H, s), 4.01 (2H, t, J=6.6 Hz), 7.34 (2H, d, J=8.0 Hz), 7.7-7.9 (2H, m) ESI MASS (Positive): 279.3 (M⁺+Na)

Preparation 93

### Cyclohexylmethyl 4-methylbenzenesulfonate

NMR (CDCl₃, δ): 0.7-1.8 (11H, m), 2.45 (3H, s), 3.81 (2H, d, J=6.0 Hz), 7.34 (2H, d, J=8.0 Hz), 7.7-7.8 (2H, m) ESI MASS (Positive): 291.3 (M⁺+Na)

Preparation 94

To a solution of tert-butyl 4-cyclohexyl-4-methoxy-1-piperidinecarboxylate (3.0 g) in a mixture of dichloromethane (60 ml) and anisole (7.67 ml) was dropwise added trifluoroacetic acid (15.5 ml) with stirring under ice-cooling. The mixture was stirred at ambient temperature for 1 hour and then concentrated in vacuo. The resulting residue was azeotropically distilled three times with toluene (50 ml) and dried in vacuo. The obtained residue was dissolved in DMSO (30 ml). To the solution were added 4-fluorobenzonitrile (1.47 g) and potassium carbonate (4.18 g) and the mixture was stirred at 140° C. for 4 hours. The reaction mixture was poured into water (100 ml) and extracted twice with ethyl acetate (100 ml). The extracts were collected, washed with saturated aqueous sodium chloride, dried over magnesium sulfate and evaporated in vacuo. The resulting residue was chromatographed on silica gel (200 ml) eluting with a mixture of n-hexane and ethyl acetate (8:1 v/v). The fractions containing the desired compound were collected and evaporated under reduced pressure to give 4-(4-cyclohexyl-4-methoxy-1-piperidyl)benzonitrile (3.08 g).

NMR (CDCl₃, δ): 0.85-1.45 (5H, m), 1.50-1.85 (10H, m), 3.00-3.20 (2H, m), 3.16 (3H, s), 3.50-3.70 (2H, m), 6.85 (2H, d, J=90.10 Hz), 7.46 (2H, d, J=9.08 Hz) ESI MASS (Positive)(m/z): 619.5 (2M++Na), 321.3 (M⁺+Na)

The following compounds [Preparation 95 to 97] were obtained according to a similar manner to that of Preparation 94.

Preparation 95

### 4-(4-Cyanophenyl)-1-(6-methoxyhexyl)piperazine

IR (KBr): 3560, 3392, 2935, 2856, 2212, 1603, 1516 cm⁻¹ NMR (CDCl₃, δ): 1.20-1.40 (4H, m), 1.40-1.70 (6H, m), 2.30-2.60 (2H, m), 2.50-2.60 (4H, m), 3.30-3.50 (4H, m), 3.33 (3H, s), 6.85 (2H, d, J=9.0 Hz), 7.48 (2H, d, J=9.0 Hz) MASS (m/z): 302 (M⁺+H)

Preparation 96

### 4-[4-(7-Methoxyheptyl)-1-piperazinyl]benzonitrile

IR (KBr): 2929, 2856, 2212, 1603, 1518, 1452, 1389, 1248, 1180, 1132, 1095, 924, 833 cm⁻¹ NMR (DMSO-d₆, δ): 1.2-1.6 (10H, m), 2.2-2.6 (6H, m), 3.20 (3H, s), 3.2-3.4 (6H, m), 7.01 (2H, d, J=9.0 Hz), 7.57 (2H, d, J=9.0 Hz) (+) APCI MASS: 316.07 (M⁺+H)

Preparation 97

### 4-[4-(5-Methoxypentyl)-1-piperazinyl]benzonitrile

IR (KBr): 2935, 2212, 1603, 1514, 1452, 1387, 1363, 1250, 1180, 1111, 947, 922, 825 cm⁻¹ NMR (DMSO-d₆, δ): 1.2-1.6 (6H, m), 2.2-2.5 (6H, m), 3.21 (3H, s), 3.3-3.4 (6H, m), 7.01 (2H, d, J=9.0 Hz), 7.57 (2H, d, J=9.0 Hz) (+) APCI MASS (m/z): 288.13 (M⁺+H)

Preparation 98

A solution of tert-butyl 4-(4-bromopentyloxymethyl)-1-piperidinecarboxylate (1.88 g) in a mixture of 28% sodium hydroxide in methanol (10.5 ml) and methanol (10 ml) was refluxed for 2 hours. The reaction mixture was concentrated in vacuo and was chromatographed on silica gel (200 ml) eluting with a mixture of n-hexane and ethyl acetate (4:1 v/v). The fractions containing the desired compound were collected and evaporated under reduced pressure to give tert-butyl 4-(5-methoxypentyloxymethyl)-1-piperidinecarboxylate (1.53 g).

NMR (CDCl₃, δ): 1.10-1.25 (2H, m), 1.30-1.45 (2H, m), 1.45 (9H, s), 1.50-1.80 (7H, m), 2.55-2.80 (2H, m), 3.24 (2H, d, J=6.07 Hz), 3.33 (3H, s), 3.34-3.45 (4H, m), 4.00-4.20 (2H, m) ESI MASS (Positive)(m/z): 338.4 (M⁺+Na)

The following compounds [Preparation 99 to 102] were obtained according to a similar manner to that of Preparation 98.

Preparation 99

### 4-(7-Methoxyheptyloxy)benzoic acid

NMR (DMSO-d₆, δ): 1.32-1.75 (10H, m), 3.30 (2H, t, J=6.4 Hz), 4.02 (2H, t, J=6.5 Hz), 6.99 (2H, d, J=6.4 Hz), 7.87 (2H, d, J=8.8 Hz) ESI MASS (Negative)(m/z): 265.4 (M⁺−H)

Preparation 100

### 8-(6-Methoxyhexyloxy)-1,4-dioxaspiro[4.5]decane

NMR (CDCl₃, δ): 1.25-1.90 (16H, m), 3.33 (3H, s), 3.35-3.45 (5H, m), 3.92 (4H, s) ESI MASS (Positive)(m/z): 295.4 (M⁺+Na)

Preparation 101

### 4-tert-Butoxycarbonyl-1-(6-methoxyhexyl)piperazine

NMR (CDCl₃, δ): 1.20-1.40 (4H, m), 1.45-1.70 (12H, m), 2.20-2.40 (6H, m), 3.33 (3H, s), 3.35-3.50 (6H, m) API-ES MASS (Positive): 323 (M⁺+Na), 301 (M⁺+H)

Preparation 102

### Methyl 4-[4-(7-methoxyheptyloxy)piperidin-1-yl]benzoate

NMR (CDCl₃, δ): 1.25-1.48 (6H, m), 1.48-1.78 (6H, m), 1.88-2.06 (2H, m), 3.00-3.20 (2H, m), 3.32 (3H, s), 3.27-3.54 (5H, m), 3.58-3.75 (2H, m), 3.86 (3H, s), 6.87 (2H, d, J=8.9 Hz), 7.90 (2H, d, J=9.1 Hz) MASS (m/z): 364 (M⁺+H)

Preparation 103

A mixture of ethyl 4-fluorobenzoate (7.88 g), 4-iodopyrazole (10 g) and potassium carbonate (7.11 g) in N,N-dimethylformamide (50 ml) was heated at 100° C. for 6 hours then cooled to room temperature. The mixture was diluted with ethyl acetate then washed with water (×5), brine, dried over magnesium sulfate, filtered and evaporated to give a crude product that was recrystallized from acetone-hexane to afford ethyl 4-(4-iodo-1H-pyrazol-1-yl)benzoate (2 crops, 6.5 g+4.6 g) as a light yellow solid.

NMR (CDCl₃, δ): 1.41 (3H, t, J=7.1 Hz), 4.40 (2H, q, J=7.1 Hz), 7.73 (2H, d, J=8.5 Hz), 7.75 (1H, s), 8.05 (1H, s), 8.14 (2H, d, J=8.5 Hz) MASS (m/z): 343 (MH⁺)

The following compounds [Preparation 104 to 111] were obtained according to a similar manner to that of Preparation 103.

Preparation 104

### Ethyl 4-[1-(8-bromooctyl)-4-piperidyloxy)benzoate

IR (Neat): 2931, 1713, 1605, 1508, 1277, 1252, 1169, 1105, 1043 cm⁻¹ NMR (DMSO-d₆, δ): 1.2-1.9 (17H, m), 1.9-2.1 (2H, m), 2.1-2.5 (2H, m), 2.6-2.9 (2H, m), 3.2-3.6 (2H, m), 3.53 (2H, t, J=6.7 Hz), 4.27 (2H, q, J=7.1 Hz), 4.4-4.6 (1H, m), 7.05 (2H, d, J=8.9 Hz), 7.88 (2H, d, J=8.8 Hz) ESI MASS (Positive): 440.2, 442.2 (M⁺+H)

Preparation 105

### Methyl 4-[1-(7-bromoheptyl)-4-piperidyl]benzoate

IR (Neat): 2933, 1720, 1281, 1111 cm⁻¹ NMR (DMSO-d₆, δ): 1.2-2.1 (16H, m), 2.2-2.4 (2H, m), 2.5-2.7 (1H, m), 2.9-3.1 (2H, m), 3.53 (2H, t, J=6.7 Hz), 3.83 (3H, s), 7.40 (2H, d, J=8.3 Hz), 7.89 (2H, d, J=8.3 Hz) ESI MASS (Positive): 396.3, 398.3 (M⁺+H)

Preparation 106

### 4-[1-(6-Methoxyhexyl)-4-piperidyl]benzonitrile

IR (Neat): 2937, 2858, 2227, 1608, 1504, 1466, 1450, 1379, 1119 cm⁻¹ NMR (DMSO-d₆, δ): 1.2-1.8 (12H, m), 1.8-2.1 (2H, m), 2.5-2.7 (1H, m), 2.9-3.0 (2H, m), 3.21 (3H, s), 3.2-3.4 (2H, m), 7.46 (2H, d, J=8.3 Hz), 7.75 (2H, d, J=8.3 Hz) ESI MASS (Positive): 301.4 (M⁺+H)

Preparation 107

### Tert-Butyl 4-(7-bromoheptyl)-1-piperazinecarboxylate

NMR (CDCl₃, δ): 1.2-1.6 (19H, m), 1.7-1.9 (2H, m), 2.3-2.4 (4H, m), 3.3-3.5 (6H, m) (+) APCI MASS: 362.60, 364.53 (M⁺+H)

Preparation 108

### Ethyl 4-(4-pentyloxypiperidin-1-yl)benzoate

IR (KBr): 2952.5, 1695.1, 1369.2, 1110.8 cm⁻¹ NMR (CDCl₃, δ): 0.90 (3H, t, J=6.8 Hz), 1.29-2.01 (13H, m), 3.04-3.72 (7H, m), 4.32 (2H, q, J=7.1 Hz), 6.84-6.89 (2H, m), 7.87-7.93 (2H, m) ESI MASS (Positive)(m/z): 320.40 (M⁺+H)

Preparation 109

### Tert-Butyl 4-(4-bromophenyl)-1-piperazinecarboxylate

NMR (CDCl₃, δ): 1.48 (9H, s), 3.05-3.15 (4H, m), 3.5-3.6 (4H, m), 6.79 (2H, d, J=9.0 Hz), 7.35 (2H, d, J=9.0 Hz) MASS (m/z): 340, 342 (M⁺+H)

Preparation 110

### Ethyl 4-[4-(4-methylpentyloxy)-1-piperidyl]benzoate

NMR (CDCl₃, δ): 0.89 (6H, d, J=6.6 Hz), 1.1-1.2 (3H, m), 1.36 (3H, t, J=7.1 Hz), 1.4-1.8 (4H, m), 1.8-2.1 (2H, m), 3.0-3.2 (2H, m), 3.4-3.6 (3H, m), 3.6-3.8 (2H, m), 4.32 (2H, q, J=7.1 Hz), 6.8-6.9 (2H, m), 7.8-8.0 (2H, m) (+) APCI MASS (Positive): 334.40 (M⁺+H)

Preparation 111

### Ethyl 4-[4-(cyclohexylmethoxy)-1-piperidyl]benzoate

NMR (CDCl₃, δ): 0.8-1.4 (6H, m), 1.36 (3H, t, J=7.2 Hz), 1.4-2.1 (9H, m), 3.0-3.2 (2H, m), 3.26 (2H, d, J=6.4 Hz), 3.3-3.5 (1H, m), 3.5-3.8 (2H, m), 4.32 (2H, q, J=7.2 Hz), 6.8-6.9 (2H, m), 7.8-8.0 (2H, m) (+) APCI MASS (Positive): 346.27(M⁺+H)

Preparation 112

To a mixture of 4-(1,2,3,6-tetrahydro-4-pyridyl)benzonitrile (1.0 g), cyclohexanone (1.1 ml) and acetic acid (0.93 ml) in methanol (10 ml) was added sodium cyanoborohydride (0.41 g). After stirring for 22 hours at room temperature, the solvent was evaporated in vacuo. The residue was poured into a mixture of ethyl acetate and water. The solution was adjusted to pH 10 with potassium carbonate. The organic layer was successively washed with brine and dried over magnesium sulfate. The solvent was evaporated in vacuo and the residue was purified by column chromatography on silica gel eluting with a mixture of dichloromethane and methanol (30:1). The eluted fractions containing the desired product were collected and evaporated in vacuo to give 4-(1-cyclohexyl-1,2,3,6-tetrahydro-4-pyridyl)benzonitrile (0.75 g).

IR (Neat): 2931, 2854, 2220, 1649, 1541, 1504, 1456 cm⁻¹ NMR (DMSO-d₆, δ): 1.0-1.9 (10H, m), 2.2-2.5 (3H, m), 2.7-2.8 (2H, m), 3.2-3.3 (2H, m), 6.3-6.5 (1H, m), 7.61 (2H, d, J=8.5 Hz), 7.78 (2H, d, J=8.5 Hz) (+) APCI MASS: 267.20 (M⁺+H)

The following compounds [Preparation 113 to 125] were obtained according to a similar manner to that of Preparation 112.

Preparation 113

### Ethyl 4-[4-[4-(6-methoxyhexyloxy)cyclohexyl]-1-piperidyl]benzoate

NMR (CDCl₃, δ): 1.20-2.10 (20H, m), 2.80-3.00 (4H, m), 3.33 (3H, s), 3.35-3.50 (9H, m), 4.34 (2H, q, J=7.10 Hz), 6.86 (2H, t, J=8.96 Hz), 7.93 (2H, d, J=8.83 Hz) ESI MASS (Positive)(m/z): 447.5 (M⁺+Na)

Preparation 114

### Ethyl 4-[4-(4-pyridylmethyl)-1-piperazinyl]benzoate

NMR (CDCl₃, δ): 1.37 (3H, t, J=7.1 Hz), 2.58-2.63 (0.4H, m), 3.32-3.37 (4H, m), 3.57 (2H, s), 4.32 (2H, q, J=7.1 Hz), 6.86 (2H, d, J=9 Hz), 7.31 (2H, d, J=5.9 Hz), 7.92 (2H, d, J=9 Hz), 8.56 (2H, d, J=5.9 Hz) APCI MASS (positive): 326.13 (MH⁺)

Preparation 115

### Ethyl 4-[4-(3,3-dimethyl-1,5-dioxaspiro[5.5]undec-9-yl)-1-piperazinyl]benzoate

NMR (CDCl₃, δ): 0.97 (6H, s), 1.33 (3H, t, J=7.1 Hz), 1.33-1.80 (5H, m), 2.0-2.5 (4H, m), 2.68-2.73 (4H, m), 3.29-3.34 (4H, m), 3.48-3.52 (4H, m), 4.32 (2H, q, J=7.1 Hz), 6.86 (2H, d, J=9 Hz), 7.92 (2H, d, J=9 Hz) APCI MASS: 417.13 (M⁺+H)

Preparation 116

### Ethyl 4-(4-cyclopentyl-1-piperazinyl)benzoate

NMR (CDCl₃, δ): 1.36 (3H, t, J=7.1 Hz), 1.40-2.0 (8H, m), 2.49-2.62 (1H, m), 2.62-2.67 (4H, m), 3.32-3.37 (4H, m), 4.32 (2H, q, J=7.1 Hz), 6.86 (2H, d, J=9 Hz), 7.92 (2H, d, J=9 Hz) API-ES MASS: 303.3 (M⁺+H)

Preparation 117

### Ethyl 4-(4-cycloheptyl-1-piperazinyl)benzoate

NMR (CDCl₃, δ): 1.36 (3H, t, J=7.1 Hz), 1.40-1.95 (12H, m), 2.50-2.70 (5H, m), 3.29-3.40 (4H, m), 4.32 (2H, q, J=7.1 Hz), 6.8.5 (2H, d, J=9 Hz), 7.91 (2H, d, J=9 Hz) APCI MASS: 331.27 (M⁺+H)

Preparation 118

### Ethyl 4-[4-(1,4-dioxaspiro[4.5]dec-8-yl)-1-piperazinyl]benzoate

NMR (CDCl₃, δ): 1.36 (3H, t, J-7.1 Hz), 1.50-2.1 (8H, m), 2.47-2.55 (1H, m), 2.70-2.75 (4H, m), 3.30-3.35 (4H, m), 3.95 (4H, s), 4.32 (2H, q, J=7.1 Hz), 6.86 (2H, d, J=9 Hz), 7.92 (2H, d, J=9 Hz) APCI MASS: 375.2 (M⁺+H)

Preparation 119

### Ethyl 4-(4-tetrahydro-2H-pyran-4-yl-1-piperazinyl)benzoate

NMR (CDCl₃, δ): 1.37 (3H, t, J=7.1 Hz), 1.5-2.0 (4H, m), 2.42-2.57 (1H, m), 2.68-2.73 (4H, m), 3.32-3.37 (4H, m), 3.60-4.20 (4H, m), 4.32 (2H, q, J=7.1 Hz), 6.86 (2H, d, J=9 Hz), 7.92 (2H, d, J=9 Hz) APCI MASS: 319.2 (M⁺+H)

Preparation 120

### 4-(1-Cyclohexyl-4-piperidyloxy)benzonitrile

IR (Nujol): 2214, 1601, 1504, 1298, 1255, 1171, 1043 cm⁻¹ NMR (DMSO-d₆, δ): 0.9-1.4 (6H, m), 1.5-2.1 (9H, m), 2.2-2.6 (2H, m), 2.6-2.9 (2H, m), 4.4-4.6 (1H, m), 7.0-7.2 (2H, m), 7.6-7.8 (2H, m) ESI MASS (Positive): 285.3 (M⁺+H)

Preparation 121

### 4-[1-[4-(6-Methoxyhexyloxy)cyclohexyl]-4-piperidyloxy]benzonitrile

NMR (CDCl₃, δ): 1.2-3.2 (24H, m), 3.33 (3H, s), 3.3-3.5 (6H, m), 4.3-4.5 (1H, m), 6.93 (2H, d, J=8.5 Hz), 7.5-7.7 (2H, m) (+) APCI MASS (Positive): 415.40 (M⁺+H)

Preparation 122

### Ethyl 4-[1-[4-(6-methoxyhexyloxy)cyclohexyl]-4-piperidyloxy]benzoate

NMR (CDCl₃, δ): 1.2-3.0 (23H, m), 3.33 (3H, s), 3.3-3.5 (10H, m), 4.34 (2H, q, J=7.1 Hz), 4.3-4.5 (1H, m), 6.90 (2H, d, J=8.9 Hzl), 7.97 (2H, d, J=8.8 Hz) (+) APCI MASS (Positive): 462.53 (M⁺+H)

Preparation 123

### Methyl 4-[1-[4-(6-methoxyhexyloxy)cyclohexyl]-4-piperidyl]benzoate

NMR (CDCl₃, δ): 1.2-2.7 (25H, m), 3.0-3.3 (2H, m), 3.33 (3H, s), 3.3-3.5 (4H, m), 3.90 (3H, s), 7.29 (2H, d, J=8.4 Hz), 7.96 (2H, d, J=8.3 Hz) (+) APCI MASS (Positive): 432.27 (M⁺+H)

### Methyl 4-[1-[4-(6-methoxyhexyloxy)cyclohexyl]-4-piperidyl]benzoate

NMR (CDCl₃, δ): 1.2-2.1 (20H, m), 2.2-2.6 (5H, m), 3.0-3.2 (2H, m), 3.33 (3H, s), 3.3-3.5 (4H, m), 3.89 (3H, s), 7.29 (2H, d, J=8.3 Hz), 7.9-8.0 (2H, m) (+) APCI MASS (Positive): 432.47 (M⁺+H), ESI MASS (Positive): 432.47 (M⁺+H)

Preparation 124

### Ethyl 4-(1-cyclohexyl-4-piperidyloxy)benzoate

IR (Nujol): 1701, 1601, 1504, 1311, 1248, 1163, 1105, 1039 cm⁻¹ NMR (DMSO-d₆, δ): 1.0-1.4 (9H, m), 1.5-2.1 (9H, m), 2.2-2.5 (2H, m), 2.7-2.9 (2H, m), 4.27 (2H, q, J=7.1 Hz), 4.4-4.6 (1H, m), 7.04 (2H, d, J=8.9 Hz), 7.88 (2H, d, J=8.9 Hz) ESI MASS (Positive): 332.4 (M⁺+H)

Preparation 125

### Methyl 4′-[4-[cis-4-methoxy-4-(1-methoxycyclohexyl-1-yl)cyclohexyl-1-yl]-1-piperazinyl]-1,1′-biphenyl-4-carboxylate

NMR (CDCl₃, δ): 1.0-2.4 (19H, m), 2.7-2.85 (4H, m), 3.25-3.35 (4H, m), 3.43 (3H, s), 3.44 (3H, s), 3.93 (3H, s), 7.00 (2H, d, J=8.9 Hz), 7.5-7.7 (4H, m), 8.0-8.1 (2H, m) MASS (m/z): 521 (M⁺+H)

### Methyl 4′-[4-[trans-4-methoxy-4-(1-methoxycyclohexyl-1-yl)cyclohexyl-1-yl]-1-piperazinyl]-1,1′-biphenyl-4-carboxylate

NMR (CDCl₃, δ): 0.8-2.3 (19H, m), 2.55-2.7 (4H, m), 3.2-3.35 (4H, m), 3.42 (3H, s), 3.43 (3H, s), 3.93 (3H, s), 7.00 (2H, d, J=8.9 Hz), 7.5-7.7 (4H, m), 8.0-8.15 (2H, m) MASS (m/z): 521 (M⁺+H)

Preparation 126

A mixture of 4-[1-(4-methoxyphenyl)-4-piperidyloxy]benzonitrile (0.59 g), thiosemicarbazide (0.44 g) and trifluoroacetic acid (3 ml) in toluene (6 ml) was stirred for 6 hours at 70° C. After being cooled to room temperature, the solvent was evaporated in vacuo. Then the residue was dissolved in tetrahydrofuran and poured into water. The solution was adjusted to pH 9 with stirring. The resulting precipitate was collected by filtration and washed with water and isopropyl ether to give 5-4-[1-(4-methoxyphenyl)-4-piperidyloxy]phenyl-1,3,4-thiadiazol-2-amine (0.71 g).

IR (KBr): 3099, 1606, 1518, 1466, 1294, 1248, 1180, 1036 cm⁻¹ NMR (DMSO-d₆, δ): 1.6-1.9 (2H, m), 2.0-2.2 (2H, m), 2.8-3.0 (2H, m), 3.3-3.4 (2H, m), 3.68 (3H, s), 4.5-4.7 (1H, m), 6.6-7.0 (4H, m), 7.07 (2H, d, J=8.8 Hz), 7.29 (2H, s), 7.67 (2H, d, J=8.8 Hz) ESI MASS (Positive): 383.3 (M⁺+H)

The following compounds [Preparation 127 to 137] were obtained according to a similar manner to that of Preparation 126.

Preparation 127

### 2-Amino-5-[4-[4-(4-methoxybutyloxymethyl)piperidin-1-yl]phenyl]-1,3,4-thiadiazole

NMR (CDCl₃+CD₃OD, δ): 1.30-1.50 (2H, m), 1.50-1.80 (6H, m), 1.90-2.10 (2H, m), 2.90-3.10 (2H, m), 3.34 (3H, s), 3.35-3.70 (7H, m), 6.93 (2H, d, J=8.91 Hz), 7.63 (2H, d, J=8.83 Hz) APCI MASS (m/z): 377 (M⁺)

Preparation 128

### 2-Amino-5-[4-(6-methoxyhexyl)piperazin-1-yl]phenyl]-1,3,4-thiadiazole

IR (KBr): 3491, 3290, 3134, 2931, 1606, 1518 cm⁻¹ NMR (DMSO-d₆, δ): 1.20-1.40 (4H, m), 1.40-1.60 (4H, m), 2.50-2.70 (4H, m), 3.22 (3H, s), 3.20-3.50 (8H, m), 7.00 (2H, d, J=8.9 Hz), 7.22 (2H, br s), 7.57 (2H, d, J=8.7 Hz) MASS: 376 (M⁺+H)

Preparation 129

### 5-[4-[1-(6-Methoxyhexyl)-4-piperidyl]phenyl]-1,3,4-thiadiazol-2-amine trifluoroacetate

IR (KBr): 3277, 3166, 2933, 2858, 1701, 1687, 1630, 1516, 1203, 1171, 1119 cm⁻¹ NMR (DMSO-d₆, δ): 1.2-2.0 (14H, m), 2.5-2.8 (5H, m), 3.22 (3H, s), 3.3-3.4 (2H, m), 7.3-7.4 (4H, m), 7.70 (2H, d, J=8.2 Hz) (+) APCI MASS: 375.13 (M⁺+H)

Preparation 130

### 5-[4-[4-(7-Methoxyheptyl)-1-piperazinyl]phenyl]-1,3,4-thiadiazol-2-amine trifluoroacetate

IR (KBr): 3277, 3114, 2931, 1606, 1512, 1466, 1238, 1120 cm⁻¹ NMR (DMSO-d₆, δ): 1.2-1.6 (10H, m), 2.3-2.6 (6H, m), 3.21 (3H, s), 3.2-3.4 (6H, m), 6.98 (2H, d, J=8.8 Hz), 7.21 (2H, s), 7.57 (2H, d, J=8.8 Hz) (+) APCI MASS: 390.56 (M⁺+H)

Preparation 131

### 5-[4-[4-(5-Methoxypentyl)-1-piperazinyl]phenyl]-1,3,4-thiadiazol-2-amine trifluoroacetate

IR (KBr): 3115, 2939, 1606, 1520, 1466, 1325, 1240, 1192, 1122, 1034, 824 cm⁻¹ NMR (DMSO-d₆, δ): 1.2-1.6 (6H, m), 2.2-2.6 (6H, m), 3.21 (3H, s), 3.2-3.4 (6H, m), 6.98 (2H, d, J=8.8 Hz), 7.20 (2H, s), 7.56 (2H, d, J=8.8 Hz), 8.05 (1H, s) (+) APCI MASS: 362.00 (M⁺+H)

Preparation 132

### 5-[4-(1-Phenyl-4-piperidyloxyphenyl]-1,3,4-thiadiazol-2-amine

IR (KBr): 3269, 3097, 1603, 1518, 1468, 1246 cm⁻¹ NMR (DMS-d₆, δ): 1.6-1.9 (2H, m), 2.0-2.2 (2H, m), 3.0-3.2 (2H, m), 3.4-3.6 (2H, m), 4.6-4.7 (1H, m), 6.76 (1H, t, J=7.2 Hz), 6.9-7.4 (8H, m), 7.67 (2H, d, J=8.8 Hz) ESI MASS (Positive): 727.2 (2M++Na)

Preparation 133

### 5-[4-(1-Cyclohexyl-1,2,3,6-tetrahydro-4-pyridyl)phenyl]-1,3,4-thiadiazol-2-amine trifluoroacetate

IR (KBr): 3155, 2945, 1682, 1504, 1205, 1132 cm⁻¹ NMR (DMSO-d₆, δ): 1.0-2.2 (10H, m), 2.7-2.9 (2H, m), 3.1-3.5 (2H, m), 3.6-3.8 (1H, m), 3.8-4.0 (2H, m), 6.2-6.4 (1H, m), 7.46 (2H, s), 7.60 (2H, d, J=8.5 Hz), 7.77 (2H, d, J=8.5 Hz), 9.72 (1H, br s) (+) APCI MASS: 340.93 (M⁺+H)

Preparation 134

### 5-[4-(1-Cyclohexyl-4-piperidyloxy)phenyl]-1,3,4-thiadiazol-2-amine

NMR (CDCl₃, δ): 1.0-3.0 (19H, m), 4.3-4.5 (1H, m), 5.2-5.4 (2H, br s), 6.8-7.0 (2H, m), 7.4-7.6 (2H, m) (+) APCI MASS (Positive): 359.27 (M⁺+H)

Preparation 135

### 5-4-[1-[4-(6-Methoxyhexyloxy)cyclohexyl]-4-piperidyloxy]phenyl-1,3,4-thiadiazol-2-amine

NMR (CDCl₃₁ 6): 1.2-3.2 (24H, m), 3.33 (3H, s), 3.3-3.5 (6H, m), 4.3-4.5 (1H, m), 5.2-5.3 (2H, br s), 6.93 (2H, d, J=8.8 Hz), 7.71 (2H, d, J=8.6 Hz) (+) APCI MASS (Positive): 489.47 (M⁺+H)

Preparation 136

### 5-4-[4-(5-Methoxypentyloxymethyl)piperidin-1-yl]phenyl-1,3,4-thiadiazol-2-ylamine

NMR (CDCl₃, δ): 1.20-2.00 (11H, m), 2.65-2.90 (2H, m), 3.27 (2H, d, J=6.04 Hz), 3.33 (3H, s), 3.36 (2H, d, J=6.58 Hz), 3.44 (2H, d, J=6.43 Hz), 3.70-3.90 (2H, m), 5.52 (2H, br s), 6.90 (2H, d, J=8.93 Hz), 7.64 (2H, d, J=8.84 Hz) ESI MASS (Positive)(m/z): 413.3 (M⁺+Na)

Preparation 137

### 5-[4-(4-Cyclohexyl-4-methoxy-1-piperidyl)phenyl]-1,3,4-thiadiazol-2-amine

NMR (DMSO-d₆, δ): 0.8-2.0 (15H, m), 2.8-3.0 (2H, m), 3.08 (3H, s), 3.5-3.7 (2H, m), 6.9-7.6 (6H, m) (+) APCI MASS: 373.27 (M⁺+H)

Preparation 138

A mixture of phenyl 4-[4-(ethoxycarbonyl)phenyl]-1-piperazine carboxylate (13 g) and hydrazine monohydrate (49 ml) in a mixture of ethanol (130 ml) and tetrahydrofuran (65 ml) was refluxed for 24 hours. The reaction mixture was poured into water and the resulting precipitate was collected, washed with water, and dried to give ethyl 4-[4-(hydrazinocarbonyl)-1-piperazinyl]benzoate (6.28 g).

IR (KBr): 3365.2, 2987.2, 1699.0, 1633.4, 1608.3, 1240.0 cm⁻¹ NMR (CDCl₃, δ): 1.37 (3H, t, J=7.1 Hz), 3.33-3.59 (8H, m), 3.83 (2H, br s), 4.34 (2H, q, J=7.1 Hz), 5.84 (1H, s), 6.81-6.87 (2H, m), 7.91-7.98 (2H, m) ESI MASS (Positive)(m/z): 315.23 (M⁺+Na)

The following compounds [Preparation 139 to 160]were obtained according to a similar manner to that of Preparation 138.

Preparation 139

### 4-[4-[4-(6-Methoxyhexyloxy)cyclohexyl]-1-piperidyl]benzohydrazide (515 mg)

NMR (CDCl₃, δ): 1.15-1.70 (16H, m), 1.80-2.40 (4H, m), 2.65-2.75 (4H, m), 3.20-3.50 (12H, m), 4.05 (2H, md, J=3.75 Hz), 6.88 (2H, d, J=8.92 Hz), 7.20-7.30 (1H, br s), 7.65 (2H, d, J=8.86 Hz) ESI MASS (Positive)(m/z): 455.4 (M⁺+Na), 433.5 (M⁺+H)

Preparation 140

### 4-[1-(8-Methoxyoctyl)-4-piperidyloxy]benzohydrazide

IR (Nujol): 3290, 3275, 1626, 1500, 1325, 1255, 1119, 1036 cm⁻¹ NMR (DMSO-d₆, δ): 1.2-1.7 (14H, m), 1.8-2.0 (2H, m), 2.1-2.3 (4H, m), 2.6-2.8 (2H, m), 3.20 (3H, s), 3.29 (2H, t, J=6.4 Hz), 4.3-4.5 (3H, m), 6.97 (2H, d, J=8.8 Hz), 7.77 (2H, d, J=8.8 Hz), 9.58 (1H, s) ESI MASS (Positive): 378.3 (M⁺+H)

Preparation 141

### 4-[1-(7-Methoxyheptyl)-4-piperidyl]benzohydrazide

IR (Nujol): 3325, 1624, 1524, 1122 cm⁻¹ NMR (DMSO-d₆, δ): 1.2-1.8 (14H, m), 1.8-2.0 (2H, m), 2.2-2.3 (2H, m), 2.4-2.7 (1H, m), 2.8-3.0 (2H, m), 3.21 (3H, s), 3.2-3.4 (2H, m), 4.44 (2H, br s), 7.30 (2H, d, J=8.2 Hz), 7.74 (2H, d, J=8.2 Hz), 9.67 (1H, s) ESI MASS (Positive): 348.5 (M⁺+H)

Preparation 142

### 4-[4-(4-Pyridylmethyl)-1-piperazinyl]benzohydrazide

NMR (DMSO-d₆, δ): 2.49-2.54 (4H, m), 3.24-3.28 (4H, m), 3.57 (2H, s), 4.35 (2H, s), 6.92 (2H, d, J=8.9 Hz), 7.35 (2H, d, J=5.9 Hz), 7.70 (2H, d, J=8.9 Hz), 8.52 (2H, d, J=5.9 Hz), 9.47 (1H, S) APCI MASS (Positive): 312 (M⁺+H)

Preparation 143

### 4-(1,4-Dioxa-8-azaspiro[4.5]dec-8-yl)benzohydrazide

NMR (DMSO-d₆, δ): 1.64-1.70 (4H, m), 3.35-3.41 (4H, m), 3.91 (4H, s), 4.35 (2H, s), 6.94 (2H, d, J=8.9 Hz), 7.68 (2H, d, J=8.9 Hz), 9.45 (1H, s) APCI MASS: 278.13 (M⁺+H)

Preparation 144

### 4-[4-(3,3-Dimethyl-1,5-dioxaspiro[5.5]undec-9-yl)-1-piperazinyl]benzohydrazide

NMR (DMSO-d₆, δ): 0.89 (6H, s), 1.20-1.70 (5H, m), 2.10-2.40 (4H, m), 2.58 (4H, br s), 3.20 (4H, br s), 3.41-3.43 (4H, m), 4.36 (2H, s), 6.91 (2H, d, J=8.9 Hz), 7.69 (2H, d, J=8.9 Hz), 9.46 (1H, s) API-EI MASS: 403.3 (M⁺+H)

Preparation 145

### 4-(4-Cyclopentyl-1-piperazinyl)benzohydrazide

NMR (DMSO-d₆, δ): 1.20-1.90 (8H, m), 2.4-2.6 (5H, m), 3.19-3.24 (4H, m), 4.36 (2H, s), 6.91 (2H, d, J=8.9 Hz), 7.69 (2H, d, J=8.9 Hz), 9.46 (1H, s) APCI MASS: 289.2 (M⁺+H)

Preparation 146

### 4-(4-Cycloheptyl-1-piperazinyl)benzohydrazide

NMR (DMSO-d₆, δ): 1.3-1.8 (12H, m), 2.5-2.65 (5H, m), 3.10-3.25 (4H, m), 4.35 (2H, s), 6.90 (2H, d, J=8.9 Hz), 7.69 (2H, d, J=8.9 Hz), 9.45 (1H, s) APCI MASS: 317.27 (M⁺+H)

Preparation 147

### 4-[4-(1,4-Dioxaspiro[4.5]dec-8-yl)-1-piperazinyl]benzohydrazide

NMR (DMSO-d₆, δ): 1.40-1.75 (8H, m), 2.22-2.45 (1H, m), 2.57-2.61 (4H, m), 3.18-3.23 (4H, m), 3.84 (4H, s), 4.35 (2H, s), 6.91 (2H, d, J=8.9 Hz), 7.69 (2H, d, J=8.9 Hz), 9.46 (1H, s) APCI MASS: 361.27 (M⁺+H)

Preparation 148

### 4-(4-Tetrahydro-2H-pyran-4-yl-1-piperazinyl)benzohydrazide

NMR (DMSO-d₆, δ): 1.30-1.50 (2H, m), 1.71-1.77 (2H, m), 2.30-2.50 (₁H, m), 2.58-2.63 (4H, m), 3.20-3.40 (6H, m), 3.86-3.91 (2H, m), 4.38 (2H, s), 6.92 (2H, d, J=8.9 Hz), 7.69 (2H, d, J=8.9 Hz), 9.46 (1H, s) APCI MASS: 305.13 (M⁺+H)

Preparation 149

### 4-(1-Phenyl-4-piperidyloxy)benzohydrazide

IR (KBr): 3261, 1601, 1498, 1250 cm⁻¹ NMR (DMSO-d₆, δ): 1.6-1.8 (2H, m), 2.0-2.2 (2H, m), 2.9-3.1 (2H, m), 3.4-3.6 (2H, m), 4.43 (2H, s), 4.6-4.7 (1H, m), 6.7-7.3 (7H, m), 7.79 (2H, d, J=8.7 Hz), 9.61 (1H, s) ESI MASS (Positive): 645.2 (2M++Na)

Preparation 150

### 4-[1-(4-Methoxyphenyl)-4-piperidyloxy]benzohydrazide

IR (KBr): 3319, 1606, 1510, 1254, 1190, 1119, 1036 cm⁻¹ NMR (DMSO-d₆, δ): 1.6-1.8 (2H, m), 2.0-2.2 (2H, m), 2.8-3.0 (2H, m), 3.2-3.4 (2H, m), 3.68 (3H, s), 4.41 (2H, s), 4.5-4.7 (1H, m), 6.7-7.0 (4H, m), 7.02 (2H, d, J=8.8 Hz), 7.78 (2H, d, J=8.8 Hz), 9.60 (1H, s) ESI MASS (Positive): 705.4 (2M++Na)

Preparation 151

### 4-[1-[4-(6-Methoxyhexyloxy)cyclohexyl]-4-piperidyloxy]benzohydrazide

This compound was used in the next reaction without further purification.

Preparation 152

### 4-[1-[4-(6-Methoxyhexyloxy)cyclohexyl]-4-piperidyl]benzohydrazide

NMR (CDCl₃, δ): 1.2-1.7 (12H, m), 1.7-2.2 (8H, m), 2.3-2.7 (5H, m), 3.0-3.3 (2H, m), 3.33 (3H, s), 3.3-3.5 (4H, m), 4.0 (1H, br s), 7.29 (2H, d, J=8.8 Hz), 7.46 (2H, br s), 7.68 (2H, d, J=8.3 Hz) (+) APCI MASS (Positive): 432.27 (M⁺+H)

Preparation 153

### 4-[1-[4-(6-Methoxyhexyloxy)cyclohexyl]-4-piperidyl]benzohydrazide

NMR (CDCl₃, δ): 1.2-2.1 (21H, m), 2.2-2.6 (3H, m), 3.0-3.2 (2H, m), 3.33 (3H, s), 3.3-3.5 (5H, m), 3.9-4.2 (1H, br s), 7.30 (2H, d, J=8.3 Hz), 7.41 (2H, br s), 7.67 (2H, d, J=8.3 Hz) (+) APCI MASS (Positive): 432.40 (M⁺+H)

Preparation 154

### 4-(1-Cyclohexyl-4-piperidyloxy)benzohydrazide

NMR (CDCl₃, δ): 1.0-1.4 (6H, m), 1.5-2.1 (8H, m), 2.2-2.6 (3H, m), 2.8-3.0 (2H, m), 3.8-4.2 (3H, br s), 4.2-4.4 (1H, m), 6.91 (2H, d, J=6.9 Hz), 7.6-7.8 (2H, m) (+) APCI MASS (Positive): 318.27 (M⁺+H)

Preparation 155

### 4-[4-(7-Methoxyheptyl)-1-piperazinyl]benzohydrazide

NMR (CDCl₃, δ): 1.2-1.7 (12H, m), 2.38 (2H, t, J=8.0 Hz), 2.58 (4H, t, J=5.2 Hz), 3.2-3.5 (4H, m), 3.33 (3H, s), 4.06 (2H, br s), 6.88 (2H, d, J=9.0 Hz), 7.68 (1H, br s), 7.92 (2H, d, J=9.0 Hz) (+) APCI MASS (Positive): 349.40 (M⁺+H)

Preparation 156

### 4-[4-(7-Methoxyheptyloxy)piperidin-1-yl]benzoylhydrazine

NMR (CDCl₃, δ): 1.22-1.46 (6H, m), 1.46-1.78 (6H, m), 1.88-2.04 (2H, m), 2.98-3.17 (2H, m), 3.33 (3H, s), 3.29-3.55 (5H, m), 3.55-3.72 (2H, m), 4.06 (2H, s), 6.89 (2H, d, J=9.0 Hz), 7.29 (1H, s), 7.64 (2H, d, J=9.0 Hz) MASS (m/z): 364 (M⁺+H)

Preparation 157

### 4-(4-Pentyloxypiperidin-1-yl)benzohydrazide

IR (KBr): 3274.5, 2937.1, 1608.3, 1108.9 cm⁻¹ NMR (CDCl₃, δ): 0.90 (3H, t, J=6.8 Hz), 1.29-2.01 (12H, m), 3.01-4.05 (7H, m), 6.86-6.91 (2H, m), 7.32 (1H, s), 7.62-7.66 (2H, m) ESI MASS (Positive)(m/z): 306.20 (M⁺+H)

Preparation 158

### 4-(4-Butoxypiperidin-1-yl)benzohydrazide

IR (KBr): 3270.7, 2952.5, 1606.4, 1103.1 cm⁻¹ NMR (CDCl₃, δ): 0.93 (3H, t, J=7.2 Hz), 1.33-2.03 (10H, m), 3.01-4.06 (7H, m), 6.86-6.92 (2H, m), 7.36 (1H, s), 7.61-7.68 (2H, m) ESI MASS (Positive)(m/z): 292.2 (M⁺+H)

Preparation 159

### 4-[4-(4-Methylpentyloxy)-1-piperidyl]benzohydrazide

NMR (CDCl₃, δ): 0.89 (6H, d, J=6.6 Hz), 1.1-1.3 (3H, m), 1.4-1.8 (4H, m), 1.8-2.1 (2H, m), 3.0-3.2 (2H, m), 3.4-3.5 (1H, m), 3.45 (2H, t, J-6.8 Hz), 3.5-3.7 (2H, m), 4.06 (2H, br s), 6.8-7.0 (2H, m), 7.33 (1H, br s), 7.5-7.7 (2H, m) (+) APCI MASS (Positive): 320.40 (M⁺+H)

Preparation 160

### 4-[4-(Cyclohexylmethoxy)-1-piperidyl]benzohydrazide

NMR (CDCl₃, δ): 0.8-2.1 (15H, m), 3.0-3.2 (2H, m), 3.26 (2H, d, J=6.4 Hz), 3.3-3.8 (3H, m), 4.07 (2H, br s), 6.88 (2H, d, J=8.9 Hz), 7.35 (1H, br s), 7.5-7.8 (2H, m) (+) APCI MASS (Positive): 332.40 (M⁺+H)

Preparation 161

To a solution of ethyl 4-(1-piperazinyl)benzoate (10 g) and pyridine (6.36 ml) in tetrahydrofuran (150 ml) was added dropwise phenylchloroformate (7.35 g) with stirring under ice-cooling, and the mixture was stirred at the ambient temperature for overnight. The reaction mixture was added water (750 ml) and the resulting precipitate collected, and dried to give phenyl 4-[4-(ethoxycarbonyl)phenyl]-1-piperazine carboxylate (13.49 g).

IR (KBr): 2987.2, 1724.0, 1697.1, 1290.1 cm⁻¹ NMR (CDCl₃, δ): 1.38 (3H, t, J=7.1 Hz), 3.38-3.43 (4H, m), 3.78 (4H, br s), 4.34 (2H, q, J=7.1 Hz), 6.90 (2H, d, J=9.0 Hz), 7.09-7.42 (7H, m) ESI MASS (Positive)(m/z): 355.0 (M⁺+H)

The following compounds [Preparation 162 to 183] were obtained according to a similar manner to that of Preparation 161.

Preparation 162

### Methyl 4-[2-[4-4-[4-(6-methoxyhexyloxy)cyclohexyl-1-piperidyl]benzoyl]hydrazinocarbonyl]benzoate

NMR (DMSO-d₆, δ): 1.00-1.55 (14H, m), 1.70-2.35 (4H, m), 2.55-2.65 (4H, m), 3.21 (3H, s), 3.22-3.45 (8H, m), 3.89 (3H, s), 6.98 (2H, d, J=8.88 Hz), 7.80 (2H, d, J=8.72 Hz), 8.00-8.15 (4H, m), 10.26 (1H, s), 10.57 (1H, s) ESI MASS (Positive)(m/z): 617.4 (M⁺+Na), 595.4 (M⁺+H)

Preparation 163

### Methyl 4-[2-[4-[1-(8-methoxyoctyl)-4-piperidyloxy]benzoyl]hydrazinocarbonyl]benzoate

IR (Nujol): 3213, 1720, 1684, 1653, 1281 cm⁻¹ NMR (DMSO-d₆, δ): 1.2-2.3 (20H, m), 2.9-3.4 (4H, m), 3.22 (3H, s), 3.90 (3H, s), 4.77 (1H, br s), 7.14 (2H, d, J=8.8 Hz), 7.92 (2H, d, J=8.8 Hz), 8.0-8.2 (4H, m) ESI MASS (Positive): 540.5 (M⁺+H)

Preparation 164

### Methyl 4-[2-[4-[1-(7-methoxyheptyl)-4-piperidyl]benzoyl]hydrazinocarbonyl)benzoate

NMR (CDCl₃, δ): 1.2-1.4 (6H, m), 1.4-1.7 (4H, m), 1.7-1.9 (4H, m), 2.0-2.2 (2H, m), 2.3-2.7 (3H, m), 3.0-3.2 (2H, m), 3.33 (3H, s), 3.3-3.4 (2H, m), 3.94 (3H, s), 4.90 (2H, br s), 7.26 (2H, d, J=8.3 Hz), 7.77 (2H, d, J=8.2 Hz), 7.89 (2H, d, J=8.5 Hz), 8.05 (2H, d, J=8.5 Hz) (+) APCI MASS (Positive): 510.60 (M⁺+H)

Preparation 165

### Methyl 4-[2-[4-[4-(4-pyridylmethyl)-1-piperazinyl]benzoyl]hydrazinocarbonyl]benzoate

NMR (DMSO-d₆, δ): 2.50-2.60 (4H, m), 3.2-3.4 (4H, m), 3.58 (2H, s), 3.89 (3H, s), 7.00 (2H, d, J=8.9 Hz), 7.37 (2H, d, J=5.9 Hz), 7.82 (2H, d, J=8.9 Hz), 8.00-8.11 (4H, m), 8.53 (2H, d, J=5.9 Hz), 10.28 (1H, s), 10.57 (1H, s) APCI MASS (Positive): 473.6 (MH⁺)

Preparation 166

### Methyl 4-[2-[4-(1,4-dioxa-8-azaspiro[4.5]dec-8-yl)benzoyl]hydrazinocarbonyl]benzoate

NMR (DMSO-d₆, δ): 1.66 (4H, m), 3.42-3.47 (4H, m), 3.90 (3H, s), 3.92 (4H, s), 7.02 (2H, d, J=9 Hz), 7.80 (2H, d, J=9 Hz), 8.00-8.12 (4H, m), 10.26 (1H, s), 10.58 (1H, s) APCI MASS: 440.2 (M⁺+H)

Preparation 167

### Methyl 4-[2-[4-(2-phenyl-2,4,6,7-tetrahydro-5H-pyrazolo[4,3-c]pyridin-5-yl]benzoyl hydrazinocarbonyl]-benzoate

NMR (DMSO-d₆, δ): 2.86-3.00 (2H, m), 3.70-3.90 (2H, m), 3.90 (3H, s), 4.45-4.51 (2H, m), 7.09-8.31 (14H, m), 10.28 (1H, s), 10.59 (1H, s) APCI MASS: 495.93 (M⁺)

Preparation 168

### Methyl 4-[2-[4-[4-(3,3-dimethyl-1,5-dioxaspiro[5.5]-undec-9-yl)-1-piperazinyl]benzoyl]hydrazinocarbonyl]benzoate

NMR (DMSO-d₆, δ): 0.89 (6H, s), 1.2-1.8 (5H, m), 2.1-2.42 (4H, m), 2.60 (4H, br s), 3.26 (4H, br s), 3.42 (4H, s), 3.90 (3H, s), 6.98 (2H, d, J=8.9 Hz), 7.80 (2H, d, J=8.9 Hz), 8.02 (2H, d, J=8.7 Hz) 8.09 (2H, d, J=8.7 Hz), 10.26 (1H, s), 10.58 (1H, s) APCI MASS: 564.07 (M⁺+H)

Preparation 169

Methyl 4-[2-[4-(4-cyclopentyl-1-piperazinyl)benzoyl]hydrazinocarbonyl]benzoate

NMR (DMSO-d₆, δ): 1.2-1.9 (8H, m), 2.4-2.6 (5H, m), 3.1-3.4 (4H, m), 3.90 (3H, s), 6.99 (2H, d, J=8.9 Hz), 7.81 (2H, d, J=8.9 Hz), 8.00-8.12 (4H, m), 10.27 (1H, s), 10.58 (1H, s) APCI MASS: 451.27 (M⁺+H)

Preparation 170

### Methyl 4-[2-[4-(4-cycloheptyl-1-piperazinyl)benzoyl]hydrazinocarbonyl]benzoate

NMR (DMSO-d₆, δ): 1.3-1.9 (12H, m), 2.5-2.7 (5H, m), 3.2-3.4 (4H, m), 3.90 (3H, s), 6.98 (2H, d, J=9 Hz), 7.80 (2H, d, J=9 Hz), 8.00-8.12 (4H, m), 10.26 (1H, s), 10.58 (1H, s) APCI MASS: 479.33 (M⁺+H)

Preparation 171

### Methyl 4-[2-[4-[4-(1,4-dioxaspiro[4.5]dec-8-yl)-1-piperazinyl]benzoyl]hydrazinocarbonyl]benzoate

NMR (DMSO-d₆, δ): 1.40-1.80 (8H, m), 2.30-2.42 (1H, m), 2.50-2.70 (4H, m), 3.20-3.30 (4H, m), 3.85 (4H, s), 3.90 (3H, s), 6.98 (2H, d, J=8.9 Hz), 7.81 (2H, d, J=8.9 Hz), 8.02 (2H, d, J=8.7 Hz), 8.09 (2H, d, J=8.7 Hz), 10.26 (1H, s), 10.58 (1H, s) APCI MASS: 523.27 (M⁺+H)

Preparation 172

### Methyl 4-[2-[4-(4-tetrahydro-2H-pyran-4-yl-1-piperazinyl)benzoyl]hydrazinocarbonyl]benzoate

NMR (DMSO-d₆, δ): 1.30-1.60 (2H, m), 1.72-1.78 (2H, m), 2.42-2.50 (1H, m), 2.62 (4H, br s), 3.20-3.40 (6H, m), 3.90 (5H, br s), 6.99 (2H, d, J=8.9 Hz), 7.81 (2H, d, J=8.9 Hz), 8.02 (2H, d, J=8.6 Hz), 8.09 (2H, d, J=8.6 Hz), 10.27 (1H, s), 10.58 (1H, s) APCI MASS: 467.2 (M⁺+H)

Preparation 173

### Methyl 4-[2-[4-(1-phenyl-4-piperidyloxy)benzoyl]hydrazinocarbonyl]benzoate

IR (Nujol): 1716, 1649, 1603, 1279, 1250 cm⁻¹ NMR (DMSO-d₆, δ): 1.6-1.9 (2H, m), 2.0-2.2 (2H, m), 3.0-3.2 (2H, m), 3.4-3.6 (2H, m), 3.90 (3H, s), 4.6-4.8 (1H, m), 6.7-7.3 (7H, m), 7.8-8.2 (6H, m), 10.43 (1H, s), 10.65 (1H, s) ESI MASS (Positive): 474.3 (M⁺+H)

Preparation 174

### Methyl 4-[2-[4-[1-(4-methoxyphenyl)-4-piperidyloxy]benzoyl]hydrazinocarbonyl]benzoate

IR (Nujol): 1720, 1649, 1601, 1512, 1286, 1254 cm⁻¹ NMR (DMSO-d₆, δ): 1.7-1.9 (2H, m), 2.0-2.2 (2H, m), 2.8-3.1 (2H, m), 3.3-3.5 (2H, m), 3.69 (3H, s), 3.90 (3H, s), 4.6-4.8 (1H, m), 6.8-7.0 (4H, m), 7.11 (2H, d, J-8.7 Hz), 7.91 (2H, d, J-8.7 Hz), 8.0-8.2 (4H, m), 10.43 (1H, s), 10.65 (1H, s) ESI MASS (Positive): 504.3 (M⁺+H)

Preparation 175

### Methyl 4-[2-[4-[1-[4-(6-methoxyhexyloxy)cyclohexyl]-4-piperidyloxy]benzoyl]hydrazinocarbonyl]benzoate

NMR (CDCl₃, δ): 1.2-3.2 (26H, m), 3.33 (3H, s), 3.3-3.5 (6H, m), 3.95 (3H, s), 4.3-4.5 (1H, m), 6.90 (2H, d, J=8.6 Hz), 7.80 (2H, d, J=8.8 Hz), 7.91 (2H, d, J=8.5 Hz), 8.08 (2H, d, J=8.5 Hz) (+) APCI MASS (Positive): 610.47 (M⁺+H)

Preparation 176

### Methyl 4-[2-[4-[1-[4-(6-methoxyhexyloxy)cyclohexyl]-4-piperidyl]benzoyl]hydrazinocarbonyl]benzoate

NMR (CDCl₃, δ): 1.2-2.2 (21H, m), 2.2-2.7 (3H, m), 3.0-3.2 (3H, m), 3.33 (3H, s), 3.3-3.5 (4H, m), 3.95 (3H, m), 7.29 (2H, d, J=8.1 Hz), 7.78 (2H, d, J=8.2 Hz), 7.91 (2H, d, J=8.4 Hz), 8.08 (2H, d, J=8.4 Hz) (+) APCI MASS (Positive): 594.33 (M⁺+H)

Preparation 177

### Methyl 4-[2-[4-[1-(4-(6-methoxyhexyloxy)cyclohexyl]-4-piperidyl]benzoyl]hydrazinocarbonyl]benzoate

NMR (CDCl₃, δ): 1.2-2.1 (21H, m), 2.2-2.7 (3H, m), 3.0-3.2 (2H, m), 3.33 (3H, s), 3.3-3.5 (5H, m), 3.95 (3H, s), 4.80 (2H, br s), 7.28 (2H, d, J=7.5 Hz), 7.77 (2H, d, J=8.2 Hz), 7.90 (2H, d, J=8.5 Hz), 8.06 (2H, d, J=8.5 Hz) ESI MASS (Positive): 594.5 (M⁺+H)

Preparation 178

### Methyl 4-[2-[4-(1-cyclohexyl-4-piperidyloxy)benzoyl]hydrazinocarbonyl]benzoate

NMR (CDCl₃, δ): 0.8-1.4 (6H, m), 1.5-2.2 (8H, m), 2.2-2.4 (3H, m), 2.8-3.0 (2H, m), 3.95 (3H, s), 4.3-4.5 (1H, m), 6.91 (2H, d, J=8.6 Hz), 7.7-8.2 (8H, m) (−) APCI MASS (Negative): 478.53 (M⁻−H)

Preparation 179

### Methyl 4-[2-[4-[4-(7-methoxyheptyl)-1-piperazinyl]benzoyl]hydrazinocarbonyl]benzoate

NMR (CDCl₃, δ): 1.2-1.7 (12H, m), 1.77 (2H, br s), 2.42 (2H, t, J=8.0 Hz), 2.62 (4H, t, J-4.9 Hz), 3.3-3.5 (4H, m), 3.33 (3H, s), 3.95 (3H, s), 6.88 (2H, d, J=9.0 Hz), 7.77 (2H, d, J=9.0 Hz), 7.92 (2H, d, J=8.4 Hz), 8.11 (2H, d, J=8.4 Hz) (+) APCI MASS (Positive): 511.47 (M⁺+H)

Preparation 180

### Methyl 4-[2-[4-(4-pentyloxypiperidin-1-yl)benzoyl]hydrazinocarbonyl]benzoate

IR (KBr): 3191.6, 1933.2, 1724.0, 1596.8, 1110.8 cm⁻¹ NMR (CDCl₃, δ): 0.91 (3H, t, J=6.8 Hz), 1.29-1.92 (10H, m), 3.03-3.69 (7H, m), 3.94 (3H, s), 6.81-6.85 (2H, m), 7.72-8.06 (6H, m), 9.51 (1H, d, J=5.2 Hz), 10.09 (1H, d, J=5.4 Hz) ESI MASS (Positive)(m/z): 468.33 (M⁺+H)

Preparation 181

### N-[4-[4-(7-Methoxyheptyloxy)piperidin-1-yl]benzoyl-N′-(4-methoxycarbonylbenzoyl)hydrazine

NMR (DMSO-d₆, δ): 1.20-1.62 (12H, m), 1.81-2.01 (2H, m), 2.97-3.16 (2H, m), 3.20 (3H, s), 3.21-3.55 (5H, m), 3.55-3.72 (2H, m), 3.90 (3H, s), 6.99 (2H, d, J=9.0 Hz), 7.80 (2H, d, J=8.8 Hz), 8.03 (2H, d, J=8.7 Hz), 8.10 (2H, d, J=8.7 Hz), 10.24 (1H, s), 10.57 (1H, s) MASS (m/z): 526 (M⁺+H)

Preparation 182

### Methyl 4-[2-[4-[4-(4-methylpentyloxy)-1-piperidyl]benzoyl]hydrazinocarbonyl]benzoate

NMR (CDCl₃, δ): 0.89 (6H, d, J=6.6 Hz), 1.1-1.3 (3H, m), 1.5-1.8 (4H, m), 1.8-2.1 (2H, m), 3.0-3.2 (2H, m), 3.4-3.8 (5H, m), 3.94 (3H, s), 6.84 (2H, d, J=9.0 Hz), 7.74 (2H, d, J-8.9 Hz), 7.90 (2H, d, J=8.5 Hz), 8.05 (2H, d, J=8.5 Hz), 9.3-9.5 (1H, m), 9.9-10.1 (1H, m) (+) APCI MASS (Positive): 482.47 (M⁺+H)

Preparation 183

### Methyl 4-[2-[4-[4-(cyclohexylmethoxy)-1-piperidyl]benzoyl]hydrazinocarbonyl]benzoate

NMR (CDCl₃, δ): 0.8-2.0 (15H, m), 3.0-3.2 (2H, m), 3.23 (2H, d, J=7.7 Hz), 3.3-3.8 (3H, m), 3.95 (3H, s), 6.87 (2H, d, J=9.0 Hz), 7.75 (2H, d, J=8.9 Hz), 7.92 (2H, d, J=8.5 Hz), 8.09 (2H, d, J=8.4 Hz), 9.2-9.4 (1H, m), 9.7-9.8 (1H, m) (+) APCI MASS (Positive): 494.53 (M⁺+H)

Preparation 184

A mixture of 5-[4-[1-(4-methoxyphenyl)-4-piperidyloxy]phenyl]-1,3,4-thiadiazol-2-amine (0.69 g) and ethyl 4-(bromoacetyl)benzoate (0.74 g) in ethanol (15 ml) was stirred for 6 hours at 90° C. After being cooled to room temperature, the reaction mixture was poured into isopropyl ether. The resulting precipitate was collected by filtration, washed with isopropyl ether and added to a solution of trifluoroacetic acid (2 ml) in xylene (20 ml). Then a mixture was stirred for 4 hours at 130° C. After being cooled to room temperature, the reaction mixture was poured into isopropyl ether. The resulting precipitate was collected by filtration and washed with isopropyl ether to give ethyl 4-[2-[4-[1-(4-methoxyphenyl)-4-piperidyloxy]phenyl]imidazo[2,1-b][,3,4]thiadiazol-6-yl]benzoate (1.06 g).

IR (KBr): 1707, 1606, 1516, 1471, 1279, 1252, 1178, 1109, 1024, 833 cm⁻¹ NMR (DMSO-d₆, δ): 1.34 (3H, t, J=7.1 Hz), 2.0-2.4 (4H, m), 3.5-3.7 (4H, m), 3.80 (3H, s), 4.33 (2H, q, J=7.1 Hz), 4.8-5.0 (1H, m), 7.0-7.3 (4H, m), 7.5-7.7 (2H, m), 7.9-8.1 (6H, m), 8.90 (1H, s) ESI MASS (Positive): 555.3 (M⁺+H)

The following compounds [Preparation 185 to 196] were obtained according to a similar manner to that of Preparation 184.

Preparation 185

Ethyl 4-[2-[4-[4-(4-methoxybutoxymethyl)-1-piperidyl]phenyl]imidazo[2,1-b][1,3,4]thiadiazol-6-yl]benzoate

IR (KBr): 2935, 2862, 1703, 1608, 1471, 1282, 1176, 1115 cm⁻¹ (+) APCI MASS: 549.47 (M⁺+H)

Preparation 186

### Ethyl 4-[2-[4-[4-(6-methoxyhexyl)piperazin-1-yl]phenyl]imidazo[2,1-b][1,3,4′-thiadiazol-6-yl]benzoate

IR (KBr): 3431, 2935, 2864, 1713, 1678, 1604, 1469 cm⁻¹ NMR (DMSO-d₆, δ): 1.20-1.40 (9H, m), 1.40-1.60 (2H, m), 1.60-1.80 (4H, m), 2.90-3.20 (6H, m), 3.26 (3H, s), 3.25-3.50 (2H, m), 4.32 (2H, q, J=7.1 Hz), 7.19 (2H, d, J=8.9 Hz), 7.84 (2H, d, J=8.8 Hz), 7.90-8.15 (4H, m), 8.86 (1H, s) MASS: 548 (M⁺+H)

Preparation 187

### Ethyl 4-[2-[4-[1-(6-methoxyhexyl)-4-piperidyl]phenyl]-imidazo[2,1-b][1,3,4]thiadiazol-6-yl]benzoate trifluoroacetate

IR (KBr): 2935, 1703, 1610, 1473, 1414, 1282, 1178, 1107 cm⁻¹ NMR (DMSO-d₆, δ): 1.2-2.2 (17H, m), 2.8-3.2 (5H, m), 3.23 (3H, s), 3.3-3.4 (2H, m), 4.33 (2H, q, J=7.0 Hz), 7.49 (2H, d, J=8.4 Hz), 7.9-8.1 (6H, m), 8.92 (1H, s), 9.32 (1H, br s) (+) APCI MASS: 547.60 (M⁺+H)

Preparation 188

### Ethyl 4-[2-[4-[4-(1-methoxyheptyl)-1-piperazinyl]phenyl]imidazo[2,1-b][1,3,4]thiadiazol-6-yl]benzoate trifluoroacetate

IR (KBr): 2933, 1701, 1606, 1471, 1404, 1281, 1178, 1109 cm⁻¹ NMR (DMSO-d₆, δ): 1.2-1.8 (13H, m), 3.0-3.2 (6H, m), 3.22 (3H, s), 3.31 (2H, t, J=6.3 Hz), 3.4-3.7 (2H, m), 4.0-4.2 (2H, m), 4.33 (2H, q, J=6.9 Hz), 7.1-7.3 (2H, m), 7.8-7.9 (2H, m), 7.9-8.1 (4H, m), 8.86 (1H, s), 9.64 (1H, br s) (+) APCI MASS: 562.47 (M⁺+H)

Preparation 189

### Ethyl 4-[2-[4-[4-(5-methoxypentyl)-1-piperazinyl]phenyl]imidazo[2,1-b][1,3,4]thiadiazol-6-yl]benzoate trifluoroacetate

IR (KBr): 2937, 1701, 1606, 1471, 1281, 1178, 1111 cm⁻¹ NMR (DMSO-d₆, δ): 1.2-1.8 (9H, m), 3.0-3.2 (6H, m), 3.24 (3H, s), 3.3-3.4 (2H, m), 3.5-4.2 (4H, m), 4.2-4.4 (2H, m), 7.1-7.2 (2H, m), 7.8-7.9 (2H, m), 7.9-8.1 (4H, m), 8.86 (1H, s), 9.54 (1H, br s) (+) APCI MASS: 534.53 (M⁺+H)

Preparation 190

### Ethyl 4-[2-[4-(1-phenyl-4-piperidyloxy)phenyl]imidazo-[2,1-b][1,3,4]thiadiazol-6-yl]benzoate

IR (KBr): 1707, 1606, 1471, 1279, 1250, 1178, 1109 cm⁻¹ NMR (DMSO-d₆, δ): 1.34 (3H, t, J=7.1 Hz), 2.0-2.4 (4H, m), 3.4-3.8 (4H, m), 4.33 (2H, q, J=7.1 Hz), 4.8-5.0 (1H, m), 7.2-8.2 (13H, m), 8.90 (1H, s) ESI MASS (Positive): 525.2 (M⁺+H)

Preparation 191

### Ethyl 4-[2-[4-(1-cyclohexyl-1,2,3,6-tetrahydro-4-pyridyl)phenyl]imidazo[2,1-b][1,3,4]thiadiazol-6-yl]benzoate trifluoroacetate

IR (KBr): 2937, 1703, 1608, 1471, 1279, 1198, 1178, 1130, 1107 cm⁻¹ NMR (DMSO-d₆, δ): 1.0-2.3 (13H, m), 2.8-3.0 (2H, m), 3.1-3.9 (3H, m), 3.9-4.0 (2H, m), 4.33 (2H, q, J=6.8 Hz), 6.3-6.5 (1H, m), 7.4-8.1 (8H, m), 8.94 (1H, s), 9.59 (1H, br s) (+) APCI MASS: 513.07 (M⁺+H)

Preparation 192

### Ethyl 4-[2-[4-(1-cyclohexyl-4-piperidyloxy)phenyl]-imidazo[2,1-b][1,3,4]thiadiazol-6-yl]benzoate

IR (KBr): 2927, 1707, 1606, 1473, 1279, 1252, 1174, 1109 cm⁻¹ NMR (CDCl₃, δ): 1.1-3.3 (22H, m), 4.2-4.5 (2H, m), 4.7-4.9 (1H, m), 6.9-7.1 (2H, m), 7.5-8.3 (7H, m) (+) APCI MASS (Positive): 531.40 (M⁺+H)

Preparation 193

### Ethyl 4-[2-[4-[1-[4-(6-methoxyhexyloxy)cyclohexyl]-4-piperidyloxy]phenyl]imidazo[2,1-b][1,3,4]thiadiazol-6-yl]benzoate

IR (KBr): 2933, 1703, 1680, 1606, 1279, 1252, 1200, 1176, 1109 cm⁻¹ NMR (CDCl₃, δ): 1.2-3.6 (36H, m), 4.40 (2H, q, J=7.1 Hz), 4.8-4.9 (1H, m), 6.9-7.1 (2H, m), 7.8-8.0 (4H, m), 8.0-8.2 (3H, m) ESI MASS (Positive): 661.3 (M⁺+H)

Preparation 194

### Ethyl 4-[2-[4-[4-(5-methoxypentyloxymethyl)-1-piperidyl]phenyl]imidazo[2,1-b][1,3,4]thiadiazol-6-yl]benzoate trifluoroacetic acid

NMR (CDCl₃, δ): 1.30-1.50 (5H, m), 1.50-1.70 (5H, m), 1.80-2.05 (5H, m), 2.90-3.10 (2H, m), 3.25-3.36 (5H, m), 3.36-3.47 (4H, m), 3.80-3.95 (2H, m), 4.38 (2H, q, J=7.14 Hz), 7.22 (2H, d, J=8.85 Hz), 7.77 (2H, d, J=8.75 Hz), 7.85 (2H, d, J=8.39 Hz), 8.08 (1H, s), 8.09 (2H, d, J=8.27 Hz) ESI MASS (Positive)(m/z): 563.3 (M⁺+H)

Preparation 195

### Ethyl 4-[2-(5-methoxypentyloxyphenyl]imidazo[2,1-b][1,3,4]thiadiazol-6-yl]benzoate

NMR (DMSO-d₆, δ): 1.20-1.40 (3H, m), 1.40-1.80 (6H, m), 3.22 (3H, s), 3.20-3.60 (2H, m), 3.90-4.20 (2H, m), 4.20-4.40 (2H, m), 6.60-7.30 (3H, m), 7.50-8.40 (7H, m) API-ES MASS (Positive): 549, 511, 477, 465 (M⁺+H)

Preparation 196

### Ethyl 4-[2-[4-(4-cyclohexyl-4-methoxy-1-piperidyl)phenyl]imidazo[2,1-b][1,3,4]thiadiazol-6-yl]benzoate

NMR (CDCl₃, δ): 0.90-2.15 (15H, m), 3.00-3.30 (4H, m), 3.30-3.85 (3H, m), 6.80-7.05 (2H, m), 7.63 (1H, d, J=8.74 Hz), 7.72 (1H, d, J=8.80 Hz), 7.89 (2H, d, J=8.06 Hz), 7.95-8.15 (3H, m)

Preparation 197

A mixture of methyl 4-[(2-(4-[1-(4-methoxyphenyl)-4-piperidyloxy]benzoyl]hydrazinocarbonyl]benzoate (1.71 g) and phosphorus pentasulfide (1.1 g) in ethylene glycol dimethyl ether (35 ml) was refluxed for 3 hours. After being added triethylamine, the reaction mixture was successively refluxed for 2.5 hours. After being cooled to room temperature, the reaction mixture was poured into ice-water. Then the solution was adjusted to pH 8 with 1N aqueous sodium hydroxide. The resulting precipitate was collected by filtration and washed with water to give methyl 4-[5-[4-[1-(4-methoxyphenyl)-4-piperidyloxy]phenyl]-1,3,4-thiadiazol-2-yl]benzoate (1.5 g).

NMR (DMSO-d₆, δ): 1.7-1.9 (2H, m), 2.0-2.2 (2H, m), 2.8-3.4 (4H, m), 3.69 (3H, s), 3.91 (3H, s), 4.6-4.8 (1H, m), 6.8-7.0 (4H, m), 7.2-7.3 (2H, m), 7.9-8.3 (6H, m)

The following compounds [Preparation 198 to 220] were obtained according to a similar manner to that of Preparation 197.

Preparation 198

### Ethyl 4-[4-[5-[4-(7-methoxyheptyloxy)phenyl]-1,3,4-thiadiazol-2-yl]-1-piperazinyl]benzoate

IR (KBr): 2942.8, 1704.8, 1608.3, 1236.1, 1110.8 cm⁻¹ ESI MASS (Positive) (m/z): 539.27 (M⁺+H)

Preparation 199

### Methyl 4-[5-[4-[4-[4-(6-methoxyhexyloxy)cyclohexyl]-1-piperidyl]phenyl]-1,3,4-thiadiazol-2-yl]benzoate

ESI MASS (Positive) (m/z): 593.4 (M⁺+H)

Preparation 200

### Methyl 4-[5-[4-[1-(8-methoxyoctyl)-4-piperidyloxy]phenyl]-1,3,4-thiadiazol-2-yl]benzoate

IR (Nujol): 1716, 1603, 1514, 1281, 1250, 1173, 1113 cm⁻¹ NMR (DMOS-d₆, δ): 1.0-2.3 (20H, m), 2.8-3.5 (4H, m), 3.21 (3H, s), 3.91 (3H, s), 4.75 (1H, br s), 7.20 (2H, d, J=8.7 Hz), 8.00 (2H, d, J=8.7 Hz), 8.1-8.2 (4H, m) ESI MASS (Positive): 538.3 (M⁺+H)

Preparation 201

### Methyl 4-[5-[4-[1-(7-methoxyheptyl)-4-piperidyl]phenyl]-1,3,4-thiadiazol-2-yl]benzoate

NMR (CDCl₃, δ): 1.2-1.4 (6H, m), 1.4-1.7 (4H, m), 1.8-2.2 (6H, m), 2.3-2.5 (2H, m), 2.5-2.7 (1H, m), 3.0-3.2 (2H, m), 3.34 (3H, m), 3.3-3.4 (2H, m), 3.96 (3H, s), 7.34 (2H, d, J=8.3 Hz), 7.95 (2H, d, J=8.3 Hz), 8.0-8.2 (4H, m) (+) APCI MASS (Positive): 508.73 (M⁺+H)

Preparation 202

### Methyl 4-[5-[4-[4-(4-pyridylmethyl)-1-piperazinyl]phenyl]-1,3,4-thiadiazol-2-yl]benzoate

NMR (CDCl₃, δ): 2.61-2.64 (4H, m), 3.35-3.40 (4H, m), 3.59 (2H, s), 3.96 (3H, s), 6.96 (2H, d, J=9 Hz), 7.33 (2H, d, J=5.9 Hz), 7.90 (2H, d, J=9 Hz), 8.06 (2H, d, J=8.6 Hz), 8.15 (2H, d, J=8.6 Hz), 8.58 (2H, d, J=5.9 Hz) APCI MASS (Positive): 472 (M⁺+H)

Preparation 203

### Methyl 4-[5-[4-(1,4-dioxa-8-azaspiro[4.5]dec-8-yl)phenyl]-1,3,4-thiadiazol-2-yl]benzoate

NMR (CDCl₃, δ): 1.81-1.87 (4H, m), 3.47-3.53 (4H, m), 3.96 (3H, s), 4.01 (4H, s), 6.97 (2H, d, J=9 Hz), 7.89 (2H, d, J=9 Hz), 8.06 (2H, d, J=8.6 Hz), 8.15 (2H, d, J=8.6 Hz) APCI MASS: 438.33 (M⁺+H)

Preparation 204

### Methyl 4-[5-[4-(2-phenyl-2,4,6,7-tetrahydro-5H-pyrazolo[4,3-c]pyridin-5-yl)phenyl]-1,3,4-thiadiazol-2-yl]benzoate

NMR (CDCl₃, δ): 3.0-3.10 (2H, m), 3.72-3.76 (2H, m), 3.96 (3H, s), 4.47 (2H, s), 7.02-8.28 (14H, m) APCI MASS: 494.4(M⁺+H)

Preparation 205

### Methyl 4-[5-[4-[4-(3,3-dimethyl-1,5-dioxaspiro[5.5]undec-9-yl)-1-piperazinyl]phenyl]-1,3,4-thiadiazol-2-yl]benzoate

NMR (CDCl₃, δ): 0.97 (6H, s), 1.2-1.9 (5H, m), 2.2-2.6 (4H, m), 2.78 (4H, br s), 3.37 (4H, br s), 3.48 (2H, s), 3.52 (2H, s), 3.96 (3H, s), 6.96 (2H, d, J=9 Hz), 7.90 (2H, d, J=9 Hz), 8.06 (2H, d, J=8.6 Hz), 8.15 (2H, d, J=8.6 Hz) APCI MASS: 563.27 (M⁺+H)

Preparation 206

### Methyl 4-[5-[4-(4-cyclopentyl-1-piperazinyl)phenyl]-1,3,4-thiadiazol-2-yl]benzoate

NMR (CDCl₃, δ): 1.40-2.0 (8H, m), 2.50-2.60 (1H, m), 2.67-2.72 (4H, m), 3.35-3.40 (4H, m), 3.96 (3H, s), 6.96 (2H, d, J=9 Hz), 7.90 (2H, d, J=9 Hz), 8.06 (2H, d, J=8.6 Hz), 8.15 (2H, d, J=8.6 Hz) APCI MASS: 449.2 (M⁺+H)

Preparation 207

### Methyl 4-[5-[4-(4-cycloheptyl-1-piperazinyl)phenyl]-1,3,4-thiadiazol-2-yl]benzoate

NMR (CDCl₃, δ): 1.2-2.4 (12H, m), 3.0-3.8 (9H, m), 3.96 (3H, s), 6.93 (2H, d, J=8.6H), 7.86 (2H, d, J=8.6H), 8.01-8.16 (4H, m) APCI MASS: 477.27 (M⁺+H)

Preparation 208

### Methyl 4-[5-[4-[4-(1,4-dioxaspiro[4.5]dec-8-yl)-1-piperazinyl]phenyl]-1,3,4-thiadiazol-2-yl]-benzoate

NMR (CDCl₃, δ): 1.4-2.0 (8H, m), 2.4-2.7 (1H, m), 2.7-2.9 (4H, m), 3.3-3.5 (4H, m), 3.95 (4H, s), 3.96 (3H, s), 6.96 (2H, d, J-9 Hz), 7.90 (2H, d, J=9 Hz), 8.06 (2H, d, J=8.6 Hz), 8.15 (2H, d, J=8.6 Hz) APCI MASS: 521.4 (M⁺+H)

Preparation 209

### Methyl 4-[5-[4-(4-tetrahydro-2H-pyran-4-yl-1-piperazinyl)phenyl]-1,3,4-thiadiazol-2-yl]benzoate

NMR (CDCl₃, δ): 1.50-1.90 (4H, m), 2.4-2.6 (1H, m), 2.7-2.8 (4H, m), 3.36-3.46 (6H, m), 3.96 (3H, s), 4.0-4.1 (2H, m), 6.97 (2H, d, J=8.9 Hz), 7.91 (2H, d, J=8.9 Hz), 8.06 (2H, d, J=8.6 Hz), 8.15 (2H, d, J=8.6 Hz) APCI MASS: 465.27 (M⁺+H)

Preparation 210

### Methyl 4-[5-[4-(1-phenyl-4-piperidyloxy)phenyl]-1,3,4-thiadiazol-2-yl)benzoate

NMR (DMSO-d₆, δ): 1.6-2.2 (4H, m), 3.0-4.0 (4H, m), 3.91 (3H, s), 4.6-4.9 (1H, m), 6.7-8.4 (13H, m)

Preparation 211

### Methyl 4-[5-[4-[1-[4-(6-methoxyhexyloxy)cyclohexyl]-4-piperidyloxy]phenyl]-1,3,4-thiadiazol-2-yl]benzoate

NMR (CDCl₃, δ): 1.2-3.2 (24H, m), 3.33 (3H, s), 3.3-3.5 (6H, m), 3.96 (3H, s), 4.3-4.5 (1H, m), 7.00 (2H, d, J=8.8 Hz), 7.94 (2H, d, J=8.7 Hz), 8.07 (2H, d, J=8.4 Hz), 8.16 (2H, d, J=8.4 Hz) (+) APCI MASS (Positive): 608.53 (M⁺+H)

Preparation 212

### Methyl 4-[5-[4-[1-[4-(6-methoxyhexyloxy)cyclohexyl]-4-piperidyl]phenyl]-1,3,4-thiadiazol-2-yl]benzoate

NMR (CDCl₃, δ): 1.2-2.2 (24H, m), 2.2-2.6 (3H, m), 3.0-3.3 (3H, m), 3.33 (3H, s), 3.3-3.5 (4H, m), 7.37 (2H, d, J=8.3 Hz), 7.95 (2H, d, J=8.2 Hz), 8.0-8.3 (4H, m) (+) APCI MASS (Positive): 592.27 (M⁺+H)

Preparation 213

### Methyl 4-[5-[4-[1-[4-(6-methoxyhexyloxy)cyclohexyl]-4-piperidyl]phenyl]-1,3,4-thiadiazol-2-yl]benzoate

NMR (CDCl₃, δ): 1.2-2.7 (24H, m), 3.0-3.2 (2H, m), 3.33 (3H, s), 3.3-3.5 (5H, m), 3.96 (3H, s), 7.38 (2H, d, J=8.3 Hz), 7.95 (2H, d, J=8.1 Hz), 8.0-8.2 (4H, m) (+) APCI MASS (Positive): 592.40 (M⁺+H)

Preparation 214

### Methyl 4-[5-[4-(1-cyclohexyl-4-piperidyloxy)]phenyl]-1,3,4-thiadiazol-2-yl)benzoate

NMR (CDCl₃, δ): 1.0-1.4 (6H, m), 1.6-2.2 (8H, m), 2.2-2.6 (3H, m), 2.8-3.0 (2H, m), 3.96 (3H, s), 4.3-4.5 (1H, m), 7.00 (2H, d, J=8.8 Hz), 7.94 (2H, d, J=8.8 Hz), 8.07 (2H, d, J=8.6 Hz), 8.16 (2H, d, J=8.6 Hz) (+) APCI MASS (Positive): 478.47 (M⁺+H)

Preparation 215

### Methyl 4-[5-[4-[4-(7-methoxyheptyl)-1-piperazinyl]phenyl]-1,3,4-thiadiazol-2-yl]benzoate

NMR (CDCl₃, δ): 1.3-1.9 (12H, m), 2.3-2.5 (2H, m), 2.5-2.7 (4H, m), 3.34 (3H, s), 3.3-3.5 (4H, m), 3.96 (3H, s), 6.96 (2H, d, J=9.0 Hz), 7.90 (2H, d, J=8.8 Hz), 8.06 (2H, d, J=8.6 Hz), 8.15 (2H, d, J=8.6 Hz) (+) APCI MASS (Positive): 509.67 (M⁺+H)

Preparation 216

### Methyl 4-(5-[4′-[4-(7-methoxyheptyloxy)piperidin-1-yl]phenyl]-1,3,4-thiadiazol-2-yl]benzoate

NMR (CDCl₃, δ): 1.22-1.47 (6H, m), 1.47-1.82 (6H, m), 1.90-2.08 (2H, m), 3.03-3.22 (2H, m), 3.33 (3H, s), 3.28-3.56 (5H, m), 3.56-3.75 (2H, m), 3.97 (3H, m), 6.90-7.02 (2H, m), 7.82-7.94 (2H, m), 8.00-8.22 (4H, m) MASS (m/z): 524 (M⁺+H)

Preparation 217

### Methyl 4-[5-[4-(4-pentyloxypiperidin-1-yl)phenyl]-1,3,4-thiadiazol-2-yl]benzoate

IR (KBr): 2931.3, 1714.4, 1604.5, 1278.6, 1106.9 cm⁻¹ NMR (CDCl₃, δ): 0.91 (3H, t, J=6.8 Hz), 1.30-1.96 (10H, m), 3.06-3.73 (7H, m), 3.96 (3H, s), 6.89-6.99 (2H, m), 7.74-8.17 (6H, m) ESI MASS (Positive)(m/z): 466.53 (M⁺+H)

Preparation 218

### Methyl 4-[5-[4-(4-butoxypiperidin-1-yl)phenyl]-1,3,4-thiadiazol-2-yl]benzoate

IR (KBr): 2954.4, 1722.1, 1276.6, 1110.8 cm⁻¹ NMR (CDCl₃, δ): 0.93 (3H, t, J=7.2 Hz), 1.30-1.96 (8H, m), 3.07-3.73 (7H, m), 3.96 (3H, s), 6.94-6.99 (2H, m), 7.86-7.91 (2H, m), 8.04-8.17 (4H, m) ESI MASS (Positive)(m/z): 452.2 (M⁺+H)

Preparation 219

### Methyl 4-[5-[4-[4-(4-methylpentyloxy)-1-piperidyl]phenyl]-1,3,4-thiadiazol-2-yl]benzoate

NMR (CDCl₃, δ): 0.90 (6H, d, J=6.6 Hz), 1.1-1.3 (2H, m), 1.4-1.9 (5H, m), 1.9-2.3 (2H, m), 3.1-3.3 (2H, m), 3.4-3.8 (5H, m), 3.96 (3H, s), 6.9-7.2 (2H, m), 7.8-8.2 (4H, m) ESI MASS (Positive): 480.2 (M⁺+H)

Preparation 220

### Methyl 4-[5-[4-[4-(cyclohexylmethoxy)-1-piperidyl]phenyl]-1,3,4-thiadiazol-2-yl]benzoate

NMR (CDCl₃, δ): 0.8-2.2 (15H, m), 3.1-3.3 (4H, m), 3.4-3.8 (3H, m), 3.96 (3H, s), 7.00 (2H, d, J-8.7 Hz), 7.89 (2H, d, J=8.8 Hz), 8.0-8.2 (4H, m)

Preparation 221

A suspension of methyl 4-[5-[4-(4-butoxypiperidin-1yl)phenyl]-1,3,4-thiadiazol-2-yl]benzoate (3.54 g) and 10% sodiumhydroxide in water (6.3 ml) in a mixture of ethanol (35 ml) and tetrahydrofuran (35 ml) was refluxed for 3.5 hours. The reaction mixture was poured into water, and the mixture was adjusted to pH 1-2 with 1N hydrochloric acid. The resulting precipitate was collected, washed with water, and dried to give 4-[5-[4-(4-butoxypiperidin-1-yl)phenyl]-1,3,4-thiadiazol-2-yl]benzoic acid (2.76 g).

IR (KBr): 2952.5, 1685.5, 1604.5, 1106.9 cm⁻¹ NMR (DMSO-d₆, δ): 0.89 (3H, t, J=7.1 Hz), 1.30-2.00 (8H, m), 3.00-3.80 (7H, m), 7.07-7.11 (2H, m), 7.82-7.87 (2H, m), 8.11 (4H, s) ESI MASS (Positive)(m/z): 438.47 (M⁺+H)

The following compounds [Preparation 222 to 234] were obtained according to a similar manner to that of Preparation 221.

Preparation 222

### 4′-(4-Cyclohexylhexahydro-1H-1,4-diazepin-1-yl)-1,1′-biphenyl-4-carboxlic acid

MASS (m/z): 379 (M⁺+H)

Preparation 223

### 4-[5-[4′-[4-(7-Methoxyheptyloxy)piperidin-1-yl]phenyl]-1,3,4-thiadiazol-2-yl]benzoic acid hydrochloride

NMR (DMSO-d₆, δ): 1.10-1.60 (12H, m), 1.81-1.99 (2H, m), 3.00-3.79 (9H, m), 3.20 (3H, s), 7.08 (2H, d, J=9.0 Hz), 7.84 (2H, d, J=8.8 Hz), 8.10 (4H, s) MASS (m/z): 510 (M⁺+H)

Preparation 224

### 4′-(5-Cyclohexyl-2,5-diazabicyclo[2.2.1]hept-2-yl)-1,1′-biphenyl-4-carboxylic acid

MASS (m/z): 377 (M⁺+H)

Preparation 225

### 4′-(4-Phenyl-1-piperidyl)-1,1′-biphenyl-4-carboxylic acid

MASS (m/z): 356 (M⁺+H)

Preparation 226

### 4′-[4-(cis-4-Methylcyclohexyl)-1-piperazinyl]-1,1′-biphenyl-4-carboxylic acid MASS (m/z): 379 (M⁺+H)

Preparation 227

### 4′-[4-(trans-4-Methylcyclohexyl)-1-piperazinyl]-1,1′-biphenyl-4-carboxylic acid MASS (m/z): 379 (M⁺+H)

Preparation 228

### 4′-[4-[4-[4-(6-Methoxyhexyl)-1-piperazinyl]phenyl]-1-piperazinyl]-1,1′-biphenyl-4-carboxylic acid

MASS (m/z): 557 (M⁺+H)

Preparation 229

### 4′-[4-[4-[1-(6-Methoxyhexyl)-4-piperidyloxy]phenyl]-1-piperazinyl]-1,1′-biphenyl-4-carboxylic acid MASS (m/z): 572 (M⁺+H)

Preparation 230

### 4′-[4-[trans-4-Methoxy-4-(1-methoxycyclohexyl-1-yl)cyclohexyl-1-yl]-1-piperazinyl)-1,1′-biphenyl-4-carboxylic acid

MASS (m/z): 507 (M⁺+H)

Preparation 231

### 4′-[4-[cis-4-Methoxy-4-(1-methoxycyclohexyl-1-yl)cyclohexyl-1-yl]-1-piperazinyl]-1,1′-biphenyl-4-carboxylic acid

MASS (m/z): 507 (M⁺+H)

Preparation 232

### 4′-[4-[4-[4-(6-Methoxyhexyl)-1-piperazinyl]phenyl]-1-piperazinyl]-1,1′-biphenyl-4-carboxylic acid MASS (m/z): 557 (M⁺+H)

Preparation 233

### 4-[5-[4-[4-(4-Methylpentyloxy)-1-piperidyl]phenyl]-1,3,4-thiadiazol-2-yl]benzoic acid

NMR (DMSO-d₆, δ): 0.86 (6H, d, J-6.5 Hz), 1.1-1.3 (2H, m), 1.3-1.6 (5H, m), 1.8-2.0 (2H, m), 2.9-3.3 (2H, m), 3.3-3.6 (3H, m), 3.6-3.8 (2H, m), 6.9-7.2 (2H, m), 7.7-8.2 (6H, m) (+) APCI MASS (Positive): 466.60 (M⁺+H)

Preparation 234

### 4-[5-[4-[4-(Cyclohexylmethoxy)-1-piperidyl)phenyl]-1,3,4-thiadiazol-2-yl]benzoic acid

NMR (DMSO-d₃, δ): 0.7-2.0 (15H, m), 3.0-4.9 (7H, m), 7.08 (2H, d, J=9.1 Hz), 7.84 (2H, d, J=8.7 Hz), 8.1-8.2 (4H, m) ESI MASS (Negative): 476.2 (M⁻−H)

Preparation 235

A suspension of ethyl 4-[4-[5-[4-(7-methoxyheptyloxy)-phenyl]-1,3,4-thiadiazol-2-yl]-1-piperazinyl]benzoate (1.7 g) and 10% sodium hydroxide in water (19 ml) in a mixture of ethanol (34 ml) and tetrahydrofuran (51 ml) was refluxed for 13 hours. The reaction mixture was evaporated under reduced pressure. The residue was diluted with water and the mixture was adjusted to pH 1-2 with 1N hydrochloric acid. The resulting precipitate was collected, washed with water, and dried to give 4-[4-[5-[4-(7-methoxyheptyloxy)phenyl]-1,3,4thiadiazol-2-yl]-1-piperazinyl]benzoic acid (0.48 g).

IR (KBr): 2935.1, 1675.8, 1602.6, 1234.2, 1116.6 cm⁻¹ ESI MASS (Positive)(m/z): 533.3 (M⁺+Na), 511.4 (M⁺+H)

The following compounds [Preparation 236 to 271] were obtained according to a similar manner to that of Preparation 235.

Preparation 236

### 4-[4-(1,1′-Biphenyl)-4-yl-1H-pyrazol-1-yl]benzoic acid

NMR (DMSO-d₆, δ): 7.37-7.48 (3H, m), 7.71-7.76 (4H, m), 7.83-7.87 (2H, m), 7.98-8.10 (4H, m), 8.36 (1H, s), 9.19 (1H, s) MASS (m/z): 341 (MH⁺)

Preparation 237

### 4-[4-(4-Hexyloxyphenyl)-1H-pyrazol-1-yl]benzoic acid

IR (KBr): 1685.5, 1652.7, 1608.3 cm⁻¹ NMR (DMSO-d₆, δ): 0.80-0.95 (3H, m), 1.20-1.50 (6H, m), 1.6-1.8 (2H, m), 3.99 (2H, t, J=6.4 Hz), 6.98 (2H, d, J=8.6 Hz), 7.64 (2H, d, J=8.6 Hz), 7.97 (2H, d, J=8.6 Hz), 8.06 (2H, d, J=8.6 Hz), 8.21 (1H, s), 9.01. (1H, s) MASS (m/z): 365 (MH⁺)

Preparation 238

### 4-[1-(4-Hexyloxyphenyl)-1H-pyrazol-4-yl]benzoic acid

NMR (DMSO-d₆, δ): 0.8-0.95 (3H, m), 1.2-1.5 (6H, m), 1.7-1.8 (2H, m), 4.01 (2H, t, J=6.42 Hz), 7.06 (2H, d, J=9 Hz), 7.63 (2H, d, J=8.1 Hz), 7.78 (2H, d, J=9 Hz), 7.87 (2H, d, J=8.1 Hz), 8.16 (1H, s), 8.89 (1H, s) EI-MS MASS (m/z): 365 (MH⁺)

Preparation 239

### 4-[5-(4-[4-(4-Methylcyclohexyl)-1-piperazinyl]phenyl]-1,3,4-oxadiazol-2-yl]benzoic acid hydrochloride

NMR (DMSO-d₆, δ): 0.97 (3H, d, J=7.1 Hz), 1.5-2.0 (9H, m), 2.4-2.6 (1H, m), 3.2-3.4 (4H, m), 3.6-3.8 (2H, m), 4.0-4.2 (2H, m), 7.23 (2H, d, J=9 Hz), 8.03 (2H, d, J=9 Hz), 8.15 (2H, d, J=8.3 Hz), 8.24 (2H, d, J=8.3 Hz), 9.68 (1H, br s), 13.37 (1H, br s) API-ES MASS: 447.3 (MH⁺, free form) (+)

Preparation 240

### 4-[5-[4-[4-[4-(6-Methoxyhexyloxy)cyclohexyl]-1-piperidyl]phenyl]-1,3,4-thiadiazol-2-yl]benzoic acid hydrochloride

ESI MASS (Negative)(m/z): 577.3 (M⁺−H)

Preparation 241

### 4-[2-[4-(6-Methoxyhexyl)piperazin-1-yl]phenyl]imidazo-[2,1-b][1,3,4]thiadiazol-6-yl]benzoic acid

NMR (DMSO-d₆, δ): 1.20-1.40 (6H, m), 1.40-1.60 (2H, m), 1.60-1.80 (4H, m), 3.10-3.30 (4H, m), 3.22 (3H, s), 3.70-3.90 (4H, m), 7.18 (2H, d, J=8.1 Hz), 7.84 (2H, d, J=7.9 Hz), 7.90-8.10 (4H, m), 8.83 (1H, s) MASS: 520 (M⁺+H)

Preparation 242

### 4-[5-[4-[1-(8-Methoxyoctyl)-4-piperidyloxy]phenyl]-1,3,4-thiadiazol-2-yl]benzoic acid hydrochloride

IR (KBr): 2931, 2856, 1699, 1605, 1514, 1439, 1410, 1250, 1174, 1115, 1038 cm⁻¹ NMR (DMSO-d₆, δ): 1.0-2.3 (20H, m), 2.8-3.8 (4H, m), 3.21 (3H, s), 4.79 (1H, br s), 7.22 (2H, d, J=8.5 Hz), 8.00 (2H, d, J=8.5 Hz), 8.13 (4H, s) ESI MASS (Positive): 524.3 (M⁺+H)

Preparation 243

### 4-[5-[4-[1-(7-Methoxyheptyl)-4-piperidyl]phenyl]-1,3,4-thiadiazol-2-yl]benzoic acid

NMR (DMSO-d₆, δ): 1.2-2.1 (14H, m), 2.8-3.6 (12H, m), 7.49 (2H, d, J=8.3 Hz), 8.03 (2H, d, J=8.3 Hz), 8.1-8.2 (4H, m) (+) APCI MASS (Positive): 494.60 (M⁺+H)

Preparation 244

### 4-[5-[4-[4-(4-Pyridylmethyl)-1-piperazinyl]phenyl]-1,3,4-thiadiazol-2-yl]benzoic acid

NMR (DMSO-d₆, δ): 2.6-4.1 (10H, m), 7.1-8.6 (12H, m) API-ES MASS (Negative): 456.3 (M⁺−H)

Preparation 245

### 4-[5-[4-(1,4-Dioxa-8-azaspiro[4.5]dec-8-yl)phenyl]-1,3,4-thiadiazol-2-yl]benzoic acid API-ES MASS: 422.2 (M⁺−H)

Preparation 246

### 4-[5-[4-(2-Phenyl-2,4,6,7-tetrahydro-5H-pyrazolo[4,3-c]pyridin-5-yl)phenyl]-1,3,4-thiadiazol-2-yl]benzoic acid API-ES MASS (Negative): 478.2 (M⁺−H)

Preparation 247

### 4-[5-[4-[4-(3,3-Dimethyl-1,5-dioxaspiro[5.5]undec-9-yl)-1-piperazinyl]phenyl]-1,3,4-thiadiazol-2-yl]benzoic acid

NMR (DMSO-d₆, δ): 0.90 (6H, s), 1.2-2.48 (9H, m), 2.8-3.8 (12H, m), 7.14-7.19 (2H, m), 7.9-7.95 (2H, m), 8.12 (4H, s) API-ES MASS: 549.3 (M⁺+H)

Preparation 248

### 4-[2-[4-[4-(4-Methoxybutoxymethyl)-1-piperidyl]phenyl]-imidazo[2,1-b][1,3,4]thiadiazol-6-yl]benzoic acid

IR (KBr): 2937, 2854, 1684, 1608, 1470, 1421, 1284, 1250, 1200, 1109 cm⁻¹ NMR (DMSO-d₆, δ): 1.1-1.8 (9H, m), 2.7-2.9 (3H, m), 3.21 (3H, s), 3.1-3.6 (5H, m), 3.8-4.0 (2H, m), 7.0-7.1 (2H, m), 7.7-7.8 (2H, m), 7.9-8.0 (4H, m), 8.80 (1H, s) (+) APCI MASS: 521.20 (M⁺+H)

Preparation 249

### 4-[5-[4-(4-Cyclopentyl-1-piperazinyl)phenyl]-1,3,4-thiadiazol-2-yl]benzoic acid

NMR (DMSO-d₆, δ): 1.4-2.2 (8H, m), 3.0-3.75 (9H, m), 7.18 (2H, d, J=8.8 Hz), 7.93 (2H, d, J=8.8 Hz), 8.12 (4H, s) API-ES MASS: 435.3 (M⁺+H)

Preparation 250

### 4-[5-[4-(4-Cycloheptyl-1-piperazinyl)phenyl]-1,3,4-thiadiazol-2-yl]benzoic acid

NMR (DMSO-d₆, δ): 1.3-2.1 (12H, m), 2.6-4.0 (9H, m), 7.1-8.2 (9H, m) APCI MASS: 463.3 (M⁺+H)

Preparation 251

### 4-[2-[4-[1-(6-Methoxyhexyl)-4-piperidyl]phenyl]-imidazo[2,1-b][1,3,4]thiadiazol-6-yl]benzoic acid hydrochloride

IR (KBr): 2935, 1709, 1610, 1473, 1414, 1371, 1255, 1221, 1176, 1099, 968 cm⁻¹ NMR (DMSO-d₆, δ): 1.2-2.1 (14H, m), 2.8-4.0 (7H, m), 3.23 (3H, s), 7.50 (2H, d, J=8.3 Hz), 7.9-8.1 (6H, m), 8.91 (1H, s) (+) APCI MASS: 519.47 (M⁺+H)

Preparation 252

### 4-[2-[4-[4-(7-Methoxyheptyl)-1-piperazinyl]phenyl]-imidazo[2,1-b][1,3,4]thiadiazol-6-yl]benzoic acid hydrochloride

IR (KBr): 2933, 1699, 1606, 1471, 1402, 1373, 1246, 1174, 1101 cm⁻¹ NMR (DMSO-d₆, δ): 1.2-1.8 (10H, m), 3.0-3.8 (12H, m), 3.22 (3H, s), 7.1-7.2 (2H, m), 7.8-7.9 (2H, m), 7.9-8.1 (4H, m), 8.84 (1H, s) (+) APCI MASS: 534.47 (M⁺+H)

Preparation 253

### 4-[2-[4-[4-(5-Methoxypentyl)-1-piperazinyl]phenyl]-imidazo[2,1-b][1,3,4]thiadiazol-6-yl]benzoic acid hydrochloride

IR (KBr): 1699, 1608, 1471, 1404, 1373, 1242, 1174, 1109 cm⁻¹ NMR (DMSO-d₆, δ): 1.2-1.8 (6H, m), 2.9-3.6 (12H, m), 3.23 (3H, s), 7.1-7.2 (2H, m), 7.7-7.9 (2H, m), 7.9-8.1 (4H, m), 8.83 (1H, s) (+) APCI MASS: 506.27 (M⁺+H)

Preparation 254

### 4-[5-[4-[4-(1,4-Dioxaspiro[4.5]dec-8-yl)-1-piperazinyl]phenyl]-1,3,4-thiadiazol-2-yl]benzoic acid

NMR (DMSO-d₆, δ): 1.4-2.2 (4H, m), 2.8-3.8 (9H, m), 3.89 (4H, s), 7.17 (2H, d, J=8.9 Hz), 7.92 (2H, d, J=8.9 Hz), 8.12 (4H, s) APCI MASS: 507.3 (M⁺+H)

Preparation 255

### 4-[5-[4-(4-Tetrahydro-2H-pyran-4-yl-1-piperazinyl)phenyl]-1,3,4-thiadiazol-2-yl]benzoic acid

NMR (DMSO-d₆, δ): 1.50-2.10 (4H, m), 2.6-4.0 (14H, m), 7.18 (2H, d, J=8.9 Hz), 7.92 (2H, d, J=8.9 Hz), 8.12 (4H, s) APCI MASS: 451.2 (M⁺+H)

Preparation 256

### 4-[2-[4-(1-Phenyl-4-piperidyloxy)phenyl]imidazo[2,1-b][1,3,4]thiadiazol-6-yl]benzoic acid

IR (KBr): 1691, 1606, 1471, 1252, 1176 cm⁻¹ NMR (DMSO-d₆, δ): 2.0-2.4 (4H, m), 3.0-4.0 (4H, m), 4.8-5.0 (1H, m), 7.1-8.1 (13H, m), 8.87 (1H, s) ESI MASS (Positive): 497.2 (M⁺+H)

Preparation 257

### 4-[2-[4-(1-Cyclohexyl-1,2,3,6-tetrahydro-4-pyridyl)phenyl]imidazo[2,1-b][1,3,4]thiadiazol-6-yl]benzoic acid hydrochloride

NMR (DMSO-d₆, δ): 1.00-2.20 (13H, m), 2.84 (2H, br s), 3.93 (2H, br s), 6.46 (1H, s), 7.75 (2H, d, J=8.2 Hz), 7.99 (2H, d, J=8.2 Hz), 8.01 (4H, s), 8.92 (1H, s) APCI-ES MASS (Positive): 485.2 (M⁺+H)

Preparation 258

### 4-[2-[4-(1-Cyclohexyl-4-piperidyloxy)phenyl]imidazo-[2,1-b][1,3,4]thiadiazol-6-yl]benzoic acid

IR (KBr): 2937, 1687, 1606, 1471, 1416, 1309, 1252, 1174, 1113 cm⁻¹ NMR (DMSO-d₆, δ): 1.0-3.6 (21H, m), 7.22 (2H, d, J=8.7 Hz), 7.9-8.2 (6H, m), 8.87 (1H, s) (+) APCI MASS (Positive): 503.47 (M⁺+H)

Preparation 259

### 4-[2-[4-[1-[4-(6-Methoxyhexyloxy)cyclohexyl]-4-piperidyloxy]phenyl]imidazo[2,1-b][1,3,4]thiadiazol-6-yl]benzoic acid

NMR (DMSO-d₆, δ): 1.0-3.6 (34H, m), 7.1-7.2 (2H, m), 7.8-8.0 (7H, m), 8.87 (1H, s) (+) APCI MASS (Positive): 633.47 (M⁺+H)

Preparation 260

### 4-[5-[4-(1-Phenyl-4-piperidyloxy)phenyl]-1,3,4-thiadiazol-2-yl]benzoic acid

IR (KBr): 1680, 1603, 1514, 1296, 1252 cm⁻¹ NMR (DMSO-d₆, δ): 1.6-1.9 (2H, m), 2.0-2.2 (2H, m), 3.0-3.8 (4H, m), 4.6-4.8 (1H, m), 6.7-7.3 (7H, m), 7.9-8.3 (6H, m) ESI MASS (Negative): 456.1 (M⁻−H)

Preparation 261

### 4-[5-[4-[1-(4-Methoxyphenyl)-4-piperidyloxy]phenyl]-1,3,4-thiadiazol-2-yl]benzoic acid

IR (Nujol): 1680, 1512, 1294, 1252 cm⁻¹ NMR (DMSO-d₆, δ): 1.7-1.9 (2H, m), 2.0-2.2 (2H, m), 2.8-3.6 (4H, m), 3.69 (3H, s), 4.6-4.8 (1H, m), 6.7-7.0 (4H, m), 7.1-7.3 (2H, m), 7.8-8.3 (6H, m) ESI MASS (Negative): 486.1 (M⁻−H)

Preparation 262

### 4-[2-[4-[1-(4-Methoxyphenyl)-4-piperidyloxy]phenyl]-imidazo[2,1-b][1,3,4]thiadiazol-6-yl]benzoic acid

IR (KBr): 1680, 1605, 1516, 1471, 1423, 1302, 1248, 1176, 1122, 1030, 964, 831 cm⁻¹ NMR (DMSO-d₆, δ): 2.0-2.4 (4H, m), 3.0-3.7 (4H, m), 3.78 (3H, s), 4.8-5.0 (1H, m), 7.0-7.2 (2H, m), 7.2-7.4 (2H, m), 7.4-7.7 (2H, m), 7.8-8.2 (6H, m), 8.88 (1H, s) ESI MASS (Positive): 527.2 (M⁺+H)

Preparation 263

### 4-[5-(4-[1-[4-[(6-Methoxyhexyloxy)cyclohexyl]-4-piperidyloxy]phenyl]-1,3,4-thiadiazol-2-yl]benzoic acid

NMR (DMSO-d₆, δ): 1.2-2.5 (20H, m), 3.0-3.6 (14H, m), 7.1-7.3 (2H, m), 8.01 (2H, d, J=8.6 Hz), 8.1-8.2 (5H, m) (+) APCI MASS (Positive): 594.40 (M⁺+H)

Preparation 264

### 4-[2-[4-[4-(5-Methoxypentyloxymethyl)-1-piperidyl]phenyl]imidazo[2,1-b][1,3,4]thiadiazol-6-yl]benzoic acid hydrochloride

NMR (CDCl₃, δ): 1.20-1.95 (11H, m), 2.70-3.00 (2H, m), 3.20-3.50 (6H, m), 3.55-3.80 (2H, m), 6.96 (2H, d, J=8.00 Hz), 7.73 (2H, d, J=8.28 Hz), 7.87 (2H, d, J=7.81 Hz), 8.00-8.15 (3H, m) APCI MASS (m/z): 535.2 (M⁺)

Preparation 265

### 4-[5-[4-[1-[4-(6-Methoxyhexyloxy)cyclohexyl]-4-piperidyl]phenyl]-1,3,4-thiadiazol-2-yl]benzoic acid

NMR (DMSO-d₆, δ): 1.1-1.7 (12H, m), 1.9-2.2 (14H, m), 2.8-3.6 (8H, m), 7.48 (2H, d, J=8.2 Hz), 8.04 (2H, d, J=8.3 Hz), 8.1-8.2 (4H, m) (+) APCI MASS (Positive): 578.33 (M⁺+H)

Preparation 266

### 4-[5-[4-[1-[4-(6-Methoxyhexyloxy)cyclohexyl)-4-piperidyl]phenyl]-1,3,4-thiadiazol-2-yl]benzoic acid

NMR (DMSO-d₆, δ): 1.2-2.2 (20H, m), 2.2-2.6 (14H, m), 7.47 (2H, d, J=8.3 Hz), 8.04 (2H, d, J=8.2 Hz), 8.1-8.2 (4H, m) (+) APCI MASS (Positive): 578.40 (M⁺+H)

Preparation 267

### 4-[5-[4-(1-Cyclohexyl-4-piperidyloxy)phenyl]-1,3,4-thiadiazol-2-yl]benzoic acid

NMR (DMSO-d₆, δ): 1.0-3.6 (19H, m), 4.7-5.0 (1H, m), 7.1-7.3 (2H, m), 7.9-8.1 (2H, m), 8.1-8.3 (4H, m), 9.4-9.6 (1H, m) (+) APCI MASS (Positive): 464.33 (M⁺+H)

Preparation 268

### 4-[5-[4-[4-(7-Methoxyheptyl)-1-piperazinyl]phenyl]-1,3,4-thiadiazol-2-yl]benzoic acid

NMR (CDCl₃, δ): 1.2-1.8 (10H, m), 3.0-3.8 (15H, m), 7.18 (2H, d, J=8.8 Hz), 7.93 (2H, d, J=8.8 Hz), 8.1-8.2 (4H, m) (+) APCI MASS (Positive): 495.60 (M⁺+H)

Preparation 269

### 4-[5-[4-(4-Pentyloxypiperidin-1-yl)phenyl]-1,3,4-thiadiazol-2-yl]benzoic acid

IR (KBr): 2931.3, 1685.5, 1604.5, 1108.9 cm⁻¹ NMR (DMSO-d₆, δ): 0.84-0.91 (3H, m), 1.29-2.00 (10H, m), 3.00-3.70 (7H, m), 7.07-7.11 (2H, m), 7.82-8.11 (6H, m) ESI MASS (Positive)(m/z): 452.40 (M⁺+H)

Preparation 270

### 4-[2-[4-(5-Methoxypentyloxy)phenyl]imidazo[1,2-b][1,3,4]thiadiazol-6-yl]benzoic acid

NMR (DMSO-d₆, δ): 1.35-1.80 (6H, m), 3.22 (3H, s), 3.10-3.40 (2H, m), 3.80-4.20 (2H, m), 6.60-7.70 (4H, m), 7.80-8.50 (6H, m) MASS: 517 (M⁺+Br), 437 (M)

Preparation 271

### 4-[2-[4-(4-Cyclohexyl-4-methoxy-1-piperidyl)phenyl]-imidazo[1,2-b][1,3,4]thiadiazol-6-yl]benzoic acid

NMR (CDCl₃+CD₃OD, 6): 0.90-1.25 (6H, m), 1.42 (3H, t, J=70.10 Hz), 1.50-2.30 (9H, m), 3.20 (3H, s), 3.25-3.80 (4H, m), 4.39 (2H, q, J=7.12 Hz), 7.37 (2H, br d, J=8.66 Hz), 7.83 (2H, d, J=8.80 Hz), 7.90 (2H, d, J=8.44 Hz), 8.09 (2H, d, J=8.36 Hz), 8.11 (1H, s)

Preparation 272

A mixture of 4-[5-[4-(2-phenyl-2,4,6,7-tetrahydro-5H-pyrazolo[4,3-c]pyridin-5-yl)phenyl]-1,3,4-thiadiazol-2-yl]benzoic acid (1.39 g), O-benzotriazol-1-yl-N,N,N′,N′-tetramethyluronium hexafluorophosphate (1.3 g) and N,N-diisopropylethylamine (1 ml) in 1-methyl-2-pyrrolidinone (30 ml) was stirred for 2 hours at 50° C. The reaction mixture was poured into water. Then the resulting precipitate was collected by filtration and washed with water to give 1-[4-[5-[4-(2-phenyl-2,4,6,7-tetrahydro-5H-pyrazolo[4,3-c]-pyridin-5-yl)phenyl]-1,3,4-thiadiazol-2-yl]benzoyloxy]-1H-1,2,3-benzotriazole (1.59 g).

IR (KBr): 1778, 1601, 1504, 1414, 1230, 1188, 985 cm⁻¹ NMR (CDCl₃, δ): 2.9-3.1 (2H, m), 3.7-3.9 (2H, m), 4.4-4.6 (2H, m), 7.0-8.5 (18H, m) (+) APCI MASS: 596.73 (M⁺+H)

The following compounds [Preparation 273 to 279] were obtained according to a similar manner to that of Preparation 272.

Preparation 273

### 1-[4′-(4-Cyclohexylhexahydro-1H-1,4-diazepin-1-yl)-1,1′-biphenyl-4-yl]carbonyloxy-1H-1,2,3-benzotriazole

NMR (CDCl₃, δ): 0.8-4.0 (21H, m), 6.7-8.5 (12H, m) MASS (m/z): 496 (M⁺+H)

Preparation 274

### 1-[4′-(5-Cyclohexyl-2,5-diazabicyclo[2.2.1]hept-2-yl)-1,1′-biphenyl-4-yl]carbonyloxy-1H-1,2,3-benzotriazole

MASS (m/z): 494 (M⁺+H)

Preparation 275

### 1-[4′-(4-Phenyl-1-piperidyl)-1,1′-biphenyl-4-yl]carbonyloxy]-1H-1,2,3-benzotriazole

IR (KBr): 1772, 1238, 1209, 976 cm⁻¹ NMR (CDCl₃, δ): 1.5-2.1 (4H, m), 2.6-3.05 (3H, m), 3.85-4.05 (2H, m), 7.09 (2H, d, J=8.9 Hz), 7.15-7.7 (10H, m), 7.80 (2H, d, J=8.6 Hz), 8.12 (1H, d, J=8.2 Hz), 8.30 (2H, d, J=8.6 Hz) MASS (m/z): 475 (M⁺+H)

Preparation 276

### 1-[4′-[4-(cis-4-Methylcyclohexyl)-1-piperazinyl]-1,1′-biphenyl-4-yl]carbonyloxy-1H-1,2,3-benzotriazole

NMR (CDCl₃, δ): 0.96 (3H, d, J=6.9 Hz), 1.4-1.9 (9H, m), 2.3-2.5 (1H, m), 2.7-2.95 (4H, m), 3.3-3.5 (4H, m), 7.03 (2H, d, J=8.8 Hz), 7.4-7.7 (5H, m), 7.79 (2H, d, J=8.5 Hz), 8.15 (1H, d, J=6.0 Hz), 8.30 (2H, d, J=8.5 Hz) MASS (m/z): 496 (M⁺+H)

Preparation 277

### 1-[4′-[4-(trans-4-Methylcyclohexyl)-1-piperazinyl]-1,1′-biphenyl-4-yl]carbonyloxy-1H-1,2,3-benzotriazole

NMR (CDCl₃, δ): 0.8-1.5 (8H, m), 1.6-2.1 (4H, m), 2.2-2.45 (1H, m), 2.7-2.9 (4H, m), 3.25-3.45 (4H, m), 7.03 (2H, d, J=8.9 Hz), 7.4-7.7 (5H, m), 7.79 (2H, d, J=8.6 Hz), 8.11 (1H, d, J=8.2 Hz), 8.30 (2H, d, J=8.6 Hz) MASS (m/z): 496 (M⁺+H)

Preparation 278

### 1-[4′-[4-[4-[4-(6-Methoxyhexyl)-1-piperazinyl)phenyl]-1-piperazinyl]-1,1′-biphenyl-4-yl]carbonyloxy-1H-1,2,3-benzotriazole

NMR (CDCl₃, δ): 1.2-2.0 (8H, m), 2.41 (2H, t, J=7.4 Hz), 2.5-2.7 (4H, m), 3.0-3.5 (17H, m), 6.85-7.15 (6H, m), 7.35-7.7 (5H, m), 7.80 (2H, d, J=8.2 Hz), 8.12 (1H, d, J=8.3 Hz), 8.31 (2H, d, J=8.2 Hz) MASS (m/z): 674 (M⁺+H)

Preparation 279

### 1-[4′-[4-[4-[1-(6-Methoxyhexyl)-4-piperidyloxy]phenyl]-1-piperazinyl]-1,1′-biphenyl-4-yl]carbonyloxy-1H-1,2,3-benzotriazole

NMR (CDCl₃, δ): 1.2-2.6 (16H, m), 2.7-2.9 (2H, m), 3.2-3.5 (13H, m), 4.2-4.35 (1H, m), 6.8-7.0 (4H, m), 7.09 (2H, d, J=8.9 Hz), 7.4-7.7 (5H, m), 7.80 (2H, d, J=8.5 Hz), 8.12 (1H, d, J=8.2 Hz), 8.31 (2H, d, J=8.5 Hz) MASS (m/z): 689 (M⁺+H)

Preparation 280

A suspension of 4-[5-[4-(4-butoxypiperidin-1-yl)phenyl]-1,3,4-thiadiazol-2-yl]benzoic acid (2.75 g) in dichloromethane (55 ml) was treated with 1-ethyl-3-(3′-dimethylaminopropyl)carbodiimide hydrochloride (959 mg) and 1-hydroxybenzotriazole (149 mg), and stirred for 16 hours at ambient temperature. The reaction mixture was extracted with dichloromethane. The extract was washed with brine and dried, and evaporated under reduced pressure to give 1-[4-[5-[4-(4-butoxypiperidin-1-yl)phenyl]-1,3,4-thiazol-2-yl]benzoyloxy]-1H-1,2,3-benzotriazole (3.35 g).

IR (KBr): 2931.3, 1778.0, 1602.6, 1228.4, 1103.1 cm⁻¹ NMR (CDCl₃, δ): 0.94 (3H, t, J=7.2 Hz), 1.34-1.99 (8H, m), 3.09-3.83 (7H, m), 6.95-7.00 (2H, m), 7.47-8.42 (10H, m) ESI MASS (Positive)(m/z): 555.40 (M⁺+H)

The following compounds [Preparation 281 to 323] were obtained according to a similar manner to that of Preparation 280.

Preparation 281

### 1-[4-[5-[4-(7-Methoxyheptyloxyphenyl]-1,3,4-thiadiazol-2-yl]-1-piperazinyl]benzoyl]-1H-1,2,3-benzotriazole

IR (KBr): 2933.2, 1768.4, 1602.6, 1230.4, 1089.6 cm⁻¹ NMR (CDCl₃, δ): 1.22-1.81 (10H, m), 3.34 (3H, s), 3.35-4.03 (12H, m), 6.92-7.03 (4H, m), 7.39-8.20 (8H, m) ESI MASS (Positive)(m/z): 627.47 (M⁺)

Preparation 282

### 1-[4-(7-Methoxyheptyloxy)benzoyl]-1H-1,2,3-benzotriazole

IR (KBr): 2933.2, 1774.2, 1602.6, 1253.5 cm⁻¹ NMR (CDCl₃, δ): 1.41-1.92 (10H, m), 3.34 (3H, s), 3.36-3.42 (2H, m), 4.09 (2H, t, J=6.5 Hz), 7.03-7.08 (2H, m), 7.40-7.63 (4H, m), 8.20-8.26 (2H, m) ESI MASS (Positive)(m/z): 383.20 (M⁺)

Preparation 283

### 1-[4-(4-[1,1′-Biphenyl]-4-yl-1H-pyrazol-1-yl)benzoyloxy]-1H-1,2,3-benzotriazole

IR (KBr): 1774.2, 1602.6, 1571.7, 1513.8, 1403.9 cm⁻¹ NMR (CDCl₃, δ): 7.37-7.71 (12H, complex m), 8.03 (2H, d, J=8.8 Hz), 8.14 (1H, s), 8.1-8.14 (1H, m), 8.35 (1H, s), 8.42 (2H, d, J=8.8 Hz) MASS (m/z): 458 (MH⁺)

Preparation 284

### 1-[4-[4-(4-Hexyloxyphenyl)-1H-pyrazol-1-yl]benzoyloxy]-1H-1,2,3-benzotriazole

IR (KBr): 1776.1, 1602.6, 1504.2, 1402, 1247.7, 1234.2, 1176.4, 1087.7, 987.4, 944.9 cm⁻¹ NMR (CDCl₃, δ): 0.9-1.0 (3H, m), 1.4-1.6 (6H, m), 1.7-1.9 (2H, m), 4.00 (2H, t, J=6.5 Hz), 6.96 (2H, d, J=8.7 Hz), 7.46-7.60 (5H, m), 7.97-8.04 (3H, m), 8.12 (1H, d, J=8.2 Hz), 8.23 (1H, s), 8.39 (2H, d, J=8.8 Hz) MASS (m/z): 482 (MH⁺)

Preparation 285

### 1-[4-[1-(4-Hexyloxyphenyl)-1H-pyrazol-4-yl]benzoyloxy]-1H-1,2,3-benzotriazole

IR (KBr): 1770.3, 1608.3, 1567.8, 1519.6, 1240.0, 991.2 cm⁻¹ NMR (CDCl₃, δ): 0.9-1.0 (3H, m), 1.3-1.6 (6H, m), 1.70-1.90 (2H, m), 4.01 (2H, t, J=6.5 Hz), 7.01 (2H, d, J=9 Hz), 7.40-7.60 (3H, m), 7.64 (2H, d, J=9 Hz), 7.77 (2H, d, J=8.4 Hz), 8.09 (1H, s), 8.12 (1H, d, J=9 Hz), 8.23 (1H, s), 8.30 (2H, d, J=8.4 Hz) EI MASS (m/z): 482 (MH⁺)

Preparation 286

### 1-[4-[5-[4-[4-(4-Methylcyclohexyl)-1-piperazinyl]-phenyl]-1,3,4-oxadiazol-2-yl]benzoyloxy)-1H-1,2,3-benzotriazole

IR (KBr): 1780, 1610, 1496, 1242, 1230, 989 cm⁻¹ NMR (CDCl₃, δ): 0.93 (3H, d, J=6.9 Hz), 1.4-1.9 (9H, m), 2.35-2.6 (1H, m), 2.75-2.90 (4H, m), 3.4-3.55 (4H, m), 7.00 (2H, d, J=9 Hz), 7.45-7.65 (2H, m), 7.95-8.20 (4H, m), 8.35-8.48 (4H, m)

Preparation 287

### 1-[4-[5-[4-[4-(6-Methoxyhexyloxy)-1-piperidyl]-phenyl]-1,3,4-oxadiazol-2-yl]benzoyloxy]-1H-1,2,3-benzotriazole

NMR (CDCl₃, δ): 1.20-1.80 (14H, m), 1.85-2.50 (4H, m), 2.70-2.90 (4H, m), 3.33 (3H, s), 3.34-3.55 (8H, m), 6.97 (2H, d, J=8.90 Hz), 7.40-7.65 (3H, m), 7.92 (2H, d, J=8.68 Hz), 8.13 (2H, d, J=8.18 Hz), 8.23 (2H, d, J=8.44 Hz), 8.39 (2H, d, J=8.44 Hz) ESI MASS (Positive)(m/z): 696.4 (M⁺+H)

Preparation 288

### 4-[2-[4-(6-Methoxyhexyl)piperazin-1-yl]phenyl]imidazo-[2,1-b][1,3,4]thiadiazol-6-yl]benzoic acid benzotriazol-1-yl ester

IR (KBr): 3458, 3425, 3404, 2931, 2854, 1776, 1603, 1471 cm⁻¹ NMR (DMSO-d₆, δ): 1.20-1.40 (6H, m), 1.40-1.60 (4H, m), 2.20-2.40 (4H, m), 3.15-3.30 (2H, m), 3.21 (3H, s), 3.70-4.00 (4H, m), 7.11 (2H, d, J=8.7 Hz), 7.20-7.50 (3H, m), 7.59 (2H, d, J=8.4 Hz), 7.70-8.20 (5H, m), 8.82 (1H, s) MASS: 637 (M⁺+H), 534 (M⁻−103),

Preparation 289

### 1-[4-[5-[4-[1-(8-Methoxyoctyl)-4-piperidyloxy]phenyl]-1,3,4-thiadiazol-2-yl]benzoyloxy]-1H-1,2,3-benzotriazole

IR (KBr): 2929, 2856, 1778, 1603, 1514, 1441, 1410, 1250, 1174, 1117, 1093 cm⁻¹ NMR (CDCl₃, δ): 1.1-1.8 (12H, m), 1.9-2.2 (2H, m), 2.2-2.4 (2H, m), 2.6-3.0 (6H, m), 3.3-3.4 (5H, m), 4.59 (1H, br s), 6.9-8.5 (12H, m)

Preparation 290

### 1-[4-[5-[4-[1-(7-Methoxyheptyl)-4-piperidyl]phenyl]-1,3,4-thiadiazol-2-yl]benzoyloxy]-1H-1,2,3-benzotriazole

IR (KBr): 2929, 2854, 1776, 1433, 1230, 1119, 1090, 985, 739 cm⁻¹ NMR (CDCl₃, δ): 1.2-2.8 (21H, m), 3.33 (3H, s), 3.3-3.5 (2H, m), 7.3-7.8 (5H, m), 7.97 (2H, d, J=8.3 Hz), 8.1-8.2 (1H, m), 8.2-8.3 (2H, m), 8.3-8.5 (2H, m) (+) APCI MASS (Positive): 611.07 (M⁺+H)

Preparation 291

### 1-[4-[5-[4-[4-(4-Pyridylmethyl)-1-piperazinyl]phenyl]-1,3,4-thiadiazol-2-yl) benzoyloxy]-1H-1, 2, 3-benzotriazole

IR (KBr): 1778, 1601, 1439, 1414, 1230 cm⁻¹ NMR (CDCl₃, δ): 2.6-2.7 (4H, m), 3.2-3.4 (4H, m), 3.60 (2H, s), 6.9-8.7 (16H, m) (+) APCI MASS: 574.93 (M⁺+H)

Preparation 292

### 8-[4-[5-[4-[(1H-1,2,3-Benzotriazol-1-yloxy)carbonyl]-phenyl]-1,3,4-thiadiazol-2-yl]phenyl]-1,4-dioxa-8-azaspiro[4.5]decane

IR (KBr): 1778, 1599, 1524, 1441, 1414, 1228, 1180, 1099, 984 cm⁻¹ NMR (CDCl₃, δ): 1.8-1.9 (4H, m), 3.5-3.6 (4H, m), 4.02 (4H, s), 6.7-8.5 (12H, m) (+) APCI MASS: 541.00 (M⁺+H)

Preparation 293

### 1-[4-[5-[4-[4-(3,3-Dimethyl-1,5-dioxaspiro[5.5]undec-9-yl)-1-piperazinyl]phenyl]-1,3,4-thiadiazol-2-yl]benzoyloxy]-1H-1,2,3-benzotriazole

IR (KBr): 2951, 1780, 1668, 1603, 1441, 1414, 1234, 1105, 982 cm⁻¹ NMR (CDCl₃, δ): 0.97 (6H, s), 1.2-2.5 (8H, m), 2.7-2.9 (5H, m), 3.3-3.6 (8H, m), 6.9-8.5 (12H, m)

Preparation 294

### 1-[4-[2-[4-[4-(Methoxybutoxymethyl)-1-piperidyl]-phenyl]imidazo[2,1-b][1,3,4]thiadiazol-6-yl]benzoyloxy]-1H-1,2,3-benzotriazole

IR (KBr): 2937, 2850, 1774, 1608, 1471, 1248, 1230, 1200, 1176, 1113, 1088, 984, 820 cm⁻¹ NMR (CDCl₃, δ): 1.1-2.1 (8H, m), 2.3-2.4 (1H, m), 2.7-3.0 (3H, m), 3.2-3.5 (5H, m), 3.28 (3H, s), 3.7-4.0 (2H, m), 6.8-8.3 (13H, m) (+) APCI MASS(Positive): 638.3 (M⁺+H)

Preparation 295

### 1-[4-[5-[4-(4-Cyclopentyl-1-piperazinyl)phenyl]-1,3,4-thiadiazol-2-yl]benzoyloxy]-1H-1,2,3-benzotriazole

IR (KBr): 2954, 1778, 1603, 1441, 1414, 1234, 985, 822 cm⁻¹ NMR (CDCl₃, δ): 1.4-2.3 (8H, m), 2.5-2.8 (5H, m), 3.3-3.5 (4H, m), 6.9-8.5 (12H, m) (+) APCI MASS: 551.93 (M⁺+H)

Preparation 296

### 1-[4-[5-[4-(4-Cycloheptyl-1-piperazinyl)phenyl]-1,3,4-thiadiazol-2-yl]benzoyloxy]-1H-1,2,3-benzotriazole

IR (KBr): 2924, 2852, 1780, 1603, 1441, 1414, 1232, 984 cm⁻¹ NMR (CDCl₃, δ): 1.2-2.1 (12H, m), 2.7-3.0 (5H, m), 3.3-3.6 (4H, m), 6.8-8.5 (12H, m) (+) APCI MASS: 580.00 (M⁺+H)

Preparation 297

### 1-[4-[2-[4-[1-(6-Methoxyhexyl)-4-piperidyl]phenyl]-imidazo[2,1-b][1,3,4]thiadiazol-6-yl]benzoyloxy]-1H-1,2,3-benzotriazole

IR (KBr): 2935, 1774, 1701, 1608, 1471, 1371, 1252, 1232, 1176, 1105, 972, 843 cm⁻¹ NMR (CDCl₃, δ): 1.2-2.3 (14H, m), 2.5-3.7 (7H, m), 3.31 (3H, s), 7.2-8.4 (13H, m) (+) APCI MASS: 636.13 (M⁺+H)

Preparation 298

### 1-[4-[2-[4-[4-(7-Methoxyheptyl)-1-piperazinyl]phenyl]-imidazo[2,1-b][1,3,4]thiadiazol-6-yl]benzoyloxy]-1H-1,2,3-benzotriazole

IR (KBr): 2929, 1774, 1606, 1471, 1387, 1232, 1200, 1173, 1117, 1088, 984, 820, 727 cm⁻¹ NMR (DMSO-d₆, δ): 1.2-1.7 (10H, m), 2.3-2.5 (2H, m), 2.5-2.7 (4H, m), 3.34 (3H, s), 3.3-3.5 (6H, m), 6.9-8.4 (13H, m) (+) APCI MASS: 651.13 (M⁺+H)

Preparation 299

### 1-[4-[2-[4-[4-(5-Methoxypentyl)-1-piperazinyl]phenyl]imidazo[2,1-b][1,3,4]thiadiazol-6-yl]benzoyloxy]-1H-1,2,3-benzotriazole

IR (KBr): 1774, 1701, 1608, 1471, 1390, 1232, 1198, 1174, 1115, 1090, 983 cm⁻¹ NMR (CDCl₃, δ): 1.2-2.0 (6H, m), 2.3-2.8 (6H, m), 3.2-3.5 (9H, m), 6.8-8.4 (13H, m) (+) APCI MASS (m/z): 623.20 (M⁺+H)

Preparation 300

### 1-[4-[5-[4-[4-(1,4-Dioxaspiro[4.5]dec-8-yl)-1-piperazinyl)phenyl]-1,3,4-thiadiazol-2-yl]benzoyloxy]-1H-1,2,3-benzotriazole

IR (KBr): 2951, 1778, 1603, 1441, 1416, 1232, 1101, 982 cm⁻¹ NMR (CDCl₃, δ): 1.5-2.6 (8H, m), 2.7-2.9 (5H, m), 3.3-3.5 (4H, m), 3.95 (4H, s), 6.9-8.5 (12H, m) (+) APCI MASS: 624.07 (M⁺+H)

Preparation 301

### 1-[4-[5-[4-(4-Tetrahydro-2H-pyran-4-yl-1-piperazinyl)phenyl]-1,3,4-thiadiazol-2-yl]benzoyloxy]-1H-1,2,3-benzotriazole

IR (KBr): 2956, 2835, 1778, 1603, 1441, 1414, 1232 cm⁻¹ NMR (CDCl₃, δ): 1.5-2.2 (4H, m), 2.4-2.6 (1H, m), 2.7-2.8 (4H, m), 3.3-3.5 (6H, m), 4.0-4.2 (2H, m), 6.9-8.5 (12H, m) (+) APCI MASS: 567.93 (M⁺+H)

Preparation 302

### 1-[4-[2-[4-(1-Phenyl-4-piperidyloxy)phenyl]imidazo[2,1-b](1,3,4]thiadiazol-6-yl]benzoyloxy]-1H-1,2,3-benzotriazole

IR (KBr): 1776, 1603, 1473, 1248, 1228, 1174, 982 cm⁻¹ NMR (DMSO-d₆, δ): 1.9-2.3 (4H, m), 3.1-3.3 (2H, m), 3.4-3.6 (2H, m), 4.5-4.7 (1H, m), 6.8-8.4 (18H, m) (+) APCI MASS: 614.13 (M⁺+H)

Preparation 303

### 1-[4-[2-[4-(1-Cyclohexyl-1,2,3,6-tetrahydro-4-pyridyl)phenyl]imidazo[2,1-b][1,3,4]thiadiazol-6-yl]benzoyloxy]-1H-1,2,3-benzotriazole

IR (KBr): 2927, 1776, 1606, 1471, 1230, 1173, 982, 845 cm⁻¹ NMR (CDCl₃, δ): 1.0-2.2 (10H, m), 2.3-3.5 (7H, m), 6.2-6.3 (1H, m), 7.1-8.4 (13H, m) (+) APCI MASS: 601.93 (M⁺+H)

Preparation 304

### 1-[4-[2-[4-(1-Cyclohexyl-4-piperidyloxy)phenyl]-imidazo[2,1-b][1,3,4]thiadiazol-6-yl]benzoyloxy]-1H-1,2,3-benzotriazole

IR (KBr): 2931, 2515, 1680, 1606, 1471, 1427, 1252, 1174, 970 cm⁻¹ NMR (DMSO-d₆, δ): 0.8-3.3 (18H, m), 3.8-4.0 (1H, m), 4.6-4.8 (1H, m), 7.02 (2H, d, J=8.8 Hz), 7.4-7.7 (3H, m), 7.85 (2H, d, J=8.7 Hz), 8.0-8.2 (3H, m), 8.21 (1H, s), 8.33 (2H, d, J=8.4 Hz) (+) APCI MASS (Positive): 620.13 (M⁺+H)

This compound was used in the next reaction without further purification.

Preparation 305

### 1-[4-[2-[4-[1-[4-(6-Methoxyhexyloxy)cyclohexyl]-4piperidyloxy]phenyl]imidazo[2,1-b][1,3,4]thiadiazol-6-yl]benzoyloxy]-1H-1,2,3-benzotriazole

This compound was used in the next reaction without further purification.

Preparation 306

### 1-[4-[5-[4-(1-Phenyl-4-piperidyloxy]phenyl]-1,3,4-thiadiazol-2-yl]benzoyloxy]-1H-1,2,3-benzotriazole

IR (KBr): 1780, 1601, 1500, 1439, 1410, 1304, 1250, 1178, 1030, 984 cm⁻¹ NMR (CDCl₃, δ): 1.9-2.3 (4H, m), 3.1-3.3 (2H, m), 3.4-3.6 (2H, m), 4.5-4.7 (1H, m), 6.8-8.5 (17H, m)

Preparation 307

### 1-[4-[5-[4-[1-(4-Methoxyphenyl)-4-piperidyloxy]phenyl]-1,3,4-thiadiazol-2-yl]benzoyloxy]-1H-1,2,3-benzotriazole

IR (KBr): 1792, 1605, 1512, 1439, 1248, 1180, 1034, 987 cm⁻¹ NMR (CDCl₃, δ): 1.9-2.3 (4H, m), 2.9-3.1 (2H, m), 3.3-3.5 (2H, m), 3.78 (3H, s), 4.5-4.7 (1H, m), 6.8-8.5 (16H, m)

Preparation 308

### 1-[4-[2-[4-[1-(4-Methoxyphenyl)-4-piperidyloxy]phenyl]imidazo[2,1-b][1,3,4]thiadiazol-6-yl]benzoyloxy]-1H-1,2,3-benzotriazole

IR (KBr): 1776, 1605, 1512, 1470, 1248, 1176, 1036, 980 cm⁻¹ NMR (CDCl₃, δ): 1.9-2.3 (4H, m), 2.9-3.1 (2H, m), 3.3-3.5 (2H, m), 3.78 (3H, s), 4.5-4.7 (1H, m), 6.8-8.4 (17H, m)

Preparation 309

### 1-[4-[5-[4-[1-[4-(6-Methoxyhexyloxy)cyclohexyl]-4-piperidyloxy]phenyl]-1,3,4-thiadiazol-2-yl]benzoyloxy]-1H-1,2,3-benzotriazole

IR (KBr): 1776, 1603, 1441, 1375, 1250, 1174, 1115, 1090, 984 cm⁻¹ NMR (CDCl₃, δ): 1.2-3.3 (20H, m), 3.32 (3H, s), 3.3-3.5 (10H, m), 4.3-4.5 (1H, m), 6.9-7.1 (2H, m), 7.4-7.7 (2H, m), 7.9-8.3 (7H, m), 8.41 (1H, d, J=8.4 Hz) ESI MASS (Positive): 711.3 (M⁺+H)

Preparation 310

### 1-[4-[2-[4-[4-(5-Methoxypentyloxymethyl)-1-piperidyl]phenyl]imidazo[2,1-b][1,3,4]thiadiazol-6-yl]benzoyloxy]-1H-1,2,3-benzotriazole

NMR (CDCl₃, δ): 1.20-1.95 (11H, m), 2.80-3.00 (2H, m), 3.25-3.50 (9H, m), 3.80-3.95 (2H, m), 6.95 (2H, d, J=8.97 Hz), 7.40-7.60 (3H, m), 7.74 (2H, d, J=8.80 Hz), 8.05 (2H, d, J=8.41 Hz), 8.11 (2H, d, J=8.29 Hz), 8.17 (1H, s), 8.31 (2H, d, J=8.43 Hz) APCI MASS (m/z): 674.3 (M⁺+Na)

Preparation 311

### 1-[4-[5-[4-[1-[4-(6-Methoxyhexyloxy)cyclohexyl]-4-piperidyl]phenyl]-1,3,4-thiadiazol-2-yl]benzoyloxy]-1H-1,2,3-benzotriazole

IR (KBr): 2933, 2860, 1776, 1605, 1471, 1381, 1250, 1174, 1113, 1095 cm⁻¹ NMR (CDCl₃, δ): 1.2-2.8 (24H, m), 3.1-3.5 (7H, m), 3.33 (3H, s), 7.2-8.2 (8H, m), 8.26 (2H, d, J=8.5 Hz), 8.41 (2H, d, J=8.5 Hz) (+) APCI MASS (Positive): 695.33 (M⁺+H)

Preparation 312

### 1-[4-[5-[4-[1-[4-(6-Methoxyhexyloxy)cyclohexyl]-4-piperidyl]phenyl]-1,3,4-thiadiazol-2-yl]benzoyloxy]-1H-1,2,3-benzotriazole

IR (KBr): 2933, 2858, 1776, 1651, 1541, 1452, 1433, 1373, 1090, 987 cm⁻¹

Preparation 313

### 1-[4-[5-[4-(1-Cyclohexyl-4-piperidyloxy)phenyl]-1,3,4-thiadiazol-2-yl]benzoyloxy]-1H-1,2,3-benzotriazole

IR (KBr): 2929, 2507, 1776, 1603, 1514, 1412, 1377, 1250, 1173 cm⁻¹ NMR (CDCl₃, δ): 1.0-3.4 (20H, m), 6.9-8.2 (8H, m), 8.25 (2H, d, J=8.4 Hz), 8.42 (2H, d, J=8.4 Hz) (+) APCI MASS (Positive): 581.20 (M⁺+H)

Preparation 314

### 1-[4-[5-[4-[4-(7-Methoxyheptyl)-1-piperazinyl]phenyl]-1,3,4-thiadiazol-2-yl]benzoyloxy]-1H-1,2,3-benzotriazole

IR (KBr): 2929, 2854, 1776, 1603, 1441, 1414, 1232, 984 cm⁻¹ NMR (CDCl₃, δ): 1.2-2.4 (10H, m), 2.4-2.6 (2H, m), 2.6-2.8 (4H, m), 3.34 (3H, s), 3.3-3.5 (6H, m), 6.98 (2H, d, J=8.9 Hz), 7.4-7.7 (3H, m), 7.93 (2H, d, J=8.8 Hz), 8.13 (1H, d, J=8.1 Hz), 8.24 (2H, d, J=8.5 Hz), 8.40 (2H, d, J=8.5 Hz) (+) APCI MASS (Positive): 612.20 (M⁺+H)

Preparation 315

### 1-[4-[5-[4-[4-(7-Methoxyheptyloxy)-1-piperidyl]phenyl]-1,3,4-thiadiazol-2-yl]benzoyloxy]-1H-1,2,3-benzotriazole

NMR (CDCl₃, δ): 1.20-1.45 (6H, m), 1.45-1.80 (6H, m), 1.80-2.35 (4H, m), 3.00-3.20 (3H, m), 3.33 (3H, s), 3.37 (2H, t, J=6.42 Hz), 3.48 (2H, t, J=6.54 Hz), 3.55-3.75 (2H, m), 6.97 (2H, d, J=8.95 Hz), 7.40-7.65 (3H, m), 7.90 (2H, d, J=8.80 Hz), 8.12 (1H, d, J=8.17 Hz), 8.23 (2H, d, J=8.44 Hz), 8.40 (2H, d, J=8.43 Hz)

Preparation 316

### 1-[4-[5-[4-(4-Pentyloxypiperidin-1-yl)phenyl]-1,3,4-thiadiazol-2-yl]benzoyloxy]-1H-1,2,3-benzotriazole

IR (KBr): 2929.3, 1778.0, 1602.6, 1105.0 cm⁻¹ NMR (CDCl₃, δ): 0.91 (3H, t, J=6.9 Hz), 1.34-2.04 (10H, m), 3.11-3.83 (7H, m), 6.96-7.00 (2H, m), 7.36-8.42 (10H, m) ESI MASS (Positive)(m/z): 569.33 (M⁺+H)

Preparation 317

### 4-[2-(5-Methoxypentyloxy)phenyl]imidazo[1,2-b][1,3]-thiazol-6-yl]benzoic acid benzotriazol-1-yl ester

IR (KBr): 2937, 2866, 1776, 1605, 1458 cm⁻¹ NMR (DMSO-d₆, δ): 1.30-1.80 (6H, m), 3.23 (3H, s), 3.50-3.70 (2H, m), 3.80-4.20 (2H, m), 6.90-7.70 (8H, m), 7.80-8.20 (4H, m), 8.30-8.60 (2H, m) MASS: 554 (M)

Preparation 318

### 1-[1-[4′-[4-[trans-4-Methoxy-4-(1-methoxycyclohexyl-1-yl)cyclohexyl-1-yl]-1-piperazinyl]-1,1′-biphenyl-4-yl]carbonyloxy]-1H-1,2,3-benzotriazole

NMR (CDCl₃, δ): 0.9-2.4 (19H, m), 2.6-2.8 (4H, m), 3.2-3.5 (10H, m), 7.03 (2H, d, J=8.9 Hz), 7.35-7.7 (5H, m), 7.79 (2H, d, J=8.6 Hz), 8.0-8.2 (1H, m), 8.30 (2H, d, J=8.6 Hz) MASS (m/z): 624 (M⁺+H)

Preparation 319

### 1-[1-[4′-[4-[cis-4-Methoxy-4-(1-methoxycyclohexyl-1-yl)cyclohexyl-1-yl]-1-piperazinyl]-1,1′-biphenyl-4-yl]carbonyloxy]-1H-1,2,3-benzotriazole

NMR (CDCl₃, δ): 0.7-2.45 (19H, m), 2.7-2.9 (4H, m), 3.2-3.6 (10H, m), 7.03 (2H, d, J=8.9 Hz), 7.3-7.7 (5H, m), 7.79 (2H, d, J=8.5 Hz), 8.12 (1H, d, J=8.2 Hz), 8.30 (2H, d, J=8.5 Hz) MASS (m/z): 624 (M⁺+H)

Preparation 320

### 1-[1-[4′-[4-[4-[4-(6-Methoxyhexyl)-1-piperazinyl]-phenyl]-1-piperazinyl]-1,1′-biphenyl-4-yl]carbonyloxy]-1H-1,2,3-benzotriazole

NMR (CDCl₃, δ): 1.2-2.0 (8H, m), 2.41 (2H, t, J=7.4 Hz), 2.5-2.7 (4H, m), 3.0-3.5 (17H, m), 6.85-7.15 (6H, m), 7.35-7.7 (5H, m), 7.80 (2H, d, J=8.2 Hz), 8.12 (1H, d, J=8.3 Hz), 8.31 (2H, d, J=8.2 Hz) MASS (m/z): 674 (M⁺+H)

Preparation 321

### 1-[4-[5-[4-[4-(4-Methylpentyloxy)-1-piperidyl]phenyl]-1,3,4-thiadiazol-2-yl]benzoyloxy]-1H-1,2,3-benzotriazole

IR (KBr): 2951, 1780, 1605, 1439, 1228, 1107, 982 cm⁻¹ NMR (CDCl₃, δ): 0.90 (6H, d, J=6.6 Hz), 1.1-1.3 (2H, m), 1.5-1.9 (5H, m), 1.9-2.1 (2H, m), 3.0-3.3 (2H, m), 3.4-3.8 (5H, m), 6.97 (2H, d, J=9.0 Hz), 7.4-7.7 (3H, m), 7.90 (2H, d, J=7.9 Hz), 8.1-8.2 (1H, m), 8.2-8.3 (2H, m), 8.3-8.5 (2H, m) ESI MASS (Negative): 464.2 (M⁻−HOBT-H)

Preparation 322

### 1-[4-[5-[4-[4-(Cyclohexylmethoxy)-1-piperidyl]phenyl]-1,3,4-thiadiazol-2-yl]benzoyloxy]-1H-1,2,3-benzotriazole

IR (KBr): 2922, 2848, 1784, 1603, 1441, 1414, 1363, 1288, 1113, 1092 cm⁻¹ NMR (CDCl₃, δ): 0.8-2.2 (15H, m), 3.0-3.3 (4H, m), 3.4-3.8 (3H, m), 6.8-7.0 (2H, m), 7.3-7.6 (3H, m), 7.90 (2H, d, J-8.9 Hz), 8.0-8.2 (1H, m), 8.2-8.3 (2H, m), 8.3-8.5 (2H, m) ESI MASS (Negative): 476.2 (M⁻-HOBT-H)

Preparation 323

### 1-[4-[2-[4-[(4-Cyclohexyl-4-methoxy)-1-piperidyl]-phenyl]imidazo[2,1-b][1,3,4]thiadiazol-6-yl]benzoyloxy]-1H-1,2,3-benzotriazole

IR (KBr): 1778, 1666, 1603, 1468, 1234 cm⁻¹ NMR (CDCl₃, δ): 0.90-2.10 (15H, m), 3.05-3.20 (2H, m), 3.20 (3H, s), 3.50-3.75 (2H, m), 6.95 (2H, d, J=90.10 Hz), 7.45-7.60 (3H, m), 7.73 (2H, d, J=8.90 Hz), 8.00-8.20 (4H, m), 8.31 (2H, d, J=8.60 Hz)

Preparation 324

To a solution of tert-butyl 4-hydroxy-1-piperidinecarboxylate (15 g) in N,N-dimethylformamide (75 ml) was added sodium hydride (60% dispersion in mineral oil) (2.33 g). The solution was stirred for 2 hours at 60° C. After cooling to ambient temperature, to the solution was added 1-bromoethane (13.9 ml) and the mixture was stirred for 16 hours. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (5:1 hexane-ethyl acetate elution) to give tert-butyl 4-ethoxy-1-piperidinecarboxylate (13.31 g).

NMR (DMSO-d₆, δ): 1.21 (3H, t, J=7.3 Hz), 1.45 (9H, s), 1.45-1.55 (2H, m), 1.75-1.9 (2H, m), 2.95-3.1 (2H, m), 3.35-3.5 (1H, m), 3.61 (2H, q, J=7.3 Hz), 3.75-3.85 (2H, m) MASS (m/z): 252.3 (M⁺+Na)

The following compounds [Preparation 325 and 326] were obtained according to a similar manner to that of Preparation 324.

Preparation 325

### Tert-Butyl 4-propoxy-1-piperidinecarboxylate

NMR (DMSO-d₆, δ): 0.92 (3H, t, J=7.4 Hz), 1.45 (9H, s), 1.45-1.65 (4H, m), 1.75-1.9 (2H, m), 3.0-3.15 (2H, m), 3.35-3.45 (3H, m), 3.7-3.85 (2H, m) MASS (m/z): 266.3 (M⁺+Na)

Preparation 326

### Tert-Butyl 4-butoxy-1-piperidinecarboxylate

NMR (DMSO-d₆, δ): 0.92 (3H, t, J=7.3 Hz), 1.3-1.6 (6H, m), 1.45 (9H, s), 1.75-1.9 (2H, m), 3.0-3.15 (2H, m), 3.35-3.5 (3H, m), 3.7-3.85 (2H, m) MASS (m/z): 280.4 (M⁺+Na)

Preparation 327

To a solution of tert-butyl 4-ethoxy-1-piperidinecarboxylate (13.31 g) and anisole (44.2 ml) in dichloromethane (66.6 ml) was added dropwise with stirring trifluoroacetic acid (89.4 ml) at 0° C. The mixture was then stirred for 1.5 hours at room temperature. The solvent was evaporated to give 4-ethoxypiperidine trifluoroacetate (57.73 g).

NMR (DMSO-d₆, δ): 1.11 (3H, t, J=7.0 Hz), 1.55-1.7 (2H, m), 1.85-2.0 (2H, m), 2.9-3.05 (2H, m), 3.1-3.25 (2H, m), 3.45 (2H, q, J=7.0 Hz), 3.5-3.6 (1H, m), 8.2-8.5 (2H, m) MASS (m/z): 130.4 (M⁺+H)

The following compounds [Preparation 328 and 329] were obtained according to a similar manner to that of Preparation 327.

Preparation 328

### 4-Propoxypiperidine trifluoroacetate

NMR (DMSO-d₆, δ): 0.87 (3H, t, J=7.4 Hz), 1.45-1.7 (4H, m), 1.85-2.0 (2H, m), 2.9-3.05 (2H, m), 3.1-3.2 (2H, m), 3.35 (2H, t, J=6.6 Hz), 3.5-3.6 (1H, m), 8.2-8.55 (2H, m) MASS (m/z): 144.3 (M⁺+H)

Preparation 329

### 4-Butoxypiperidine trifluoroacetate

NMR (DMSO-d₆, δ): 0.88 (3H, t, J=7.3 Hz), 1.25-1.55 (4H, m), 1.55-1.7 (2H, m), 1.85-2.0 (2H, m), 2.9-3.05 (2H, m), 3.1-3.2 (2H, m), 3.40 (2H, t, J=6.4 Hz), 3.45-3.55 (1H, m), 8.15-8.4 (2H, m) MASS (m/z): 158.4 (M⁺+H)

Preparation 330

To a suspension of 4-ethoxypiperidine trifluoroacetate (4 g) and potassium bicarbonate (6.14 g) in dimethylsulfoxide (16.5 ml) was added 4-fluorobenzonitrile (2.39 g) and stirred for 5 hours at 150° C. The reaction mixture was added to a mixture of water and ethyl acetate. The organic layer was washed with brine and dried over magnesium sulfate. The magnesium sulfate was filtered off, and the filtrate was concentrated under reduced pressure. The residue was purified by silica gel chromatography (5:1 hexane-ethyl acetate elution) to give 4-(4-ethoxy-1-piperidyl)benzonitrile (3.64 g).

NMR (CDCl₃, δ): 1.22 (3H, t, J=7.0 Hz), 1.6-1.75 (2H, m), 1.9-2.0 (2H, m), 3.05-3.2 (2H, m), 3.5-3.7 (5H, m), 6.8-6.9 (2H, m), 7.45-7.5 (2H, m) MASS (m/z): 253.4 (M⁺+Na)

The following compounds [Preparation 331 and 332] were obtained according to a similar manner to that of Preparation 330.

Preparation 331

### 4-(4-Propoxy-1-piperidyl)benzonitrile

NMR (CDCl₃, δ): 0.93 (3H, t, J=7.4 Hz), 1.55-1.75 (4H, m), 1.9-2.0 (2H, m), 3.1-3.2 (2H, m), 3.43 (2H, t, J=6.7 Hz), 3.5-3.7 (3H, m), 6.8-6.9 (2H, m), 7.45-7.5 (2H, m) MASS (m/z): 267.3 (M⁺+Na)

Preparation 332

### 4-(4-Butoxy-1-piperidyl)benzonitrile

NMR (CDCl₃, δ): 0.93 (3H, t, J=7.3 Hz), 1.3-1.45 (2H, m), 1.5-1.75 (4H, m), 1.9-2.0 (2H, m), 3.1-3.2 (2H, m), 3.4-3.7 (5H, m), 6.8-6.9 (2H, m), 7.45-7.5 (2H, m) MASS (m/z): 281.2 (M⁺+Na)

Preparation 333

To a solution of 4-(4-ethoxy-1-piperidyl)benzonitrile (3.64 g) and hydrazinecarbothioamide (2.88 g) in toluene (36 ml) was added dropwise with stirring trifluoroacetic acid (18 ml) at room temperature. The mixture was then stirred for 10 hours at 65° C. After cooling to ambient temperature, the reaction mixture was poured into water and tetrahydrofuran and the mixture was adjusted to pH 9 with sodium hydroxide solution. The resulting precipitates were filtered, washed with water, diisopropyl ether, then dried to give 5-[4-(4-ethoxy-1-piperidyl)phenyl]-1,3,4-thiadiazol-2-amine (4.132 g).

NMR (CDCl₃, δ): 1.23 (3H, t, J=7.0 Hz), 1.6-1.8 (2H, m), 1.9-2.1 (2H, m), 2.9-3.1 (2H, m), 3.4-3.7 (5H, m), 5.08 (2H, br s), 6.92 (2H, d, J=8.9 Hz), 7.66 (2H, d, J=8.9 Hz) MASS (m/z): 327.3 (M⁺+Na)

The following compounds [Preparation 334 and 335] were obtained according to a similar manner to that of Preparation 333.

Preparation 334

### 5-[4-(4-Propoxy-1-piperidyl)phenyl]-1,3,4-thiadiazol-2-amine

NMR (CDCl₃, δ): 0.94 (3H, t, J=7.4 Hz), 1.5-2.1 (6H, m), 2.95-3.15 (2H, m), 3.35-3.7 (5H, m), 5.22 (2H, br s), 6.92 (2H, d, J=8.9 Hz), 7.66 (2H, d, J=8.9 Hz) MASS (m/z): 341.2 (M⁺+Na)

Preparation 335

### 5-[4-(4-Butoxy-1-piperidyl)phenyl]-1,3,4-thiadiazol-2-amine

NMR (CDCl₃, δ): 0.93 (3H, t, J=7.4 Hz), 1.35-1.8 (6H, m), 1.95-2.05 (2H, m), 3.0-3.1 (2H, m), 3.4-3.7 (5H, m), 5.13 (2H, s), 6.9-6.95 (2H, m), 7.65-7.7 (2H, m) MASS (m/z): 355.2 (M⁺+Na)

Preparation 336

To a solution of 5-[4-(4-ethoxy-1-piperidyl)phenyl]-1,3,4-thiadiazol-2-amine (4.13 g) in ethanol (62 ml) was added ethyl 4-(bromoacetyl)benzoate (5.52 g). The mixture was stirred for 5 hours at 90° C. To the reaction mixture was added diisopropyl ether. The resulting precipitate was collected by filtration and washed by diisopropyl ether. To a solution of the crude in xylene (124 ml) was added trifluoroacetic acid (12 ml). The mixture was stirred for 6 hours at 130° C. To the reaction mixture was added diisopropyl ether. The resulting precipitate was collected by filtration and washed by diisopropyl ether to give ethyl 4-[2-[4-(4-ethoxy-1-piperidyl)phenyl]imidazo[2,1-b][1,3,4]-thiadiazol-6-yl]benzoate (6.864 g).

NMR (CDCl₃, δ): 1.27 (3H, t, J=7.0 Hz), 1.42 (3H, t, J=7.1 Hz), 1.95-2.15 (2H, m), 2.5-2.8 (2H, m), 3.35-3.5 (2H, m), 3.45 (2H, q, J=7.0 Hz), 3.65-3.8 (3H, m), 4.41 (2H, q, J=7.1 Hz), 7.7-8.0 (6H, m), 7.1-7.2 (3H, m) MASS (m/z): 477.2 (M⁺+H)

The following compounds [Preparation 337 and 338 were obtained according to a similar manner to that of Preparation 336.

Preparation 337

### Ethyl 4-[2-[4-(4-propoxy-1-piperidyl)phenyl]imidazo-[2,1-b][1,3,4]thiadiazol-6-yl]benzoate

NMR (CDCl₃, δ): 0.98 (3H, t, J=7.4 Hz), 1.42 (3H, t, J=7.1 Hz), 1.6-1.75 (2H, m), 1.95-2.15 (2H, m), 2.5-2.75 (2H, m), 3.35-3.55 (4H, m), 3.65-3.8 (3H, m), 4.41 (2H, q, J=7.1 Hz), 7.7-8.0 (6H, m), 8.1-8.2 (3H, m)

Preparation 338

### Ethyl 4-[2-[4-(4-butoxy-1-piperidyl)phenyl]imidazo-[2,1-b][1,3,4]thiadiazol-6-yl]benzoate

NMR (CDCl₃, δ): 0.96 (3H, t, J=7.2 Hz), 1.3-1.7 (7H, m), 1.9-2.1 (2H, m), 2.4-2.7 (2H, m), 3.3-3.9 (7H, m), 4.41 (2H, q, J=7.1 Hz), 7.6-8.0 (6H, m), 8.1-8.2 (3H, m) MASS (m/z): 505.4 (M⁺+H)

Preparation 339

A mixture of methyl ethyl 4-[2-[4-(4-ethoxy-1-piperidyl)phenyl]imidazo[2,1-b][1,3,4]thiadiazol-6-yl]benzoate (1 g) and 4 mol/l sodium hydroxide solution (10 ml) in a mixed solvent of methanol (20 ml) and tetrahydrofuran (10 ml) was refluxed for 5 hours. After cooling to ambient temperature, the reaction mixture was poured into cold water and the mixture was adjusted to pH 2 with 1.0 mol/l hydrochloric acid. The resulting precipitates were filtered, washed with water, isopropyl alcohol and diisopropyl ether, then dried to give 4-[2-[4-(4-ethoxy-1-piperidyl)phenyl]imidazo[2,1-b][1,3,4]thiadiazol-6-yl]benzoic acid (690.7 mg).

MASS (m/z): 447.1 (M⁻−H)

The following compounds [Preparation 340 and 341] were obtained according to a similar manner to that of Preparation 339.

Preparation 340

### 4-[2-[4-(4-Propoxy-1-piperidyl)phenyl]imidazo[2,1-b]-[1,3,4]thiadiazol-6-yl]benzoic acid

MASS (m/z): 461.2 (M⁻−H)

Preparation 341

### 4-[2-[4-(4-Butoxy-1-piperidyl)phenyl]imidazo[2,1-b]-[1,3,4]thiadiazol-6-yl]benzoic acid

MASS (m/z): 475.5 (M⁻−H)

The Starting Compounds used and the Object Compounds obtained in the following Examples 1 to 79 are given in the table as below, in which the formulas of the starting compounds are in the upper column, and the formulas of the object compounds are in the lower column, respectively.

| Ex- | |
|---|---|
| ample | |
| No. | Formula |
| 1 | 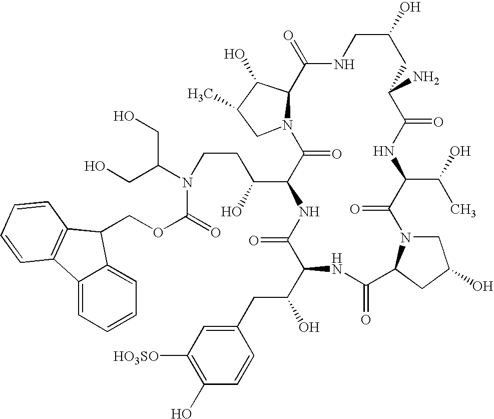 |
| | 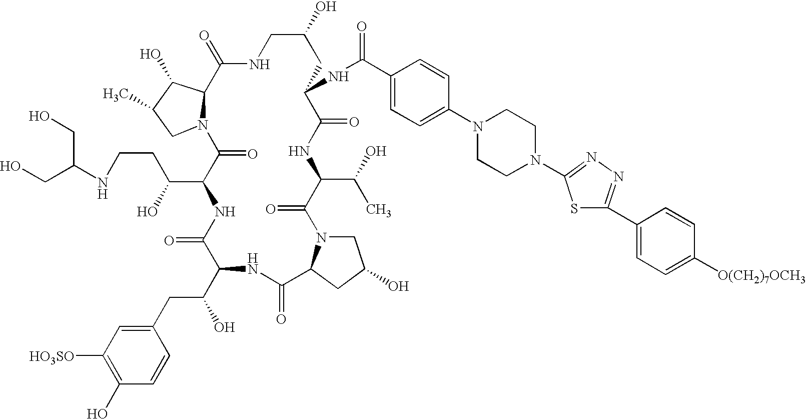 |
| 2 | 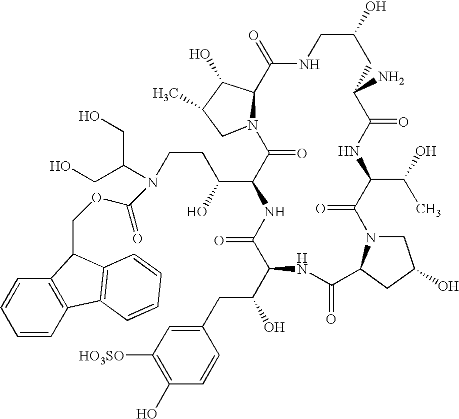 |
| | 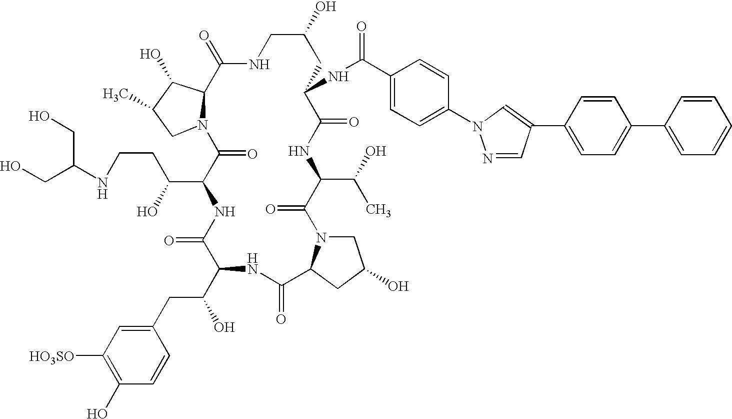 |
| 3 | 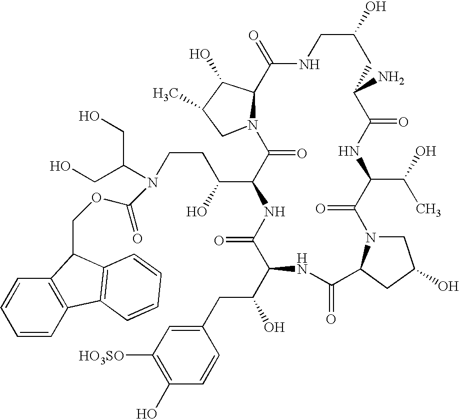 |
| | 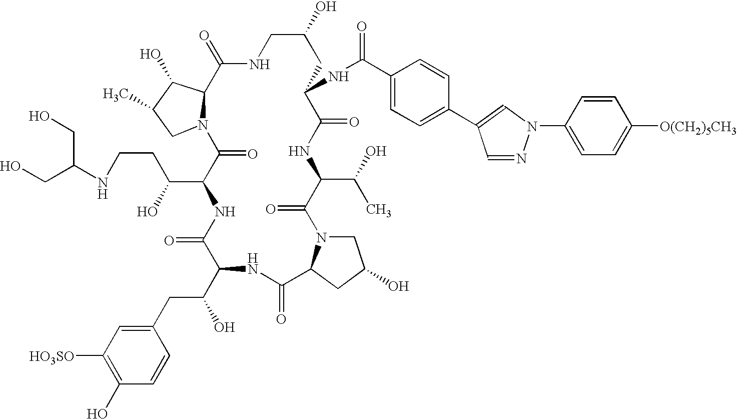 |
| 4 | 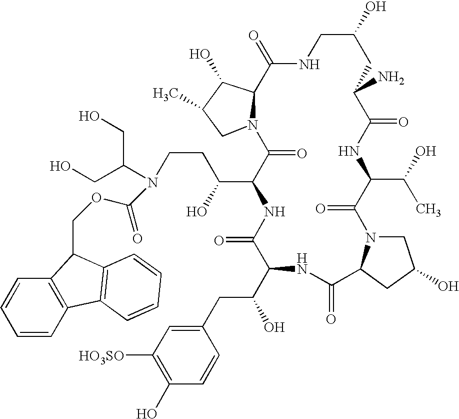 |
| | 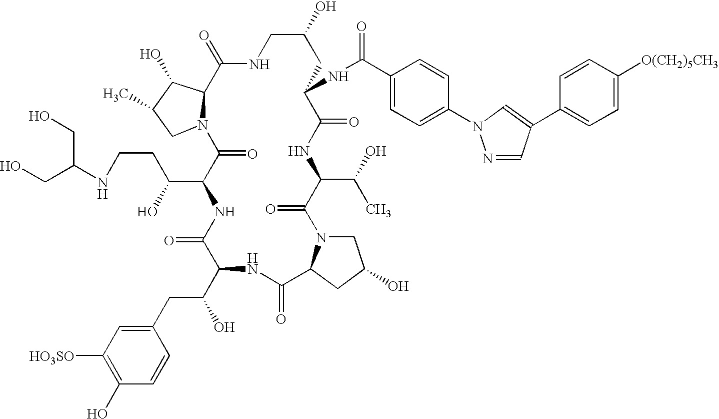 |
| 5 | 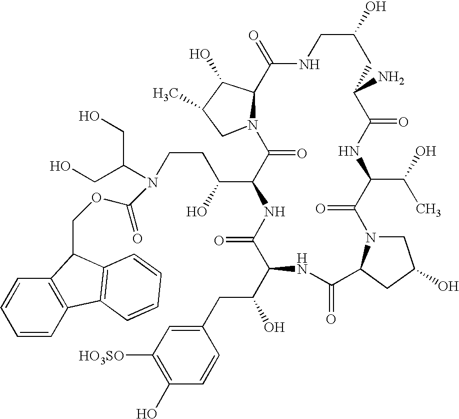 |
| | 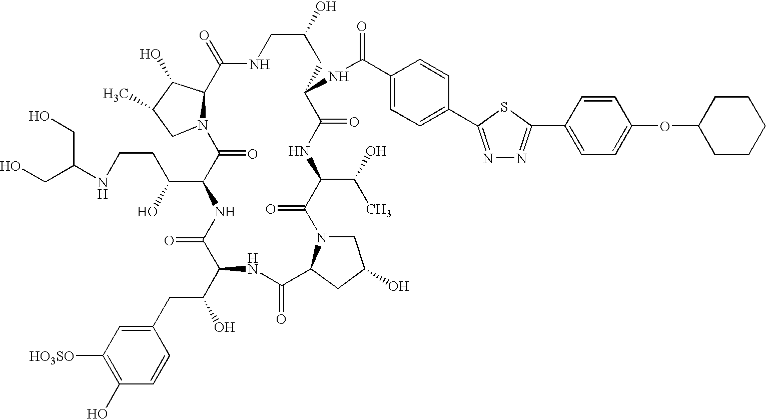 |
| 6 | 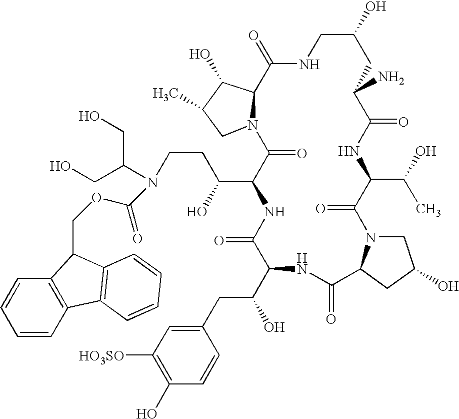 |
| | 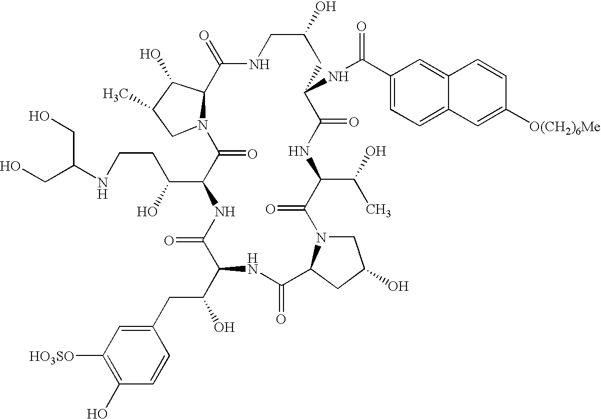 |
| 7 | 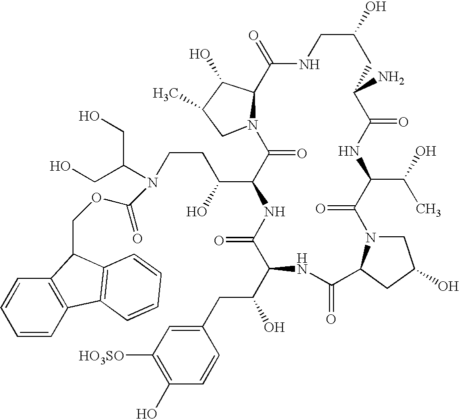 |
| | 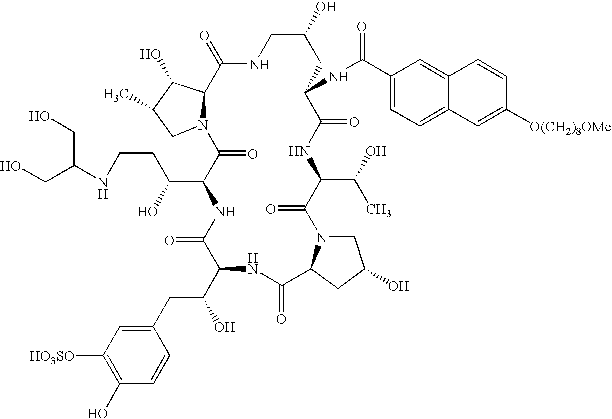 |
| 8 | 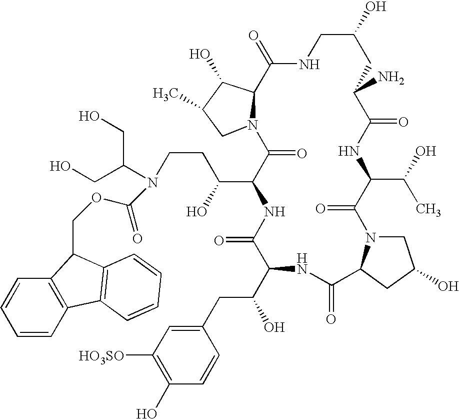 |
| | 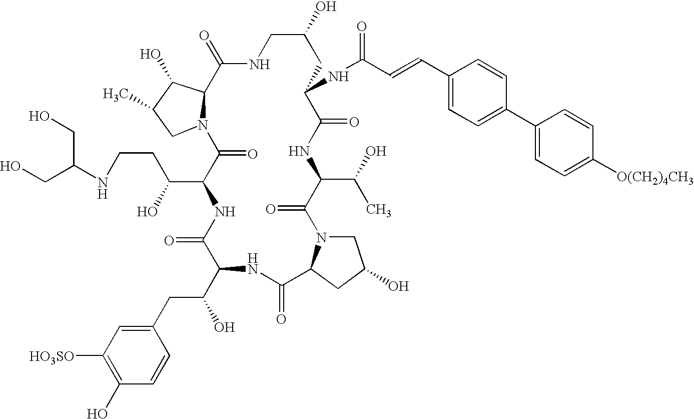 |
| 9 | 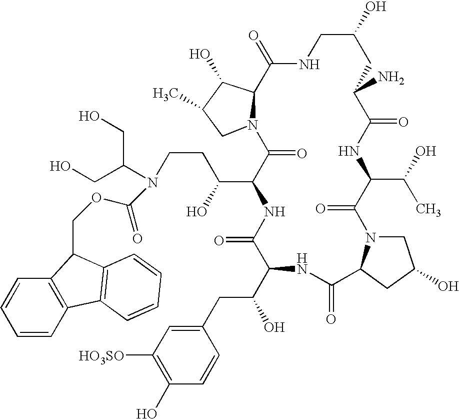 |
| | 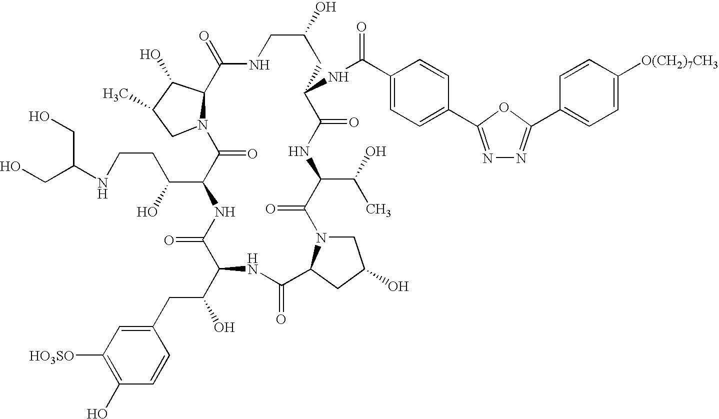 |
| 10 | 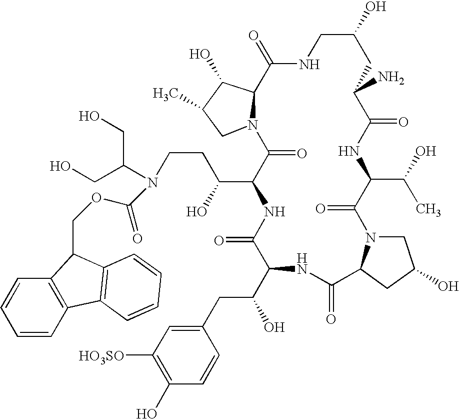 |
| | 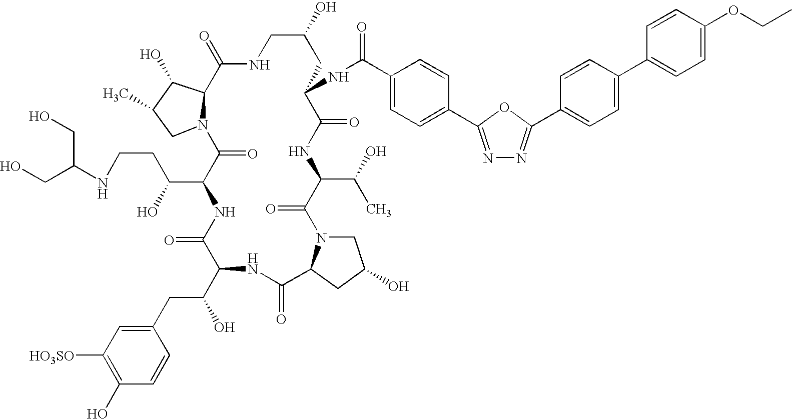 |
| 11 | 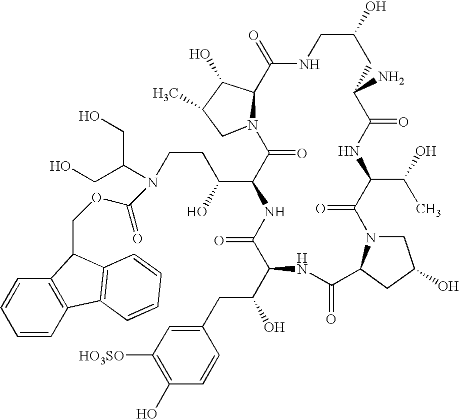 |
| | 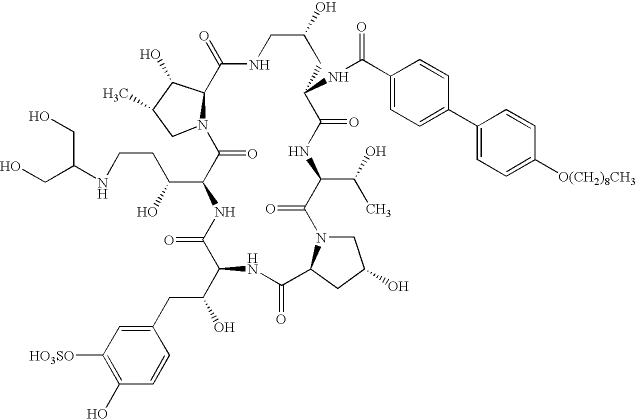 |
| 12 | 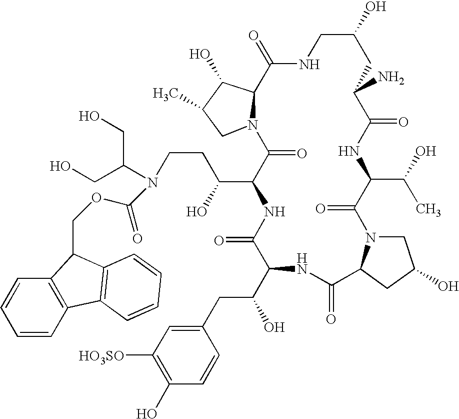 |
| | 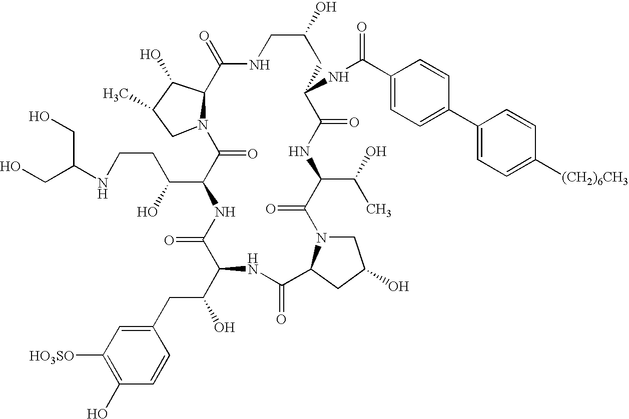 |
| 13 | 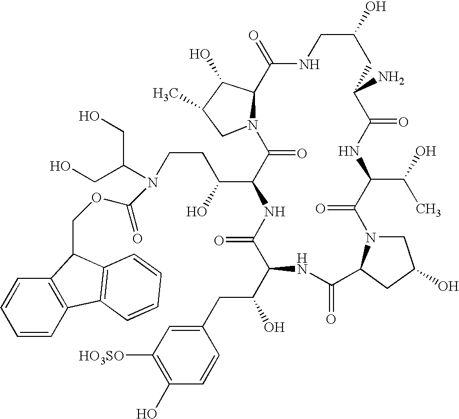 |
| | 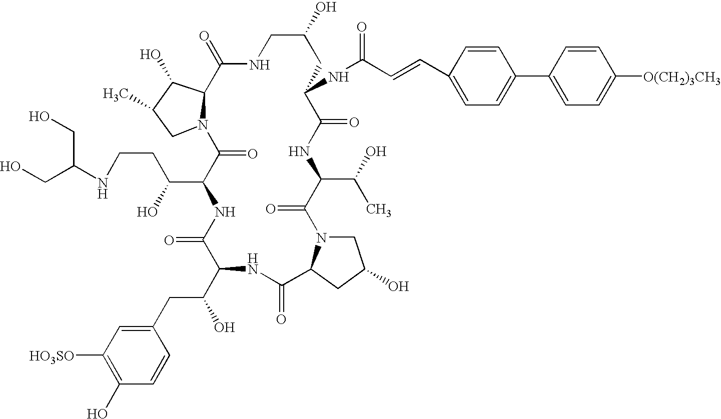 |
| 14 | 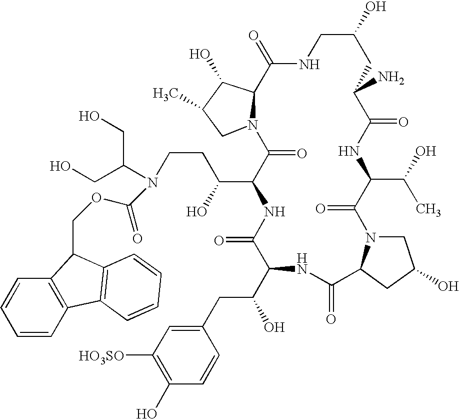 |
| | 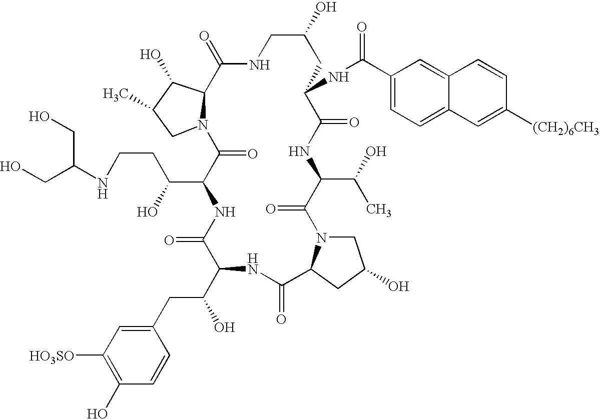 |
| 15 | 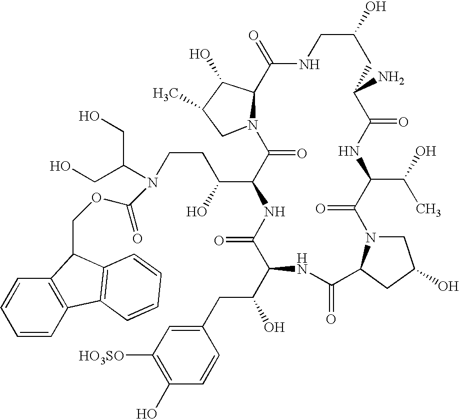 |
| | 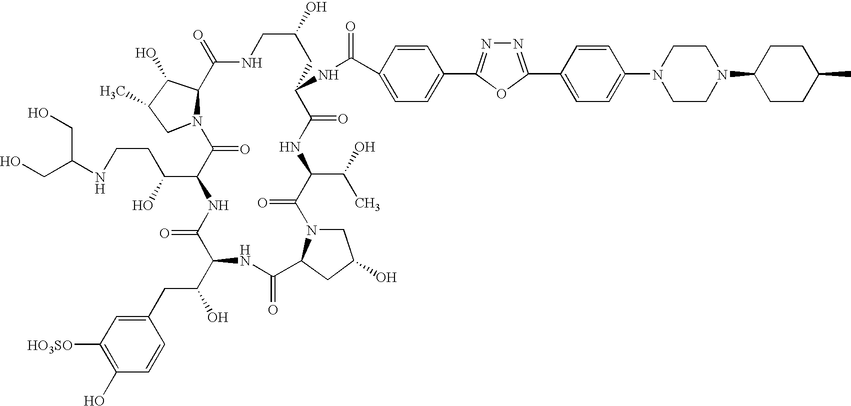 |
| 16 | 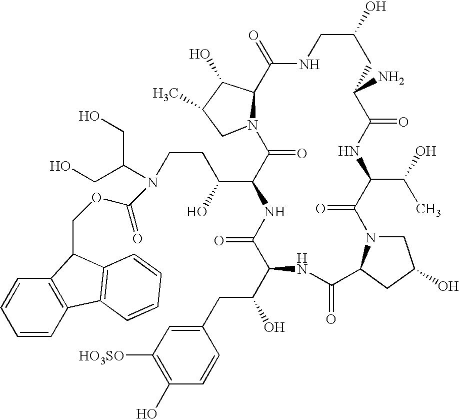 |
| | 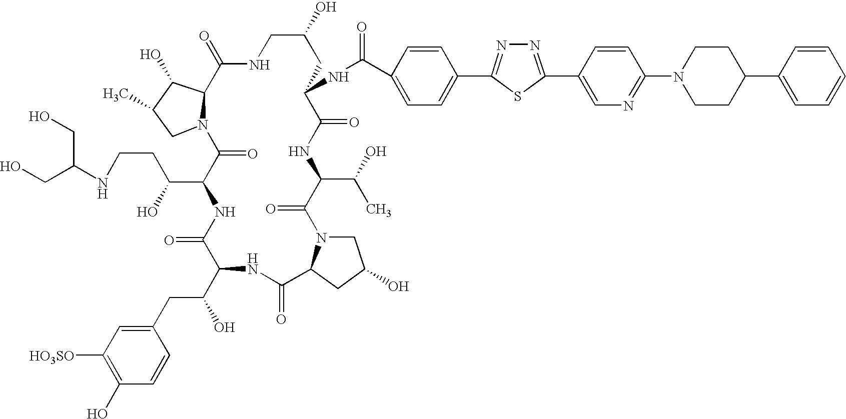 |
| 17 | 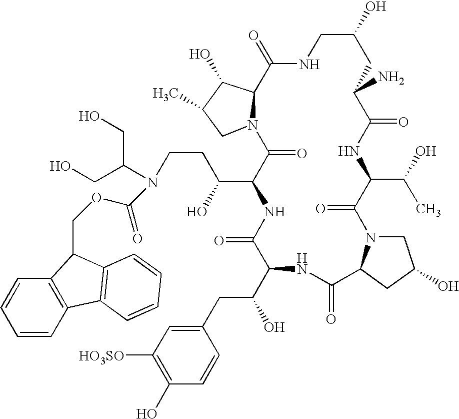 |
| | 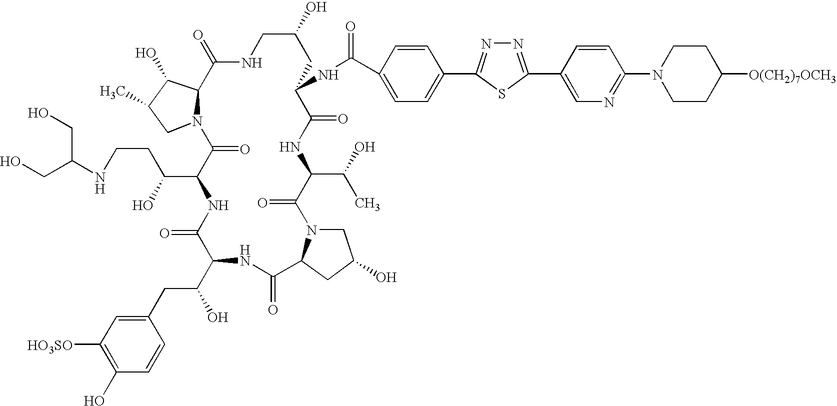 |
| 18 | 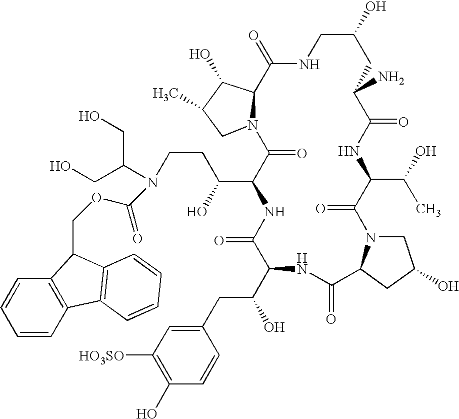 |
| | 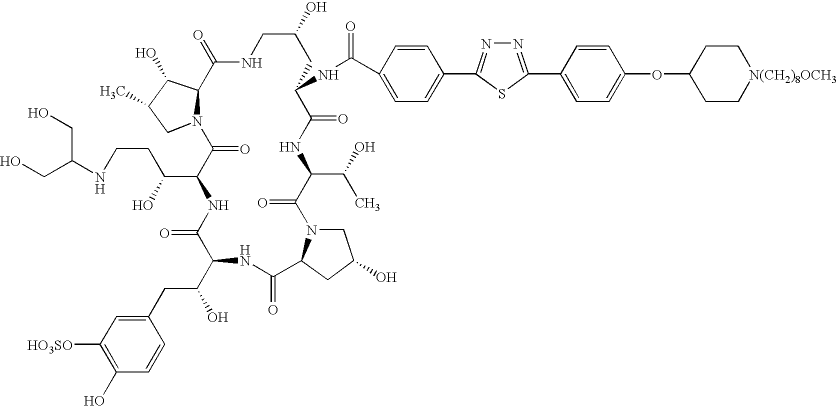 |
| 19 | 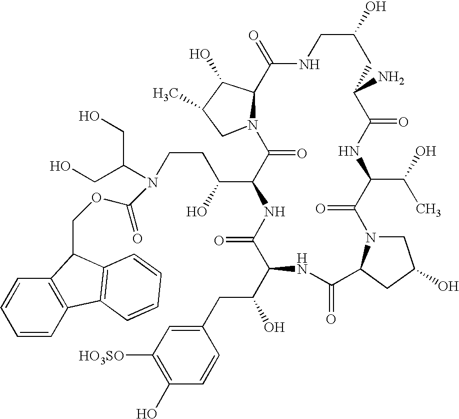 |
| | 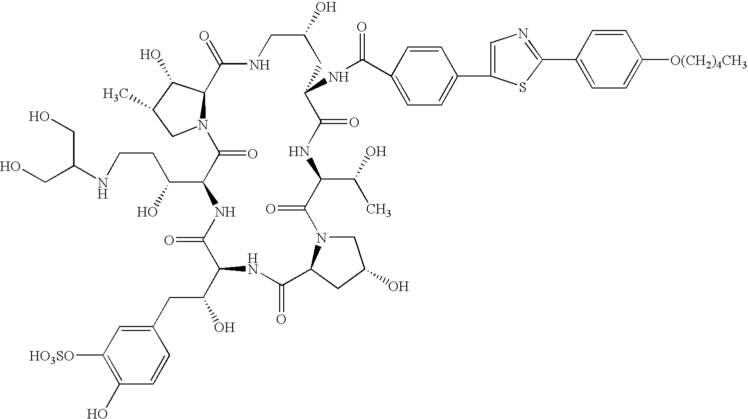 |
| 20 | 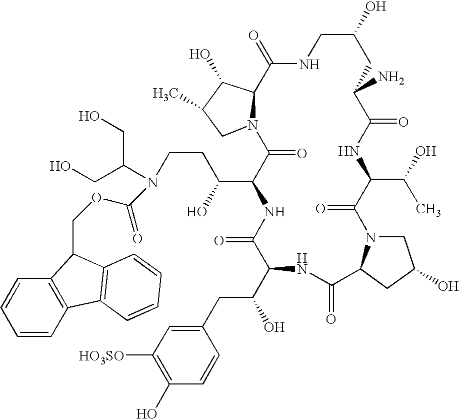 |
| | 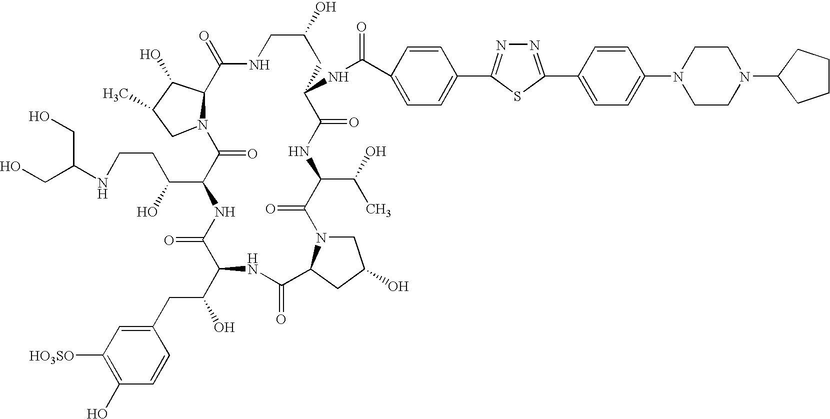 |
| 21 | 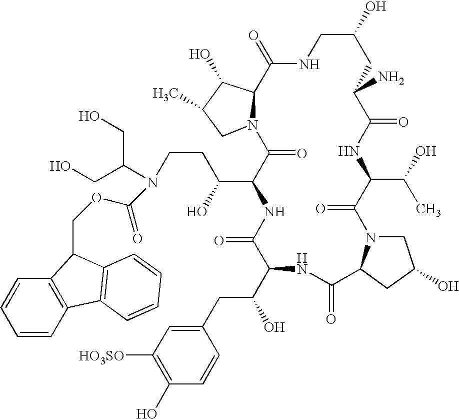 |
| | 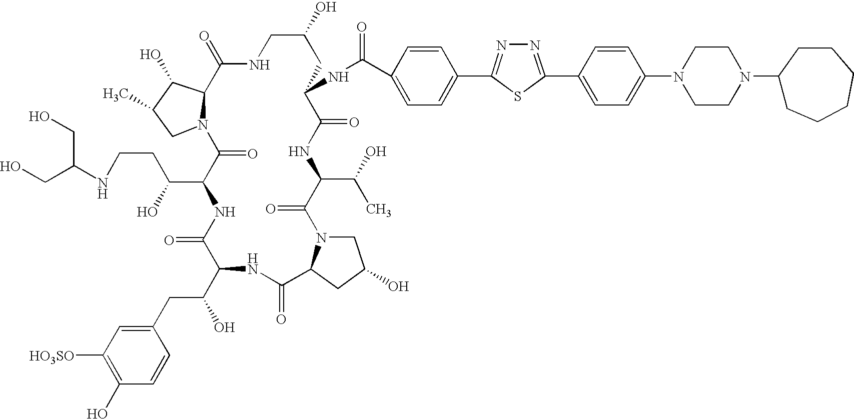 |
| 22 | 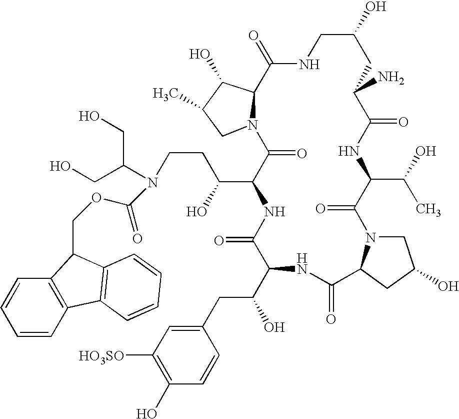 |
| | 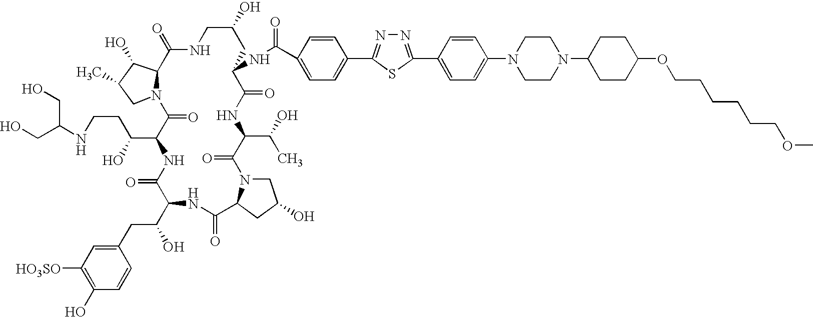 |
| 23 | 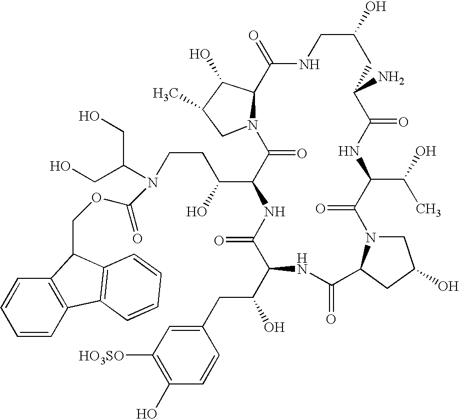 |
| | 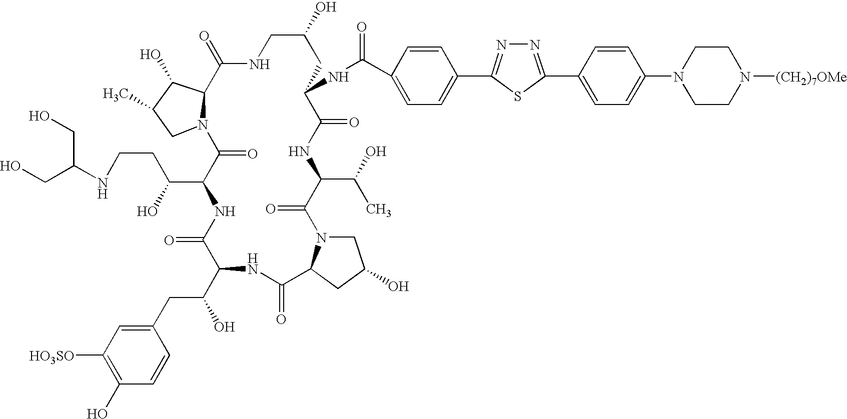 |
| 24 | 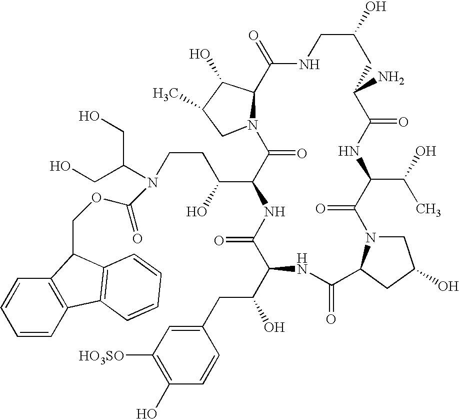 |
| | 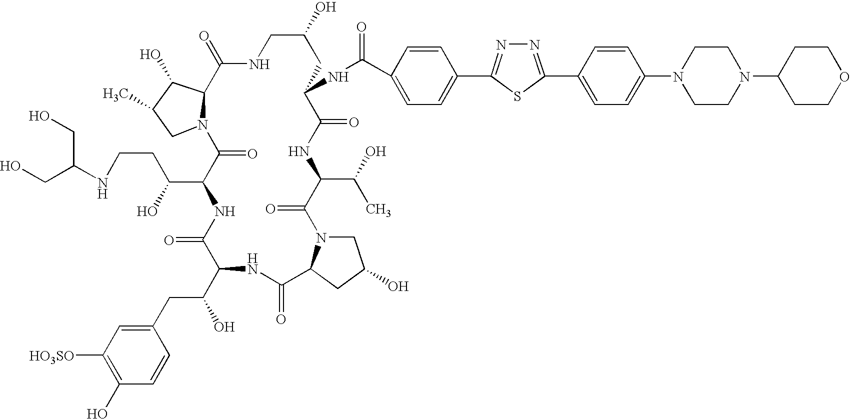 |
| 25 | 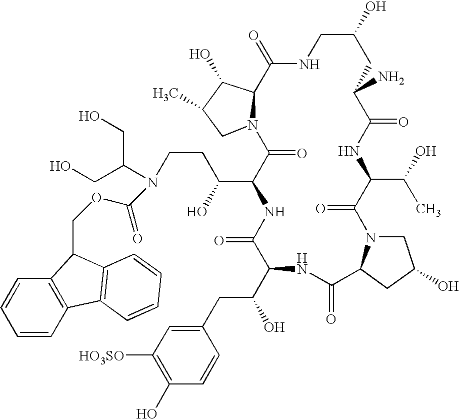 |
| | 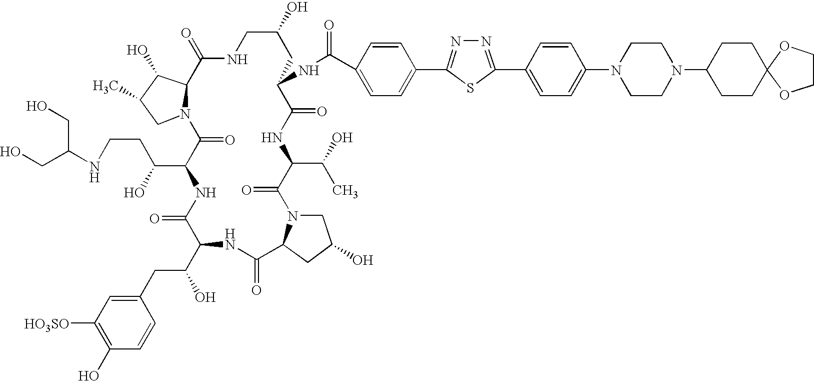 |
| 26 | 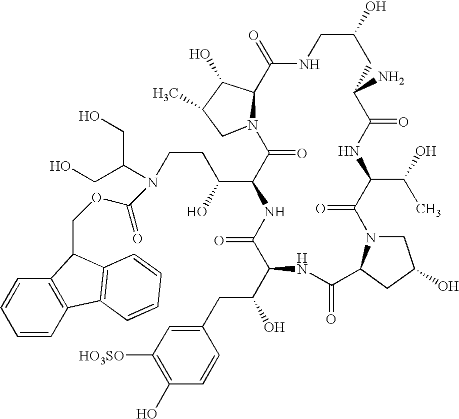 |
| | 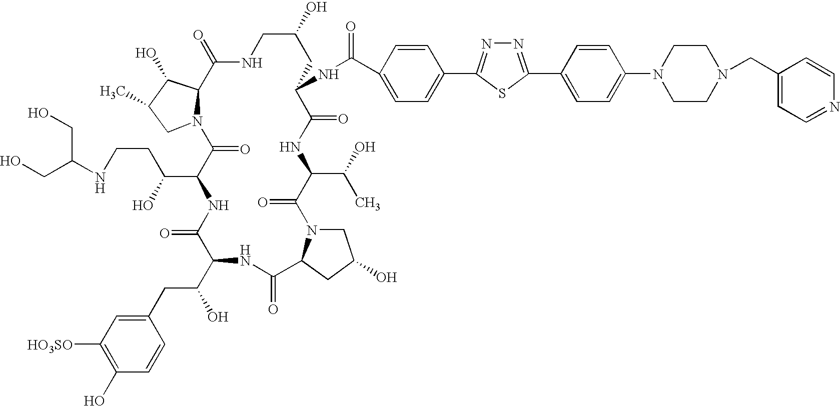 |
| 27 | 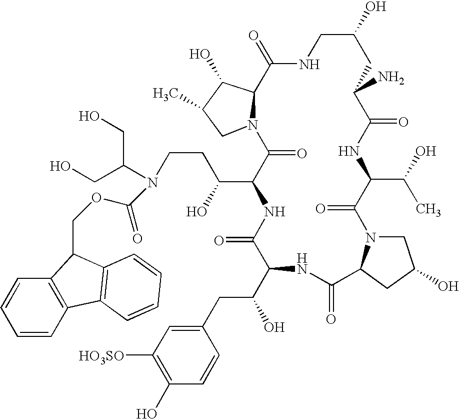 |
| | 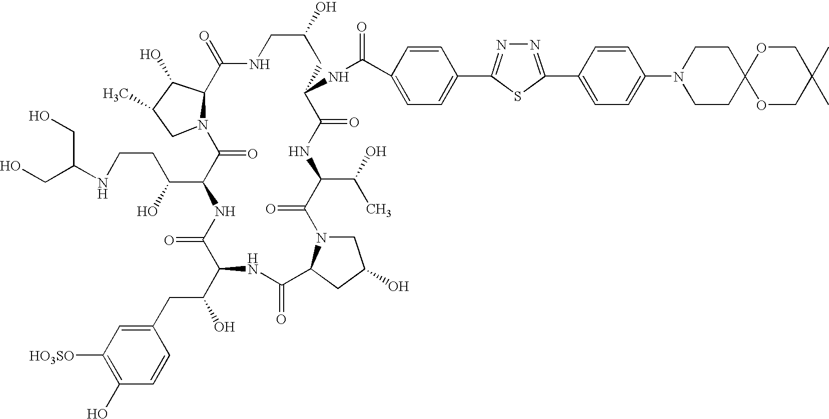 |
| 28 | 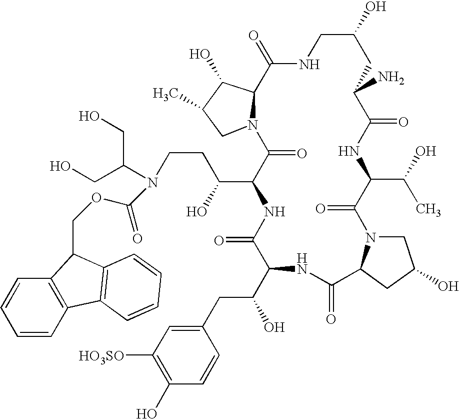 |
| | 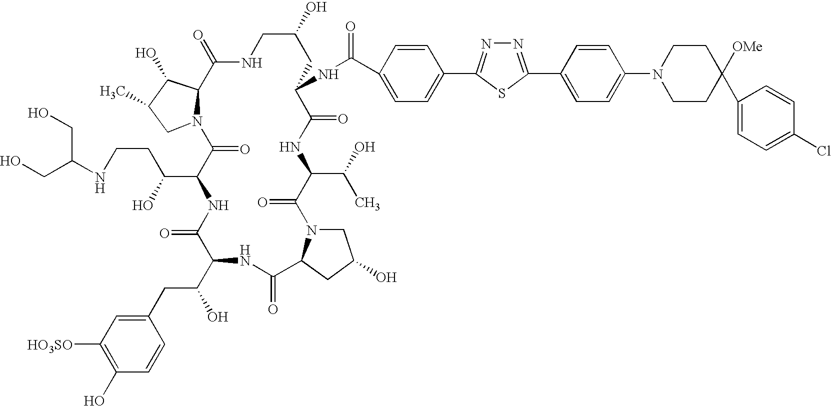 |
| 29 | 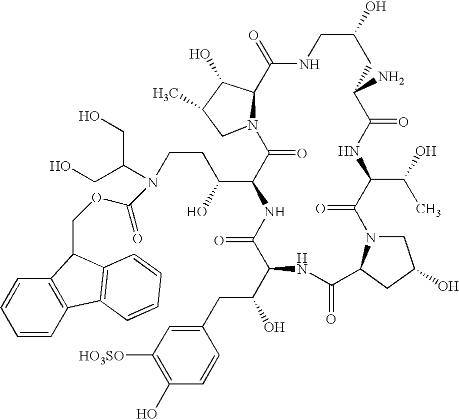 |
| | 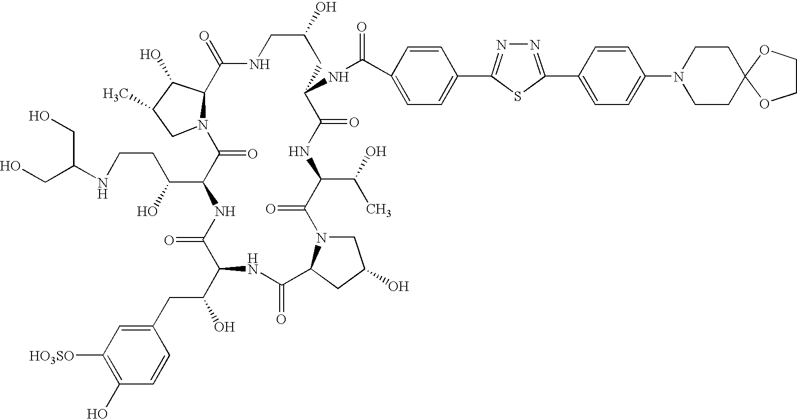 |
| 30 | 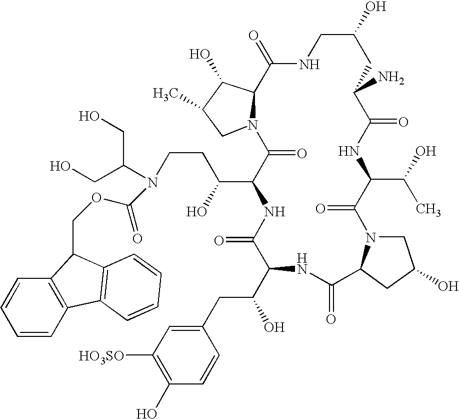 |
| | 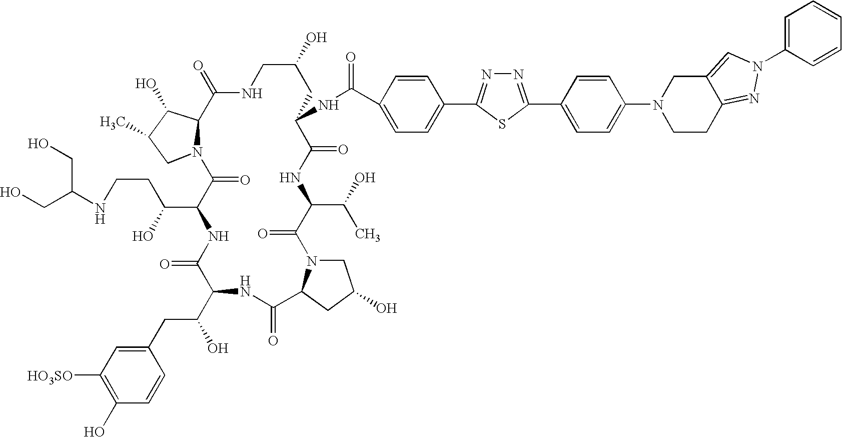 |
| 31 | 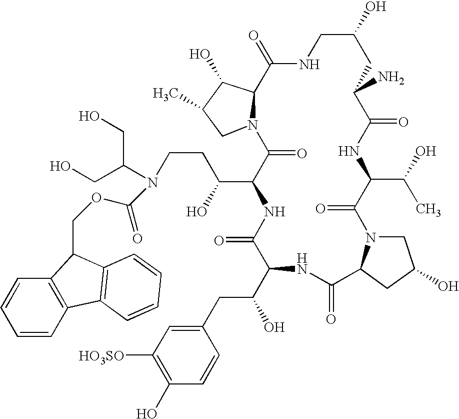 |
| | 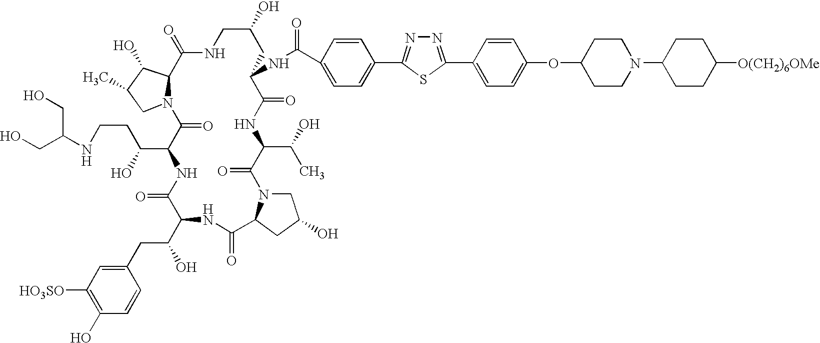 |
| 32 | 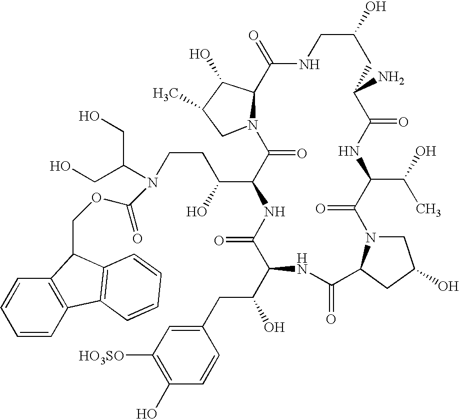 |
| | 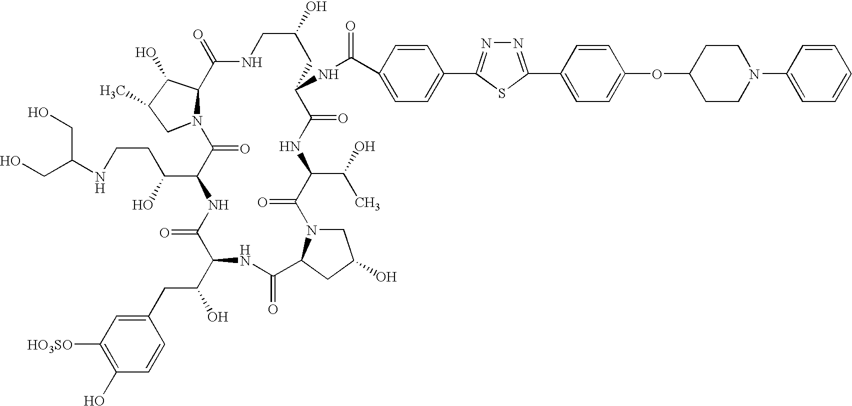 |
| 33 | 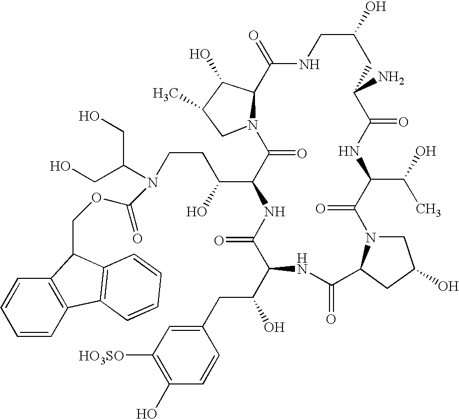 |
| | 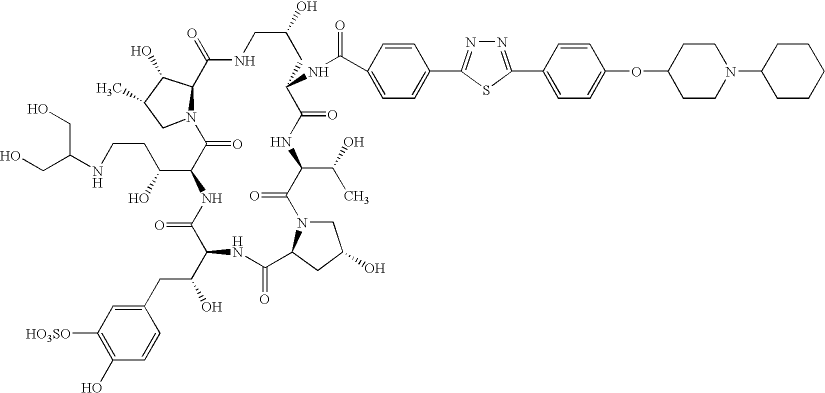 |
| 34 | 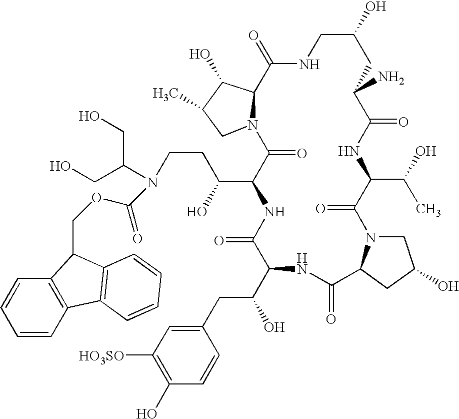 |
| | 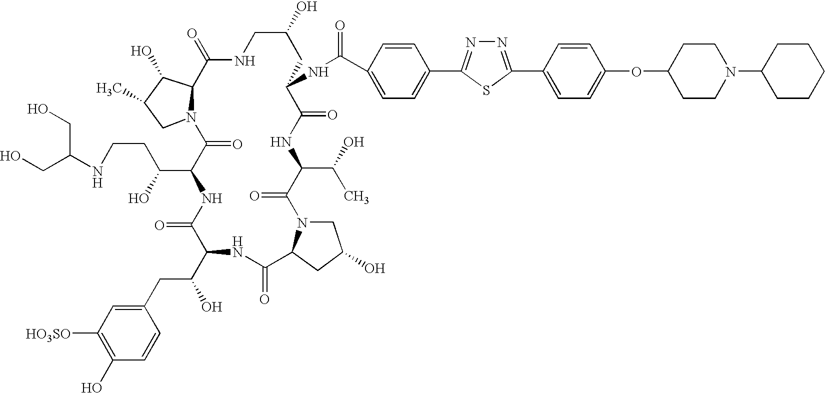 |
| 35 | 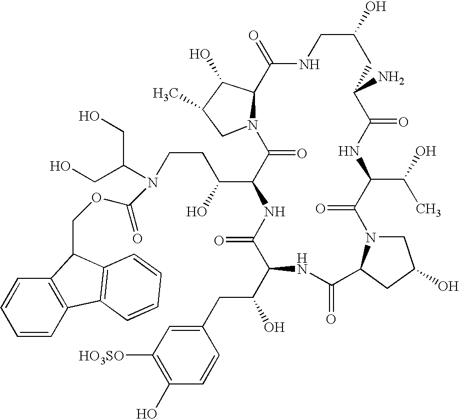 |
| | 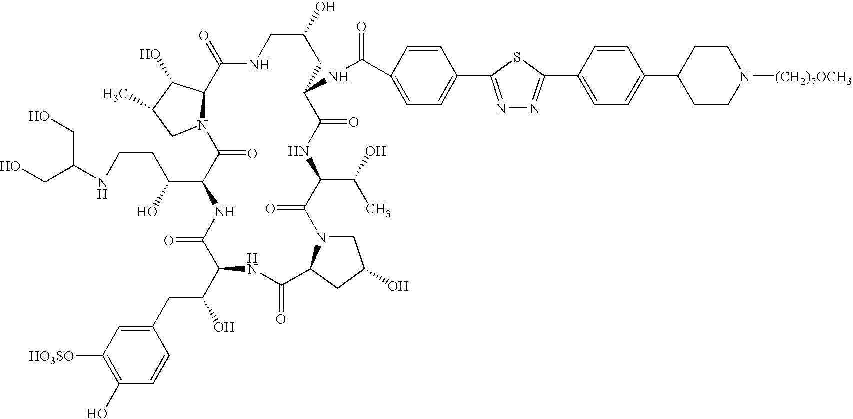 |
| 36 | 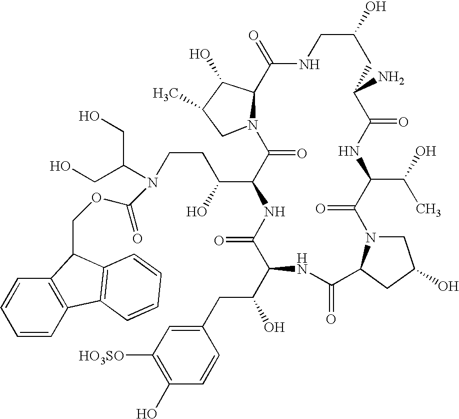 |
| | 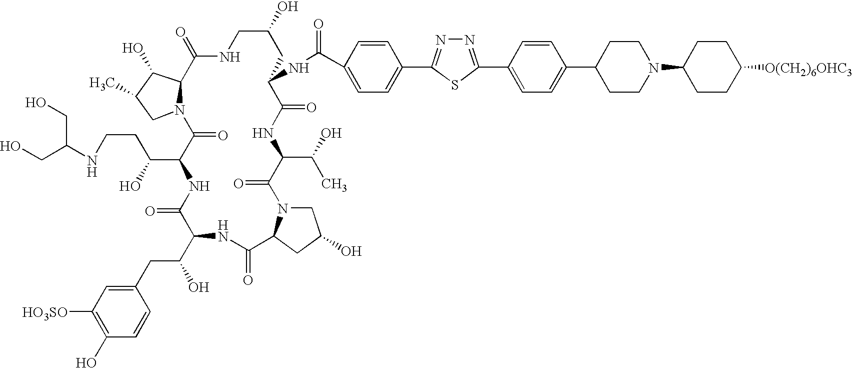 |
| 37 | 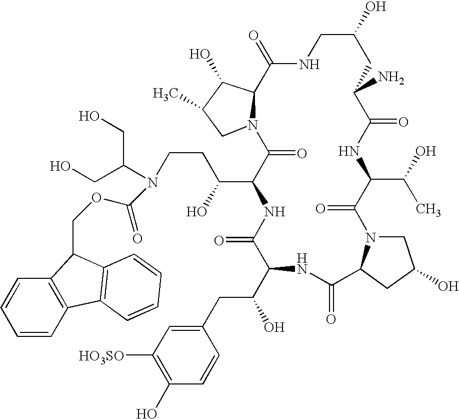 |
| | 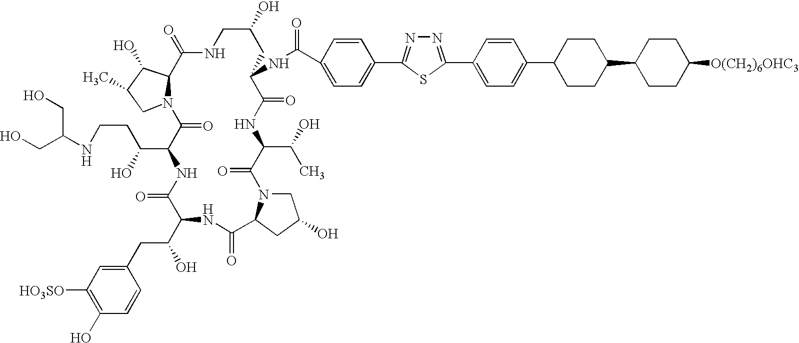 |
| 38 | 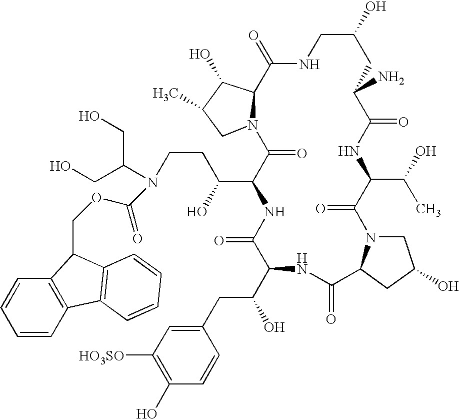 |
| | 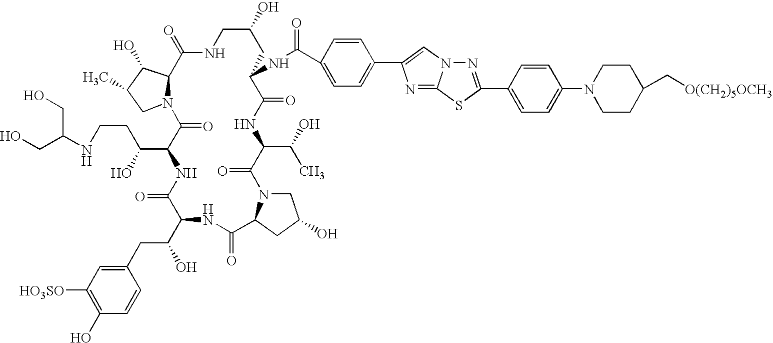 |
| 39 | 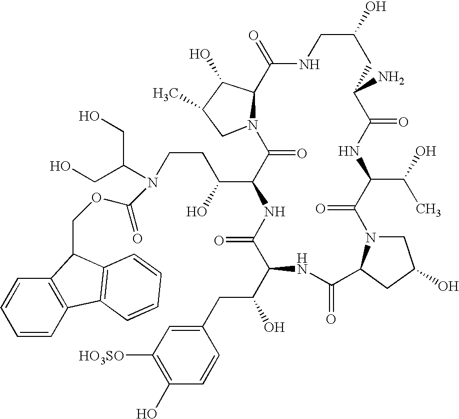 |
| | 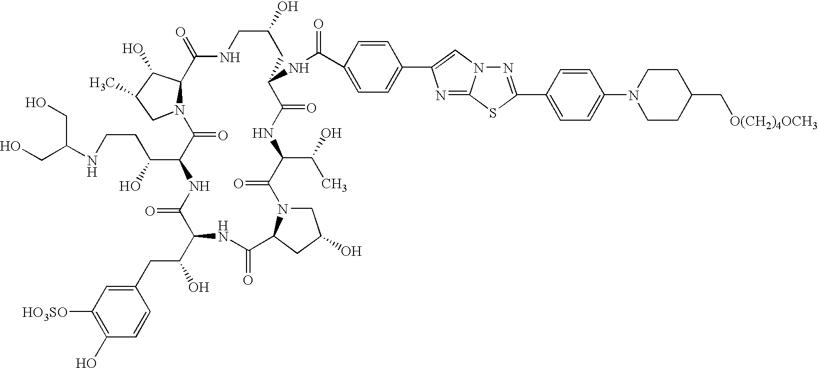 |
| 40 | 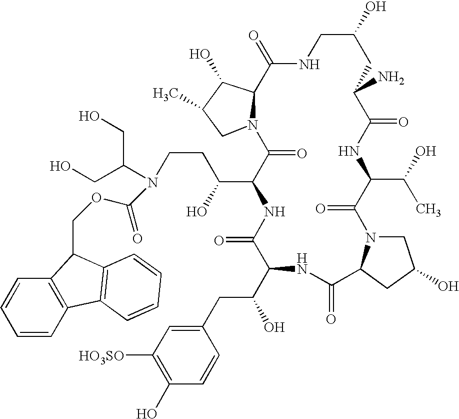 |
| | 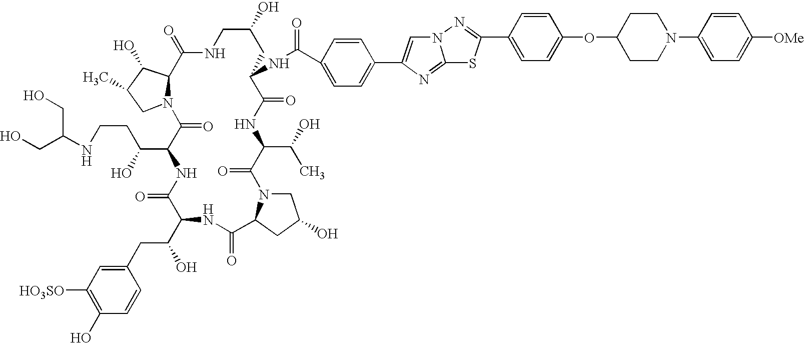 |
| 41 | 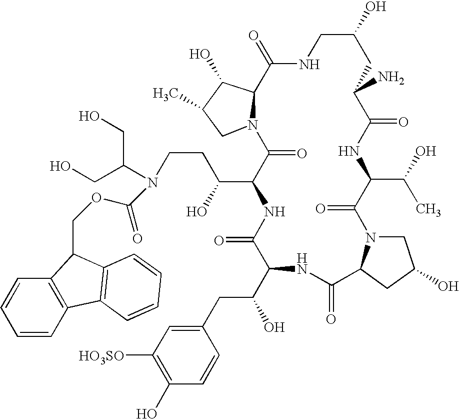 |
| | 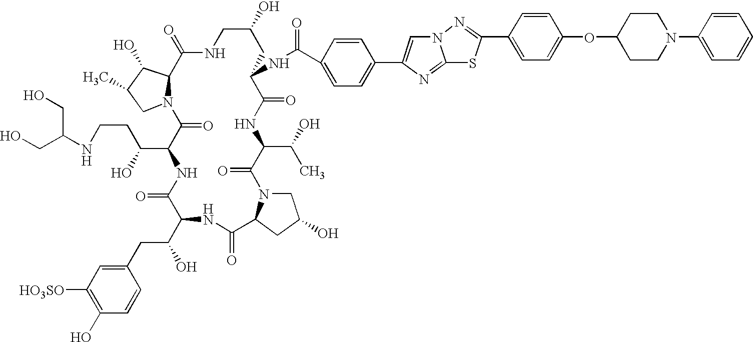 |
| 42 | 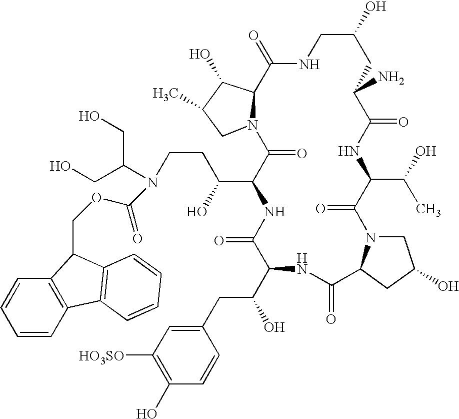 |
| | 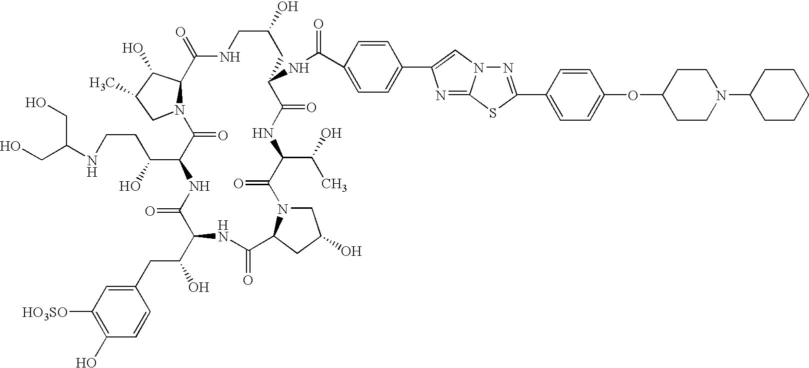 |
| 43 | 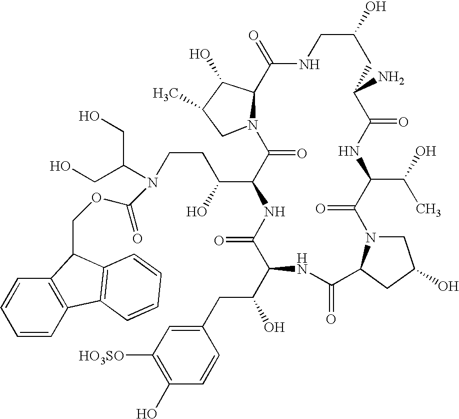 |
| | 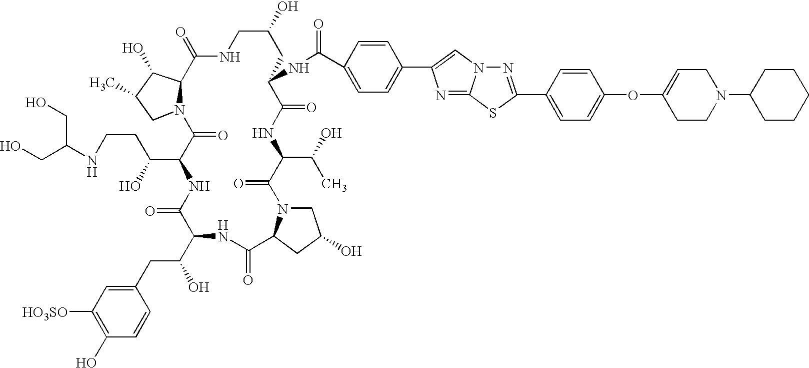 |
| 44 | 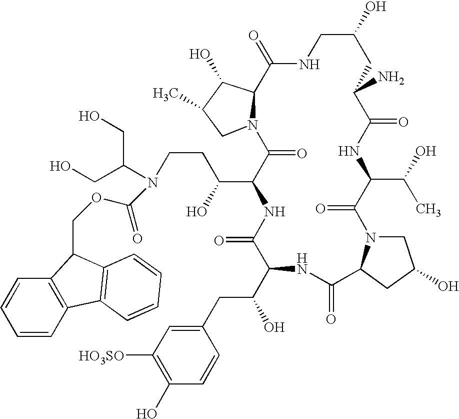 |
| | 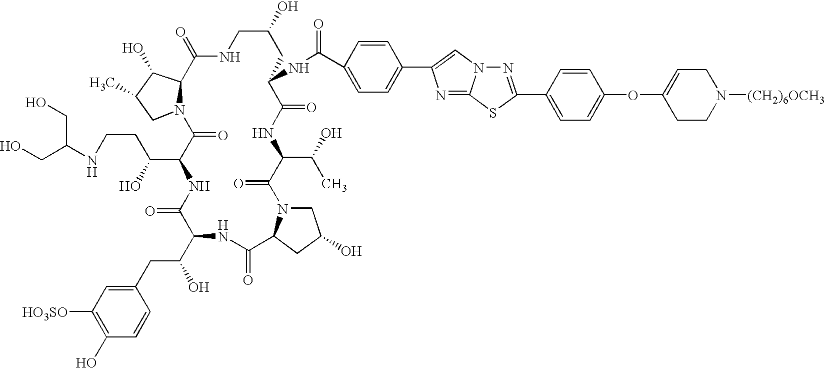 |
| 45 | 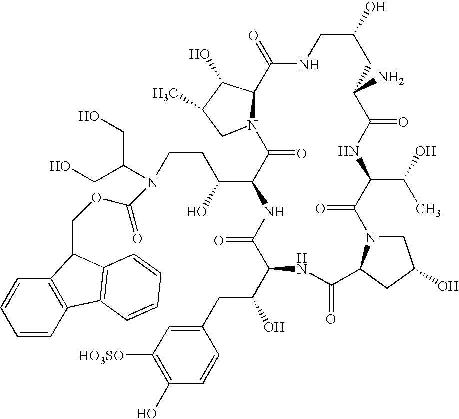 |
| | 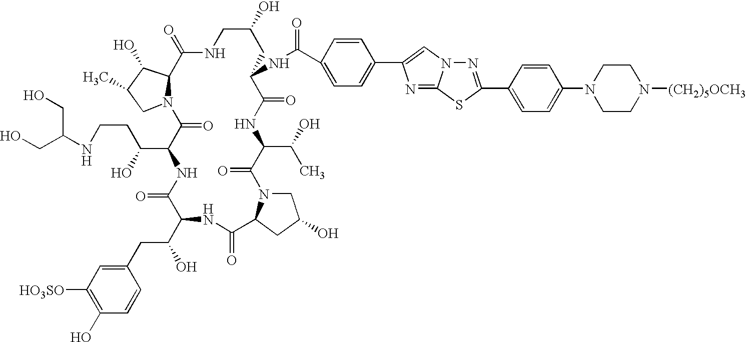 |
| 46 | 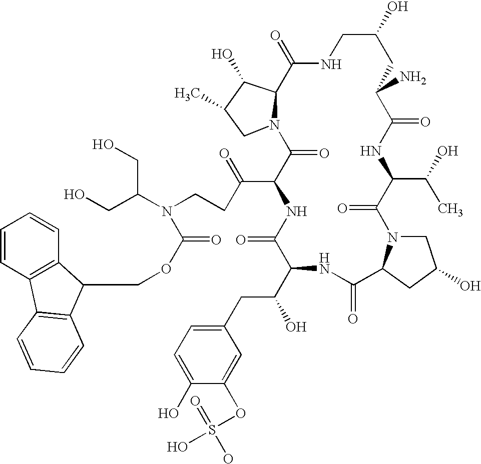 |
| | 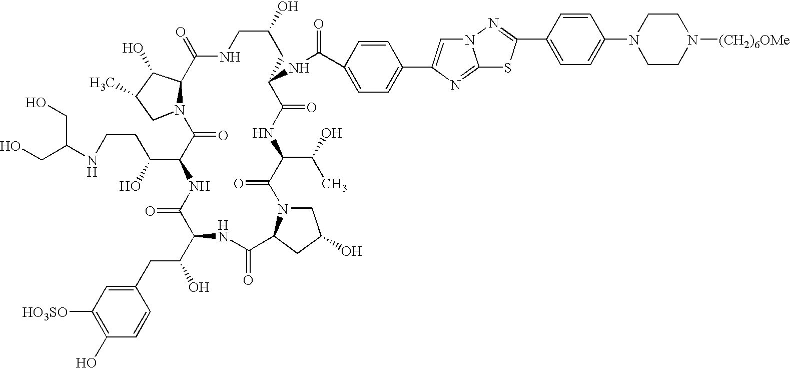 |
| 47 | 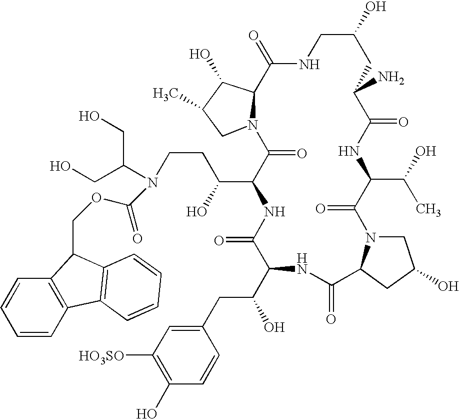 |
| | 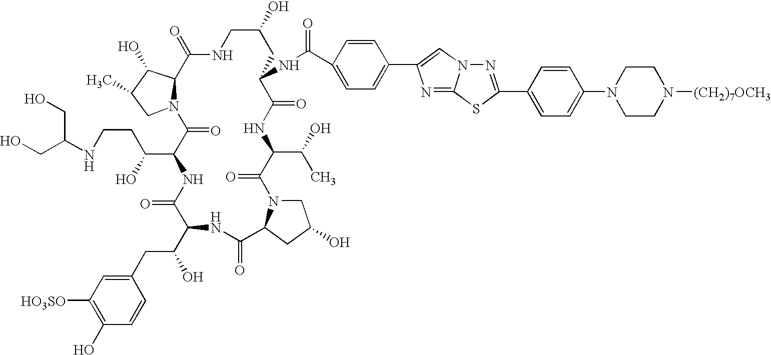 |
| 48 | 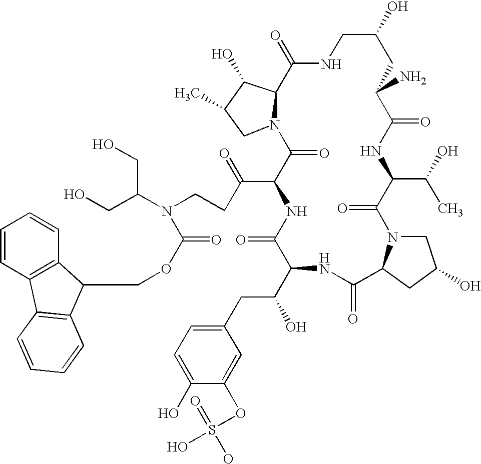 |
| | 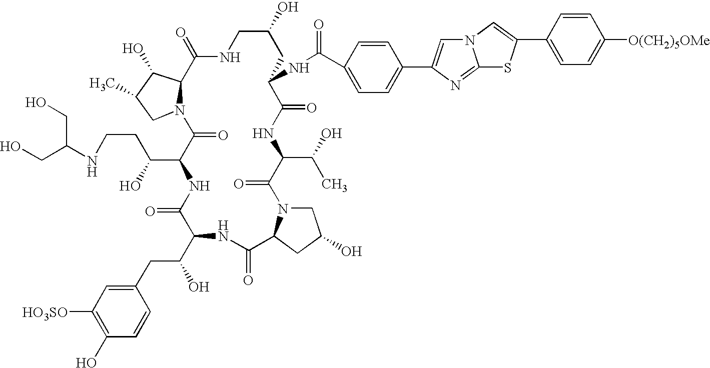 |
| 49 | 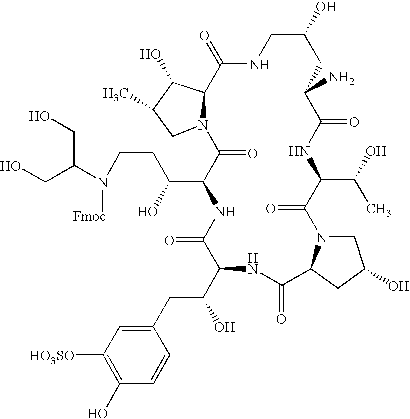 |
| | 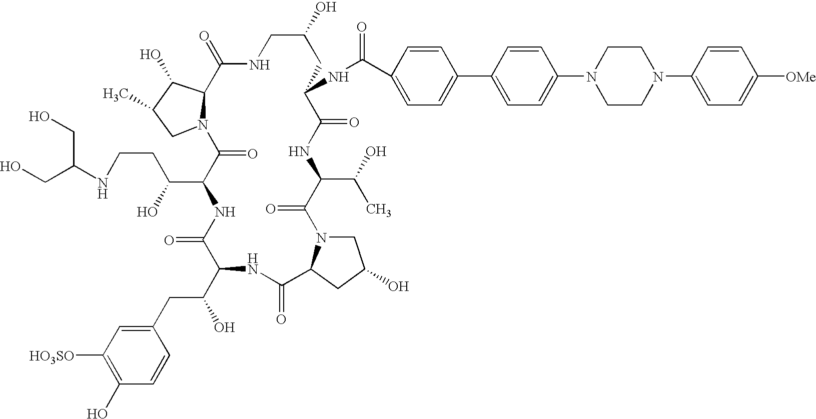 |
| 50 | 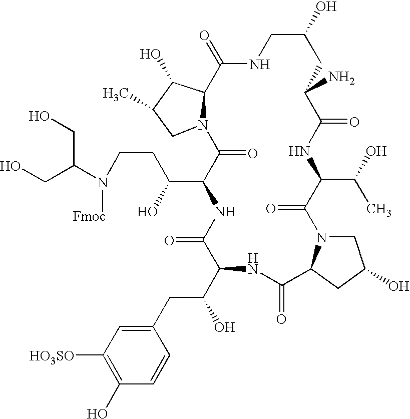 |
| | 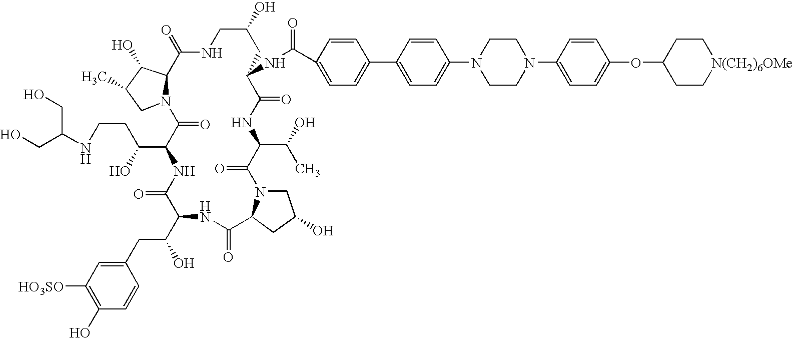 |
| 51 | 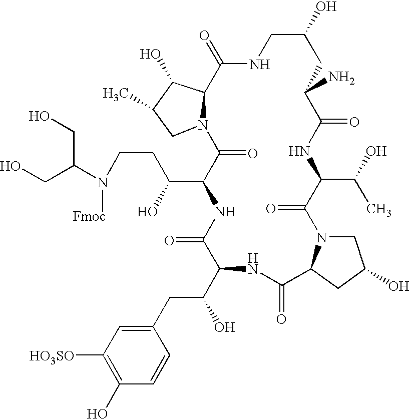 |
| | 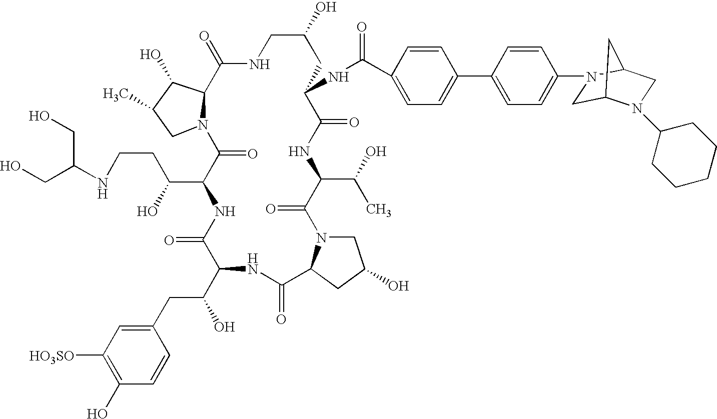 |
| 52 | 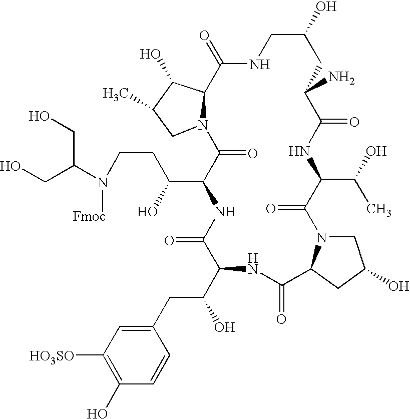 |
| | 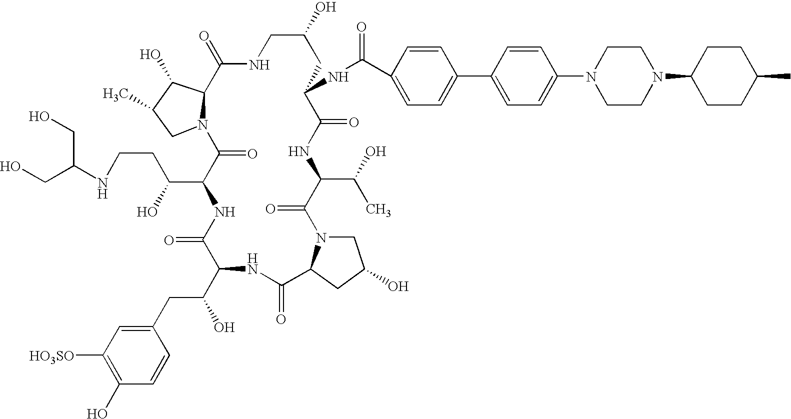 |
| 53 | 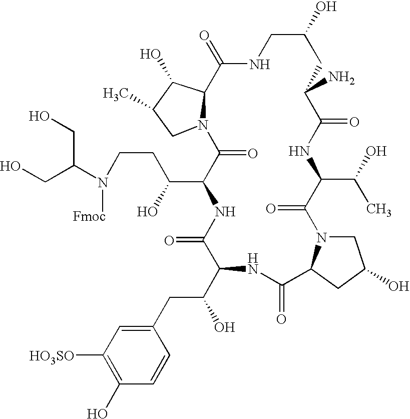 |
| | 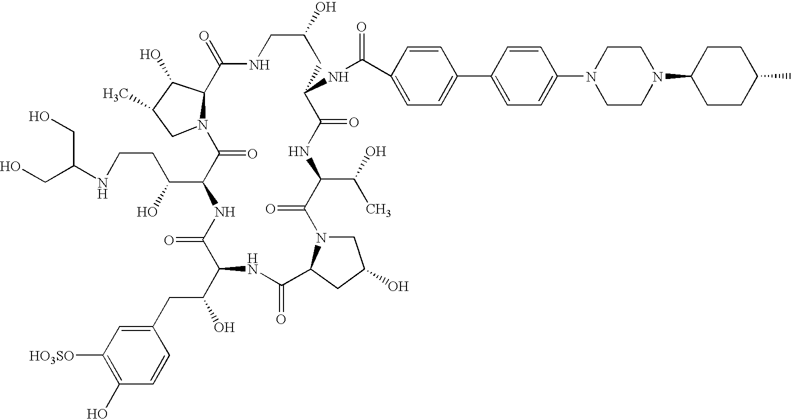 |
| 54 | 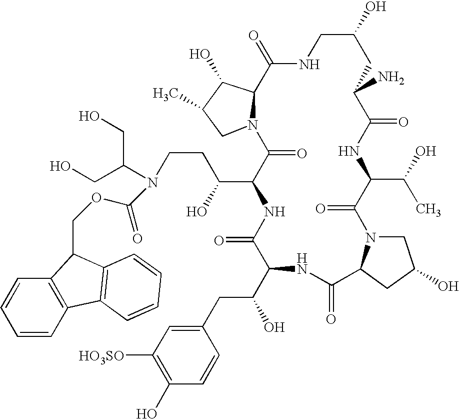 |
| | 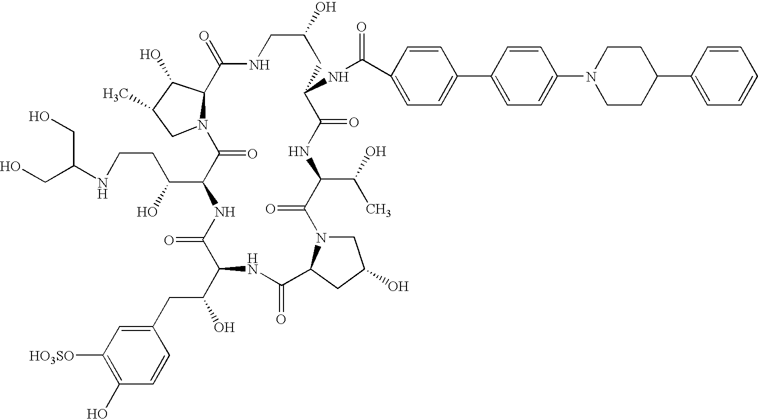 |
| 55 | 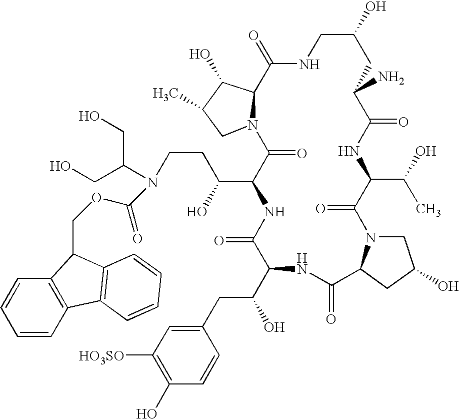 |
| | 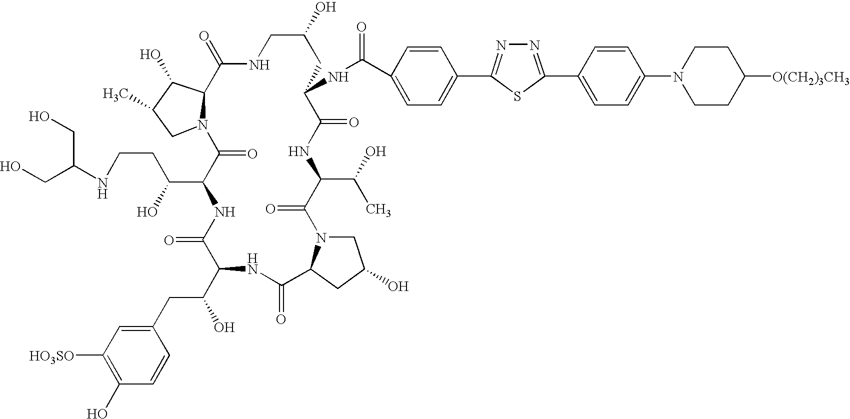 |
| 56 | 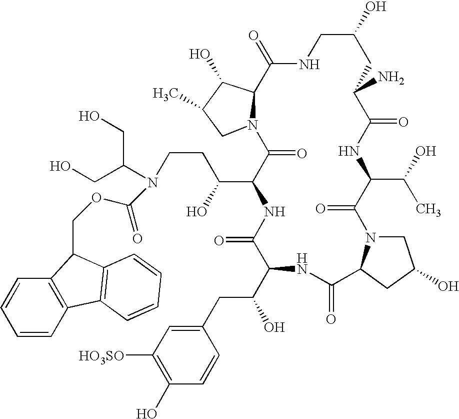 |
| | 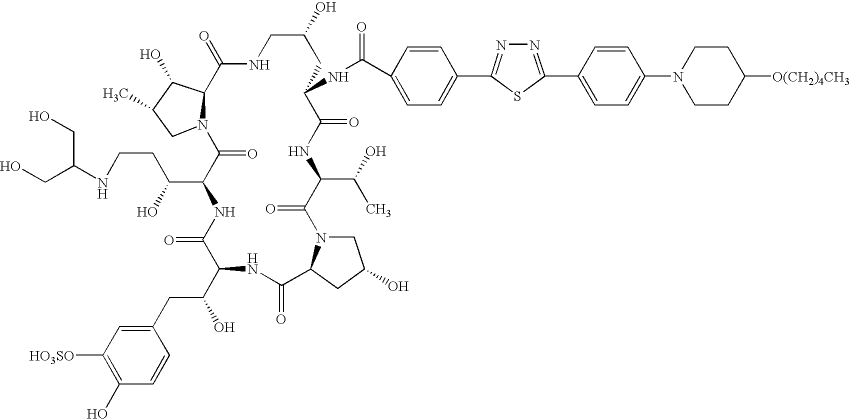 |
| 57 | 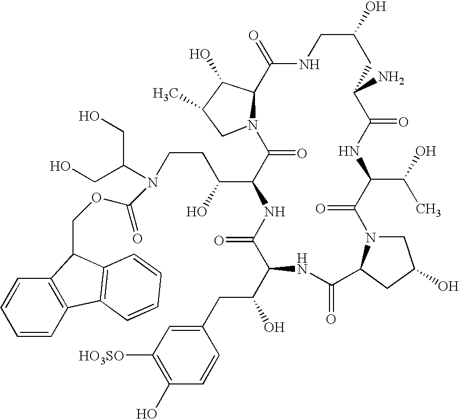 |
| | 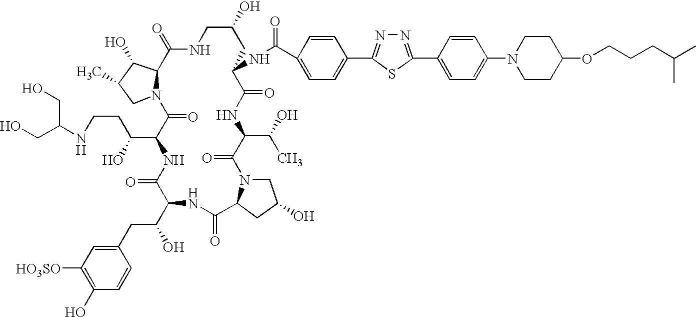 |
| 58 | 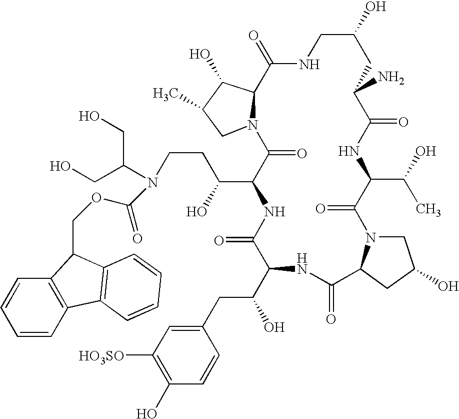 |
| | 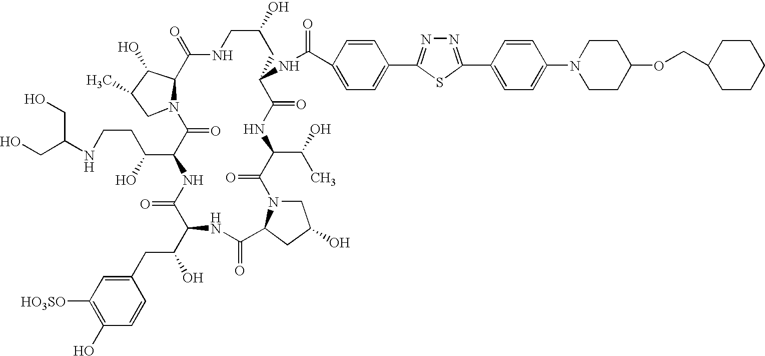 |
| 59 | 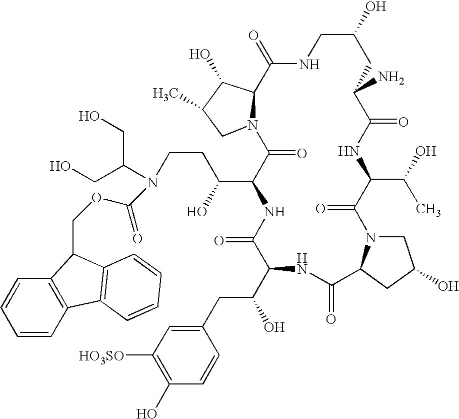 |
| | 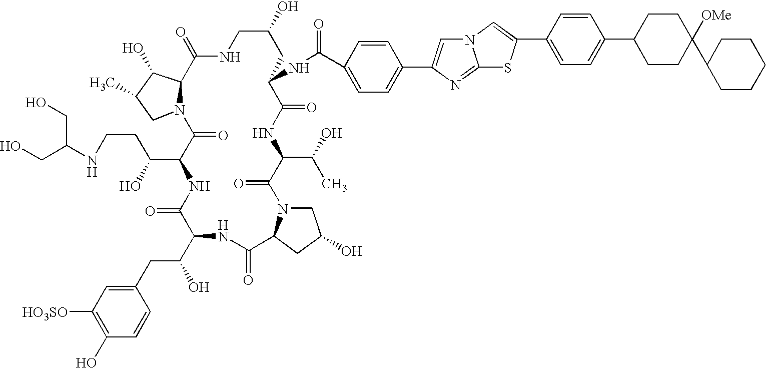 |
| 60 | 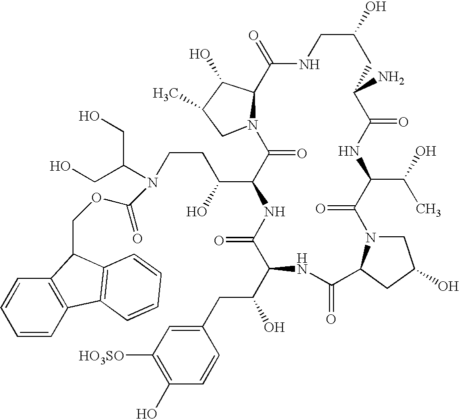 |
| | 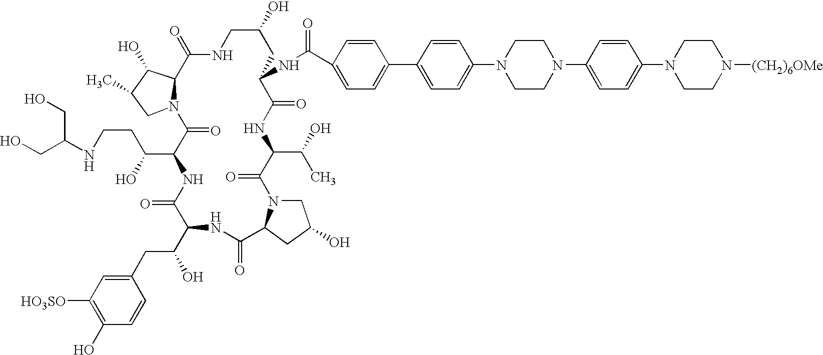 |
| 61 | 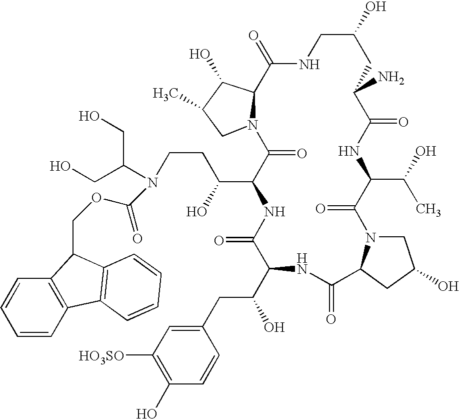 |
| | 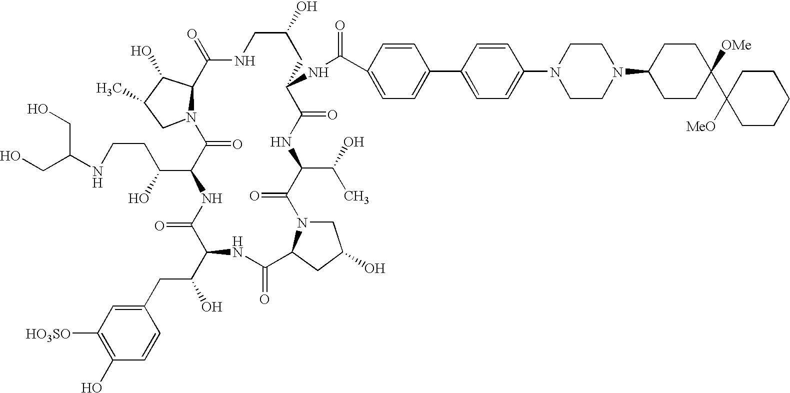 |
| 62 | 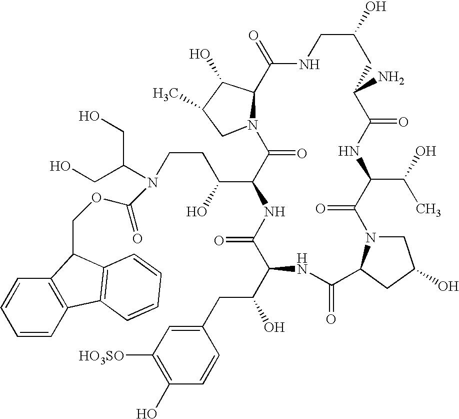 |
| | 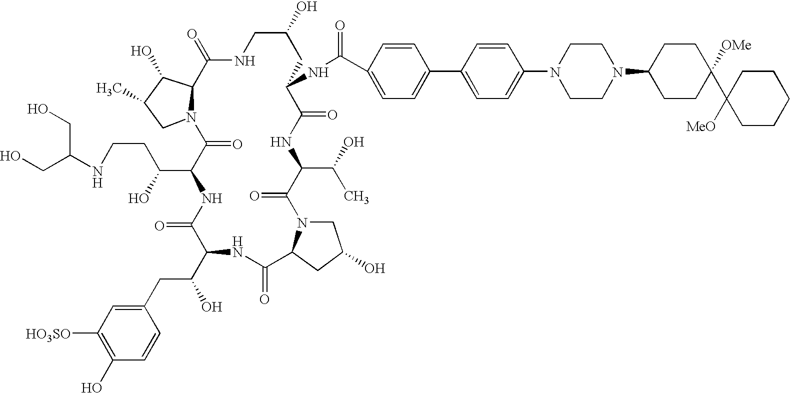 |
| 63 | 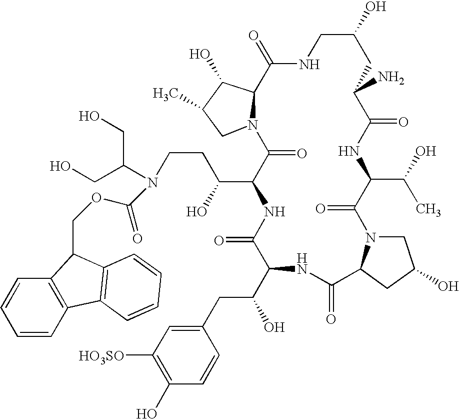 |
| | 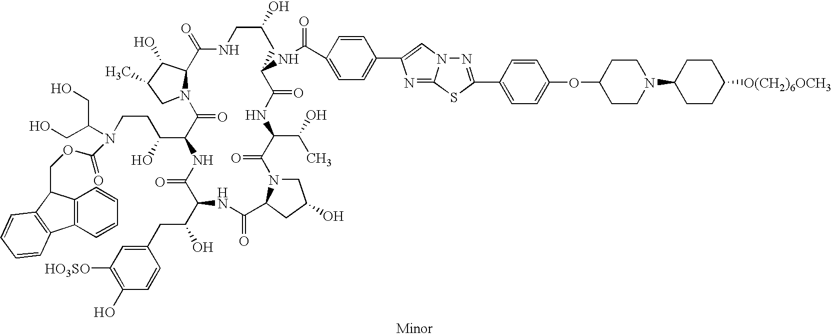 |
| | 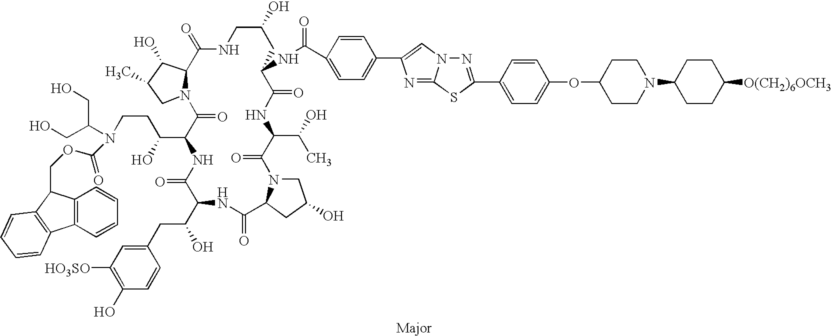 |
| 64 | 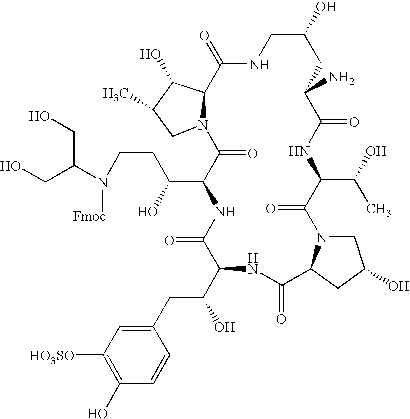 |
| | 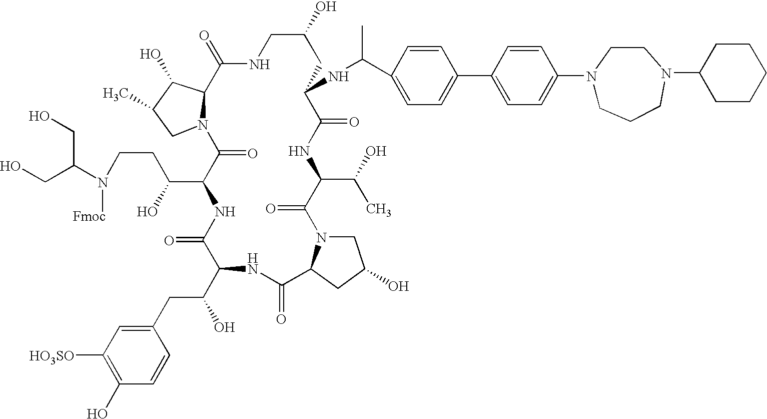 |
| 65 | 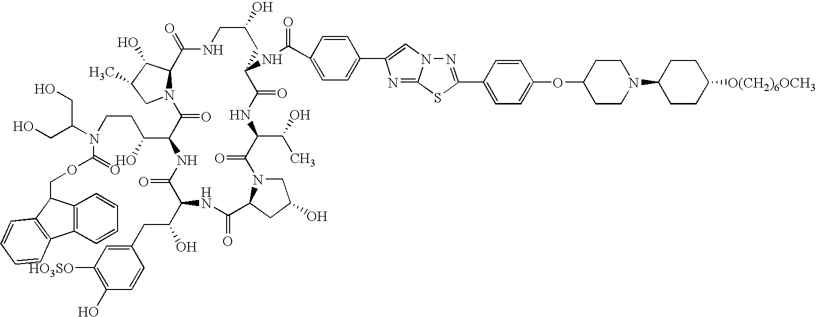 |
| | 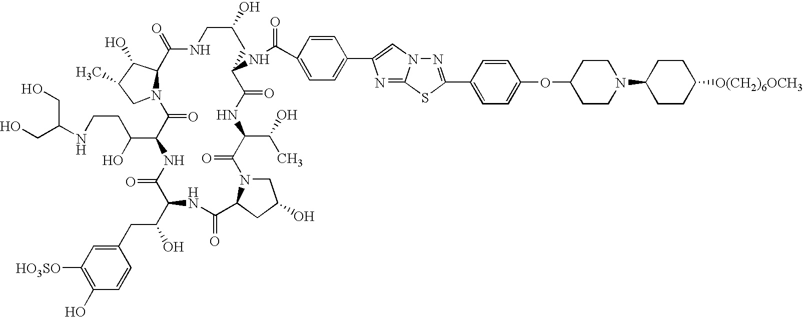 |
| 66 | 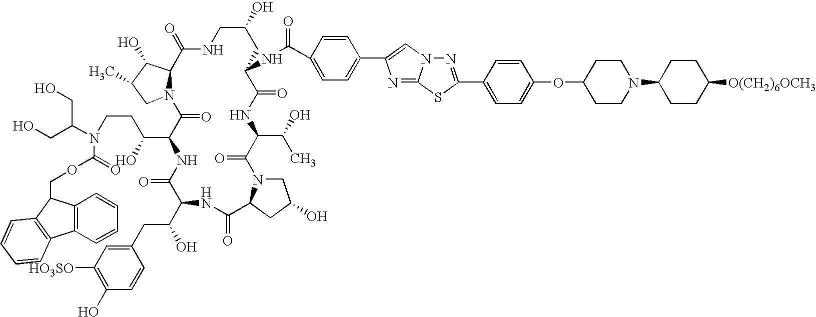 |
| | 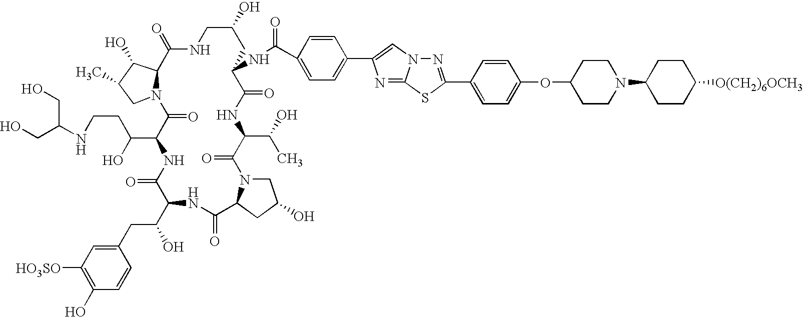 |
| 67 | 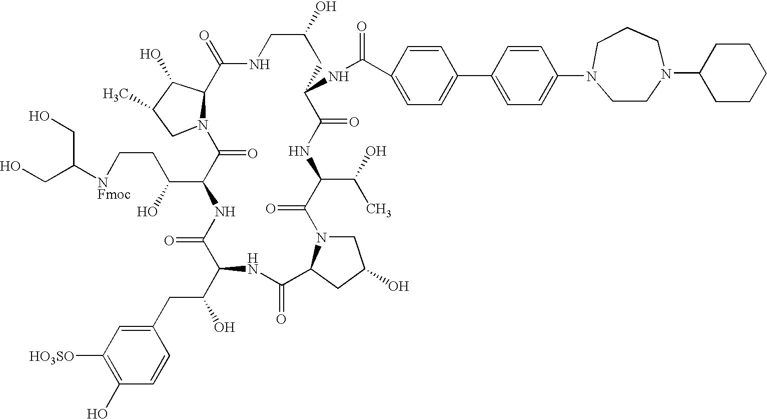 |
| | 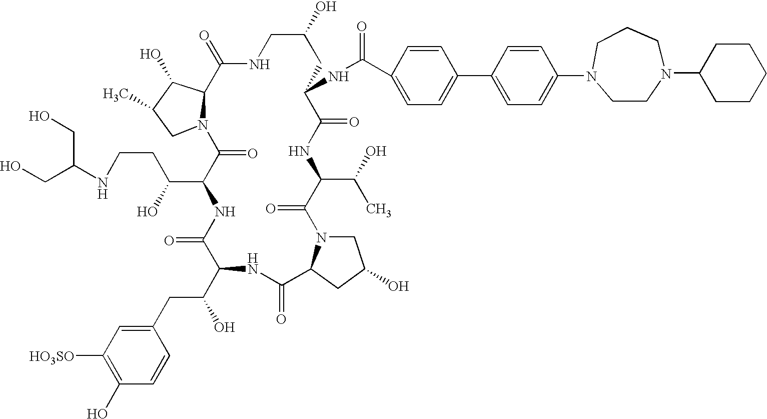 |
| 68 | 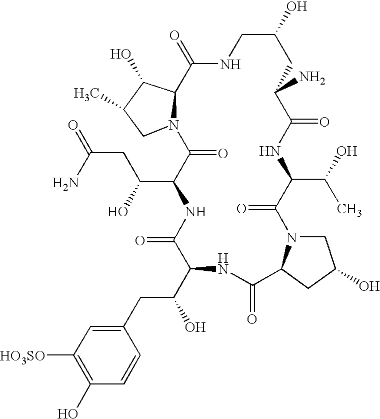 |
| | 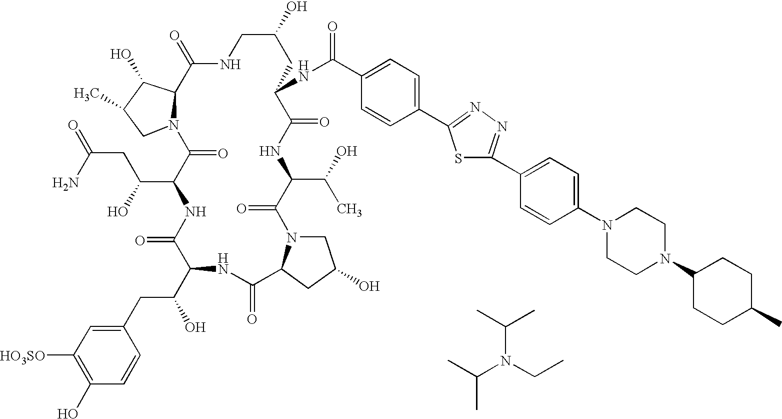 |
| 69 | 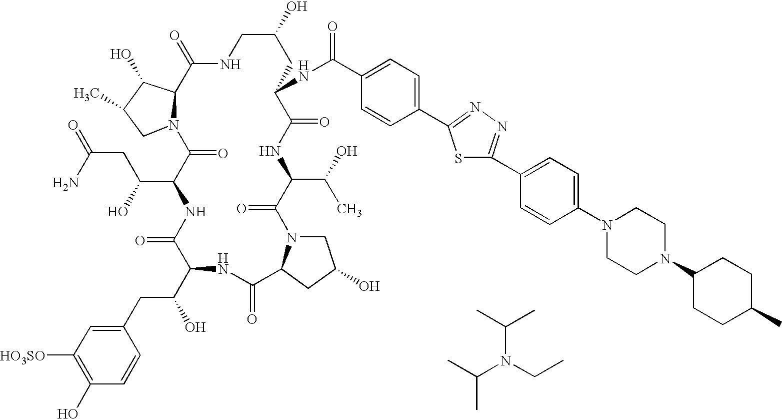 |
| | 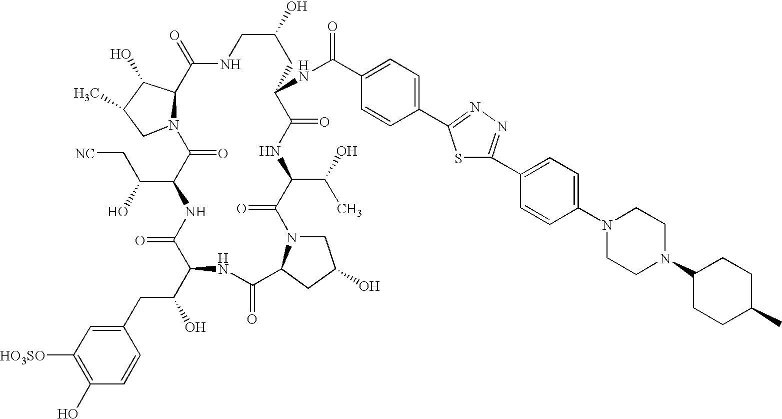 |
| 70 | 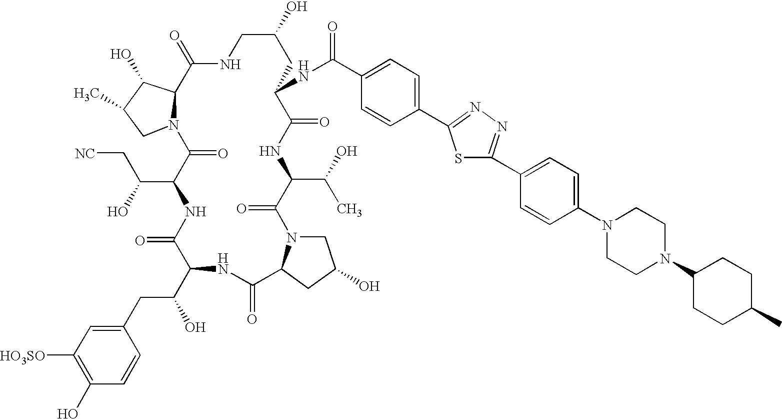 |
| | 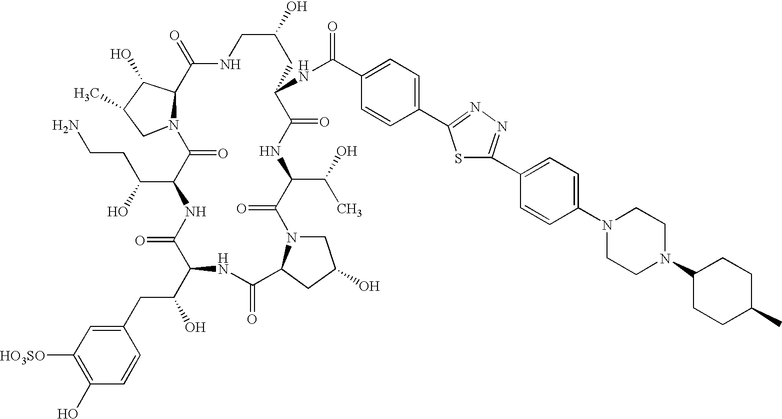 |
| 71 | 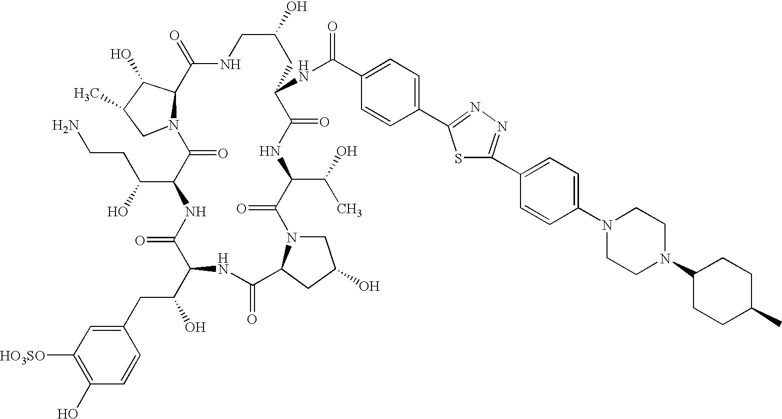 |
| | 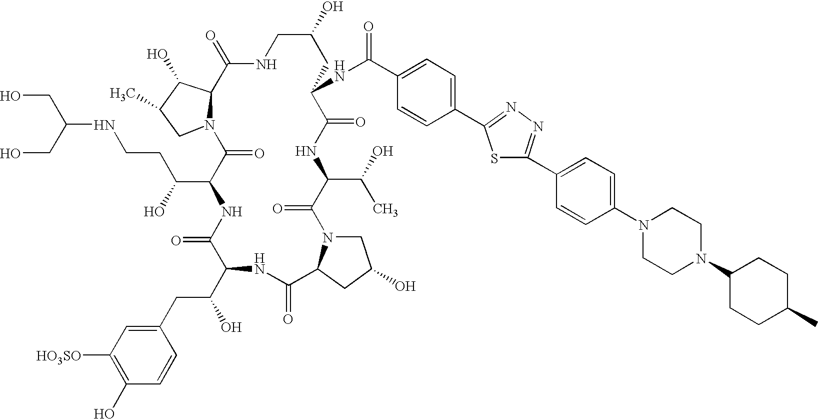 |
| 72 | 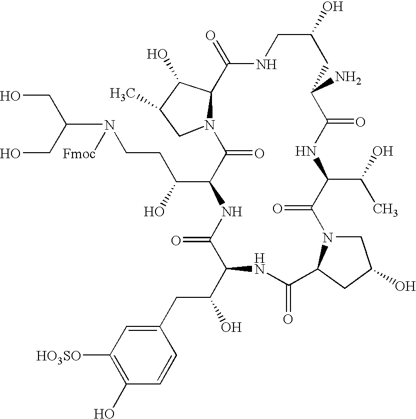 |
| | 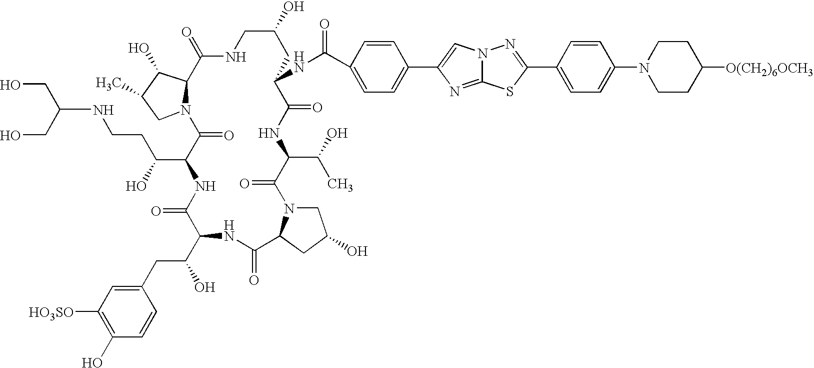 |
| 73 | 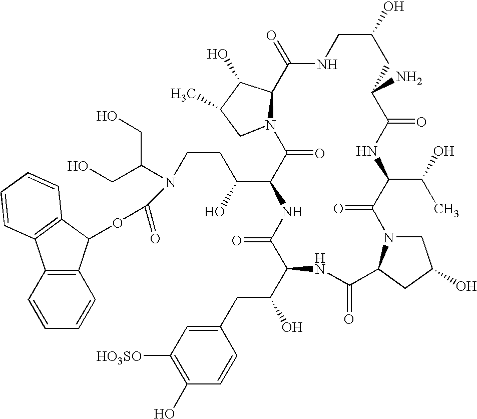 |
| | 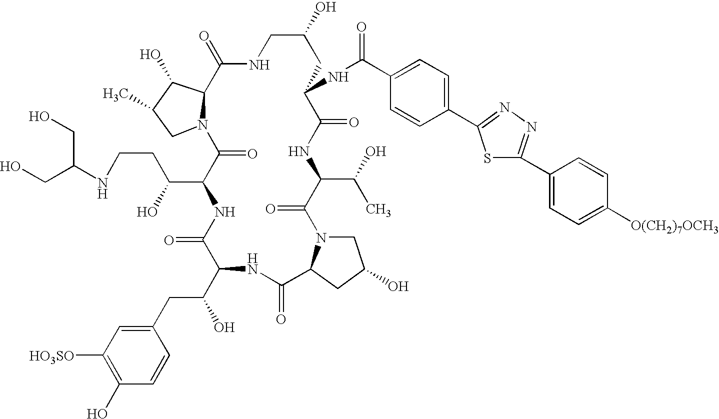 |
| 74 | 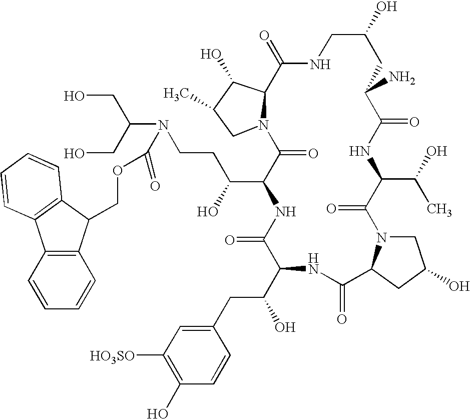 |
| | 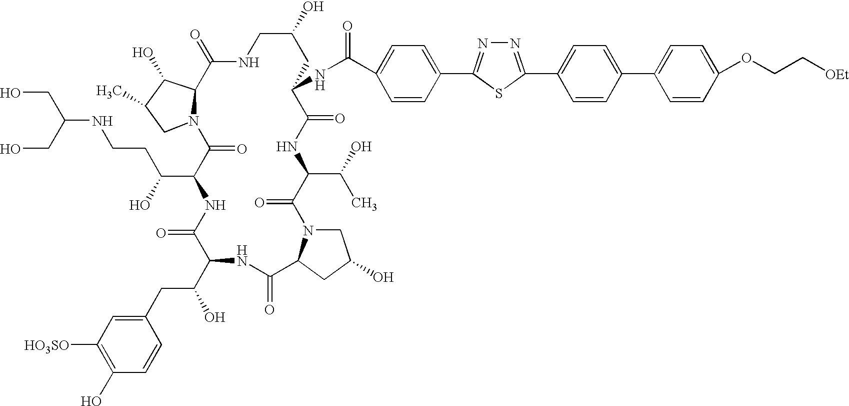 |
| 75 | 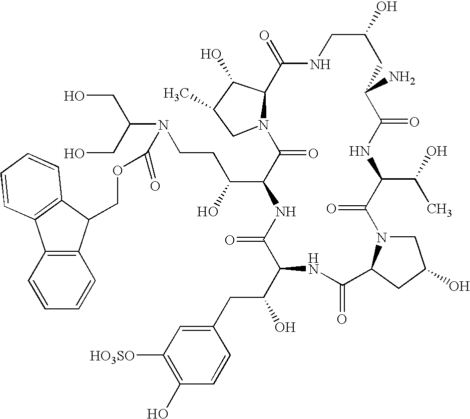 |
| | 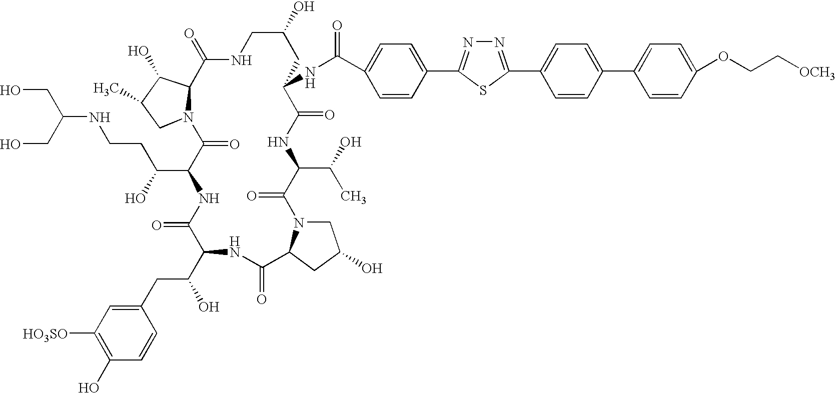 |
| 76 | 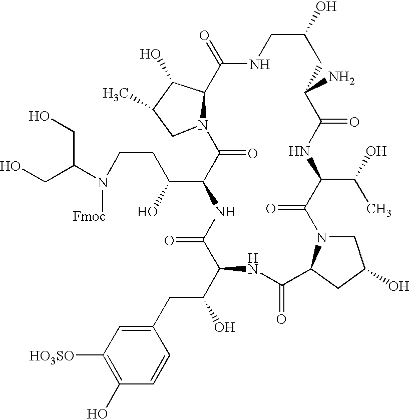 |
| | 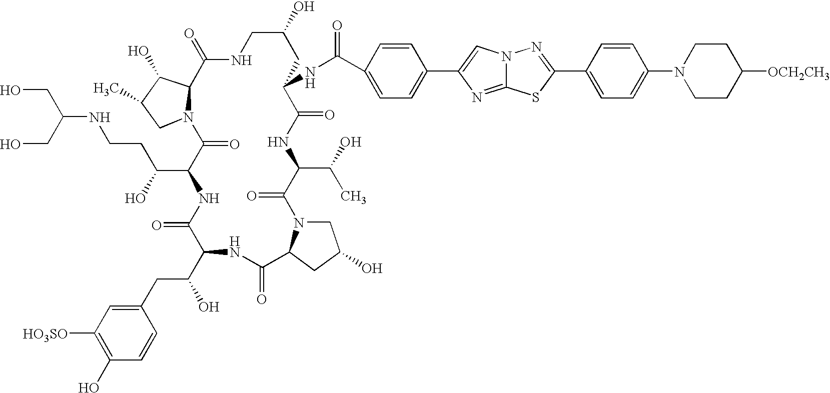 |
| 77 | 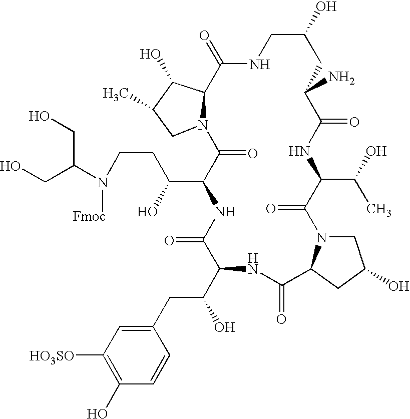 |
| | 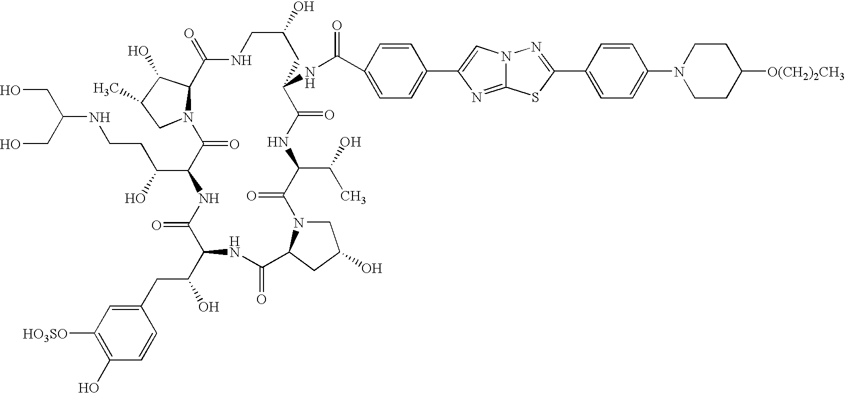 |
| 78 | 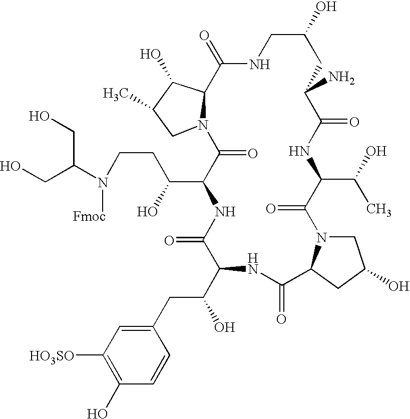 |
| | 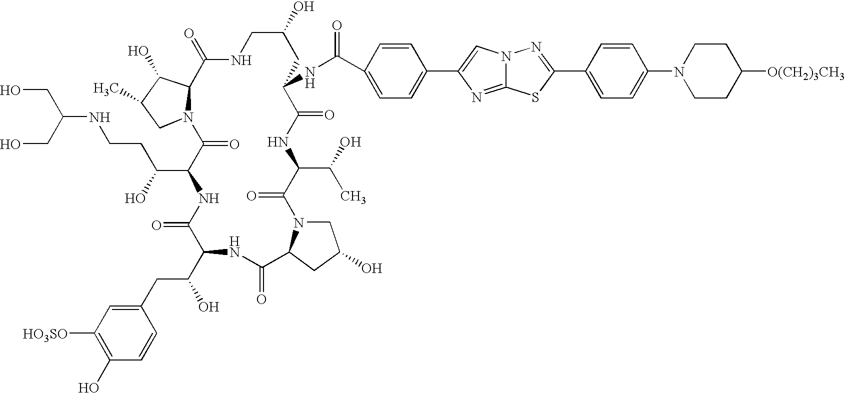 |
| 79 | 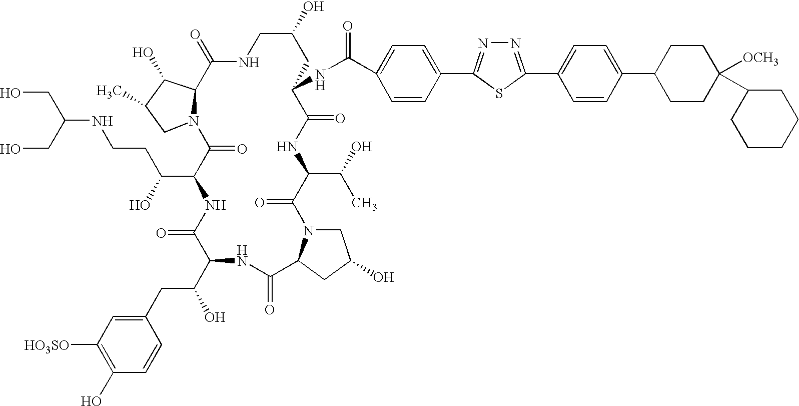 |
| | 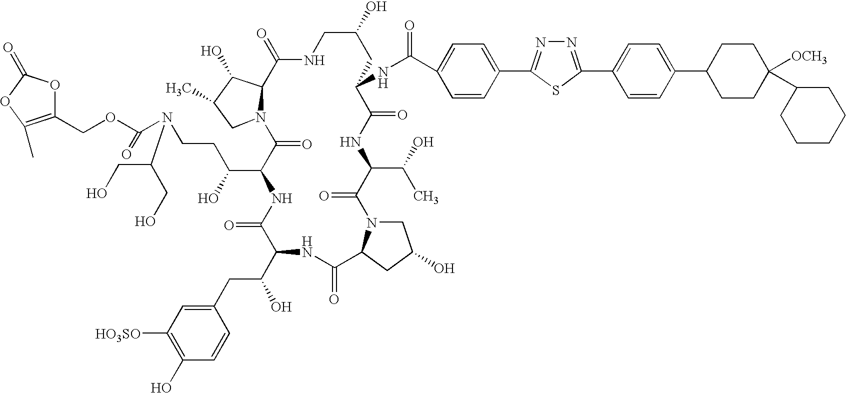 |

### EXAMPLE 1

A solution of Starting Compound (190 mg) in N,N-dimethylformamide (2 ml) was treated with 4-[4-[5-[4-(7-methoxyheptyloxy)phenyl]-1,3,4-thiadiazol-2-yl]-1-piperazinyl]benzoyl-1H-1,2,3-benzotriazole (100 mg) and stirred for 4 hours at ambient temperature. Piperidine (0.16 ml) was added the reaction mixture, and stirred for 2 hours at ambient temperature. Ethyl acetate (10 ml) was added, and resulting precipitate was collected, the precipitate was dissolved in a mixture of 10% acetonitrile in water and the solution was subjected to column chromatography on ODS (Daiso-gel, SP-120-40/60-ODS-B (Trademark: prepared by Daiso Co., Ltd.)) (60 ml) eluting with 40% acetonitrile in water. The fractions containing the object compound were collected and evaporated under reduced pressure to remove acetonitrile. The residue was lyophilized to give Object Compound (1).

IR (KBr): 3361.3, 1666.2, 1631.5, 1610.3, 1511.9, 1234.2 cm⁻¹ NMR (DMSO-d₆+D₂O, δ): 0.97 (3H, d, J=6.8 Hz), 1.08 (3H, d, J=6.0 Hz), 1.33-4.80 (59H, m), 6.70-7.87 (11H, m) ESI MASS (Negative)(m/z): 1455.6 (M⁺−H), 1456.6 (M⁺) Elemental Analysis Calcd. for C₆₅H₉₂N₁₂O₂₂S₂.6H₂O: C, 49.86; H, 6.69; N, 10.74 Found: C, 49.71; H, 6.70; N, 10.57

The following compounds [Example 2 to 62] were obtained according to a similar manner to that of Example 1.

### EXAMPLE 2

IR (KBr): 1664, 1628, 1444, 1431, 1408, 1269, 1192, 1043 cm⁻¹ NMR (DMSO-d₆+D₂O, δ): 0.98 (3H, d, J=6.8 Hz), 1.11 (3H, d, J=5.8 Hz), 1.6-2.6 (7H, m), 2.8-4.6 (25H, m), 4.7-4.9 (2H, m), 6.7-6.9 (2H, m), 7.0-7.11 (1H, m), 7.3-7.6 (3H, m), 7.7-8.2 (10H, m), 8.36 (1H, s), 9.16 (1H, s) ESI MASS (Negative): 1286.3 (M⁻−H) Elemental Analysis Calcd. for C₆₀H₇₄N₁₀O₂₀S_{4.5}H₂O: C, 52.66; H, 6.11; N, 10.24 Found: C, 52.60; H, 6.09; N, 10.10

### EXAMPLE 3

IR (KBr): 2933, 1659, 1628, 1547, 1462, 1444, 1246, 1196, 1041 cm⁻¹ NMR (DMSO-d₆+D₂O, δ): 0.8-1.2 (9H, m), 1.2-1.6 (6H, m), 1.6-2.6 (9H, m), 2.8-4.5 (27H, m), 4.7-4.9 (2H, m), 6.7-6.9 (2H, m), 7.0-7.2 (3H, m), 7.7-8.1 (6H, m), 8.26 (1H, s), 8.98 (1H, s) ESI MASS (Positive): 1333.2 (M⁺+Na) Elemental Analysis Calcd. for C₆₀H₈₂N₁₀O₂₁S-4.5H₂O: C, 51.75; H, 6.59; N, 10.06 Found: C, 51.75; H, 6.58; N, 10.04

### EXAMPLE 4

IR (KBr): 3356, 2933, 1633, 1539, 1508, 1435, 1246, 1043 cm⁻¹ NMR (DMSO-d₆+D₂O, δ): 0.89 (3H, t, J=6.6 Hz), 0.98 (3H, d, J=6.8 Hz), 1.10 (3H, d, J=5.6 Hz), 1.2-1.5 (6H, m), 1.6-2.6 (9H, m), 2.8-4.5 (27H, m), 4.8-4.9 (2H, m), 6.7-6.9 (2H, m), 6.9-7.1 (3H, m), 7.65 (2H, d, J=8.6 Hz), 7.9-8.1 (4H, m), 8.21 (1H, s), 8.99 (1H, s) ESI MASS: 1333.3 (M⁺+Na) (Positive), 1310.4 (M⁻−H) (Negative) Elemental Analysis Calcd. for C₆₀H₈₂N₁₀O₂₁S.6H₂O: C, 50.77; H, 6.67; N, 9.87 Found: C, 50.72; H, 6.77; N, 9.87

### EXAMPLE 5

IR (KBr): 2935, 1659, 1635, 1529, 1518, 1444, 1412, 1255, 1043 cm⁻¹ NMR (DMSO-d₆+D₂O, δ): 0.98 (3H, d, J=6.7 Hz), 1.09 (3H, d, J=5.9 Hz), 1.2-2.6 (17H, m), 2.8-4.6 (26H, m), 4.7-4.9 (2H, m), 6.7-6.9 (2H, m), 7.0-7.1 (1H, m), 7.14 (2H, d, J=8.9 Hz), 7.97 (2H, d, J=8.7 Hz), 8.0-8.2 (4H, m) ESI MASS (Negative): 1326.3 (M⁻−H) Elemental Analysis Calcd. for C₅₉H₇₈N₁₀O₂₁S₂:.5H₂O: C, 49.99; H, 6.26; N, 9.88 Found: C, 50.06; H, 6.14; N, 9.79

### EXAMPLE 6

IR (KBr): 3356, 2931, 1630, 1529, 1516, 1439, 1271, 1217, 1043 cm⁻¹ NMR (DMSO-d₆+D₂O, δ): 0.88 (3H, t, J=6.9 Hz), 0.97 (3H, d, J=6.7 Hz), 1.10 (3H, d, J=5.8 Hz), 1.2-1.6 (8H, m), 1.6-2.6 (9H, m), 2.8-4.6 (27H, m), 4.7-4.9 (2H, m), 6.6-6.8 (2H, m), 7.0-7.1 (1H, m), 7.2-7.3 (1H, m), 7.3-7.4 (1H, m), 7.8-8.1 (3H, m), 8.43 (1H, s) ESI MASS: 1255.3 (M⁺+Na) (Positive), 1232.4 (M⁻−H)(Negative) Elemental Analysis Calcd. for C₅₆H₈₀N₈O₂₁S.5H₂O: C, 50.82; H, 6.85; N, 8.47 Found: C, 51.05; H, 6.48; N, 8.57

### EXAMPLE 7

IR (KBr): 3352, 2933, 1630, 1531, 1516, 1441, 1271, 1236, 1217, 1043 cm⁻¹ NMR (DMSO-d₆+D₂O, δ): 0.98 (3H, d, J=6.8 Hz), 1.09 (3H, d, J=5.7 Hz), 1.2-1.6 (8H, m), 1.6-2.5 (11H, m), 2.8-4.5 (32H, m), 4.7-4.9 (2H, m), 6.7-6.9 (2H, m), 7.0-7.1 (1H, m), 7.2-7.3 (1H, m), 7.3-7.4 (1H, m), 7.8-8.0 (3H, m), 8.43 (1H, s) ESI MASS (Negative): 1276.4 (M⁻−H) Elemental Analysis Calcd. for C₅₈H₈₄N₈O₂₂S.4.5H₂O: C, 51.28; H, 6.90; N, 8.25 Found: C, 51.37; H, 7.05; N, 8.28

### EXAMPLE 8

IR (KBr): 3352, 1659, 1628, 1529, 1516, 1437, 1248, 1190, 1043 cm⁻¹ NMR (DMSO-d₆+D₂O, δ): 0.8-1.2 (9H, m), 1.3-1.5 (4H, m), 1.6-2:6 (9H, m), 2.8-4.5 (27H, m), 4.8-4.9 (2H, m), 6.7-6.9 (3H, m), 7.0-7.1 (3H, m), 7.46 (1H, d, J=15.7 Hz), 7.6-7.8 (6H, m) ESI MASS: 1279.3 (M⁺+Na) (Positive), 1256.3 (M⁻−H) (Negative) Elemental Analysis Calcd. for C₅₈H₈₀N₈O₂₁S.5H₂O: C, 51.70; H, 6.73; N, 8.32 Found: C, 51.67; H, 6.81; N, 8.29

### EXAMPLE 9

IR (KBr): 3356, 2929, 1633, 1539, 1514, 1495, 1437, 1257, 1043 cm⁻¹ NMR (DMSO-d₆+D₂O, δ): 0.8-0.9 (3H, m), 0.98 (3H, d, J=6.8 Hz), 1.10 (3H, d, J=6.2 Hz), 1.2-1.5 (10H, m), 1.6-2.5 (9H, m), 2.8-4.5 (27H, m), 4.8-4.9 (2H, m), 6.7-6.9 (2H, m), 7.0-7.1 (1H, m), 7.18 (2H, d, J=8.9 Hz), 8.10 (4H, d, J=8.7 Hz), 8.22 (2H, d, J=8.5 Hz) ESI MASS (Negative): 1340.4 (M⁻−H) Elemental Analysis Calcd. for C₆₁H₈₄N₁₀O₂₂S.6H₂O: C, 50.54; H, 6.68; N, 9.66 Found: C, 50.89; H, 6.71; N, 9.69

### EXAMPLE 10

IR (KBr): 3356, 1633, 1543, 1516, 1489, 1452, 1439, 1271, 1248 cm⁻¹ NMR (DMSO-d₆+D₂O, δ): 0.9-1.2 (9H, m), 1.6-2.6 (9H, m), 2.8-4.5 (27H, m), 4.8-4.9 (2H, m), 6.7-6.9 (2H, m), 7.0-7.2 (3H, m), 7.75 (2H, d, J=8.7 Hz), 7.92 (2H, d, J=8.4 Hz), 8.12 (2H, d, J=8.5 Hz), 8.2-8.3 (4H, m) ESI MASS (Negative): 1246.4 (M⁻−H) Elemental Analysis Calcd. for C₆₂H₇₈N₁₀O₂₂S.6H₂O: C, 51.16; H, 6.23; N, 9.62 Found: C, 51.06; H, 6.29; N, 9.58

### EXAMPLE 11

IR (KBr): 3354, 2927, 1632, 1537, 1513, 1495, 1450, 1439, 1271, 1248 cm⁻¹ NMR (DMSO-d₆+D₂O, δ): 0.8-0.9 (3H, m), 0.98 (3H, d, J=6.7 Hz), 1.09 (3H, d, J=5.8 Hz), 1.2-1.6 (12H, m), 1.6-2.6 (9H, m), 2.8-4.6 (27H, m), 4.8-4.9 (2H, m), 6.7-6.9 (2H, m), 7.0-7.1 (3H, m), 7.6-7.8 (4H, m), 7.9-8.1 (2H, d, J=8.4 Hz) ESI MASS (Negative): 1286.4 (M⁻−H) Elemental Analysis Calcd. for C₆₀H₈₆N₈O₂₁S.5H₂O: C, 52.31; H, 7.02; N, 8.13 Found: C, 52.27; H, 7.07; N, 8.14

### EXAMPLE 12

IR (KBr): 3356, 2927, 1632, 1539, 1514, 1439, 1273, 1242, 1043 cm⁻¹ NMR (DMSO-d₆+D₂O, δ): 0.8-0.9 (3H, m), 0.98 (3H, d, J=6.8 Hz), 1.09 (3H, d, J=6.1 Hz), 1.2-1.4 (8H, m), 1.5-2.6 (11H, m), 2.8-4.5 (25H, m), 4.8-4.9 (2H, m), 6.7-6.9 (2H, m), 7.0-7.1 (1H, m), 7.31 (2H, d, J=8.2 Hz), 7.65 (2H, d, J=8.2 Hz), 7.75 (2H, d, J=8.3 Hz), 7.97 (2H, d, J=8.4 Hz) ESI MASS (Positive): 1265.3 (M⁺+Na) Elemental Analysis Calcd. for C₅₈H₈₂N₈O₂₀S.5H₂O: C, 52.24; H, 6.95; N, 8.40 Found: C, 52.35; H, 7.06; N, 8.43

### EXAMPLE 13

IR (KBr): 1676, 1651, 1622, 1556, 1541, 1522, 1514, 1456 cm⁻¹ NMR (DMSO-d₆+D₂O, δ): 0.8-1.2 (9H, m), 1.3-1.6 (2H, m), 1.6-2.6 (9H, m), 2.7-4.5 (27H, m), 4.8-4.9 (2H, m), 6.7-6.9 (3H, m), 7.0-7.1 (3H, m), 7.4-7.8 (7H, m) ESI MASS (Positive): 1265.3 (M⁺+Na) Elemental Analysis Calcd. for C₅₇H₇₈N₈O₂₁S.6H₂O: C 50.66, H 6.71, N 8.29 Found: C, 50.64; H, 6.67; N, 8.22

### EXAMPLE 14

IR (KBr): 1676, 1649, 1632, 1554, 1539, 1514, 1456, 1439 cm⁻¹ NMR (DMSO-d₆+D₂O, δ): 0.8-0.9 (3H, m), 0.97 (3H, d, J=6.8 Hz), 1.11 (3H, d, J=6.0 Hz), 1.2-1.4 (8H, m), 1.5-2.6 (9H, m), 2.4-4.6 (27H, m), 4.7-4.9 (2H, m), 6.7-6.9 (2H, m), 7.0-7.1 (1H, m), 7.4-7.5 (1H, m), 7.75 (1H, s), 7.9-8.1 (3H, m), 8.46 (1H, s) ESI MASS (Positive): 1239.3 (M⁺+Na) Elemental Analysis Calcd. for C₅₆H₈₀N₈O₂₀S.6H₂O: C, 50.75; H, 7.00; N, 8.45 Found: C, 50.85; H, 6.79; N, 8.39

### EXAMPLE 15

IR (KBr): 2935, 1666, 1649, 1632, 1541, 1504, 1454, 1437, 1273 cm⁻¹ NMR (DMSO-d₆+D₂O, δ): 0.91 (3H, d, J=6.7 Hz), 0.98 (3H, d, J=6.7 Hz), 1.10 (3H, d, J=5.5 Hz), 1.3-2.7 (20H, m), 2.8-4.5 (30H, m), 4.7-4.9 (2H, m), 6.7-6.9 (2H, m), 7.0-7.1 (1H, m), 7.13 (2H, d, J=9.1 HZ), 7.97 (2H, d, J=8.8 Hz), 8.09 (2H, d, J=8.4 Hz), 8.20 (2H, d, J=8.6 Hz) ESI MASS (Negative): 1392.4 (M⁻−H) Elemental Analysis Calcd. for C₆₄H₈₈N₁₂O₂₁S.9H₂O: C, 49.41; H, 6.87; N, 10.80 Found: C

### EXAMPLE 16

IR (KBr): 1649, 1632, 1603, 1541, 1514, 1450, 1514, 1450, 1439, 1275, 1228 cm⁻¹ NMR (DMSO-d₆+D₂O, δ): 0.98 (3H, d, J=6.7 Hz), 1.10 (3H, d, J=5.8 Hz), 1.5-2.5 (12H, m), 2.7-4.7 (29H, m), 4.7-4.9 (2H, m), 6.7-6.9 (2H, m), 7.0-7.1 (2H, m), 7.1-7.4 (5H, m), 7.9-8.2 (5H, m), 8.74 (1H, d, J=2.5 Hz) ESI MASS (Negative): 1388.4 (M⁻−H) Elemental Analysis Calcd. for C₆₃H₈₀N₁₂O₂₀S₂.9.5H₂O: C, 48.48; H, 6.39; N, 10.77 Found: C, 48.54; H, 6.20; N, 10.76

### EXAMPLE 17

IR (KBr): 2931, 2856, 1676, 1651, 1608, 1556, 1541, 1514, 1452, 1441, 1419 cm⁻¹ NMR (DMSO-d₆+D₂O, δ): 0.98 (3H, d, J=6.8 Hz), 1.10 (3H, d, J=5.6 Hz), 1.2-1.6 (10H, m), 1.6-2.6 (11H, m), 2.8-4.6 (37H, m), 4.7-4.9 (2H, m), 6.7-6.9 (2H, m), 7.0-7.2 (3H, m), 7.85 (2H, d, J=8.8 Hz), 8.0-8.2 (4H, m) ESI MASS (Positive): 1478.4 (M⁺+Na) Elemental Analysis Calcd. for C₆₆H₉₃N₁₁O₂₂S₂.6H₂O: C, 50.66; H, 6.76; N, 9.85 Found: C, 50.75; H, 6.77; N, 9.78

### EXAMPLE 18

IR (KBr): 2927, 2856, 1678, 1651, 1556, 1541, 1514, 1456, 1439 cm⁻¹

NMR (DMSO-d₆+D₂O, δ): 0.98 (3H, d, J=6.7 Hz), 1.0-1.2 (3H, m), 1.2-2.6 (23H, m), 2.6-4.6 (37H, m), 4.7-4.9 (2H, m), 6.7-6.9 (2H, m), 7.0-7.1 (1H, m), 7.16 (2H, d, J=8.8 Hz), 7.97 (2H, d, J=8.7 Hz), 8.0-8.2 (4H, m) ESI MASS (Positive): 1471.4 (M⁺+H) Elemental Analysis Calcd. for C₆₇H₉₅N₁₁O₂₂S₂.5H₂O: C, 51.56; H, 6.78; N, 9.87 Found: C, 51.53; H, 6.84; N, 9.69

### EXAMPLE 19

IR (KBr): 3458, 3423, 3398, 3388, 3367, 2937, 1635, 1520, 1440, 1252 cm⁻¹

NMR (DMSO-d₆, δ): 0.80-1.00 (6H, m), 1.10 (3H, d, J=5.5 Hz), 1.20-1.50 (4H, m), 1.60-2.10 (7H, m), 2.20-2.50 (3H, m), 3.00-3.40 (4H, m), 3.40-4.60 (22H, m), 4.70-5.40 (10H, m), 6.60-6.80 (2H, m), 7.00 (1H, s), 7.08 (2H, d, J=8.8 Hz), 7.43 (1H, d, J=8.7 Hz), 7.55 (1H, d, J=8.9 Hz), 7.80 (2H, d, J=8.3 Hz), 7.80-8.05 (5H, m), 8.10-8.25 (2H, m), 8.25-8.40 (2H, m), 8.65-8.85 (2H, m) API-ES MASS (Negative): 1314 (M⁺), 1313 (M⁻−H), 1312 (M-2) Elemental Analysis Calcd. for C₅₉H₇₉N₉O₂₁S₂.6H₂O: C, 49.79; H, 6.40; N, 8.80 Found: C, 50.02; H, 6.41; N, 8.83

### EXAMPLE 20

IR (KBr): 1676, 1651, 1632, 1556, 1541, 1524, 1514, 1452, 1441, 1419 cm⁻¹ NMR (DMSO-d₆+D₂O, δ): 0.98 (3H, d, J=6.7 Hz), 1.10 (3H, d, J=6.1 Hz), 1.3-2.6 (15H, m), 2.7-4.5 (34H, m), 4.7-4.9 (2H, m), 6.7-6.9 (2H, m), 7.0-7.2 (3H, m), 7.86 (2H, d, J=8.6 Hz), 8.0-8.2 (4H, m) ESI MASS (Positive): 1425.3 (M²⁺+2 Na) Elemental Analysis Calcd. for C₆₂H₈₄N₁₂O₂₀S₂.7H₂O: C, 49.39; H, 6.55; N, 11.15 Found: C, 49.44; H, 6.43; N, 10.98

### EXAMPLE 21

IR (KBr): 3444, 3421, 1699, 1678, 1651, 1558, 1541, 1524, 1514, 1456 cm⁻¹ NMR (DMSO-d₆+D₂O, δ): 0.98 (3H, d, J=6.7 Hz), 1.10 (3H, d, J=5.7 Hz), 1.2-2.7 (20H, m), 2.7-4.5 (33H, m), 4.7-4.9 (2H, m), 6.7-6.9 (2H, m), 7.0-7.2 (3H, m), 7.86 (2H, d, J=8.9 Hzl), 8.0-8.2 (4H, m) ESI MASS (Positive): 1454.5 (M²⁺+2 Na) Elemental Analysis Calcd. for C₆₄H₈₈N₁₂O₂₀S₂.7H₂O: C, 50.05; H, 6.69; N, 10.94 Found: C, 50.29; H, 6.66; N, 10.81

### EXAMPLE 22

IR (KBr): 3363, 1633, 1529, 1518, 1444, 1419, 1238, 1088, 1045 cm⁻¹ NMR (DMSO-d₆+D₂O, δ): 0.98 (3H, d, J=6.7 Hz), 1.0-1.5 (11H, m), 1.6-2.7 (15H, m), 2.8-4.5 (42H, m), 4.7-4.9 (2H, m), 6.7-6.9 (2H, m), 7.02 (1H, br s), 7.08 (2H, d, J=9.0 Hz), 7.86 (2H, d, J=8.7 Hz), 8.0-8.2 (4H, m) ESI MASS (Negative): 1523.5 (M⁻−H) Elemental Analysis Calcd. for C₇₀H₁₀₀N₁₂O₂₂S₂.7H₂O: C, 50.90; H, 6.96; N, 10.18 Found: C, 50.70; H, 6.76; N, 10.04

### EXAMPLE 23

IR (KBr): 3498, 3466, 3435, 1659, 1635, 1606, 1547, 1529, 1518, 1444, 1417 cm⁻¹ NMR (DMSO-d₆+D₂O, δ): 0.98 (3H, d, J=6.7 Hz) 1.10 (3H, d, J=5.9 Hz), 1.2-2.6 (17H, m), 2.8-4.6 (40H, m), 4.7-4.9 (2H, m), 6.7-6.9 (2H, m), 7.0-7.2 (3H, m), 7.87 (2H, d, J=8.7 Hz), 8.0-8.2 (4H, m) ESI MASS (Negative): 1440.5 (M⁻−H) Elemental Analysis Calcd. for C₆₅H₉₂N₁₂O₂₁S₂.8H₂: C, 49.23; H, 6.86; N, 10.60 Found: C, 49.54; H, 6.76; N, 10.41

### EXAMPLE 24

IR (KBr): 3352, 1659, 1635, 1606, 1529, 1444, 1419, 1277, 1238 cm⁻¹ NMR (DMSO-d₆+D₂O, δ): 0.98 (3H, d, J=6.8 Hz), 1.10 (3H, d, J=5.9 Hz), 1.2-2.8 (11H, m), 2.8-4.5 (38H, m), 4.7-4.9 (2H, m), 6.7-6.9 (2H, m), 7.0-7.2 (3H, m), 7.87 (2H, d, J=8.8 Hz), 8.0-8.2 (4H, m) ESI MASS (Negative): 1396.4 (M⁻−H) Elemental Analysis Calcd. for C₆₂H₈₄N₁₂O₂₁S₂.7H₂O: C, 48.87; H, 6.48; N, 11.03 Found: C, 48.88; H, 6.50; N, 10.83

### EXAMPLE 25

IR (KBr): 1659, 1628, 1606, 1529, 1444, 1417, 1281, 1240 cm⁻¹ NMR (DMSO-d₆+D₂O, δ): 0.98 (3H, d, J=6.8 Hz), 1.10 (3H, d, J=5.7 Hz), 1.4-2.7 (15H, m), 2.7-4.5 (38H, m), 4.7-4.9 (2H, m), 6.7-6.9 (2H, m), 7.0-7.2 (3H, m), 7.86 (2H, d, J=8.9 Hz), 8.0-8.2 (4H, m) ESI MASS (Negative): 1452.4 (M⁻−Na) Elemental Analysis Calcd. for C₆₅H₈₈N₁₂O₂₂S₂.8H₂O: C, 48.86; H, 6.56; N, 10.52 Found: C, 48.99; H, 6.47; N, 10.20

### EXAMPLE 26

IR (KBr): 3464, 3429, 3373, 1659, 1628, 1606, 1529, 1444, 1419, 1281, 1238 cm⁻¹ NMR (DMSO-d₆+D₂O, δ): 0.98 (3H, d, J=6.8 Hz), 1.10 (3H, d, J=5.8 Hz), 1.5-2.6 (7H, m), 2.8-4.5 (35H, m), 4.7-4.9 (2H, m), 6.7-6.9 (2H, m), 7.0-7.2 (3H, m), 7.39 (2H, d, J=5.9 Hz), 7.87 (2H, d, J=8.8 Hz), 8.0-8.2 (4H, m), 8.53 (2H, d, j=5.8 Hz) ESI MASS (Negative): 1403.4 (M⁻−H) Elemental Analysis Calcd. for C₆₃H₈₁N₁₃O₂₀S₂.8H₂O: C, 48.86; H, 6.31; N, 11.76 Found: C, 49.02; H, 6.15; N, 11.42

### EXAMPLE 27

IR (KBr): 3466, 3433, 3398, 2360, 2337, 1664, 1635, 1605, 1446, 1408, 1350 cm⁻¹ ESI MASS (Negative): 1494.3 (M⁻−H)

### EXAMPLE 28

IR (KBr): 1664, 1628, 1605, 1529, 1444, 1417, 1279 cm⁻¹ NMR (DMSO-d₆+D₂O, δ): 0.98 (3H, d, J=6.9 Hz), 1.10 (3H, d, J=5.9 Hz), 1.6-2.5 (11H, m), 2.8-4.5 (32H, m), 4.7-4.9 (2H, m), 6.7-6.9 (2H, m), 7.0-7.1 (1H, m), 7.15 (2H, d, J=8.8 Hz), 7.4-7.5 (4H, m), 7.87 (2H, d, J=8.8 Hz), 8.0-8.2 (4H, m) ESI MASS (Negative): 1452.3 (M⁻−H) Elemental Analysis Calcd. for C₆₅H₈₂N₁₁O₂₁S₂.7H₂O: C, 49.44; H, 6.13; N, 9.76 Found: C, 49.80; H, 6.06; N, 9.56

### EXAMPLE 29

IR (KBr): 1645, 1632, 1608, 1539, 1514, 1443, 1419, 1273, 1232 cm⁻¹ NMR (DMSO-d₆+D₂O, δ): 0.98 (3H, d, J=6.8 Hz), 1.10 (3H, d, J=5.9 Hz), 1.6-2.6 (11H, m), 2.7-4.5 (33H, m), 4.7-4.9 (2H, m), 6.7-6.9 (2H, m), 7.0-7.1 (1H, m), 7.12 (2H, d, J=8.8 Hz), 7.86 (2H, d, J=8.9 Hz), 8.0-8.2 (4H, m) ESI MASS (Positive): 1392.3 (M⁺+Na) Elemental Analysis Calcd. for C₆₀H₇₉N₁₁O₂₂S₂.6H₂O: C, 48.74; H, 6.20; N, 10.42 Found: C, 48.37; H, 6.25; N, 10.19

### EXAMPLE 30

IR (KBr): 1649, 1633, 1608, 1539, 1512, 1450, 1443, 1419, 1238 cm⁻¹ NMR (DMSO-d₆+D₂O, δ): 0.97 (3H, d, J=6.8 Hz), 1.11 (3H, d, J=5.2 Hz), 1.4-2.6 (7H, m), 2.7-4.6 (31H, m), 4.7-4.9 (2H, m), 6.6-6.9 (2H, m), 7.0-7.1 (1H, m), 7.21 (2H, d, J=9.2 Hz), 7.3-7.7 (6H, m), 7.90 (2H, d, J=8.7 Hz), 8.0-8.2 (4H, m) ESI MASS (Positive): 1470.2 (M²⁺+2 Na) Elemental Analysis Calcd. for C₆₅H₇₉N₁₃O₂₀S₂.7.5H₂O: C, 49.99; H, 6.07; N, 11.66 Found: C, 49.90; H, 5.97; N, 11.34

### EXAMPLE 31

ESI MASS (Negative): 1539.6 (M⁻−H)

### EXAMPLE 32

IR (KBr): 1666, 1649, 1632, 1554, 1541, 1514, 1450, 1441, 1254 cm⁻¹ NMR (DMSO-d₆+D₂O, δ): 0.98 (3H, d, J=6.8 Hz), 1.10 (3H, d, J=6.0 Hz), 1.6-2.6 (11H, m), 2.8-4.5 (29H, m), 4.6-4.8 (3H, m), 6.7-6.9 (2H, m), 6.9-7.1 (3H, m), 7.1-7.3 (4H, m), 7.9-8.2 (7H, m) ESI MASS (Negative): 1403.4 (M⁻−H) Elemental Analysis Calcd. for C₆₄H₈₁N₁₁O₂₁S₂.6.5H₂O: C, 50.52; H, 6.23; N, 10.13 Found: C, 50.47; H, 6.19; N, 9.98

### EXAMPLE 33

IR (KBr): 1676, 1649, 1632, 1556, 1541, 1514, 1452, 1441, 1250 cm⁻¹ NMR (DMSO-d₆+D₂O, δ): 0.98 (3H, d, J=6.8 Hz), 1.10 (3H, d, J=5.9 Hz), 1.6-2.6 (11H, m), 2.8-4.8 (33H, m), 4.8-4.9 (2H, m), 6.6-7.1 (7H, m), 7.21 (2H, d, J=8.9 Hz), 7.99 (2H, d, J=8.7 Hz), 8.0-8.2 (4H, m) ESI MASS (Negative): 1433.4 (M⁻−Na) Elemental Analysis Calcd. for C₆₅H₈₃N₁₁O₂₂S₂.6.5H₂O: C, 50.31; H, 6.24; N, 9.93 Found: C, 50.22; H, 6.23; N, 9.81

### EXAMPLE 34

IR (KBr): 3492, 3471, 3431, 3396, 1664, 1628, 1606, 1446, 1254 cm⁻¹ NMR (DMSO-d₆+D₂O, δ): 0.9-1.3 (12H, m), 1.3-2.6 (15H, m), 2.8-4.6 (31H, m), 4.7-4.9 (2H, m), 6.7-6.9 (2H, m), 7.0-7.1 (1H, m), 7.2-7.4 (2H, m), 7.9-8.2 (6H, m) ESI MASS: 1432.3 (M⁺+Na)(Positive), 1409.6 (M⁻−H)(Negative) Elemental Analysis Calcd. for C₆₄H₈₇N₁₁O₂₁S₂.7H₂O: C, 50.02; H, 6.62; N, 10.03 Found: C, 50.00; H, 6.69; N, 9.85

### EXAMPLE 35

IR (KBr): 2933, 2862, 1697, 1676, 1651, 1556, 1541, 1514, 1454, 1439 cm⁻¹ NMR (DMSO-d₆+D₂O, δ): 0.98 (3H, d, J=6.6 Hz), 1.0-1.2 (3H, m), 1.2-2.6 (22H, m), 2.6-4.5 (36H, m), 4.7-4.9 (2H, m), 6.7-6.9 (2H, m), 7.0-7.1 (1H, m), 7.49 (2H, d, J=8.2 Hz), 7.98 (2H, d, J=8.4 Hz), 8.0-8.2 (4H, m) ESI MASS (Negative): 1439.6 (M⁻−H) Elemental Analysis Calcd. for C₆₆H₉₃N₁₁O₂₁S₂.6H₂O: C, 51.18; H, 6.83; N, 9.95 Found: C, 51.19; H, 6.89; N, 9.88

### EXAMPLE 36

ESI MASS (Negative): 1523.6 (M⁻−H)

### EXAMPLE 37

IR (KBr): 3494, 3466, 3433, 3394, 2935, 1659, 1628, 1531, 1444, 1279 cm⁻¹ NMR (DMSO-d₆+D₂O, δ): 0.98 (3H, d, J=6.7 Hz), 1.0-2.6 (26H, m), 2.7-4.5 (43H, m), 4.7-4.9 (2H, m), 6.7-6.9 (2H, m), 7.0-7.1 (1H, m), 7.48 (2H, d, J=8.4 Hz), 7.9-8.2 (6H, m), 7.0-7.1 (1H, m), 7.48 (2H, d, J=8.4 Hz), 7.9-8.2 (6H, m) ESI MASS (Negative): 1523.6 (M⁻−H) Elemental Analysis Calcd. for C₇₁H₁₀₁N₁₁O₂₂S₂.8H₂O: C, 51.10; H, 7.07; N, 9.23 Found: C, 51.32; H, 7.04; N, 9.14

### EXAMPLE 38

IR (KBr): 2935, 1632, 1608, 1535, 1516, 1464, 1439, 1248, 1084, 1045 cm⁻¹ NMR (DMSO-d₆+D₂O, δ): 0.98 (3H, d, J=6.8 Hz), 1.09 (3H, d, J=5.8 Hz), 1.2-1.6 (11H, m), 1.6-2.6 (7H, m), 2.7-4.5 (38H, m), 4.7-4.9 (2H, m), 6.7-6.9 (2H, m), 7.0-7.2 (3H, m), 7.75 (2H, d, J=8.8 Hz), 7.9-8.0 (4H, m), 8.78 (1H, s) ESI MASS (Negative): 1480.5 (M⁻−H) Elemental Analysis Calcd. for C₆₇H₉₂N₁₂O₂₂S₂.6H₂O: C, 50.62; H, 6.59; N, 10.57 Found: C, 50.51; H, 6.65; N, 10.46

### EXAMPLE 39

IR (KBr): 1659, 1635, 1606, 1529, 1518, 1466, 1446, 1277, 1248 cm⁻¹ NMR (DMSO-d₆+D₂O, δ): 0.98 (3H, d, J=6.7 Hz), 1.09 (3H, d, J=6.1 Hz), 1.1-2.6 (16H, m), 2.7-4.5 (38H, m), 4.7-4.9 (2H, m), 6.7-6.9 (2H, m), 7.0-7.2 (3H, m), 7.75 (2H, d, J=8.6 Hz), 7.9-8.0 (4H, m), 8.77 (1H, s) ESI MASS (Negative): 1466.5 (M⁻−H) Elemental Analysis Calcd. for C₆₆H₉₀N₁₂O₂₂S₂.6H₂O: C, 50.31; H, 6.52; N, 10.67 Found: C, 54.61; H, 6.18; N, 11.45

### EXAMPLE 40

IR (KBr): 3464, 3435, 3394, 1659, 1628, 1529, 1514, 1468, 1444, 1250 cm⁻¹ NMR (DMSO-d₆+D₂O, δ): 0.98 (3H, d, J=6.7 Hz), 1.10 (3H, d, J=5.8 Hz), 1.5-2.6 (11H, m), 2.8-4.7 (33H, m), 4.7-4.9 (2H, m), 6.6-7.1 (7H, m), 7.22 (2H, d, J=8.9 Hz), 7.8-8.1 (6H, m), 8.83 (1H, s) ESI MASS (Negative): 1472.4 (M⁻−H) Elemental Analysis Calcd. for C₆₇H₈₄N₁₂O₂₂S₂.7H₂O: C, 50.30; H, 6.17; N, 10.51 Found: C, 50.12; H, 6.19; N, 10.33

### EXAMPLE 41

IR (KBr): 3494, 3465, 3433, 3367, 1659, 1628, 1529, 1518, 1468, 1444, 1254 cm⁻¹ NMR (DMSO-d₆+D₂O, δ): 0.98 (3H, d, J=6.7 Hz), 1.11 (3H, d, J=5.5 Hz), 1.6-2.6 (11H, m), 2.8-4.6 (30H, m), 4.7-4.9 (2H, m), 6.7-6.9 (3H, m), 6.9-7.1 (3H, m), 7.2-7.4 (4H, m), 7.8-8.1 (6H, m), 8.83 (1H, s) ESI MASS: 1465.3 (M⁺+Na) (Positive), 1442.4 (M⁻−H) (Negative) Elemental Analysis Calcd. for C₆₆H₈₂N₁₂O₂₁S₂.7.5H₂O: C, 50.21; H, 6.19; N, 10.65 Found: C, 50.37; H, 6.23; N, 10.54

### EXAMPLE 42

IR (KBr): 1666, 1649, 1632, 1554, 1539, 1516, 1466, 1458, 1250 cm⁻¹ NMR (DMSO-d₆+D₂O, δ): 0.97 (3H, d, J=6.8 Hz), 1.0-1.3 (9H, m), 1.5-2.6 (15H, m), 2.7-4.6 (31H, m), 4.7-4.9 (2H, m), 6.7-6.9 (2H, m), 7.0-7.1 (1H, m), 7.16 (2H, d, J=8.9 Hz), 7.89 (2H, d, J=9.0 Hz), 7.9-8.1 (4H, m), 8.83 (1H, s) ESI MASS (Negative): 1448.5 (M⁻−H) Elemental Analysis Calcd. for C₆₆H₈₈N₁₂O₂₁S₂.8H₂O: C, 49.74; H, 6.58; N, 10.55 Found: C, 54.68; H, 6.12; N, 11.59

### EXAMPLE 43

IR (KBr): 1676, 1649, 1633, 1556, 1541, 1514, 1471, 1458, 1435 cm⁻¹ NMR (DMSO-d₆+D₂O, δ): 0.98 (3H, d, J=6.8 Hz), 1.0-2.6 (22H, m), 2.7-4.5 (30H, m), 4.7-4.9 (2H, m), 6.3-6.5 (1H, m), 6.7-6.9 (2H, m), 7.0-7.1 (1H, m), 7.6-9.2 (9H, m) ESI MASS (Positive): 1475.7 (M²⁺+2 Na) Elemental Analysis Calcd. for C₆₆H₈₆N₁₂O₂₀S₂.8H₂O: C, 50.31; H, 6.52; N, 10.67 Found: C, 50.55; H, 6.30; N, 10.58

### EXAMPLE 44

NMR (DMSO-d₆+D₂O, δ): 0.98 (3H, d, J=6.7 Hz), 1.10 (3H, d, J=5.9 Hz), 1.2-2.5 (19H, m), 2.7-4.5 (37H, m), 4.7-4.9 (2H, m), 6.7-6.9 (2H, m), 7.0-7.1 (1H, m), 7.50 (2H, d, J=8.6 Hz), 7.8-8.1 (6H, m), 8.86 (1H, s) ESI MASS (Negative): 1464.4 (M⁻−H)

### EXAMPLE 45

IR (KBr): 3493, 3469, 3435, 1664, 1635, 1606, 1446 cm⁻¹ NMR (DMSO-d₆+D₂O, δ): 0.98 (3H, d, J=6.6 Hz), 1.09 (3H, d, J=6.0 Hz), 1.2-2.6 (13H, m), 2.8-4.5 (40H, m), 4.7-4.9 (2H, m), 6.7-6.9 (2H, m), 7.0-7.2 (3H, m), 7.78 (2H, d, J=8.7 Hz), 7.9-8.0 (4H, m), 8.78 (1H, s) ESI MASS (Negative): 1451.4 (M⁻−H) Elemental Analysis Calcd. for C₆₅H₈₉N₁₃O₂ₗS₂.7H₂O: C, 49.45; H, 6.58; N, 11.53 Found: C, 49.34; H, 6.64; N, 11.18

### EXAMPLE 46

IR (KBr): 3464, 3425, 3386, 3365, 2935, 1635, 1614, 1523 cm⁻¹ NMR (DMSO-d₆, δ): 0.97 (3H, d, J-6.8 Hz), 1.10 (3H, d, J-5.7 Hz), 1.20-1.40 (4H, m), 1.40-1.60 (5H, m), 1.70-2.10 (4H, m), 2.20-2.40 (6H, m), 2.80-3.00 (1H, m), 3.22 (3H, s), 3.40-4.50 (18H, m), 4.70-5.10 (4H, m), 5.10-5.40 (6H, m), 6.71 (1H, d, J=8.1 Hz), 6.70-6.90 (1H, m), 7.00 (1H, br s), 7.08 (2H, d, J=9.0 Hz), 7.40-7.60 (2H, m), 7.77 (2H, d, J=8.8 Hz), 7.80-8.00 (6H, m), 8.20-8.40 (1H, m), 8.60-8.80 (2H, m), 8.80 (1H, s) API-ES MASS (Negative): 1466(M), 1465(M⁺), 1464(M⁻−2) Elemental Analysis Calcd. for C₆₆H₉₁N₁₃O₂₁S₂.6H₂O: C, 50.32; H, 6.54; N, 11.56 Found: C, 50.38; H, 6.66; N, 11.43

### EXAMPLE 47

IR (KBr): 1658, 1635, 1606, 1529, 1518, 1468, 1446, 1431, 1238 cm⁻¹ NMR (DMSO-d₆+D₂O, δ): 0.98 (3H, d, J=6.9 Hz), 1.09 (3H, d, J=5.5 Hz), 1.2-2.6 (17H, m), 2.8-4.5 (40H, m), 4.7-4.9 (2H, m), 6.7-6.9 (2H, m), 7.0-7.2 (3H, m), 7.78 (2H, d, J=8.7 Hz), 7.9-8.1 (4H, m), 8.78 (1H, s) ESI MASS (Negative): 1479.4 (M⁻−H) Elemental Analysis Calcd. for C₆₇H₉₃N₁₃O₂₁S₂.6H₂O: C, 50.65; H, 6.66; N, 11.46 Found: C, 50.82; H, 6.90; N, 11.17

### EXAMPLE 48

IR (KBr): 3464, 3460, 3425, 3400, 3367, 2939, 1633, 1522, 1454, 1248 cm⁻¹ NMR (DMSO-d₆, δ): 0.98 (3H, d, J=6.7 Hz), 1.10 (3H, d, J=5.6 Hz), 1.30-2.00 (10H, m), 3.23 (3H, s), 2.80-4.45 (20H, m), 4.60-5.40 (10H, m), 6.60-6.80 (1H, m), 6.71 (1H, d, J=8.1 Hz), 7.00 (1H, s), 7.04 (2H, d, J=8.9 Hz), 7.46 (1H, m), 7.60 (2H, d, J=8.7 Hz), 7.70-8.00 (3H, m), 8.37 (1H, br s), 8.71 (1H, s) API-ES MASS (Negative): 1383(M), 1382(M⁻−H), 1381(M⁻−2) Elemental Analysis Calcd. for C₆₂H₈₂N₁₀O₂₂S₂.10H₂O: C, 47.60; H, 6.52; N, 8.96 Found: C, 47.36; H, 6.12; N, 8.78

### EXAMPLE 49

IR (KBr): 1632, 1539, 1514, 1452, 1236 cm⁻¹ NMR (DMSO-d₆, δ): 0.98 (3H, d, J=6.7 Hz), 1.10 (3H, d, J=5.6 Hz), 1.6-2.6 (9H, m), 2.8-4.6 (35H, m), 4.7-5.4 (9H, m), 6.65-7.05 (7H, m), 7.11 (2H, d, J=8.8 Hz), 7.3-8.0 (9H, m), 8.0-8.45 (3H, m), 8.6-8.8 (2H, m) MASS (m/z): 1333 (M⁺−H) Elemental Analysis Calcd. for C₆₂H₈₂N₁₀O₂₁S-9H₂O: C, 49.73; H, 6.73; N, 9.35 Found: C, 49.78; H, 6.54; N, 9.60

### EXAMPLE 50

IR (KBr): 1666, 1649, 1632, 1539, 1512, 1452, 1232 cm⁻¹ NMR (DMSO-d₆, δ): 0.97 (2H, d, J=6.6 Hz), 1.0-2.6 (29H, m), 2.6-4.6 (40H, m), 4.7-5.4 (9H, m), 6.65-7.2 (9H, m), 7.4-8.05 (9H, m), 8.1-8.8 (5H, m) MASS (m/z): 1516 (M⁺−H) Elemental Analysis Calcd. for C₇₃H₁₀₃N₁₁O₂₂S-7H₂O: C, 53.31; H, 7.17; N, 9.37 Found: C, 53.18; H, 7.14; N, 9.56

### EXAMPLE 51

NMR (DMSO-d₆, δ): 0.97 (3H, d, J=6.9 Hz), 0.9-1.4 (8H, m), 1.4-2.7 (18H, m), 2.8-4.6 (29H, m), 4.7-5.5 (9H, m), 6.6-7.1 (5H, m), 7.4-8.4 (12H, m), 8.5-8.8 (2H, m) MASS (m/z): 1321 (M⁺−H)

### EXAMPLE 52

IR (KBr): 1668, 1649, 1632, 1539, 1514, 1456, 1238 cm⁻¹ NMR (DMSO-d₆, δ): 0.90 (3H, d, J=6.8 Hz), 0.97 (3H, d, J=6.8 Hz), 1.10 (3H, d, J=5.9 Hz), 1.3-2.75 (23H, m), 2.8-4.6 (28H, m), 4.7-5.4 (9H, m), 6.6-7.2 (5H, m), 7.3-8.5 (12H, m), 8.6-8.8 (2H, m) MASS (m/z): 1325 (M⁺+H) Elemental Analysis Calcd. for C₆₂H₈₈N₁₀O₂₀S-9H₂O: C, 50.06; H, 7.18; N, 9.42 Found: C, 50.14; H, 7.11; N, 9.36

### EXAMPLE 53

IR (KBr): 1645, 1632, 1539, 1514, 1454, 1236 cm⁻¹ NMR (DMSO-d₆, δ): 0.86 (3H, d, J=6.4 Hz), 0.97 (3H, d, J=6.8 Hz), 1.0-1.4 (8H, m), 1.6-3.0 (19H, m), 3.1-3.6 (27H, m), 3.7-5.4 (9H, m), 6.7-7.1 (5H, m), 7.3-8.8 (14H, m) MASS (m/z): 1325 (M⁺+H) Elemental Analysis Calcd. for C₆₂H₈₈N₁₀O₂₀S-9H₂O: C, 50.06; H, 7.18; N, 9.42 Found: C, 50.33; H, 7.13; N, 9.37

### EXAMPLE 54

IR (KBr): 1664, 1628, 1605, 1547, 1531, 1497, 1446, 1277 cm⁻¹

NMR (DMSO-d₆+D₂O, δ): 0.98 (3H, d, J=6.8 Hz), 1.09 (3H, d, J=5.7 Hz), 1.6-4.5 (41H, m), 4.7-4.9 (2H, m), 6.7-6.9 (2H, m), 7.0-7.2 (3H, m), 7.2-7.4 (5H, m), 7.63 (2H, d, J=8.8 Hz), 7.71 (2H, d, J=8.3 Hz), 7.94 (2H, d, J=8.4 Hz) ESI MASS (Negative): 1303.3 (M⁻−H) Elemental Analysis Calcd. for C₆₂H₈₁N₉O₂₀S.6H₂O: C, 52.72; H, 6.64; N, 8.92 Found: C, 52.90; H, 6.71; N, 8.82

### EXAMPLE 55

IR (KBr): 1659, 1628, 1605, 1529, 1444, 1417, 1277, 1228 cm⁻¹ NMR (DMSO-d₆+D₂O, δ): 0.89 (3H, t, J=7.2 Hz), 0.98 (3H, d, J=6.8 Hz), 1.10 (3H, d, J=6.0 Hz), 1.2-1.6 (4H, m), 1.7-2.6 (11H, m), 2.8-4.5 (32H, m), 4.7-4.9 (2H, m), 6.6-6.8 (2H, m), 7.0-7.1 (1H, m), 7.09 (2H, d, J=9.0 Hz), 7.85 (2H, d, J=8.7 Hz), 7.9-8.2 (4H, m) ESI MASS (Negative): 1383.4 (M⁻−H) Elemental Analysis Calcd. for C₆₂H₈₅N₁₁O₂₁S₂.7H₂O: C, 49.29; H, 6.61; N, 10.20 Found: C, 49.69; H, 6.37; N, 10.29

### EXAMPLE 56

IR (KBr): 1664, 1628, 1605, 1529, 1444, 1417, 1277, 1228 cm⁻¹ NMR (DMSO-d₆+D₂O, δ): 0.87 (3H, t, J=6.7 Hz), 0.98 (3H, d, J=6.6 Hz), 1.10 (3H, d, J=5.8 Hz), 1.2-1.6 (6H, m), 1.7-2.6 (11H, m), 2.8-4.5 (32H, m), 4.7-4.9 (2H, m), 6.6-6.8 (2H, m), 7.0-7.1 (1H, m), 7.09 (2H, d, J=8.9 Hz), 7.85 (2H, d, J=8.7 Hz), 8.0-8.2 (4H, m) ESI MASS (Negative): 1396.4 (M²—2H) Elemental Analysis Calcd. for C₆₃H₈₇N₁₁O₂₁S₂.7H₂O: C, 49.63; H, 6.68; N, 10.11 Found: C, 49.82; H, 6.55; N, 10.10

### EXAMPLE 57

IR (KBr): 1664, 1635, 1605, 1446, 1412, 1350, 1281, 1043 cm⁻¹ NMR (DMSO-d₆+D₂O, δ): 0.86 (6H, d, J=6.5 Hz), 0.98 (3H, d, J=6.8 Hz), 1.1-1.3 (5H, m), 1.4-2.6 (14H, m), 2.8-4.5 (32H, m), 4.7-4.9 (2H, m), 6.7-6.9 (2H, m), 7.0-7.2 (3H, m), 7.85 (2H, d, J=8.6 Hz), 8.0-8.2 (4H, m) ESI MASS (Negative): 1410.3 (M²—2H) Elemental Analysis Calcd. for C₆₄H₈₉N₁₁O₂₁S₂.7H₂O: C, 49.96; H, 6.75; N, 10.01 Found: C, 49.89; H, 6.53; N, 9.92

### EXAMPLE 58

IR (KBr): 1662, 1628, 1605, 1529, 1444, 1417, 1277, 1227, 1043 cm⁻¹ NMR (DMSO-d₆+D₂O, δ): 0.8-1.4 (12H, m), 1.4-2.6 (16H, m), 2.8-4.5 (32H, m), 4.7-4.9 (2H, m), 6.7-6.9 (2H, m), 7.0-7.1 (1H, m), 7.09 (2H, d, J=9.1 Hz), 7.84 (2H, d, J=8.8 Hz), 8.0-8.2 (4H, m) ESI MASS (Negative): 1423.5 (M⁻−H) Elemental Analysis Calcd. for C₆₅H₈₉N₁₁O₂₁S₂.7H₂O: C, 50.34; H, 6.69; N, 9.94 Found: C, 50.75; H, 6.56; N, 9.96

### EXAMPLE 59

IR (KBr): 2360, 1662, 1635, 1606, 1529, 1466, 1446, 1240 cm⁻¹ NMR (DMSO-d₆+D₂O, δ): 0.8-1.3 (12H, m), 1.5-2.6 (16H, m), 2.8-4.5 (32H, m), 4.7-4.9 (2H, m), 6.7-6.9 (2H, m), 7.0-7.2 (3H, m), 7.75 (2H, d, J=8.8 Hz), 7.9-8.0 (4H, m), 8.78 (1H, s) ESI MASS (Negative): 1462.5 (M⁻−H) Elemental Analysis Calcd. for C₆₇H₉₀N₁₂O₂₁S₂.8H₂O: C, 50.05; H, 6.65; N, 10.45 Found: C, 50.15; H, 6.38; N, 10.45

### EXAMPLE 60

NMR (DMSO-d₆+D₂O, δ): 0.98 (3H, d, J=6.7 Hz), 1.10 (3H, d, J=5.5 Hz), 1.2-2.6 (15H, m), 2.8-4.5 (48H, m), 4.7-4.9 (2H, m), 6.7-7.0 (6H, m), 7.0-7.2 (3H, m), 7.6-7.8 (4H, m), 7.94 (2H, d, J=8.1 Hz) ESI MASS (Negative): 1502.6 (M⁻−H)

### EXAMPLE 61

IR (KBr): 1664, 1635, 1605, 1589, 1446, 1408, 1350 cm⁻¹ NMR (DMSO-d₆+D₂O, δ): 0.98 (3H, d, J=6.7 Hz), 1.09 (3H, d, J=5.7 Hz), 1.2-2.8 (25H, m), 2.8-4.5 (40H, m), 4.7 4.9 (2H, m), 6.7-6.9 (2H, m), 6.9-7.1 (3H, m), 7.63 (2H, d, J=8.8 Hz), 7.71 (2H, d, J=8.8 Hz), 7.92 (2H, d, J=8.0 Hz) ESI MASS (Negative): 1452.6 (M⁻−H)

### EXAMPLE 62

IR (KBr): 2972, 1664, 1628, 1606, 1446, 1279, 1240, 1082, 1047 cm⁻¹ NMR (DMSO-d₆+D₂O, δ): 0.98 (3H, d, J=6.7 Hz), 1.09 (3H, d, J=5.9 Hz), 1.2-2.6 (25H, m), 2.8-4.5 (40H, m), 4.7-4.9 (2H, m), 6.7-6.9 (2H, m), 6.9-7.1 (3H, m), 7.63 (2H, d, J=8.8 Hz), 7.71 (2H, d, J=8.3 Hz), 7.93 (2H, d, J=8.3 Hz) ESI MASS (Negative): 1452.6 (M⁻−H) Elemental Analysis Calcd. for C₆₉H₁₀₀N₁₀O₂₂S.7H₂O: C, 52.46; H, 7.27; N, 8.87 Found: C, 57.01; H, 6.93; N, 9.64

### EXAMPLE 63

To a solution of Starting Compound (190 mg) was added 1-[4-[2-[4-[4-[1-[4-(6-methoxyhexyloxy)cyclohexyl]-piperidyloxy]phenyl]imidazo[2,1-b][1,3,4]thiadiazol-6-yl]benzoyloxy]-1H-1,2,3-benzotriazole (120 mg) and hunings base (0.042 ml) and the mixture was stirred for 16 hours. 1-[4-[2-[4-[4-[1-[4-(6-methoxyhexyloxy)cyclohexyl]-piperidyloxy]phenyl]imidazo[2,1-b][1,3,4]thiadiazol-6-yl]benzoyloxy]-1H-1,2,3-benzotriazole (240 mg) was added to the above mixture to complete the reaction. After stirring for 5 hours, EtOAc (100 ml) was added dropwise to the solution to give crude precipitation. Usual workup followed by preparative liquid chromatography (ODS, CH₃CN: H₂O=40:60) gave, in order of elution, Minor Compound (63) (11 mg) and Major Compound (63) (23 mg), which were used in the next reaction without further purification.

The following compound was obtained according to a similar manner to that of Example 63.

### EXAMPLE 64

MASS (m/z): 1545 (M⁺−H)

### EXAMPLE 65

To a solution of Minor Compound (63) (11 mg) was added piperidine (0.006 ml) and the solution was stirred for 1 hour. EtOAc (100 ml) was added dropwise to the above solution to give crude precipitation, which was collected and usual workup followed by chromatography (ODS, CH₃CN:H₂O=50:50) gave Object Compound (65) (1 mg). ESI MASS (Negative): 1578.6 (M⁻−H)

The following compounds [Example 66: and 67] were obtained according to a similar manner to that of Example 65.

### EXAMPLE 66

ESI MASS (Negative): 1578.8 (M⁻−H)

### EXAMPLE 67

MASS (m/z): 1323 (M⁺−H)

### EXAMPLE 68

To a solution of 4-[5-[4-[4-(cis-4-methylcyclohexyl)piperazinyl]phenyl]-1,3,4-thiadiazol-2-yl]benzoic acid (8.29 kg) in N-methyl-2-pyrrolidone (140 l) were added N,N-diisopropylethylamine (DIPEA) (4.0 kg) and 0-benzotriazol-1-yl-N,N,N′,N′-tetramethyl-uronium hexafluorophosphate (HBTU) (7.05 kg) at room temperature, and the mixture was stirred at 40-50° C. for 2.5 hours. After cooling to 20° C., DIPEA (2.0 kg) and Starting Compound (68) (14.0 kg) were added and stirring was continued at 25-30° C. for 2 hours. The resulting mixture was poured into water (980 l) at 30-35° C. for 1 hour and stirred for 0.5 hour. The resulting crystals were filtered and washed with water (140 l). The crystals were dried overnight in vacuo to give Object Compound (68) (20.6 kg). The product was used in the next step without further purification.

IR (KBr): 1676, 1645, 1635, 1630, 1533, 1515, 1446, 1439, 1425 cm⁻¹ NMR (DMSO-d₆, δ): 0.90-1.26 (12H, m), 0.96 (6H, d, J=7.0 Hz), 1.10 (3H, d, J=5.8 Hz), 1.23-5.37 (61H, m), 6.41-9.15 (17H, m) ESI MASS (m/z)(Negative): 1348.4 (M-DIPEA+)

### EXAMPLE 69

To a solution of Starting Compound (69) (17.0 kg) in N-methyl-2-pyrrolidone (85 l) were added pyridine (3.87 kg) and 1-[3-(dimethylamino)propyl]-3-ethylcarbodiimide hydrochloride (8.82 kg) with stirring at 0-10° C. After stirring for 20 hours at 50-60° C., the mixture was added to water (850 l). The pH value of the mixture were maintained at 4-5 during the addition with 1N—HCl (about 50 l) at 20-30° C. and the stirring was kept for 1 hour. The pH of the suspension was raised to 10.4-10.6 with 1N-NaOH (55 l) and then the suspension was heated to 40-45° C. and stirred for 7 hours. The resulting solution was cooled to 20-30° C. and 1N—HCl was added to the pH range 6.5-7.0 to precipitate the product. After stirring for 1 hour, the precipitate was filtered and washed with water (170 l).

The precipitate without drying was dissolved in alkaline water (pH 9.5-9.7: about 700 l) and acetonitrile (170 l) mixture and the solution was chromatographed on HP20SS (Mitsubishi Chemical Corporation) (340 l) using 20-40% aqueous acetonitrile as an eluting solvent to be fractionated. The fractions that contained desired product were combined, adjusted to pH 6.8-7.0 with 1N—HCl and concentrated under reduced pressure at 30-45° C. until 1500 l. The condensed solution was adjusted to pH 5.5-5.7 with 1N—HCl and the resulting yellow suspension was stirred at 30-35° C. for 30 minutes and at 15-20° C. for 1 hour. Yellow precipitates were filtered, washed with water (85 l) and dried at 35-45° C. for 18 hours. Object Compound (69) was obtained as yellow powder (8.42 kg).

IR (KBr): 1645, 1635, 1533, 1516, 1446, 1269, 1200 cm⁻¹ NMR (DMSO-d₆, δ): 0.96 (6H, d, J=6.9 Hz), 1.11 (3H, d, J=5.5 Hz), 1.20-5.85 (52H, m), 6.41-9.15 (17H, m) ESI MASS (m/z)(Negative): 1330.4 (M⁺)

### EXAMPLE 70

To a solution of tetrahydrofuran (160 l), water (40 l), 25% aqueous NH₃ solution (26.4 l) and Starting Compound (70) (6.4 kg) was added a slurry of tetrahydrofuran (20 l), water (51) and Rh/Al₂O₃ (5% Rh, 6.4 kg). The resulting black slurry was treated with hydrogen (4.0 kg/cm²) at 30° C. for 35.5 hours. After completion of the reaction, the reaction mixture was filtered through a pad of KC-floc (powder of cellulose 2 kg), and washed with a mixture of tetrahydrofuran (51 l) and water (13 l). The filtrate was concentrated at 30-40° C. under reduced pressure to 80 l and yellow crystals precipitated in the residual solution. The precipitates were re-dissolved at pH 9.5-10.0 with 4N-NaOH (about 25 l) at 35-40° C. The pH of the solution was adjusted to 6.4-6.6 by the slow addition of 1N-HCl (2bout 24 l) at 35-40° C. to precipitate the product. It took more than 30 minutes to adjust the pH. After stirring for more than 30 minutes at 15-20° C., the precipitates were filtered with centrifuge, washed and dried at 35-45° C. under reduced pressure for 15 hours. Object Compound (70) was obtained as yellow powder (7.22 kg).

IR (KBr): 1645, 1635, 1630, 1533, 1516, 1446, 1269, 1240 cm⁻¹ NMR (DMSO-d₆, δ): 0.90 (3H, d, J=6.8 Hz), 0.98 (3H, d, J=6.8 Hz), 1.11 (3H, d, J=5.6 Hz), 1.43-5.22 (56H, m), 6.69-8.92 (17H, m) ESI MASS (m/z)(Negative): 1334.5 (M⁺)

### EXAMPLE 71

To a solution of dimethylformamide (47 l), dihydroxyacetone (1.06 kg) and Starting Compound (71) (5.2 kg) was added a slurry of Pt/C (5% Pt, 1.04 kg) in dimethylformamide (5 l). The resulting black slurry was treated with hydrogen (4.0 kg/cm²) at 30° C. for 34 hours. After completion of the reaction, methanol (52 l) was added under N₂ atmosphere, and the reaction mixture was filtered through a pad of KC-floc (powder of cellulose, 2 kg) and washed with dimethylformamide (10 l). The filtrate was added slowly to acetonitrile (745 l) in 1000-liter reactor. The mixture was stirred for 0.5 hour, and resulting precipitate was filtered and washed with acetonitrile (26 l). The precipitate was dried overnight in vacuo to give crude Object Compound (71) (5.07 kg). The crude-Object Compound (71) (4.90 kg) and water (260 l) were stirred for 0.5 hour, and the precipitate was filtered and washed with water (52 l). The precipitate was dissolved in a mixture of water (104 l) and 1N sodium hydroxide and the solution was subjected to column chromatography on adsorption resin (HP20SS (Trademark: prepared by Mitsubishi Chemical Co., Ltd.)) (260 l) eluting with 75% methanol in water. The fractions containing the object compound were collected and evaporated under reduced pressure to remove methanol. The suspension was stirred at 5° C. for 1 hour, and resulting precipitate was filtered and washed with water (140 l). The precipitate was dried overnight in vacuo to give pure-Object Compound (71) (1.36 kg).

IR (KBr): 1645, 1635, 1630, 1533, 1516, 1446, 1425, 1271, 1238 cm⁻¹ NMR (DMSO-d₆, δ): 0.90 (3H, d, J=6.8 Hz), 0.98 (3H, d, J=6.8 Hz), 1.11 (3H, d, J=5.6 Hz), 1.43-5.23 (62H, m), 6.69-8.88 (17H, m) ESI MASS (m/z) (Negative): 1408.5 (M⁺)

### EXAMPLE 72

To a suspension of Starting Compound (72) (100 mg) in N,N-dimethylformamide (1 ml) was added 4-[2-[4-[4-(6-methoxyhexyloxy)piperadin-1-yl]phenyl]imidazo[2,1-b][1,3,4]thiadiazol-6-yl]benzoyloxy-1H-1,2,3-benzotriazole (53.6 mg) and N,N-diisopropylethylamine (22 μl), and stirred for overnight at ambient temperature. To the reaction mixture was added piperidine (83.3 μl), and stirred for 3.5 hours at ambient temperature, then added ethyl acetate (100 ml). The resulting precipitate was collected by filtration, washed with diisopropylethylether (10 ml) to give a crude yellow powder (124.3 mg). The crude powder was purified by column chromatography on ODS (Daisogel SP-120 (40/60 μm)-ODS-B (Trademark: prepared by Daiso Co., Ltd.)) (40% acetonitrile aqueous solution). The fractions containing the object compound were combined, and evaporated under reduced pressure to remove acetonitrile. The residue was lypophilized to give Object Compound (72) (42.6 mg).

IR (KBr): 3353.6, 1631.1, 1606.4, 1517.7, 1463.7, 1436.7, 1268.9, 1228.4, 1195.6, 1087.7, 1045.2 cm⁻¹ NMR (DMSO-d₆+D₂O, δ): 0.98 (3H, d, J=6.8 Hz), 1.10 (3H, d, J=6.1 Hz), 1.2-1.45 (4H, m), 1.4-1.6 (6H, m), 1.6-4.9 (48H, m), 6.73 (1H, d, J=8.0 Hz), 6.75-6.85 (1H, m), 7.03 (1H, d, J=1.7 Hz), 7.09 (2H, d, J=9.1 Hz), 7.76 (2H, d, J=8.8 Hz), 7.94 (2H, d, J=8.8 Hz), 7.97 (2H, d, J=8.6 Hz), 8.76 (1H, s) MASS (m/z): 1479.4 (M⁻−H)

### EXAMPLE 73

A solution of Starting Compound (73) (500 mg) 1-[4-[5-[4-(7-methoxyheptyloxy)phenyl]-1,3,4-thiadiazol-2-yl]benzoyloxy]-1H-1,2,3-benzotriazole (240 mg) and N,N-diisopropylethylamine (0.11 ml) in DMF (5 ml) was stirred at room temperature for 5.5 hours. To the reaction mixture was added piperizine (0.42 ml) and stirred at room temperature for 1 hour. To the reaction mixture was added ethyl acetate (50 ml). The resulting precipitate was collected by filtration. The precipitate was dissolved in 20% acetonitrile in water (10 ml), and the solution was subjected to column chromatography on ODS (Daiso-gel, SP-120-40/60-ODS-B (Trademark: prepared by Daiso Co., Ltd.)) (25 ml) eluting with 35% acetonitrile in water. The fractions containing the object compound were collected and evaporated under reduced pressure to remove acetonitrile. The residue was lyophilized to give Object Compound (73) (500 mg).

IR (KBr): 3342.0, 1631.5, 1515.8, 1442.5, 1257.4 cm⁻¹ NMR (DMSO-d₆+D₂O, δ): 0.98 (3H, d, J=6.8 Hz), 1.10 (3H, d, J=5.9 Hz), 1.33-4.83 (51H, m), 6.71-8.14 (11H, m) ESI MASS (m/z)(Negative): 1372.4, 1371.4 (M⁻−H) Elemental Analysis Calcd. for C₆₁H₈₄N₁₀O₂₂S₂.5H₂O: C, 50.06; H, 6.47; N, 9.57 Found: C 49.83, H 6.71, N 9.49

### EXAMPLE 74

A solution of Starting Compound (74) (100 mg), 1-[4-[5-[4-[4-(2-ethoxyethoxy)phenyl]phenyl]-1,3,4-thiadiazol-2-yl]benzoyloxy]-1H-1,2,3-benzotriazole (49.8 mg) and N,N-diisopropylethylamine (16.3 mg) in DMF (1 ml) was stirred at room temperature overnight. To the reaction mixture was added piperizine (0.08 ml) and stirred at room temperature for 4 hours. To the reaction mixture was added ethyl acetate (10 ml). The resulting precipitate was collected by filtration. The precipitate was dissolved in 20% acetonitrile in water (10 ml), and the solution was subjected to column chromatography on ODS (Daiso-gel, SP-120-40/60-ODS-B (Trademark: prepared by Daiso Co., Ltd.)) (25 ml) eluting with 40% acetonitrile in water. The fractions containing the object compound were collected and evaporated under reduced pressure to remove acetonitrile. The residue was lyophilized to give Object Compound (74) (110 mg).

IR (KBr): 3371.0, 1633.4, 1535.1, 1442.5, 1249.6 cm⁻¹ NMR (DMSO-d₆+D₂O, δ): 0.99 (3H, d, J=6.7 Hz), 1.10 (3H, d, J=5.5 Hz), 1.15-4.83 (47H, m), 6.72-8.18 (11H, m) ESI MASS (m/z)(Negative): 1392.4, 1391.4 (M⁻−H) Elemental Analysis Calcd. for C₆₃H₈₀N₁₀O₂₂S₂.5.5H₂O: C, 50.70; H, 6.14; N, 9.38 Found: C, 50.71; H, 6.48; N, 9.35

### EXAMPLE 75

A solution of Starting Compound (75) (100 mg), 1-[4-[5-[4-[4-(2-methoxyethoxy)phenyl]phenyl]-1,3,4-thiadiazol-2-yl]benzoyloxy]-1H-1,2,3-benzotriazole (69.4 mg) and N,N-diisopropylethylamine (21.8 mg) in DMF (1 ml) was stirred at room temperature overnight. To the reaction mixture was added piperizine (0.08 ml) and stirred at room temperature for 4 hours. To the reaction mixture was added ethyl acetate (10 ml). The resulting precipitate was collected by filtration. The precipitate was dissolved in 20% acetonitrile in water (10 ml), and the solution was subjected to column chromatography on ODS (Daiso-gel, SP-120-40/60-ODS-B (Trademark: prepared by Daiso Co., Ltd.)) (25 ml) eluting with 35% acetonitrile in water. The fractions containing the object compound were collected and evaporated under reduced pressure to remove acetonitrile. The residue was lyophilized to give Object Compound (75) (100 mg).

IR (KBr): 3351.7, 1633.4, 1537.0, 1511.9, 1442.5, 1249.6 cm⁻¹ NMR (DMSO-d₆+D₂O, δ): 0.98 (3H, d, J=6.8 Hz), 1.11 (3H, d, J=5.9 Hz), 1.21-4.81 (45H, m), 6.70-8.18 (11H, m) ESI MASS (m/z)(Negative): 1378.5, 1377.4 (M⁻−H) Elemental Analysis Calcd. for C₆₂H₇₈N₁₀O₂₂S₂.6H₂O: C, 50.06; H, 6.10; N, 9.42 Found: C, 49.99; H, 6.29; N, 9.24

### EXAMPLE 76

To a solution of Starting Compound (76) (100 mg) and 4-[2-[4-(4-ethoxy-1-piperidyl)phenyl]imidazo[2,1-b][1,3,4]-thiadiazol-6-yl]benzoic acid (37.8 mg) and 1-hydroxybenzotriazole (17.1 mg) and 1-ethyl-3-(3′-dimethylaminopropyl)carbodiimide hydrochloride (32.3 mg) in N,N-dimethylformamide (1 ml) was added diisopropylethylamine (44 μl) at room temperature. The solution was stirred for 24 hours at the same temperature. Then to the reaction mixture was added piperidine and the mixture was stirred for 4 hours. Ethyl acetate was added to the reaction mixture. The resulting precipitates were collected by filtration and dried in vacuo. The precipitates were purified by column chromatography on ODS to give Object Compound (76) (51.1 mg).

NMR (DMSO-d₆+D₂O, δ): 0.98 (3H, d, J=6.8 Hz), 1.0-1.2 (6H, m), 1.35-4.55 (43H, m), 4.75-4.9 (2H, m), 6.7-6.85 (2H, m), 7.0-7.2 (3H, m), 7.77 (2H, d, J=8.9 Hz), 7.85-8.05 (4H, m), 8.76 (1H, s) MASS (m/z): 1393.4 (M⁻−H) Elemental Analysis Calcd. for C₆₂H₈₂N₁₂O₂₁S₂.8H₂O: C, 48.37; H, 6.42; N, 10.92 Found: C, 48.64; H, 6.39; N, 10.89

The following compounds [Example 77 and 78] were obtained according to a similar manner to that of Example 76.

### EXAMPLE 77

NMR (DMSO-d₆+D₂O, δ): 0.88 (3H, t, J=7.4 Hz), 0.98 (3H, d, J=6.7 Hz), 1.10 (3H, d, J=5.9 Hz), 1.4-4.55 (45H, m), 4.7-4.9 (2H, m), 6.65-6.85 (2H, m), 6.95-7.15 (3H, m), 7.76 (2H, d, J=8.8 Hz), 7.85-8.05 (4H, m), 8.77 (1H, s) MASS (m/z): 1407.4 (M⁻−H) Elemental Analysis Calcd. for C₆₃H₈₄N₁₂O₂₁S₂.7H₂O: C, 49.28; H, 6.43; N, 10.95 Found: C, 49.37; H, 6.54; N, 11.03

### EXAMPLE 78

NMR (DMSO-d₆+D₂O, δ): 0.89 (3H, t, J=7.2 Hz), 0.98 (3H, d, J=6.8 Hz), 1.10 (3H, d, J=5.8 Hz), 1.2-4.5 (47H, m), 4.75-4.9 (2H, m), 6.65-6.85 (2H, m), 7.0-7.2 (3H, m), 7.76 (2H, d, J=8.8 Hz), 7.85-8.05 (4H, m), 8.78 (1H, s) MASS (m/z): 1421.5 (M⁻−H) Elemental Analysis Calcd. for C₆₄H₈₆N₁₂O₂₁S₂.7H₂O: C, 49.60; H, 6.50; N, 10.85 Found: C, 49.52; H, 6.49; N, 10.75

### EXAMPLE 79

A solution of Starting Compound (79) (100 mg) 1-(5-methyl-2-oxo-1,3-dioxol-4-yl)methoxycarbonyloxy-2,5-pyrrolidinedione (28.6 mg) and N,N-diisopropylethylamine (13.6 mg) in DMF (1 ml) was stirred at room temperature overnight. To the reaction mixture was added ethyl acetate (10 ml). The resulting precipitate was collected by filtration. The precipitate was dissolved in phosphoric acid buffer solution (pH 6.86) (10 ml), and the solution was subjected to column chromatography on ODS (Daiso-gel, SP-120-40/60-ODS-B (Trademark: prepared by Daiso Co., Ltd.)) (25 ml) eluting with 30% acetonitrile in water. The fractions containing the object compound were collected and evaporated under reduced pressure to remove acetonitrile. To the residue was added diluted HCl, and lyophilized to give Object Compound (79) (40 mg).

IR (KBr): 3417.2, 1814.7, 1639.2, 1515.8, 1442.5, 1238.1 cm⁻¹ NMR (DMSO-d₆+D₂O, δ): 0.96 (3H, d, J=6.7 Hz), 1.08 (3H, d, J=5.8 Hz), 1.33-4.79 (54H, m), 6.64-8.14 (11H, m) ESI MASS (m/z)(Negative): 1578.6 (M⁻−H)

Elemental Analysis Calcd. for C₇₁H₉₃N₁₁O₂₆S₂.8H₂O: C, 49.44; H, 6.37; N, 8.93 Found: C, 49.29; H, 6.41; N, 8.89

1. A polypeptide compound of the following general formula (I): 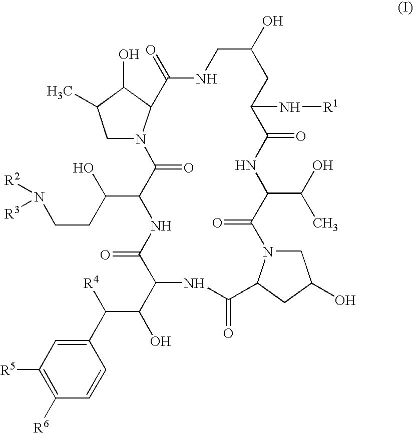
wherein
R¹ is acyl group,
R² is hydrogen or acyl group,
R³ is lower alkyl which has one or more hydroxy or protected hydroxy,
R⁴ is hydrogen or hydroxy,
R⁵ is hydrogen, hydroxy, lower alkoxy or hydroxy sulfonyloxy, and
R⁶ is hydroxy or acyloxy,
or a salt thereof:

2. A compound of claim 1, wherein
R¹ is phenyl(lower)alkenoyl substituted with one or more suitable substituent(s), benzoyl substituted with one or more suitable substituent(s) or naphthoyl substituted with one or more suitable substituent(s),
R² is hydrogen,
R³ is lower alkyl which has one or more hydroxy,
R⁴ is hydrogen or hydroxy,
R⁵ is hydroxy or hydroxysulfonyloxy and
R⁶ is hydroxy.

3. A compound of claim 2, wherein
R¹ is phenyl(lower)alkenoyl substituted with one or more suitable substituent(s), benzoyl substituted with one ore more suitable substituent(s) or naphthoyl substituted with one or more suitable substituent (s),
R² is hydrogen,
R³ is lower alkyl which has two hydroxy,
R⁴ is hydrogen or hydroxy;
R⁵ is hydroxy or hydroxysulfonyloxy; and
R⁶ is hydroxy.

4. A compound of claim 3, wherein
R¹ is naphthoyl substituted with higher alkoxy,
naphthoyl substituted with lower
alkoxy(higher)alkoxy,
naphthoyl substituted with higher alkyl,
phenyl(lower)alkenoyl substituted with lower alkoxy,
benzoyl substituted with a suitable substituent
selected from the group consisting of phenyl substituted with a suitable substituent selected from the group consisting of lower alkoxy, higher alkoxy and higher alkyl,
thiadiazolyl substituted with phenyl which has a suitable substituent selected from the group consisting of piperazinyl substituted with cyclo(lower)alkyl which may have lower alkoxy(lower)alkoxy, piperazinyl substituted with lower alkoxy(higher)alkyl, piperazinyl substituted with tetrahydropyran, piperazinyl substituted with dioxaspiro(higher)alkyl which may have lower alkyl, piperazinyl substituted with lower alkyl having pyridyl, piperidyl substituted with lower alkoxy and chlorophenyl, piperidyl substituted with lower alkoxy, piperidyl substituted with lower alkoxy having cyclo(lower)alkyl, piperidyl substituted with lower alkoxy(higher)alkoxy, dioxaazaspiro(higher)alkyl, tetrahydropyrazolopyridyl substituted with phenyl, cyclo(lower)alkyloxy, piperidyloxy substituted with cyclo(lower)alkyl which may have lower alkoxy(lower)alkoxy, piperidyloxy substituted with lower alkoxy(higher)alkyl, piperidyloxy substituted with phenyl which may have lower alkoxy, piperidyl substituted with lower alkoxy higher alkyl, and piperidyl substituted with lower alkoxy(lower)alkoxy,
thiadiazolyl substituted with pyridyl having piperidyl substituted with phenyl,
imidazothiadiazolyl substituted with phenyl having lower alkoxy(lower)alkoxy(lower)alkyl,
imidazothiadiazolyl substituted with phenyl having lower alkoxy and cyclo(lower)alkyl,
imidazothiadiazolyl substituted with phenyl having piperidyloxy substituted with phenyl which may have lower alkoxy,
imidazothiadiazolyl substituted with phenyl having piperidyloxy substituted with cyclo(lower)alkyl which may have lower alkoxy(lower)alkoxy,
imidazothiadiazolyl substituted with phenyl having tetrahydropyridyl substituted with cyclo(lower)alkyl,
imidazothiadiazolyl substituted with phenyl having piperidyl substituted with lower alkoxy(lower)alkyl,
imidazothiadiazolyl substituted with phenyl having piperazinyl substituted with lower alkoxy(lower)alkyl,
imidazothiadiazolyl substituted with phenyl having lower alkoxy(higher)alkyl,
imidazothiazolyl substituted with phenyl having lower alkoxy(lower)alkoxy,
phenyl substituted with piperazinyl having phenyl substituted with lower alkoxy,
phenyl substituted with piperazinyl having phenyl substituted with piperidyloxy having lower alkoxy(lower)alkyl,
phenyl substituted with diazabicyclo(higher)alkyl having cyclo(lower)alkyl,
phenyl substituted with hexahydrodiazepinyl having cyclo(lower)alkyl,
phenyl substituted with piperidyl having phenyl,
phenyl substituted with piperazinyl having phenyl substituted with piperazinyl having lower alkoxy(lower)alkyl,
piperazinyl substituted with thiadiazolyl having phenyl substituted with lower alkoxy(higher)alkoxy, thiazolyl substituted with phenyl having lower alkoxy,
oxadiazolyl substituted with phenyl having higher alkoxy,
oxadiazolyl substituted with phenyl having phenyl substituted with lower alkoxy,
oxadiazolyl substituted with phenyl having piperazinyl substituted with cyclo(lower)alkyl having lower alkyl,
pyrazolyl substituted with phenyl having phenyl, and
pyrazolyl substituted with phenyl having lower alkoxy,
R² is hydrogen,
R³ is lower alkyl which has two hydroxy,
R⁴ is hydrogen or hydroxy;
R⁵ is hydroxy or hydroxysulfonyloxy; and
R⁶ is hydroxy.

5. A process for preparing a polypeptide compound (I) of claim 1, or a salt thereof,
which comprises,
1) reacting a compound (II) of the formula: 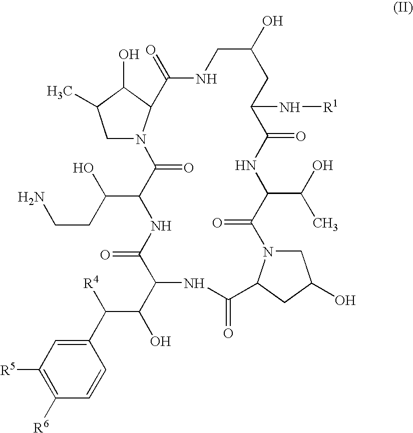
wherein R¹, R⁴, R⁵ and R⁶ are defined in claim 1,
or its reactive derivative at the amino group or a salt thereof, with a compound (III) of the formula:
R³═O (III)
wherein R³ is defined in claim 1,
or its reactive derivative or a salt thereof, to give a compound (Ia) of the formula: 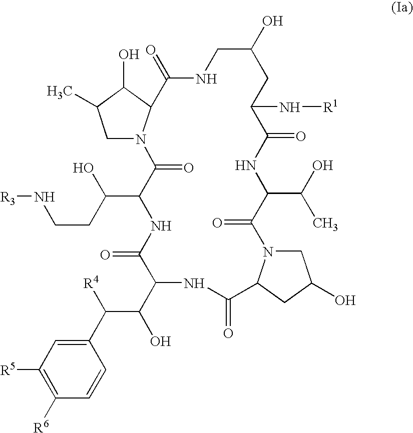
wherein R¹, R³, R⁴, R⁵ and R⁶ are defined above,
or a salt thereof, or
ii) reacting a compound (Ia) of the formula: 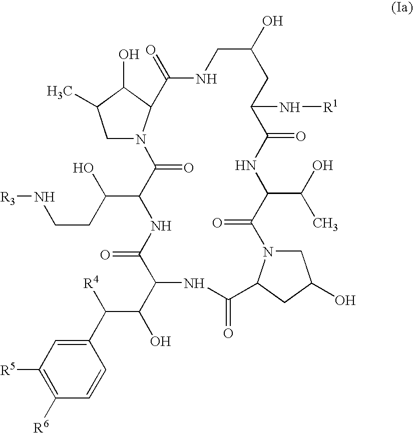
wherein R¹, R³, R⁴, R⁵ and R⁶ are defined in claim 1,
or its reactive derivative at the amino group or a salt thereof, with a compound (IV) of the formula:
Rₐ²—OH (IV)
wherein R¹, Rₐ² is acyl group,
or its reactive derivative at the carboxy group or a salt thereof, to give a compound (Ib) of the formula: 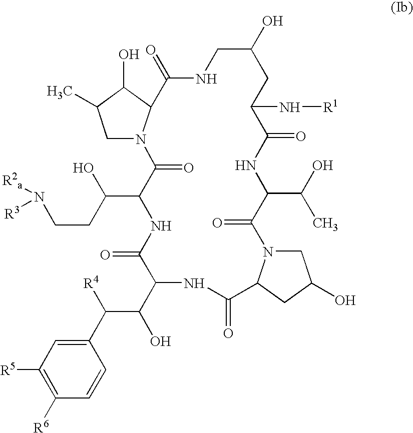
wherein R¹, Rₐ², R³, R⁴, R⁵ and R⁶ are defined above,
or a salt thereof, or
iii) subjecting a compound (Ib) of the formula: 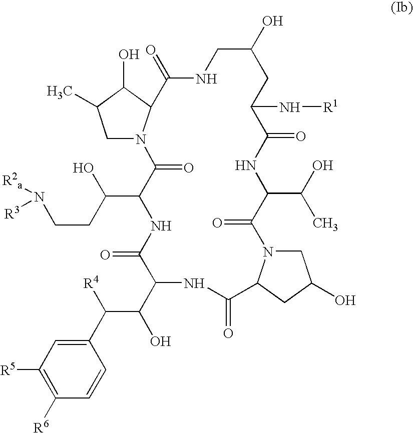
wherein R¹, R³, R⁴, R⁵ and R⁶ are defined in claim 1,
Rₐ² is acyl group,
or a salt thereof, to elimination reaction of the acyl group, to give a compound (Ia) of the formula: 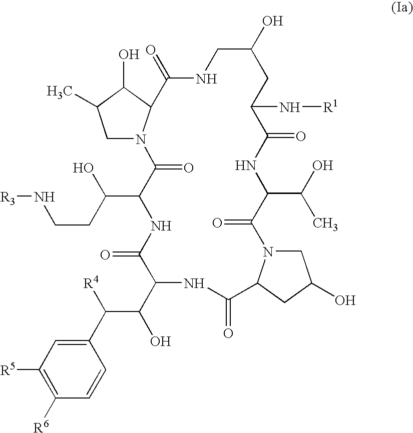
wherein R¹, R³, R⁴, R⁵ and R⁶ are defined above,
or a salt thereof, or
iv) reacting a compound (Ic) of the formula: 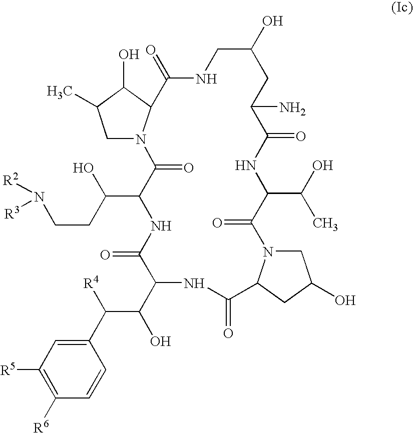
wherein R², R³, R⁴, R⁵ and R⁶ are defined in claim 1,
or its reactive derivative at the amino group or a salt thereof, with a compound (V) of the formula:
Rₐ¹—OH (V)
wherein Rₐ¹ is acyl group,
or its reactive derivative at the carboxy group or a salt thereof, to give a compound (Id) of the formula: 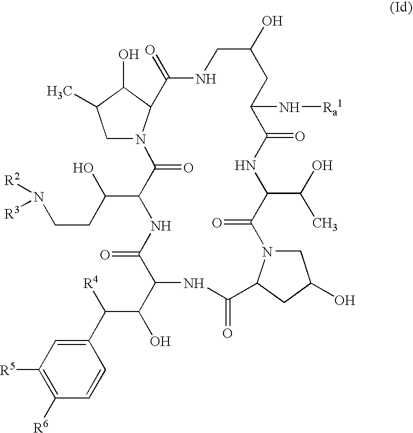
wherein R², R³, R⁴, R⁵ and R⁶ are defined in claim 1,
Rₐ¹ is defined above, or a salt thereof.

6. A pharmaceutical composition which comprises, as an active ingredient, a compound of claim 1 or a pharmaceutically acceptable salt thereof in admixture with pharmaceutically acceptable carriers or excipients.

7. Use of a compound of claim 1 or a pharmaceutically acceptable salt thereof for the manufacture of a medicament.

8. A compound of claim 1 or a pharmaceutically acceptable salt thereof for use as a medicament.

9. A method for the prophylactic and/or therapeutic treatment of infectious diseases caused by pathogenic microorganisms, which comprises administering a compound of claim 1 or a pharmaceutically acceptable salt thereof to a human being or an animal.

10. A commercial package comprising the pharmaceutical composition of claim 7 and a written matter associated therewith, wherein the written matter states that the pharmaceutical composition can or should be used for preventing or treating infections disease.

11. An article of manufacture, comprising packaging material and the compound (I) identified in claim 1 contained within said packaging material, wherein said the compound (I) is therapeutically effective for preventing or treating infectious diseases, and wherein said packaging material comprises a label or a written material which indicates that said compound (I) can or should be used for preventing or treating infectious diseases.
